(19) United States
(12) Patent Application Publication
(10) Pub. No.: US 2022/0119864 A1
(43) Pub. Date: Apr. 21, 2022

(54) CANID MICROBIOME MONITORING TOOLS AND DIAGNOSTIC METHODS

(71) Applicant: MARS, INCORPORATED, McLean, VA (US)

(72) Inventors: Zoe V. MARSHALL-JONES, Melton Mowbray, Leicestershire (GB); David J. WRIGGLESWORTH, Melton Mowbray, Leicestershire (GB); Ruth STAUNTON, Melton Mowbray, Leicestershire (GB); Zoe LONSDALE, Melton Mowbray, Leicestershire (GB); Phillip WATSON, Melton Mowbray, Leicestershire (GB); Krusha PATEL, Melton Mowbray, Leicestershire (GB)

(73) Assignee: MARS, INCORPORATED, McLean, VA (US)

(21) Appl. No.: 17/423,751

(22) PCT Filed: Jan. 20, 2020
(86) PCT No.: PCT/US2020/014292

§ 371 Date: Jul. 16, 2021

(30) Foreign Application Priority Data
Jan. 18, 2019 (GB) 1900744.2

(51) Int. Cl. C12Q 1/689

(52) U.S. Cl. CPC C12Q 1/689; C12Q 2600/124

(57) ABSTRACT

Methods for assessing a canid's microbiome health are provided. The methods include, inter alia, detecting at least four bacterial taxa in a sample obtained from the canid.

### CROSS-REFERENCE TO RELATED APPLICATION

This application claims priority to UK Patent Application No. 1900744.2, filed on Jan. 18, 2019, the contents of which are incorporated herein by reference in its entirety.

### TECHNICAL FIELD

This present disclosure is in the field of monitoring tools and diagnostic methods for determining the health of a canid's microbiome.

### BACKGROUND TO THE INVENTION

The understanding of the microbiome and its impact on health has increased significantly in recent years. Changes in the microbiome, and its interaction with the immune, endocrine and nervous systems are correlated with a wide array of illnesses, ranging from inflammatory bowel disease [1-3] to cancer [4] and to behavioral aspects of host health [5;6].

The establishment of the microbiome occurs at the same time as immune system maturation and plays a role in intestinal physiology and regulation. The initial establishment of the gut microbiota is an essential step in neonatal development, influencing immunological development in infancy and health throughout life. As such in humans and many mammals a rapid increase in diversity occurs in the early establishment phase of gut microbiome development [7].

The adult gut microbiome can be resilient to large shifts in community structure. In humans and other mammals, it is considered to be relatively stable throughout adult life. This “adult microbiome” is considered to represent a healthy gut microbiome for dogs with enhanced resilience compared to other lifestages. In early lifestages, puppies have an undeveloped gut barrier, which includes the gastrointestinal microbiome as well as histological and gut associated immune functions. Puppies and young dogs are therefore are more prone to gastrointestinal illnesses such as diarrhoea and sickness, etc. Senior and geriatric dogs are also more prone to diarrhoea and gastrointestinal complications, which can occur in part as a result of a deterioration in the gut microbiome.

Given the importance of the microbiome to health and wellbeing, it is important to find ways to determine the health of the microbiome of an animal.

### SUMMARY OF THE INVENTION

The presently disclosed subject matter provides novel developed methods which allow the determination of the health of a canid's microbiome. The methods of the present disclosure can achieve this with high accuracy, as shown in the examples.

In one aspect, the present disclosure provides a method of determining the health of a canid's microbiome, comprising quantitating four or more bacterial species to determine their abundance; and comparing the abundance to the abundance of the same species in a control data set; wherein an increase or decrease in the abundance of the four or more bacterial species relative to the control data set is indicative of an unhealthy microbiome. As discussed above, an unhealthy microbiome is associated with a number of health conditions and it is therefore desirable to monitor the health of the gut microbiome or to diagnose an unhealthy microbiome.

In another aspect, the present disclosure features a method of determining the health of a canid's microbiome, comprising detecting at least four bacterial taxa in a sample obtained from the canid; wherein the presence of at least four bacterial taxa is indicative of a healthy microbiome.

In another aspect, the present disclosure features a method of determining the health of a canid's microbiome by a method comprising the steps of calculating the diversity index for the species within the canid's microbiome and comparing the diversity index to the diversity index of a control data set.

In another aspect, the present disclosure provides a method of monitoring a canid, comprising a step of determining the health of the canid's microbiome by a method of the present disclosure on at least two time points. This is particularly useful where a canid is receiving treatment to shift the microbiome as it can monitor the progress of the therapy. It is also useful for monitoring the health of the canid.

In some embodiments, the methods of the present disclosure comprise a further step of changing the composition of the microbiome. This can be achieved through a dietary change or a functional food or supplement and/or through administration of a nutraceutical or pharmaceutical composition comprising bacteria. This will usually be done where the microbiome is deemed to require or benefit from enhancement or where it is unhealthy, but can also be undertaken preemptively.

In another aspect, also provided is a method of monitoring the health of the microbiome in a canid who has undergone a dietary change or who has received a functional food, supplement, nutraceutical or pharmaceutical composition which is able to change the microbiome composition, comprising determining the health of the microbiome by a method according to the present disclosure. Such methods allow a skilled person to determine the success of the treatment. Preferably these methods comprise determining the health of the microbiome before and after treatment as this helps to evaluate the success of the treatment.

In a particular embodiment, the presently disclosed subject matter provides a method of determining the health of a canid's microbiome, comprising detecting at least four bacterial taxa in a sample obtained from the canid; wherein the presence of the at least four bacterial taxa is indicative of a healthy microbiome. In certain embodiments of the method, the bacterial taxa are bacterial species from genera selected from the group consisting of Blautia, Lactobacillus, Faecalibacterium, Terrisporobacter, Lachnospiraceae novel sp., Butyricicoccus, Lachnoclostridium, Clostridium, Holdemanella, Cellulosilyticum, Romboutsia, Lachnospiraceae_NK4A136 group, Peptostreptococcus, Sellimonas, Ruminococcaceae_UCG-014, Finegoldia, and Candidatus Dorea. In another embodiment, the bacterial taxa are species selected from the group consisting of Blautia [Ruminococcus] gnavus, Blautia [Ruminococcus] torques, Blautia [Ruminococcus] torques group sp., Blautia producta, Blautia sp., Butyricicoccus pullicaecorum, Cellulosilyticum sp., Clostridium hiranonis, Dorea massiliensis, Faecalibacterium prausnitzii, Finegoldia sp., Finegoldia magna, Fusobacterium mortiferum, gauvreauii group Clostridium sp., Holdemanella [Eubacterium] biforme, Lachnoclostridium sp., Lachnospiraceae novel sp., Lachnospiraceae_NK4A136 group sp., Lactobacillus ruminis, Lactobacillus sp., Romboutsia sp., Roseburia faecis, Ruminococcaceae_UCG-015 sp., Sellimonas sp., Clostridium sp., Lactobacillus saerimneri, Terrisporobacter sp. SN1, Terrisporobacter sp. SN9, Terrisporobacter glycolicus, Terrisporobacter mayombei, Terrisporobacter petrolearius, Terrisporobacter sp., and Terrisporobacter sporobacter. In a particular embodiment, the bacterial taxa have a 16 S rDNA with at least about 95%, at least about 96%, at least about 97%, at least about 98%, at least about 99% or 100% identity to the sequence of any one of SEQ ID NOs 6, 7, 11, 12, 14, 16, 21, 23, 24, 28, 29, 30, 32, 37, 39, 41-43, 46-49, 52, 55-57, 61, 67, 71, 75, 77, 78 and 80.

The presently disclosed subject matter also provides a method of determining the health of a canid's microbiome, comprising quantitating four or more bacterial species in a sample obtained from the canid to determine their abundance; and comparing the abundance to the abundance of the same species in a control data set; wherein an increase or decrease in the abundance of the four or more bacterial species relative to the control data set is indicative of an unhealthy microbiome. In one embodiment of the claimed method, the bacterial species are from genera selected from the group consisting of Absiella [Eubacterium], Anaerostipes, Anaerotruncus, Bifidobacterium, Blautia, Blautia [Ruminococcus] torques group, Butyricicoccus, Candidatus, Dorea, Cellulosilyticum, Clostridium, Clostridium sensu_stricto 1, Collinsella, Enterococcus, Erysipelatoclostridium, Faecalibacterium, Finegoldia, Flavonifractor, Fusobacterium, Holdemanella [Eubacterium], Lachnoclostridium, Lachnospiraceae novel sp., Lachnospiraceae_NK4A136 group, Lactobacillus, Megamonas, Peptostreptococcus, Romboutsia, Roseburia, Ruminococcaceae, Ruminococcaceae_UCG-O14, Ruminococcus, Sellimonas, Terrisporobacter, Turicibacter, and Lachnospiraceae. In another embodiment, the bacterial species are selected from the group consisting of Absiella [Eubacterium] dolichum, Anaerostipes caccae, Anaerostipes indolis, Anaerostipes rhamnosivorans, Anaerotruncus sp., Bifidobacterium sp., Blautia [Ruminococcus] gnavus, Blautia [Ruminococcus] torques, Blautia [Ruminococcus] torques group sp., Blautia producta, Blautia sp., Butyricicoccus pullicaecorum, Butyricicoccus sp., Cellulosilyticum sp., Clostridium hiranonis, Clostridium sp., Clostridium sp., Collinsella sp., Dorea massiliensis, Enterococcus sp., Erysipelatoclostridium sp., Faecalibacterium prausnitzii, Finegoldia magna, Finegoldia sp., Fusobacterium sp., Holdemanella [Eubacterium] biforme, Lachnoclostridium sp., Lachnoclostridium [Clostridium] sp., Lachnoclostridium hylemonae, Lachnoclostridium leptum, Lachnoclostridium sp., Lachnospiraceae novel sp., Lachnospiraceae sp., Lachnospiraceae_NK4A136 group sp., Lactobacillus animalis, Lactobacillus apodemi, Lactobacillus faecis, Lactobacillus murinus, Lactobacillus plantarum, Lactobacillus reuteri, Lactobacillus ruminis, Lactobacillus saerimneri, Lactobacillus sp., Megamonas funiformis, Megamonas sp., Megamonas rupellensis, Pseudoflavonifractor capillosus; Pseudoflavonfractor sp., Romboutsia sp., Roseburia faecis, Roseburia sp., Ruminococcaceae novel sp., Ruminococcaceae_UCG-015 sp., Sellimonas sp., Terrisporobacter glycolicus, Terrisporobacter mayombei, Terrisporobacter petrolearius, Terrisporobacter sp., Terrisporobacter sp. SN1, Terrisporobacter sp. SN9, Terrisporobacter sporobacter, Turicibacter sanguinis, and Turicibacter sp.

In certain embodiments of the claimed methods, a decrease in abundance relative to the control data set is indicative of an unhealthy microbiome. In a specific embodiment of the claimed methods, the bacterial species is Fusobacterium mortiferum. In certain embodiments of the claimed methods, an increase in abundance relative to the control data set is indicative of an unhealthy microbiome.

In a particular embodiment of the claimed methods, the bacterial taxa have a 16 S rDNA sequence selected from the group consisting of SEQ ID Nos: 3-85.

In certain embodiments of the claimed methods, the control data set comprises microbiome data of a canid at the same life stage.

In a particular embodiment of the claimed methods, the canid is a puppy.

In yet another embodiment of the claimed methods, the bacterial taxa are species from the genera selected from the group consisting of Ruminococcus, Clostridiales sp., Paraprevotella, Adlercreutzia, Allobaculum, Allobaculum/Dubosiella, Bacteroides, Bifidobacterium, Blautia, Clostridales, Clostridium, Collinsella, Dorea, Enterococcus, Erysipelotrichaceae, Faecalibacterium, Fusobacterium, Holdemanella [Eubacterium], Lachnoclostridium, Lactobacillus, Megamonas, Megasphaera, Peptostreptococcus, Phascolarctobacterium, Prevotella, Sarcina, Terrisporobacter, and Turicibacter.

In a specific embodiment, the bacterial taxa have a 16 s rDNA with at least about 95%, at least about 96%, at least about 97%, at least about 98%, at least about 99% or 100% identity to the sequence of any one of SEQ ID NOs: 86-166.

In particular embodiments of the claimed methods, the canid is an adult, senior or geriatric canid.

In certain embodiments of the claimed methods, the methods further comprise a step of changing the microbiome composition of the canid. In other embodiments of the claimed methods, the method further comprises a step of changing the diet of the canid and/or administering a pharmaceutical composition or a nutraceutical composition to the canid.

In yet another embodiment, the disclosed subject matter provides a method of determining the health of a canid's microbiome, comprising calculating the diversity index for the species within the canid's microbiome and comparing the diversity index to the diversity index of a control data set.

In particular embodiments of the claimed methods, the canid is a pre-weaned puppy and the microbiome is considered healthy if the diversity index falls in the range of about 0.123 to about 1.744. In particular embodiments of the claimed methods, the canid is a post-weaned puppy and the microbiome is considered healthy if the diversity index falls in the range of about 1.294 to about 2.377. In particular embodiments of the claimed methods, the canid is an adult and the microbiome is considered healthy if the diversity index falls in the range of about 1.83 to about 3.72. In particular embodiments of the claimed methods, the canid is a senior and the microbiome is considered healthy if the diversity index falls in the range of about 1.24 to about 3.55. In particular embodiments of the claimed methods, the canid is geriatric and the microbiome is considered healthy if the diversity index falls in the range of about 2.16 to about 3.47.

In another embodiment, the disclosed subject matter provides a method of monitoring a canid, comprising a step of determining the health of the canid's microbiome by the method of any preceding claim on at least two time points. In certain embodiments, the two time points are at least 6 months apart.

In certain embodiments of the claimed methods, the sample is from the gastrointestinal tract. In certain embodiments, the sample is a faecal sample, an ileal sample, a jejunal sample, a duodenal sample or a colonic sample.

In certain embodiments of the claimed methods, the methods further comprise a step of changing the composition of the microbiome. In particular embodiments, the step of changing the microbiome composition comprises the administration of a pharmaceutical composition, a nutraceutical composition, a functional food, a supplement or a step of changing the canid's diet.

In another embodiment, the disclosed subject matter provides a method of monitoring the microbiome health in a canid who has received a pharmaceutical composition, a nutraceutical composition, a functional food, a supplement which is able to change the microbiome composition or who has undergone a step of changing the canid's diet that can change the microbiome composition, comprising determining the health of the microbiome by the method of any preceding claim. In particular embodiments, the health of the microbiome is determined before and after administration of the pharmaceutical composition. In certain embodiments, the pharmaceutical composition comprises bacteria.

In another embodiment of the claimed methods, the bacterial species is detected by means of DNA sequencing, RNA sequencing, protein sequence homology or another biological marker indicative of the bacterial species.

In the embodiments of the claimed methods, the canid is a dog.

### BRIEF DESCRIPTION OF DRAWINGS

FIGS. 1A and 1B: Each of FIGS. 1A and 1B each depict multigroup principal components (PCA) and t-distributed stochastic neighbour embedding (t-SNE) data visualisation of the bacterial community composition characteristics in faeces of puppies with advancing age.

FIGS. 2A and 2B: FIG. 2A provides a summary phylum level taxon represented in faeces from puppies (mean proportion of the total OTUs for the cohort, with age in days post-partum). FIG. 2B provides the Shannon diversity (mean and 95% CI) of the microbial content in faeces puppies of puppies with age (in days) after birth.

FIG. 3: FIG. 3 provides the Shannon diversity (mean and 95% CI) of the microbial content in faeces puppies of puppies with age (in days) after birth.

FIG. 4: FIG. 4 provides the Shannon diversity of the faecal microflora in adult Beagle dogs by life stage group.

FIGS. 5A and 5B: FIGS. 5A and 5B provide Phylum level summary data, showing changes in phylum level microbial proportions across time from birth for two independent studies of the puppy faecal microbiota.

FIGS. 6A-6H: FIGS. 6A through 6H provide stacked bar plots detailing the genus level faecal microbial composition of adult dogs prior to, during and following antibiotic treatment with metronidazole. Data from from eight representative dogs within the cohort of 22 dogs are shown demonstrating the distribution in the abundant taxonomic groups (genera) at each sampling point. Each of FIGS. 6A-6H represent a different set of data for an individual dog.

FIG. 7: FIG. 7 is a partial least Square discriminate analysis (PLS-DA) correlation plot based on likeness in bacterial abundance data for the 25 OTUs displaying the greatest influence on clustering of the samples (variable importance in projection scores>1).

FIG. 8 corresponds to Table 1.1, which provides the bacterial taxa that are detected in faeces from puppies.

FIG. 9 corresponds to Table 1.3, which provides the bacterial taxa that are indicative of a healthy microbiome in puppies and their abundance in the microbiome.

FIG. 10 corresponds to Table 2.1, which provides the bacterial taxa that are detected in faeces from adult, senior, and geriatric dogs.

FIG. 11 corresponds to Table 2.3, which provides the bacterial taxa that are indicative of a healthy microbiome in mature canids and their abundance in the microbiome.

FIG. 12 corresponds to Table 3.1, which provides the Shannon diversity of the microbiota in faeces from puppies prior to and throughout the weaning period.

FIG. 13 corresponds to Table 4, which provides the bacterial taxa that are detected in the gut following treatment with antibiotics.

### DETAILED DESCRIPTION

### The Health of the Microbiome

The methods of the present disclosure can be used to determine the health of a canid's microbiome. This can be achieved by quantitating four or more bacterial species in a sample obtained from the canid to determine their abundance; and comparing the abundance to the abundance of the same species in a control data set. Differences in the abundance of at least four bacterial species, compared to a control data set, suggest that the microbiome is unhealthy or can be becoming unhealthy, and that the canid will benefit from an intervention (e.g., a treatment) to bring the microbiome back to its healthy state or alternatively that health can be better than the control data set.

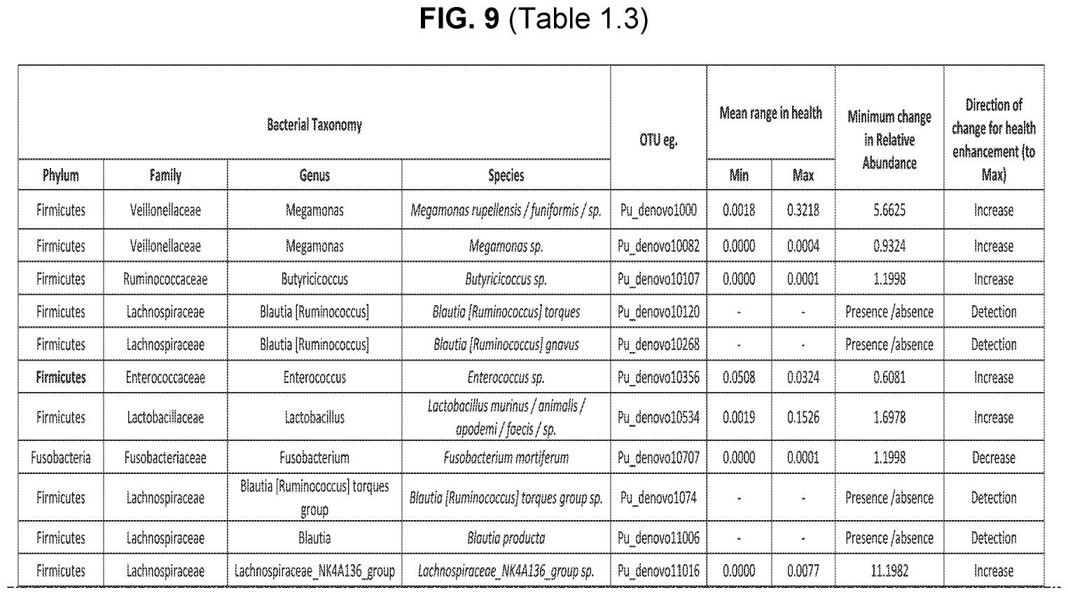

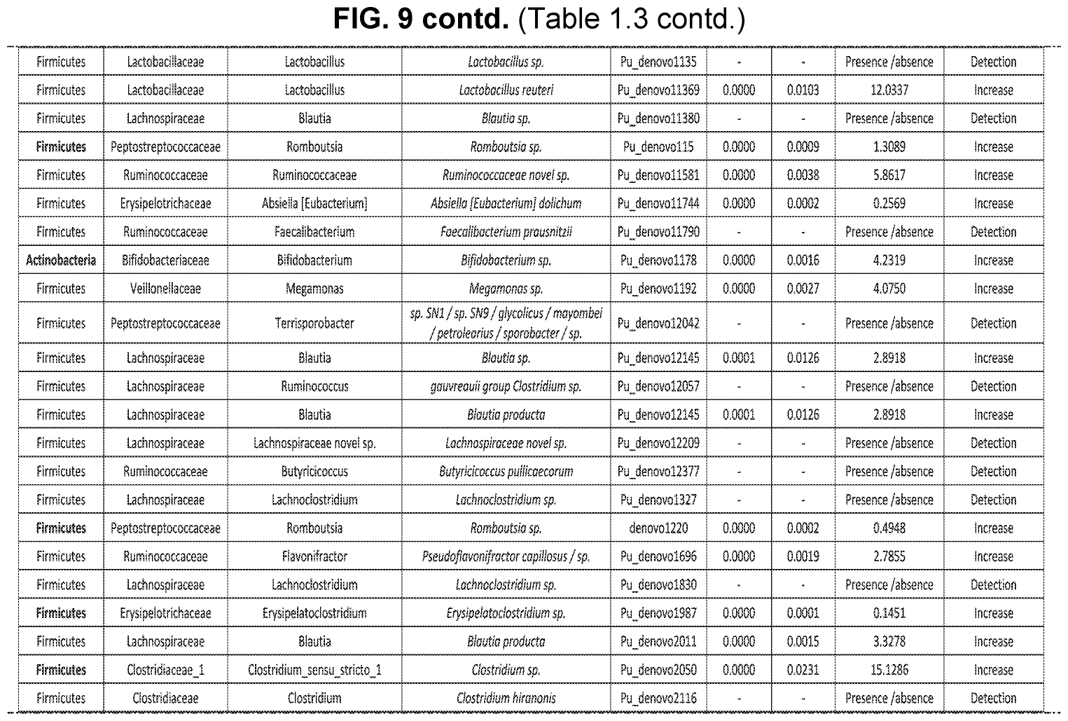

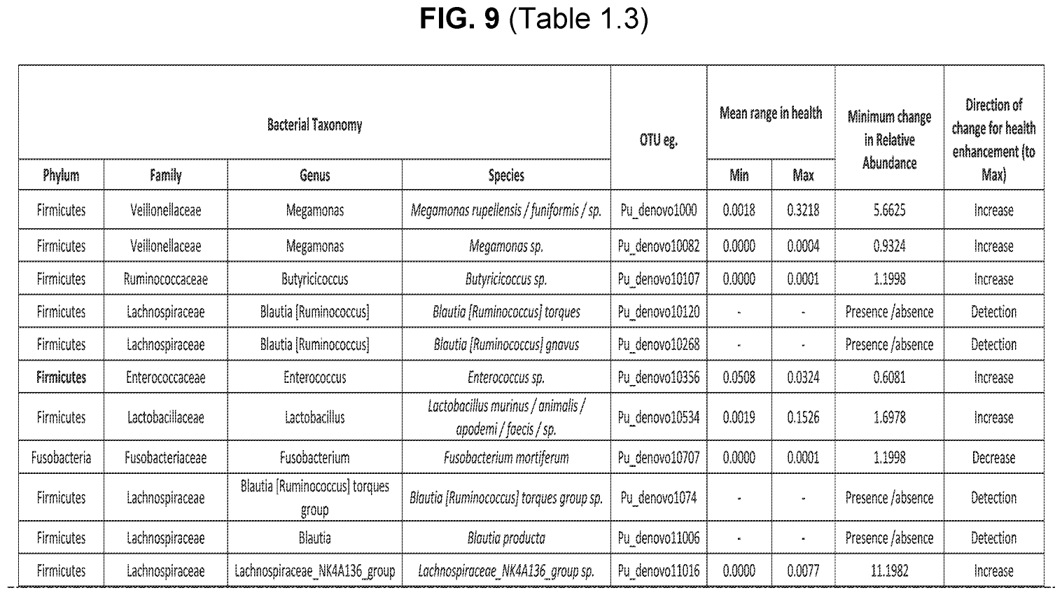

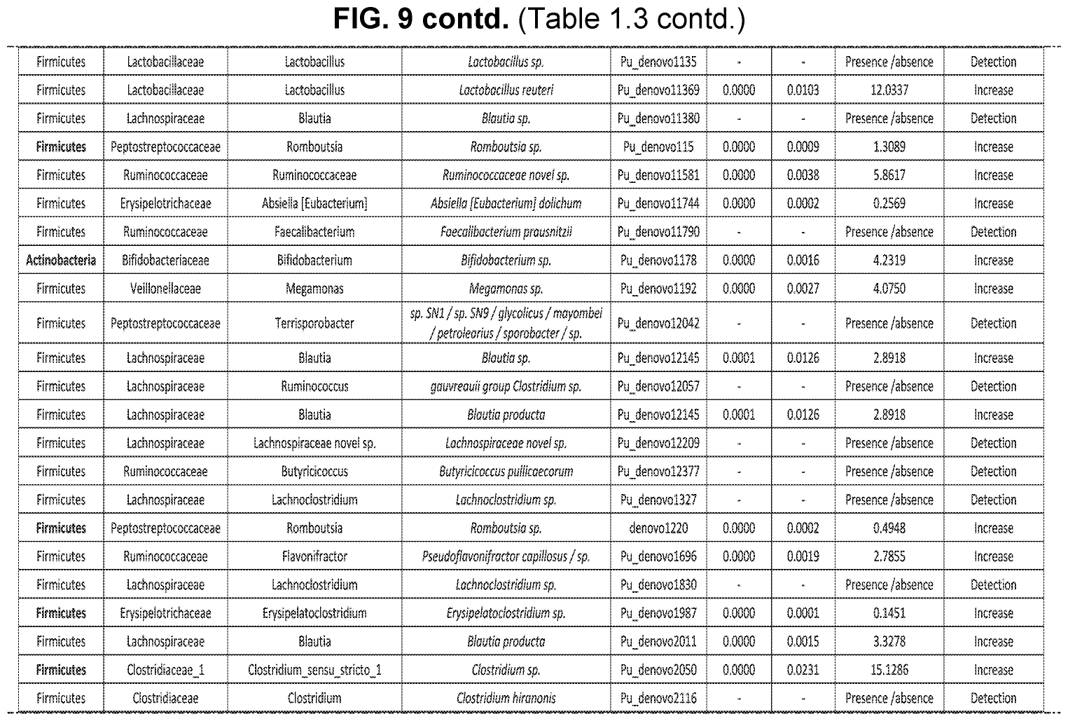

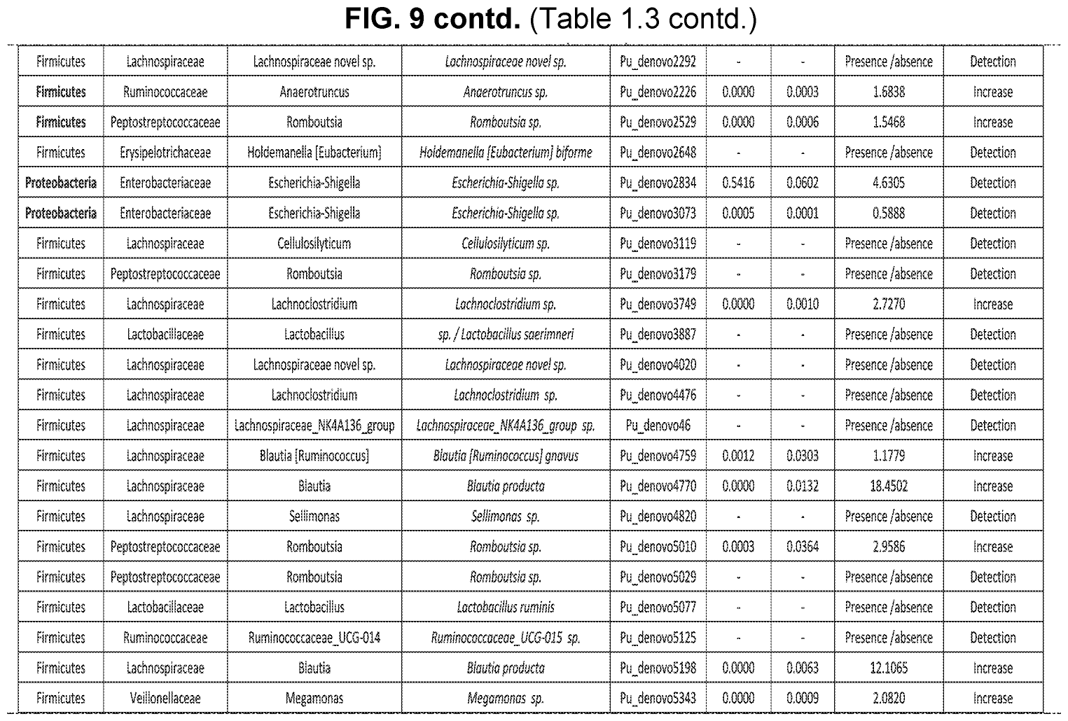

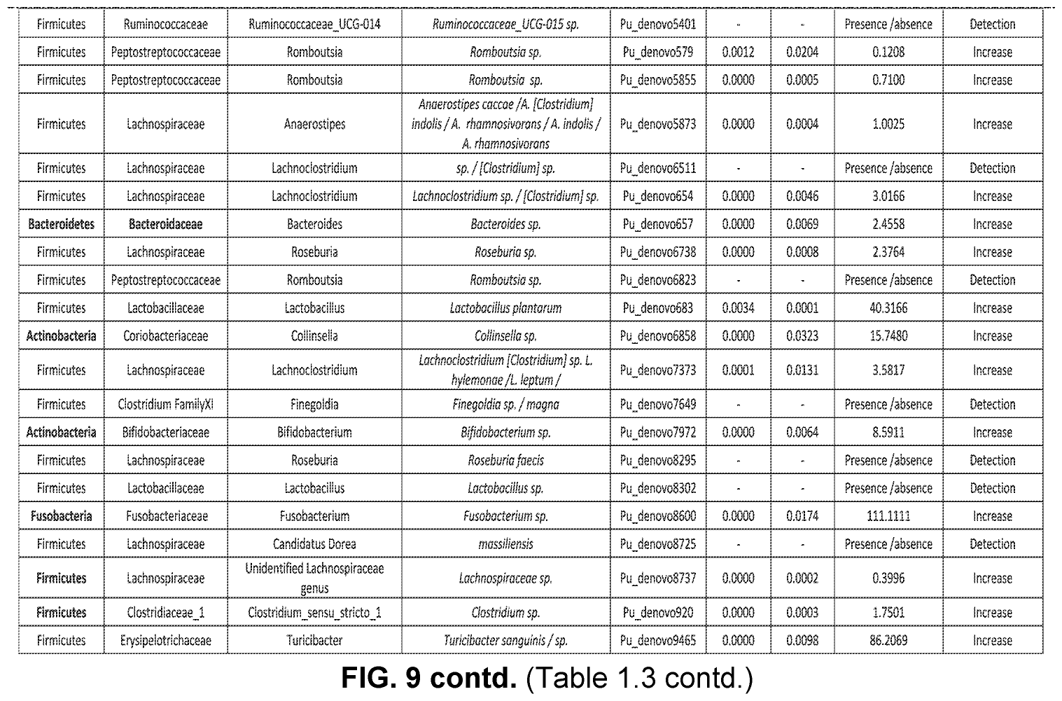

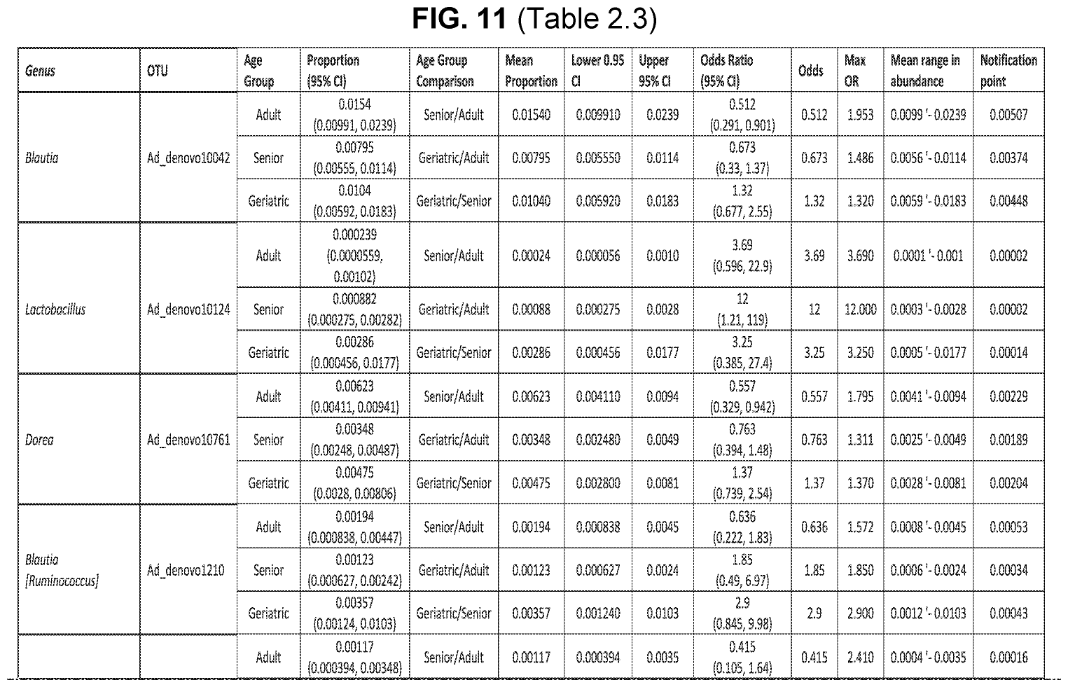

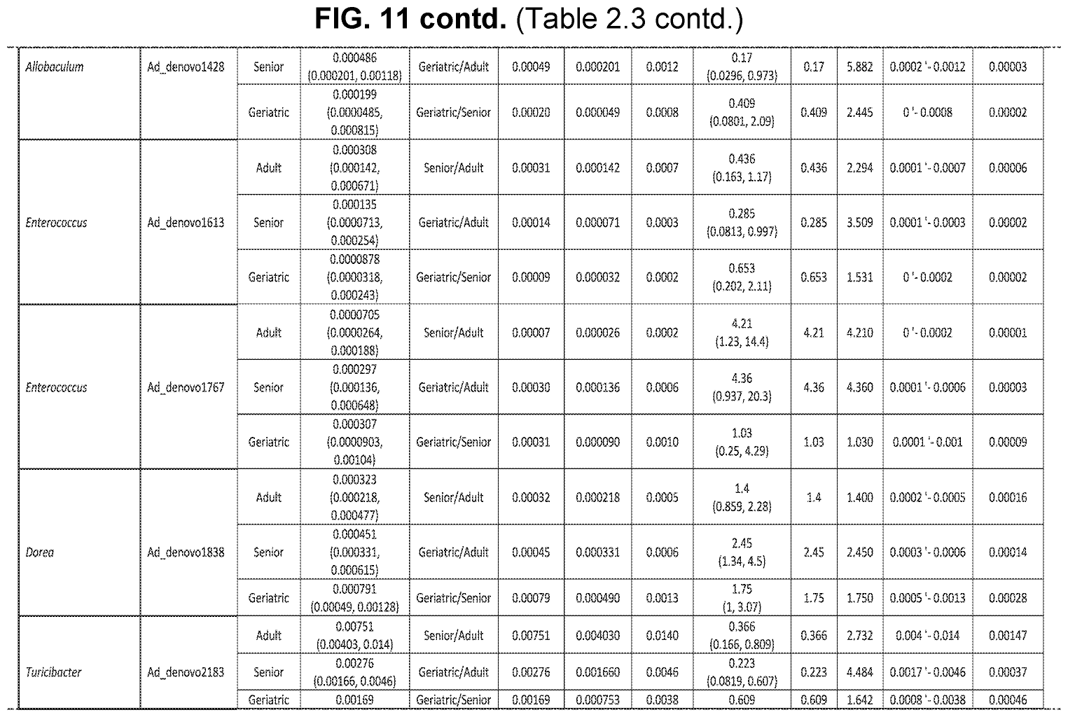

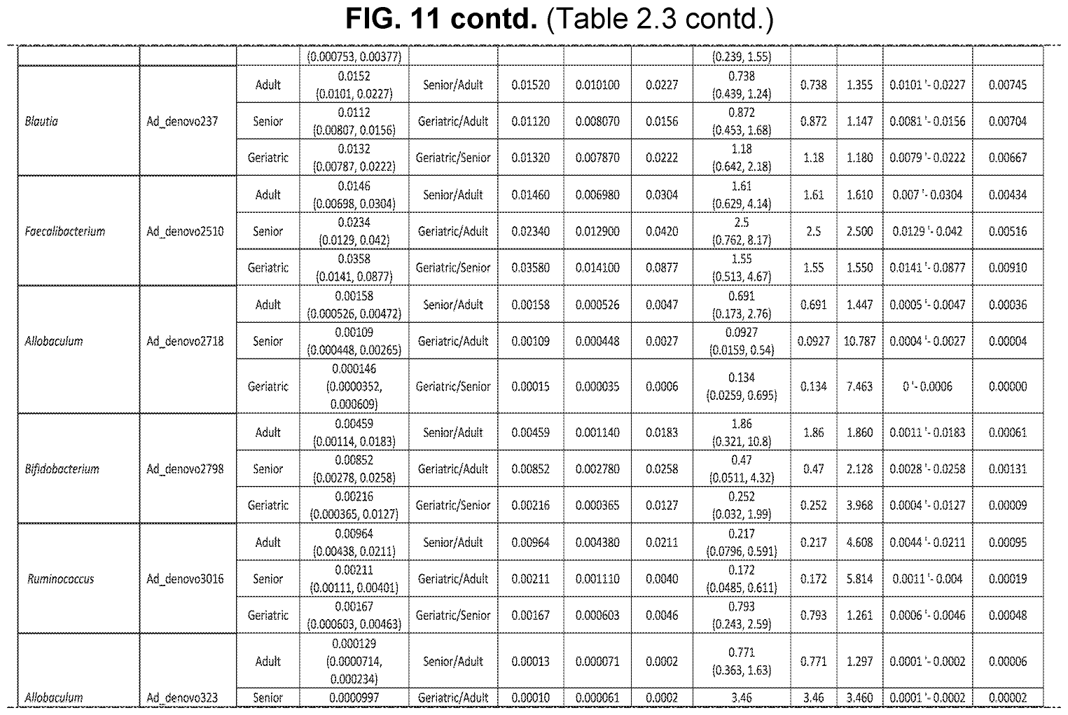

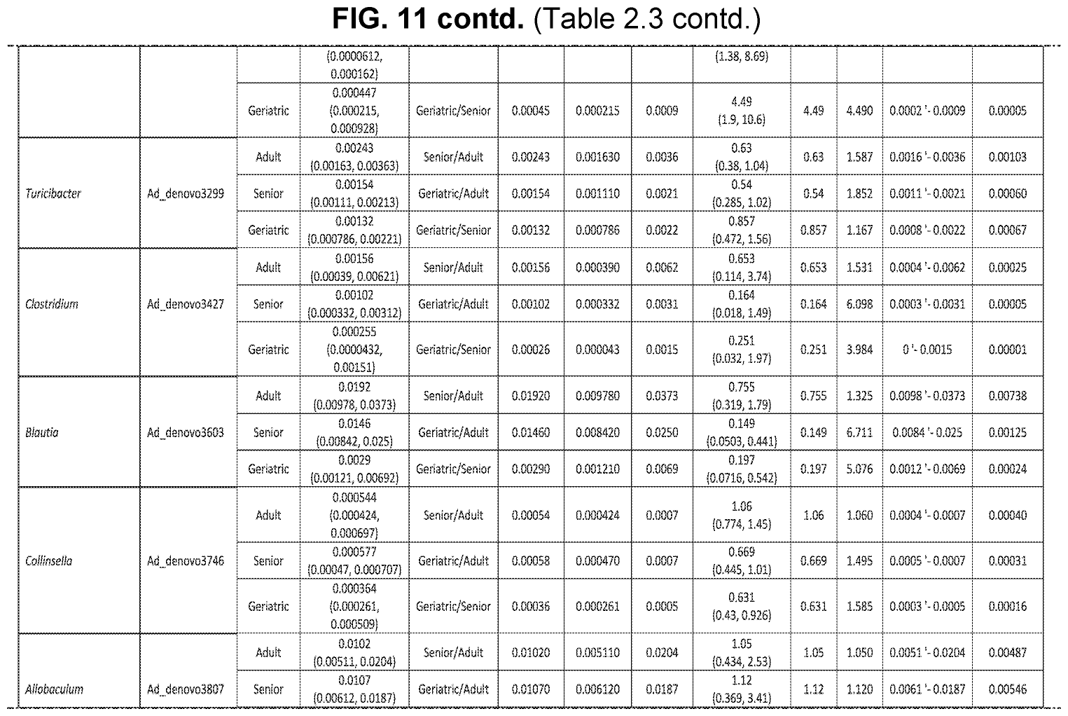

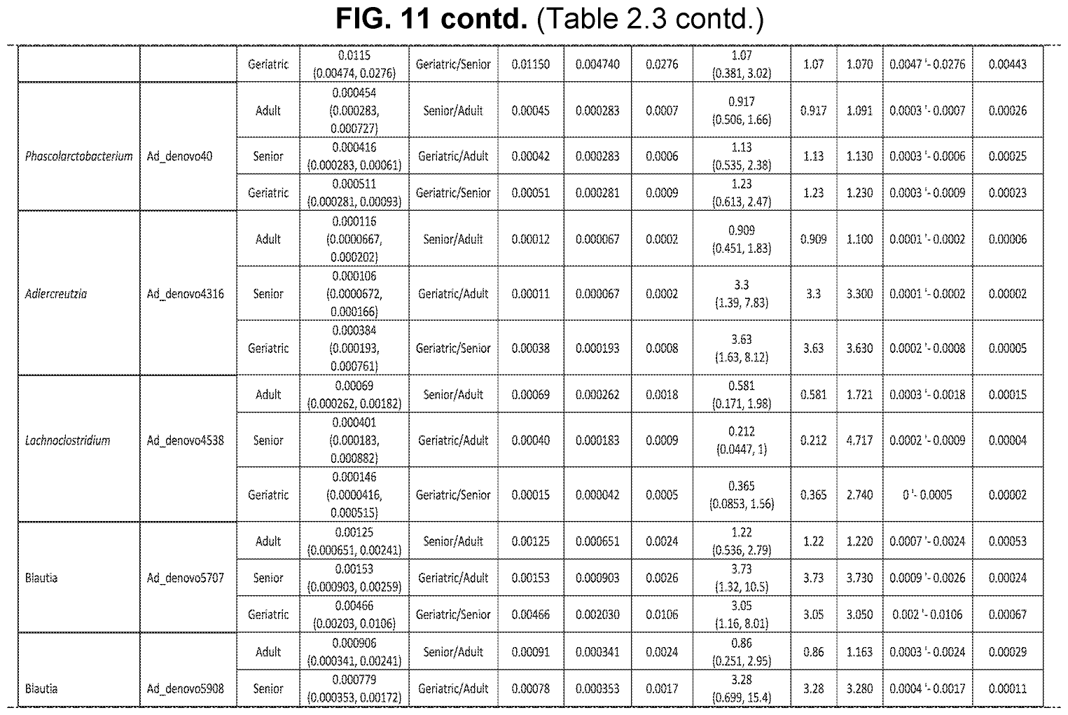

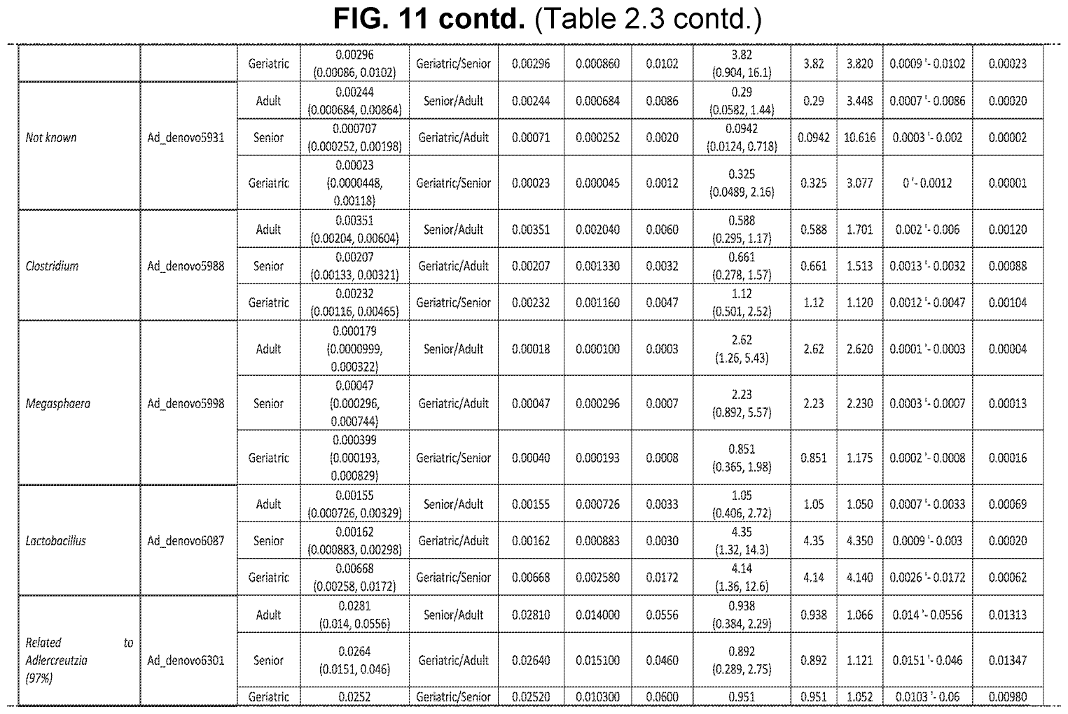

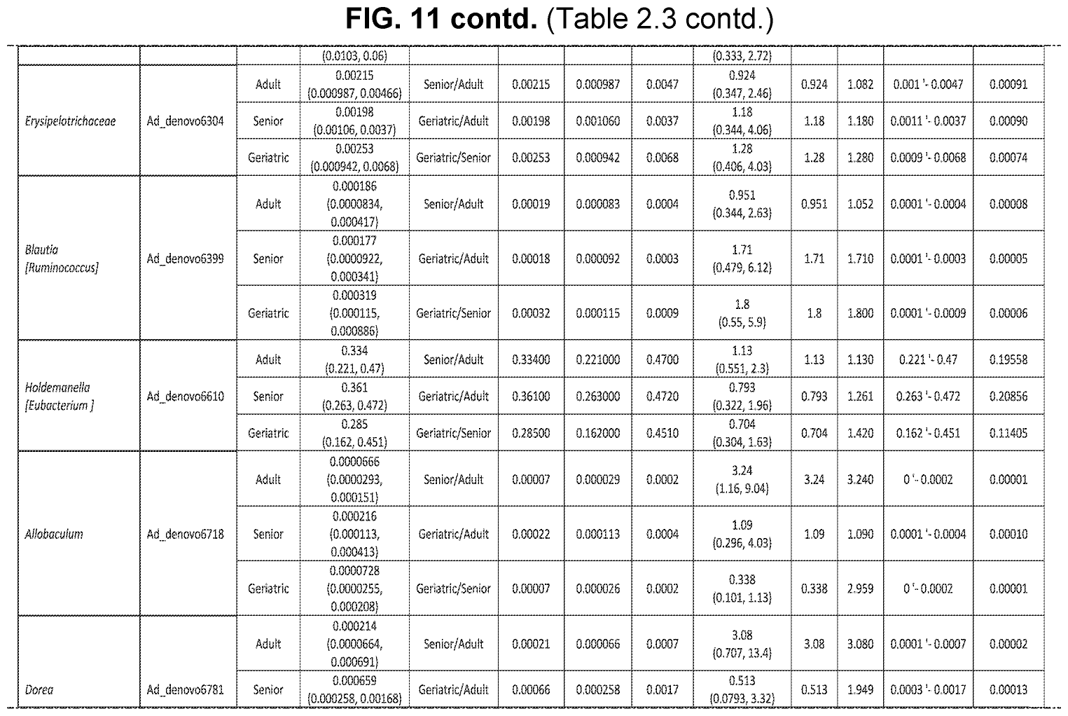

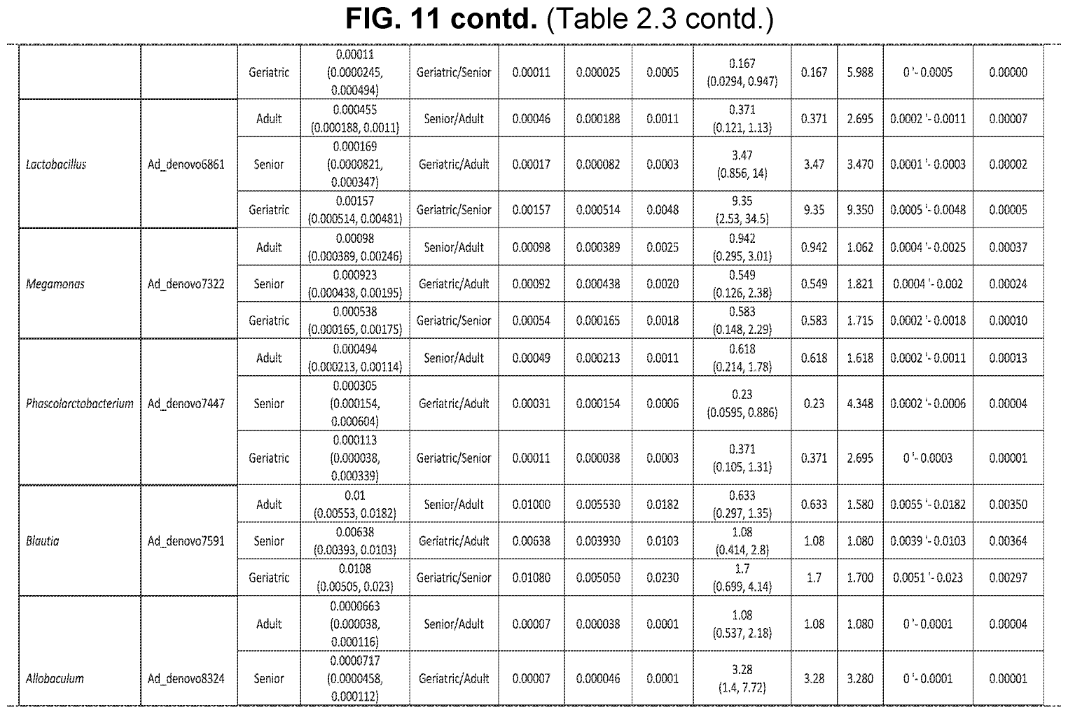

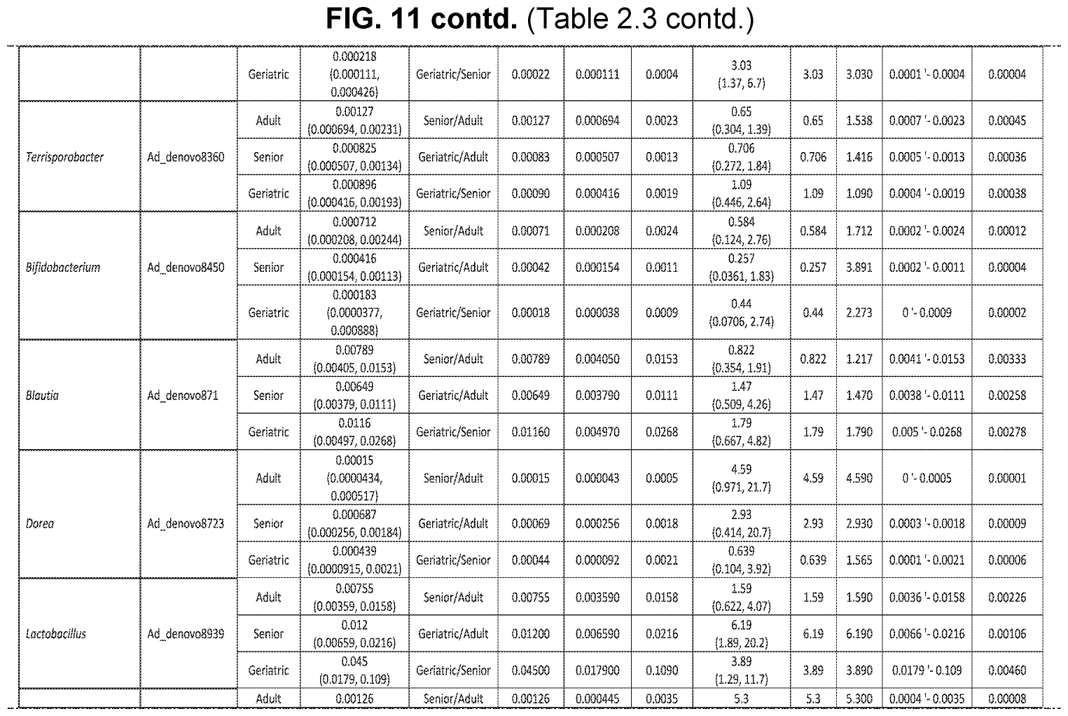

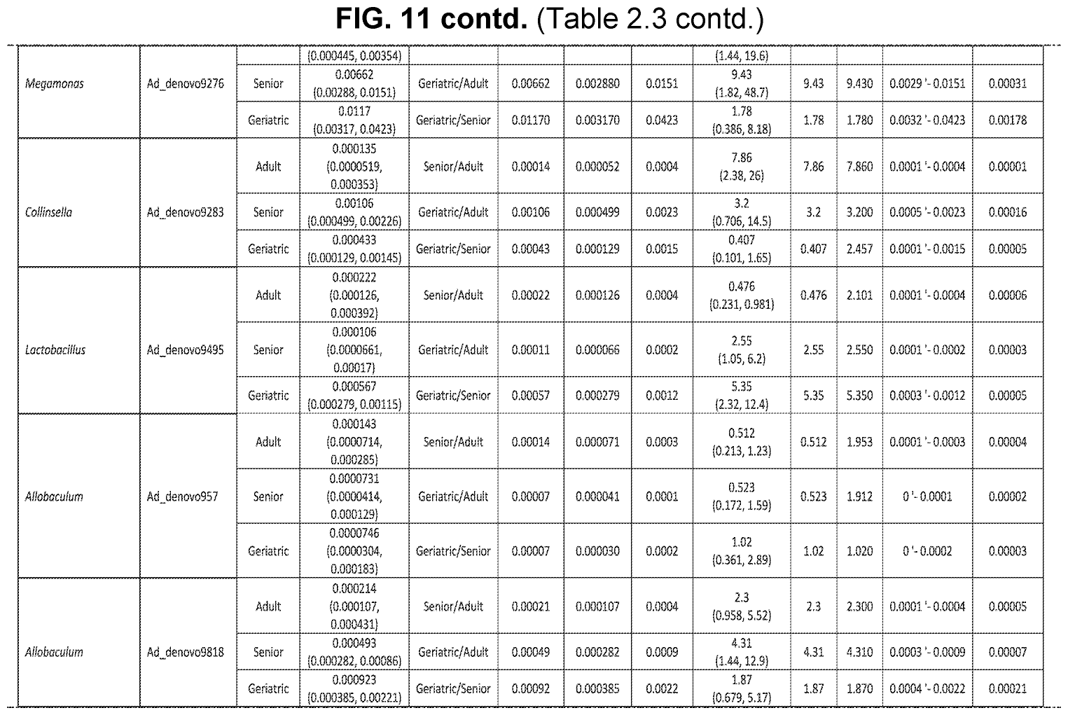

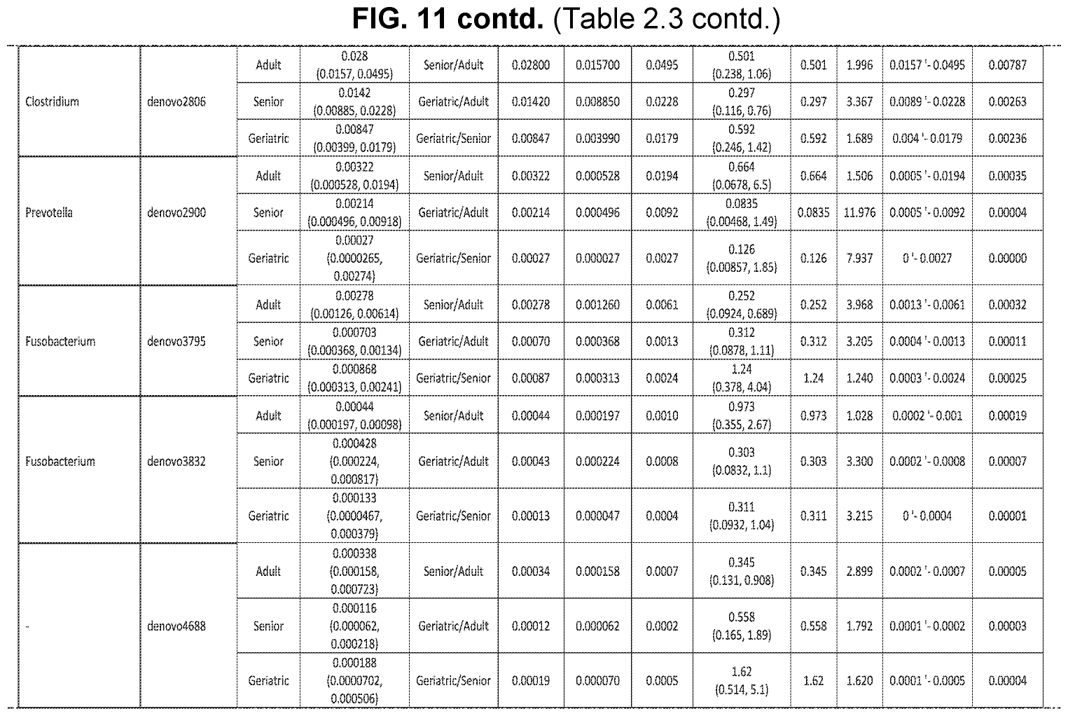

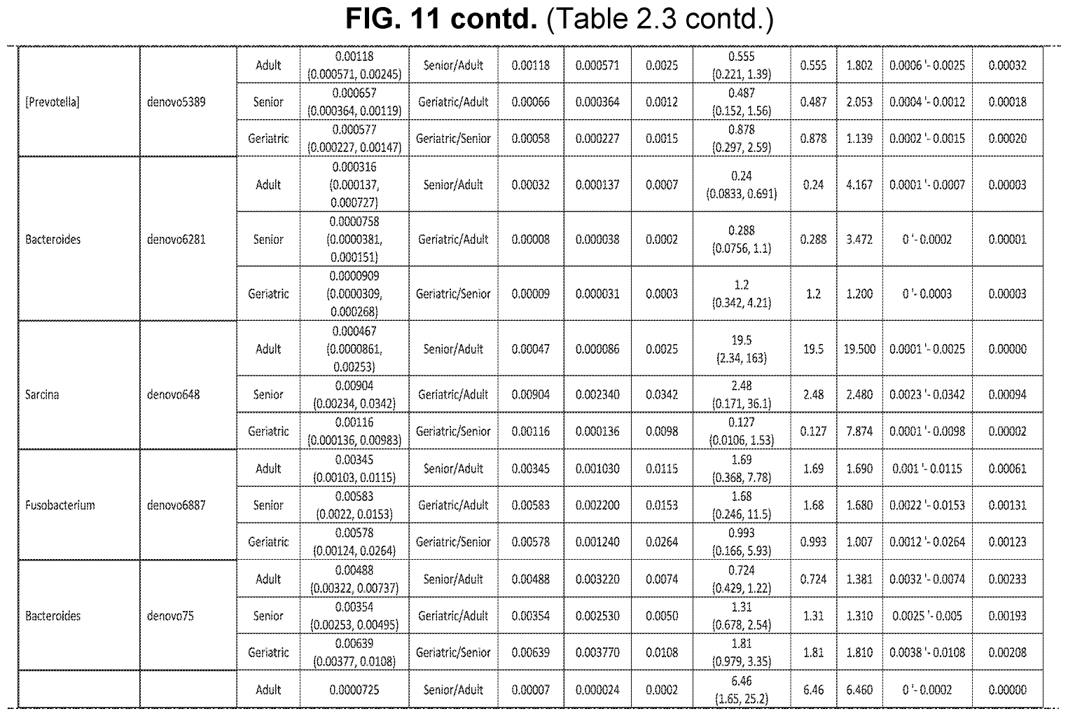

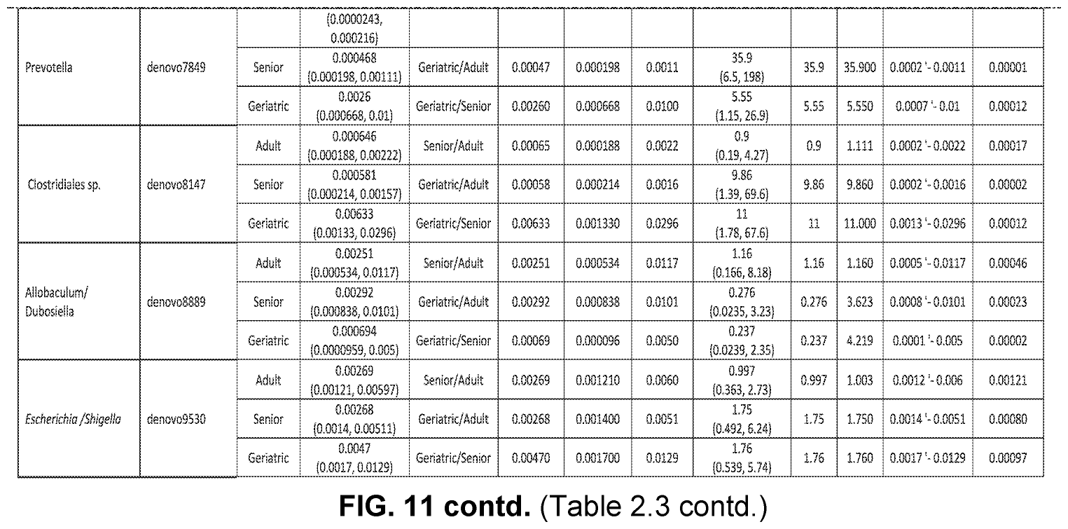

The presently disclosed subject matter provides that bacterial species from certain bacterial taxa are indicative of a healthy microbiome in canids. These taxa are shown in FIG. 9 and FIG. 11 (Tables 1.3 and 2.3) for puppies and mature canids, respectively. Tables 7 and 8 (below) also show bacterial taxa indicative of a healthy microbiome. As will be apparent to a skilled person, the abundance of these taxa in the microbiome will vary between different healthy individuals, but can generally be found within the range shown in FIGS. 9 and 11 (Tables 1.3 and 2.3) and Table 8. Thus, a bacterial taxa will be considered within a healthy range if it falls within the range shown in FIGS. 9 and 11 (Tables 1.3 and 2.3) and Table 8. In such embodiments, the abundance of the bacterial taxa which is analysed will be compared to the “90%” value shown in FIG. 9 (Table 1.3) for the same bacterial taxa. For example, when bacteria of the genus Anaerostipes are analysed, they will be deemed to be in a healthy range if they are in the range shown for Anaerostipes in FIG. 9 (Table 1.3), i.e., 0-0.0004. Thus, the abundance of bacterial genus or family can be increased or decreased relative to the abundance shown in FIG. 9 (Table 1.3). Furthermore, in some embodiments, when there are different ranges across a genus in FIG. 9 (Table 1.3), the ranges specific to a particular OTU is used in the methods disclosed herein, rather than using the values for the genus.

In some cases, the abundance of the bacterial species will fall outside these ranges. The presently disclosed subject matter, however, provides that a bacterial species' abundance can still be considered to be indicative of a healthy microbiome if its abundance is increased or decreased relative to the ranges shown in FIG. 9 (Table 1.3). Thus, a particular species within a puppy's microbiome will still be considered within a healthy range if its abundance is above or below the range indicated in FIG. 9 (Table 1.3), as indicated in the table.

For example, an abundance which is above the range shown in FIG. 9 (Table 1.3) is still considered healthy for species from a genus selected from the group consisting of Absiella [Eubacterium], Anaerostipes, Anaerotruncus, Bifidobacterium, Blautia, Butyricicoccus, Clostridium_sensu_stricto_1, Collinsella, Enterococcus, Erysipelatoclostridium, Flavonifractor, Fusobacterium, Lachnoclostridium, Lachnospiraceae_NK4A136 group, Lactobacillus, Megamonas, Romboutsia, Roseburia, Ruminococcaceae, and Lachnospiraceae. In some embodiments, the bacterial species are selected from the group consisting of Absiella [Eubacterium] dolichum, Anaerostipes caccae, Anaerostipes indolis, Anaerostipes rhamnosivorans, Anaerotruncus sp., Bifidobacterium sp., Blautia [Ruminococcus] gnavus, Blautia [Ruminococcus] torques, Blautia [Ruminococcus] torques group sp., Blautia producta, Blautia sp., Butyricicoccus pullicaecorum, Butyricicoccus sp., Cellulosilyticum sp., Clostridium hiranonis, Clostridium sp., Clostridium sp., Collinsella sp., Dorea massiliensis, Enterococcus sp., Erysipelatoclostridium sp., Faecalibacterium prausnitzii, Finegoldia magna, Finegoldia sp., Fusobacterium sp., Holdemanella [Eubacterium] biforme, Lachnoclostridium sp., Lachnoclostridium [Clostridium] sp., Lachnoclostridium hylemonae, Lachnoclostridium leptum, Lachnoclostridium sp., Lachnospiraceae novel sp., Lachnospiraceae sp., Lachnospiraceae_NK4A136_group sp., Lactobacillus animalis, Lactobacillus apodemi, Lactobacillus faecis, Lactobacillus murinus, Lactobacillus plantarum, Lactobacillus reuteri, Lactobacillus ruminis, Lactobacillus saerimneri, Lactobacillus sp., Megamonas funiformis, Megamonas sp., Megamonas rupellensis, Pseudoflavonifractor capillosus; Pseudoflavonifractor sp., Romboutsia sp., Roseburia faecis, Roseburia sp., Ruminococcaceae novel sp., Ruminococcaceae_UCG-015 sp., Sellimonas sp., Terrisporobacter glycolicus, Terrisporobacter mayombei, Terrisporobacter petrolearius, Terrisporobacter sp., Terrisporobacter sp. SN1, Terrisporobacter sp. SN9, Terrisporobacter sporobacter, Turicibacter sanguinis, and Turicibacter sp.

In contrast, a decrease in abundance compared to the range indicated in FIG. 9 (Table 1.3) is considered healthy for a species from the genus Fusobacterium, in particular Fusobacterium mortiferum.

In some embodiments, the methods of the present disclosure do not comprise a step of testing for a bacterial species from the genera selected from the group consisting of Lactobacillus, Enterococcus, Turicibacter and/or Streptococcus.

Likewise, FIG. 11 (Table 2.3) indicates the range of abundance for various bacterial species which is considered healthy for a mature (i.e., an adult, senior or geriatric) canid. The abundance of the particular species can fall within the upper and lower 5% range shown in FIG. 11 (Table 2.3). Similar to the situation in puppies, a decrease in the abundance of a particular species can still be considered healthy provided it does not decrease below the “notification point” shown in FIG. 11 (Table 2.3). The microbiome will be deemed unhealthy if one or more species (e.g., 2, 3, 4, 5, 10, 13, 15, 18, 20, 22, or more) fall below this point. In some embodiments, the microbiome will be deemed unhealthy if one-fifth to one-third of the species from FIG. 11 (Table 2.3) falls below the “notification” point shown in FIG. 11 (Table 2.3). For such animals, it can be beneficial to seek veterinary advice and to consider an intervention (e.g., a treatment). In some embodiments, preferred species for detecting a mature canid's health are from genera selected from the group consisting of Adlercreutzia, Allobaculum, Bacteroides, Bifidobacterium, Blautia, Clostridiales sp., Collinsella, Dorea, Enterococcus, Erysipelotrichaceae, Faecalibacterium, Fusobacterium, Holdemanella [Eubacterium], Lachnoclostridium, Lactobacillus, Megamonas, Megasphaera, Phascolarctobacterium, Prevotella, Ruminococcus, Sarcina, Terrisporobacter, and Turicibacter.

In addition, or alternatively, the methods of the present disclosure can be practised using genera selected from the group consisting of Prevotella, Allobaculum, Blautia and Paraprevotella. It has been found that these taxa are particularly useful for determining the health of a canid's microbiome. Thus, in some embodiments, the methods of the present disclosure comprise a step of testing for a bacterial species from the genus Prevotella. In further embodiments, a method of the present disclosure comprises a step of testing for a bacterial species selected from at least one, at least two, at least three or at least four of the genera Prevotella, Allobaculum, Blautia and Paraprevotella. The exception for Prevotella is if the Prevotella species is Prevotella copri (for reasons stated below). If the only Prevotella identified is Prevotella copri, then Prevotalla should not be considered as a health indicator.

In additional embodiments, the methods of the present disclosure can include testing for a bacterial species selected from the group consisting of a bacterial species of Lactobacillus, a bacterial species of Ruminococcaceae, a bacterial species of Megamonas, a bacterial species of Holdemanella, a bacterial species of Lachnospiraceae, a bacterial species of Turicibacter, a bacterial species of Dorea, a bacterial species of Enterococcus, a bacterial species of Bifdobacterium, and bacterial species of Butyricicoccus, Clostridium hiranonis and Ruminococcus gauvreauii.

In additional embodiments, the methods of the present disclosure can involve testing selected bacterial sequence types from within a bacterial genus representing markers of the microbiome health in dogs across all lifestages from puppy through youth, adult senior and geriatric animals. Table 8 indicates the range of relative abundance or proportion of the sequences within the 90% range for various bacterial genera which are considered healthy or signs of dysbiosis across all lifestages for a canid. The abundance of the particular genus can fall within the upper and lower 5% range of the relative proportions shown in Table 8. A decrease or increase in the abundance of a particular species can still be considered to demonstrate that the animal's microbiome is healthy provided it does not decrease below the “notification point” shown in Table 8 (i.e., below the ‘Lower 5% range’ or above the ‘Upper 5% range’). The microbiome will be deemed unhealthy if four or more genera (e.g., 5, 10, 13, 15, 18, 20, 22 or more) fall below or above these points. In some embodiments, the microbiome is deemed unhealthy if one-fifth to one-third of the species from Table 8 falls above or below the “notification” points shown in Table 8. For such animals, it can be beneficial to seek veterinary advice and/or to consider an intervention (e.g., a treatment) such as a dietary intervention or treatment prescribed by a veterinary professional.

In addition, or alternatively, a method of the present disclosure can include a step of testing bacterial species from taxa selected from the group consisting of Enterobacteriaceae, Escherichia/Shigella, Mogibacterium, Fusobacterium, Lachnoclostridium, and Prevotella copri. Prevotella copri is an exception to the general finding that the Prevotella genus is a health indicator. Prevetella copri, specifically, is thought to be associated with RA (arthritis and particularly reactive arthritis/rheumatoid arthritis). It has been found that the abundance of bacteria from these genera is increased in dysbiosis. Thus, preferably, the abundance of such species falls within the range indicated in FIG. 9 (Table 1.3), FIG. 11 (Table 2.3), or Table 8 as discussed above.

In addition, or alternatively, the canid's microbiome health can be assessed by determining the diversity of bacterial species within a canid's microbiome. To this end, the diversity index of the bacterial species within the canid's microbiome is determined and compared to the diversity index of a control data set. For a healthy pre-weaned puppy, the diversity index will generally be in the range of about 0.123 to about 1.744; for a post-weaned puppy, the healthy range is from about 1.294 to about 2.377; for a healthy adult, the mean range of the diversity index is from about 2.3755 to about 3.1534; for a healthy senior canid, the average range is from about 2.1971 to about 2.8263; and for a healthy geriatric canid, the average range is from about 2.3339 to about 3.3273. Where the microbiome diversity index falls outside these ranges, the microbiome will be considered less healthy. However, it may not always be necessary to seek treatment. This will generally be useful, however, where the diversity index falls above or below a certain “intervention point”. These intervention points are listed in Table 1.0-A below:

TABLE 1.0-A

| | Lower intervention | Upper intervention |
|---|---|---|
| Life stage | point | point |
| Pre-weaned puppy | <0.2059 | >2.0240 |
| Post-weaned puppy | <0.6351 | >2.8786 |
| Adult | <1.83 | >3.72 |
| Senior | <1.24 | >3.55 |
| Geriatric | <2.16 | >3.47 |

In some embodiments, when the diversity index falls outside the range discussed above, the method can comprise a further step of changing the composition of the microbiome, as discussed below. This is particularly preferred where the diversity index falls above or below the notification point, as shown above.

### The Control Data Set

The abundance of the bacterial species is compared to a control data set from a canid with a similar chronological age or lifestage, e.g. a puppy, an adult canid, a senior canid or a geriatric canid. FIGS. 9 and 11 (Tables 1.3 and 2.3) provide suitable control data sets against which the microbiome composition from the canid can be compared.

Alternatively, or in addition, a control data set can be prepared. To this end, the microbiome of two or more (e.g., 3, 4, 5, 10, 15, 20 or more) healthy canids can be analysed for the abundance of the species contained in the microbiome. A healthy canid in this context is a canid who has not been diagnosed with a disease that is known to affect the microbiome. Examples of such diseases include irritable bowel syndrome, ulcerative colitis, Crohn's and inflammatory bowel disease. The two or more canids will generally be from a particular life stage. For example, they can be puppies, adult canids, senior canids or geriatric canids. This is useful because the microbiome changes in a canid's lifetime and the microbiome therefore needs to be compared to a canid at the same lifestage. Where the canid is a dog, the control data set can further be from a dog of the same breed or, where the dog is a mongrel, the same breed as one of the direct ancestors (parents or grandparents) of the dog.

The control data set can also from the same canid who is diagnosed or monitored by a method of the present disclosure. For example, the microbiome of the canid can be analysed and the data can subsequently be used as a control data set to evaluate whether the dog's microbiome health has changed.

Specific steps to prepare the control data set can comprise analysing the microbiome composition of at least two (e.g., 3, 4, 5, 6, 7, 8, 9, 10, 15, 20 or more) puppies, and/or at least two (e.g., 3, 4, 5, 6, 7, 8, 9, 10, 15, 20 or more) adult canids, and/or at least two (e.g., 3, 4, 5, 6, 7, 8, 9, 10, 15, 20 or more) senior canids and/or at least two (e.g., 3, 4, 5, 6, 7, 8, 9, 10, 15, 20 or more) geriatric canids; determining the abundance of bacterial species (in particular those discussed above); and compiling these data into a control data set.

For embodiments where the diversity index of the microbiome is determined, the control data set can be prepared in a similar manner. In particular, the diversity index can be determined in two or more (e.g., 3, 4, 5, 6, 7, 8, 9, 10, 15, 20 or more) healthy canids at a particular life stage (puppy, adult, senior or geriatric). The results can then be used to identify the mean range for the diversity index in a canid at that life stage.

It will be understood that the control data set does not need to be prepared every time the method of the present disclosure is performed. Instead, a skilled person can rely on an established control set.

In addition to those described herein, techniques which allow a skilled person to detect and quantitate bacterial taxa are well known in the art. These include, for example, polymerase chain reaction (PCR), quantitative PCR, 16 S rDNA amplicon sequencing, shotgun sequencing, metagenome sequencing, Illumina sequencing, and nanopore sequencing. Preferably, the bacterial taxa are determined by sequencing the 16 s rDNA sequence. Other methods would include shotgun sequencing to determine characteristic non-16 SrDNA gene sequences or other metabolites and biomarkers for identification of the species.

In some embodiments, the bacterial taxa are determined by sequencing the V4-V6 region, for example using Illumina sequencing. These methods can use the primers 319F: CAAGCAGAAGACGGCATACGAGATGTGACTGGAGTTCAGACGTGTGCTCTTCCGATCT (SEQ ID NO: 1) and 806R: AATGATACGGCGACCACCGAGATCTACACTCTTTCCCTACACG ACGCTCTTCCGATCT (SEQ ID NO: 2).

The bacterial species can also be detected by other means known in the art such as, for example, RNA sequencing, protein sequence homology or other biological marker indicative of the bacterial species.

The sequencing data can then be used to determine the presence or absence of different bacterial taxa in the sample. For example, the sequences can be clustered at about 98%, about 99% or 100% identity and abundant taxa (e.g., those representing more than 0.001 of the total sequences) can then be assessed for their relative proportions. Suitable techniques are known in the art and include, for example, logistic regression, partial least squares discriminate analysis (PLSDA) or random forest analysis and other multivariate methods.

### The Canid

The methods of the present disclosure can be used to determine the microbiome health of a canid. This genus comprises domestic dogs (Canis lupus familiaris), wolves, coyotes, foxes, jackals, dingoes and the present disclosure can be used for all these animals. In some embodiments, the subject is a domestic dog, herein referred to simply as a dog.

In some embodiments, the canid is healthy. “Healthy,” as used herein, refers to a canid who has not been diagnosed with a disease that is known to affect the microbiome. Examples of such diseases include, but are not limited to, irritable bowel syndrome, ulcerative colitis, Crohn's and inflammatory bowel disease. Preferably, the canid does not suffer from dysbiosis. Dysbiosis refers to a microbiome imbalance inside the body, resulting from an insufficient level of keystone bacteria (e.g., bifidobacteria, such as B. longum subsp. infantis) or an overabundance of harmful bacteria in the gut. Methods for detecting dysbiosis are well known in the art.

One advantage of the methods of the present disclosure is that they allow a skilled person to determine whether the canid's microbiome is healthy, taking into account the canid's lifestage.

There are numerous different breeds of domestic dogs, which show a diverse habitus. Different breeds also have different life expectancies with smaller dogs generally being expected to live longer than bigger breeds. Accordingly, different breeds are considered to be puppies, adult, senior or geriatric at different time points in their life. A summary of the different life stages is provided in Table 1.0-B below.

TABLE 1.0-B

| | Youth | Adult | | Senior | Geriatric |
|---|---|---|---|---|---|
| Toy | Up to 7 years | 8-11 | years | 12-13 years | 14+ years |
| Small | Up to 7 years | 8-11 | years | 12-13 years | 14+ years |
| Medium | Up to 5 years | 6-9 | years | 10-13 years | 14+ years |
| Large | Up to 5 years | 6-9 | years | 10-11 years | 12+ years |

The distinction between toy, small, medium and large breeds is known in the art. In particular, toy breeds comprise distinct breeds including but not limited to Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy and Yorkshire Terrier.

Small breeds are larger on average than toy breeds with an average body weight of up to about 10 kg. Non-limiting exemplary breeds include French Bulldog, Beagle, Dachshund, Pembroke Welsh Corgi, Miniature Schnautzer, Cavalier King Charles Spaniel, Shih Tzu, and Boston Terrier.

Medium dog breeds have an average weight of about 11 kg to about 26 kg. These dog breeds include, but are not limited to, Bulldog, Cocker Spaniel, Shetland Sheepdog, Border Collie, Basset Hound, Siberian Husky and Dalmatian.

Large breed are those with an average body weight of at least about 27 kg. Non-limiting examples include Great Dane, Neapolitan mastiff, Scottish Deerhound, Dogue de Bordeaux, Newfoundland, English mastiff, Saint Bernard, Leonberger and Irish Wolfhound.

Cross-breeds can generally be categorised into toy, small, medium and large dogs depending on their body weight.

### The Sample

According to the methods of the present disclosure, the sample from which the bacterial species are analysed can be, in some embodiments, a fecal sample or a sample from the gastrointestinal lumen of the canid. Fecal samples are convenient because their collection is non-invasive, and it also allows for easy repeated sampling of individuals over a period of time. However, other samples can also be used in the methods disclosed herein, including, but not limited to, ileal, jejunal, duodenal samples and colonic samples.

In some embodiments, the sample is a fresh sample. In further embodiments, the sample is frozen or is stabilised by other means, such as addition to preservation buffers, or by dehydration using methods such as freeze drying, before use in the methods of the present disclosure.

Before use in the disclosed methods, in some embodiments, the sample is processed to extract DNA. Methods for isolating DNA are well known in the art, as reviewed in reference [8], for example. These methods include, for example, the Qiagen DNeasy Kit™, the MoBio PowerFecal Kit™, Qiagen QIAamp Cador Pathogen Mini Kit™, the Qiagen QIAamp DNA Stool Mini Kit™ as well as Isopropanol DNA Extraction. A further useful tool to use with the methods of the present disclosure is the QIAamp Power Faecal DNA kit (Qiagen).

### Changing the Microbiome

In some embodiments, the methods of the present disclosure comprises a further step of changing the composition of the microbiome. The composition of the microbiome can be changed by administering to the canid a dietary change, a functional food, a supplement, or a nutraceutical or pharmaceutical composition that is capable of changing the composition of the microbiome. Such functional foods, nutraceuticals, live biotherapeutic products (LBPs) and pharmaceutical compositions are well known in the art and comprise bacteria [9]. They can comprise single bacterial species selected from the group consisting of Bifidobacterium sp. such as B. animalis (e.g., B. animalis subsp. animalis or B. animalis subsp. lactis), B. bifidum, B. breve, B. longum (e.g., B. longum subsp. infantis or B. longum subsp. longum), B. pseudolongum, B. adolescentis, B. catenulatum, or B. pseudocatanulatum; single bacterial species of Lactobacillus, such as L. acidophilus, L. antri, L. brevis, L. casei, L. coleohominis, L. crispatus, L. curvatus, L. fermentum, L. gasseri, L. johnsonii, L. mucosae, L. pentosus, L. plantarum, L. reuteri, L. rhamnosus, L. sakei, L. salivarius, L. paracasei, L. kisonensis, L. paralimentarius, L. perolens, L. apis, L. ghanensis, L. dextrinicus, L. shenzenensis, L. harbinensis; or single bacterial species of Pediococcus, such as P. parvulus, P. loii, P. acidilactici, P. argentinicus, P. claussenii, P. pentosaceus, or P. stilesii; or similarly species of Enterococcus such as E. faecium, or Bacillus species such as Bacillus subtilis, B. coagulans, B. indicus, or B. clausii. Additionally, combinations of these and other bacterial species can be used. The amount of the dietary change, the functional food, the supplement, the nutraceutical composition, or the pharmaceutical composition that is administered to the canid can be an amount that is effective to effect a change in the composition of the microbiome.

The further step of changing the composition of the microbiome can be performed in instances where a canid's biological microbiome is found to be unhealthy. In that case, it can be highly desirable to make a dietary change and/or to administer a nutraceutical or pharmaceutical composition to shift the microbiome back to a healthy state, as determined by a method of the present disclosure.

The methods of the present disclosure can also be used to assess the success of a treatment as described above. To this end, a canid can undergo a dietary change and/or receive a nutraceutical or pharmaceutical composition, which is capable of changing the composition of the microbiome. Following commencement of the treatment (e.g., administration of the pharmaceutical composition), for example, after about 1 day, 2 days, 5 days, 1 week, 2 weeks, 3 weeks, 1 month, etc., the health of the microbiome can be assessed using any of the methods of the present disclosure. Preferably, the health of the microbiome is determined before and after administration of the pharmaceutical or nutraceutical composition.

### Monitoring

In some embodiments, the methods described herein are performed once to determine a canid's microbiome health. In other embodiments, the methods of the present disclosure are performed more than once, for example, two times, three times, four times, five times, six times, seven times, or more than seven times. This allows the biological age of the microbiome to be monitored over time. This can be useful, for example, where a canid is receiving treatment to shift the microbiome. The first time the method is performed, the health of the microbiome is determined and, following a dietary change or administration of a nutraceutical or pharmaceutical composition, the method is repeated to assess the influence of the pharmaceutical composition on the health of the microbiome. The health of the microbiome can also be determined for the first time after the canid has received treatment, and the method repeated afterwards, to assess whether there is a change in the health of the microbiome.

The methods described herein can be repeated about one week, two weeks, three weeks, one month, two months, three months, four months, five months, six months, 12 months, 18 months, 24 months, 30 months, 36 months, or more than 36 months apart.

### Treatment

In some embodiments, the methods of the present disclosure can also relate to methods for treating a canid having an unhealthy microbiome. In some embodiments, the methods for treating include: (i) identifying the canid as requiring treatment by determining the unhealthy status of the microbiome according to any of the methods disclosed herein, and (ii) administering to the canid a dietary change, a functional food, a supplement, a nutraceutical, or a pharmaceutical composition as disclosed herein that is capable of changing the composition of the microbiome. The amount of the dietary change, the functional food, the supplement, the nutraceutical composition, or the pharmaceutical composition that is administered to the canid can be an amount that is effective to effect a change in the composition of the microbiome, or to improve any symptoms relating to the canid having an unhealthy microbiome status. Optionally, in some embodiments, the method further includes determining the microbiome health of the canid following the administration of the dietary change, the functional food, the supplement, the nutraceutical, or the pharmaceutical composition to evaluate the effectiveness of the treatment.

### General

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

The practice of the present disclosure will employ, unless otherwise indicated, conventional methods of chemistry, biochemistry, molecular biology, immunology and pharmacology, within the skill of the art. Such techniques are explained fully in the literature. See, e.g., references [10-17], etc.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref [18]. A preferred alignment is determined using the BLAST (basic local alignment search tool) algorithm or the Smith-Waterman homology search algorithm using an affine gap search with a gap open penalty of 12 and a gap extension penalty of 2, BLOSUM matrix of 62. The Smith-Waterman homology search algorithm is disclosed in ref. [19]. The alignment can be over the entire reference sequence, i.e. it can be over 100% length of the sequences disclosed herein.

### Definitions

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “containing,” and “comprising” are interchangeable, and one of skill in the art is cognizant that these terms are open ended terms. Further, the term “comprising” encompasses “including” as well as “consisting,” e.g., a composition “comprising” X can consist exclusively of X or can include something additional, e.g., X+Y.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. In certain embodiments, the term “about” in relation to a numerical value x is optional and means, for example, x±10%.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition (e.g., a dietary change, a functional food, a supplement, a nutraceutical composition, or a pharmaceutical composition) to change the composition of a microbiome in a feline having an unhealthy microbiome, the effective amount is an amount sufficient to bring the health status of the microbiome back to a healthy state, which is determined according to one of the methods disclosed herein. In certain embodiments, an effective treatment as described herein can also include administering a treatment in an amount sufficient to decrease any symptoms associated with an unhealthy microbiome. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of an unhealthy microbiome. An effective amount can be administered in one or more administrations. A likelihood of an effective treatment described herein is a probability of a treatment being effective, i.e., sufficient to alter the microbiome, or treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. In certain embodiments, the decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

The word “substantially” does not exclude “completely”, e.g., a composition which is “substantially free” from Y can be completely free from Y. Where necessary, the word “substantially” can be omitted from the definition of the present disclosure.

Unless specifically stated, a process or method comprising numerous steps can comprise additional steps at the beginning or end of the method, or can comprise additional intervening steps. Also, steps can be combined, omitted or performed in an alternative order, if appropriate.

Various embodiments of the methods of the present disclosure are described herein. It will be appreciated that the features specified in each embodiment can be combined with other specified features, to provide further embodiments. In particular, embodiments highlighted herein as being suitable, typical or preferred can be combined with each other (except when they are mutually exclusive).

### EXAMPLES

The presently disclosed subject matter will be better understood by reference to the following Example, which is provided as exemplary of the invention, and not by way of limitation.

### Example 1: Assessment of Microbiome Characteristics in Dogs

### Background

Changes in the gut microbiota occur during the period between birth and maturity, with an increase in diversity and stability seen in the human faecal microbiota until maturity [20; 21]. Perturbations in the neonatal gut microbiota during this period, either through birth [22], introduction of novel foods [20], or disease [23] can change the course of microbiota development and composition of the microbiota and microbiome at maturity, with the potential to affect the long term health of the host [24,25]. By comparison, the mature microbial community in humans appears to be stable over time and more resilient to challenge [26].

The initial establishment of the gut microbiota is an essential step in neonatal development, influencing immunological development in infancy and health throughout life. In human studies, there is some evidence that this initial microbial colonisation of the infant gut can occur via prenatal inoculation in utero [27]. Bacteria and bacterial DNA have been identified in the amniotic fluid [28], placenta, and in the meconium of the neonate [29,30,31]. Initial colonisation can therefore occur through ingestion of amniotic fluid [32] or through placental transfer and translocation though the maternal blood supply. Evidence also exists for the direct inoculation of the infant gut via colostrum and maternal milk in human infants [33]. Recent studies indicate that human milk contains a diverse microbiota that is reflected in the early colonisers found in the infant gut [34,35].

Human studies have demonstrated that within the first days of life, Bacteroides and Bifidobacterium species become the most abundant genera in the gut of breastfed infants [36,37]. These initial colonising species can provide favorable conditions to enable other microbes to establish through production of an anaerobic environment and provision of substrates for bacterial growth [38]. Few bacteria can gain access to the energetic content of maternal milk as it is presented in the colon, but species of both Bacteroides and Bifidobacterium are able to utilize human milk oligosaccharides (HMOs) as an energy source [39]. In particular, Bifidobacterium longum subspecies infantis (B. infantis) is unique among gut bacteria in its capacity to digest and consume any HMO structure, and has been shown to predominate in the intestinal microbiota throughout the first year of life in human breast-fed infants with potential long term effects on the health of the host [40]. In vitro studies have demonstrated that B. infantis grows more rapidly than other bacterial strains in the presence of HMOs, and demonstrates a number of beneficial effects, including promoting anti-inflammatory activity in premature intestinal cells, and decreasing intestinal permeability [41,40].

Given that early inoculation of the gastrointestinal tract of dogs can also occur prior to birth in utero, the early neonate microbiota can be enriched for species giving an evolutionary advantage to the infant, and hence can be enriched for bacterial species actively transferred to the infant from the mother via biological processes evolved to conferred an advantage to the survival of the offspring. Such organisms could therefore be enriched in the first days following birth and associated with health over the lifetime of the animal. To investigate this hypothesis the gut microbiota was assessed in a cohort of puppies in the days immediately following birth. Data on the faecal microbiota was derived by analysis of the microbiota in freshly produced faecal samples from 39 puppies with samples taken at 12 time points. These time points were grouped into, early postpartum puppyhood—2, 4, 6, 8, 10, 12 days; mid puppyhood (during-weaning) 17, 24 and 31 days and later (rapid growth phase of) puppyhood (post-weaning) late 38, 45 and 52 days.

### Study Cohort

A cohort of 6 litters of puppies bred by Canine Companions for Independence (CCI) were recruited to the study and were housed in volunteer homes during birth, early development and weaning including the period throughout the sample collection. Puppy faecal samples were collected on days 2, 4, 6, 8, 10, 12, 17, 24, 31 and 38 post parturition. All puppies were maintained by their maternal dam who was fed on Eukanuba Premium Performance throughout gestation and lactation. Puppies were weaned onto Eukanuba Large Breed Puppy starting at day 28. Diet details are provided in Table 5 and below.

TABLE 5
Diet Details
Guaranteed Nutritional Analyses

| | Eukanuba Premium | Eukanuba Large |
|---|---|---|
| Nutrient | Performance | Breed Puppy |
| Crude Protein | 30.00% | 26.0% |
| Crude Fat | 20.00% | 14% |
| Crude Fibre | 4.00% | 5.0.% |
| Moisture | 10.00% | 10.0% |
| Ash | 7.60% | — |
| Vitamin E | 140 IU/kg | — |
| Omega-3-Fatty Acids | 0.89% | 0.39% |
| Omega-6-Fatty Acids | 3.18% | 1.93% |
| Calcium | 1.30% | 0.65% |
| Phosphorus | 1.10% | 0.55% |

### Ingredients

Eukanuba Premium Performance

Chicken, Chicken By-Product Meal (Natural source of Chondroitin Sulfate and Glucosamine), Corn Meal, Brewers Rice, Ground Whole Grain Sorghum, Chicken Fat (preserved with mixed Tocopherols, a source of Vitamin F), Dried Beet Pulp, Chicken Flavor, Fish Meal, Dried Egg Product, Fish Oil (preserved with mixed Tocopherols, a source of Vitamin F), Brewers Dried Yeast, Potassium Chloride, Fructooligosaccharides, Salt, Sodium Hexametaphosphate, Choline Chloride, Minerals (Ferrous Sulfate, Zinc Oxide, Manganese Sulfate, Copper Sulfate, Manganous Oxide, Potassium Iodide, Cobalt Carbonate), Calcium Carbonate, Vitamins (Ascorbic Acid, Vitamin A Acetate, Calcium Pantothenate, Biotin, Thiamine Mononitrate (source of vitamin B1), Vitamin B12 Supplement, Niacin, Riboflavin Supplement (source of vitamin B32), Inositol, Pyridoxine Hydrochloride (source of vitamin B6), Vitamin D3 Supplement, Folic Acid), DL-Methionine, Vitamin E Supplement, L-Carnitine, Beta-Carotene, Rosemary Extract.

Eukanuba Large Breed Puppy

Chicken, Corn Meal, Chicken By-Product Meal (Natural source of Chondroitin Sulfate and Glucosamine), Ground Whole Grain Sorghum, Brewers Rice, Dried Beet Pulp, Chicken Flavor, Dried Egg Product, Fish Oil (preserved with mixed Tocopherols, a source of Vitamin E), Brewers Dried Yeast, Fish Meal, Potassium Chloride, Chicken Fat (preserved with mixed Tocopherols, a source of Vitamin E), Salt, Calcium Carbonate, Choline Chloride, Fructooligosaccharides, Minerals (Ferrous Sulfate, Zinc Oxide, Manganese Sulfate, Copper Sulfate, Manganous Oxide, Potassium Iodide, Cobalt Carbonate), DL-Methionine, Vitamins (Ascorbic Acid, Vitamin A Acetate, Calcium Pantothenate, Biotin, Thiamine Mononitrate (source of vitamin B1), Vitamin B12 Supplement, Niacin, Riboflavin Supplement (source of vitamin B2), Inositol, Pyridoxine Hydrochloride (source of vitamin B6), Vitamin D3 Supplement, Folic Acid), Vitamin E Supplement, Marigold, Beta-Carotene, Rosemary Extract.

### Methods

Following DNA extraction from the freshly produced faeces samples, Illumina sequencing of the V4-V6 region was conducted on amplicons generated from the faecal DNA using primer sequences (319F: CAAGCAGAAG ACGGCATACG AGATGTGACT GGAGTTCAGA CGTGTGCTCT TCCGATCT and 806R: AATGATACGG CGACCACCGA GATCTACACT CTTTCCCTAC ACGACGCTCT TCCGATCT). The resulting DNA sequences were clustered at 98% identity, representing approximately species level bacterial clusters, and abundant taxa (representing>0.001 of the total sequences) were then assessed for their relative proportions. The taxonomic groups of bacteria represented by the sequences detected were determined by interrogation of the Greengenes or Silva v132 16 S rDNA databases. Comparison of the taxonomic group to organisms associated with health and disease in other mammals was utilised to highlight bacterial taxa present in the dog and representative of health of the microbiome.

Analysis of each taxa or OTU was performed first using a generalised linear mixed effects model with the OTU count+2 and sample total+4 as the response variable, age as a factor and an intercept as fixed effects and a random effect of puppy nested within dam. These models were used to determine the mean proportion by age of each OTU and to statistically compare the all consecutive ages and 2 vs. 45 weeks, as by 45 weeks all puppies had been weaned. Contrasts were performed by permuting testing, permuting ages within each litter 1,000 times. All contrasts were corrected to have a false discovery rate of 5% using the Benjamini-Hochberg procedure.

Mann-Whitney tests were also performed on data for each taxon/OTU. This test was used to compare proportions of all consecutive ages and 2 vs. 45 weeks. This is a non-parametric alternative to a t-test with fewer requirements, such as normally distributed errors. As with the generalised linear model the Benjamini-Hochberg procedure was used to correct the p-values. Due to the high proportion of 0 s in the data, and in spite of the +2/+4 proportion calculation, the generalised linear model permutation test is known to be more conservative than the non-parametric Mann-Whitney test due to issues with the error distribution assumption. The Mann-Whitney test on the other hand avoids the error distribution assumption however requires independent samples. For the majority of compared time points, especially the earlier ones, this assumption was valid as few puppies had a complete set of samples.

All analyses were performed using R version 3.3.3 and the lme4, mixOmics and multcomp libraries.

### Results

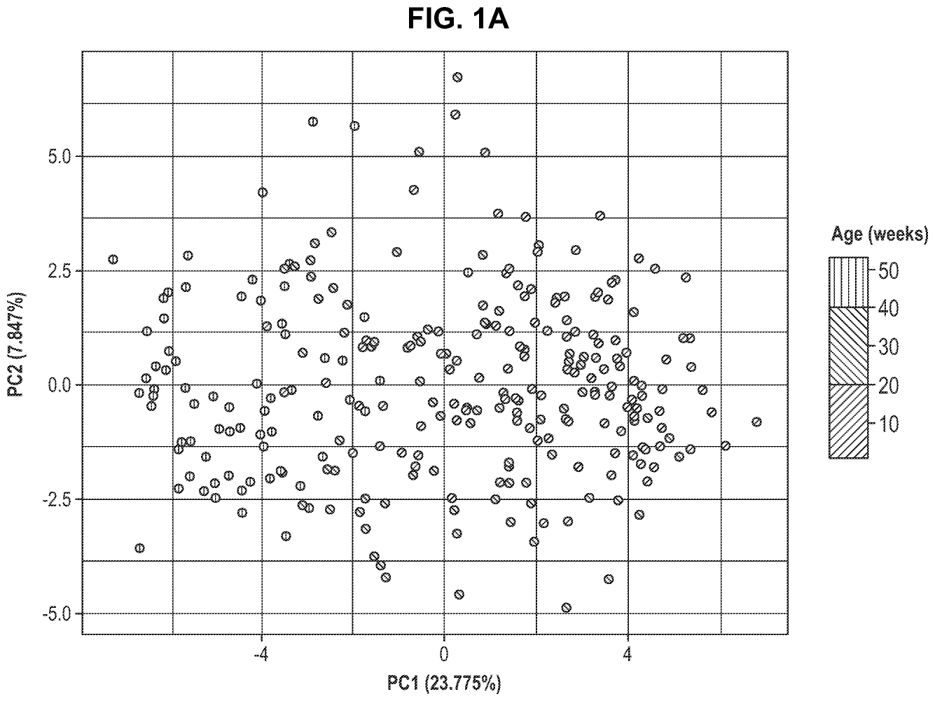

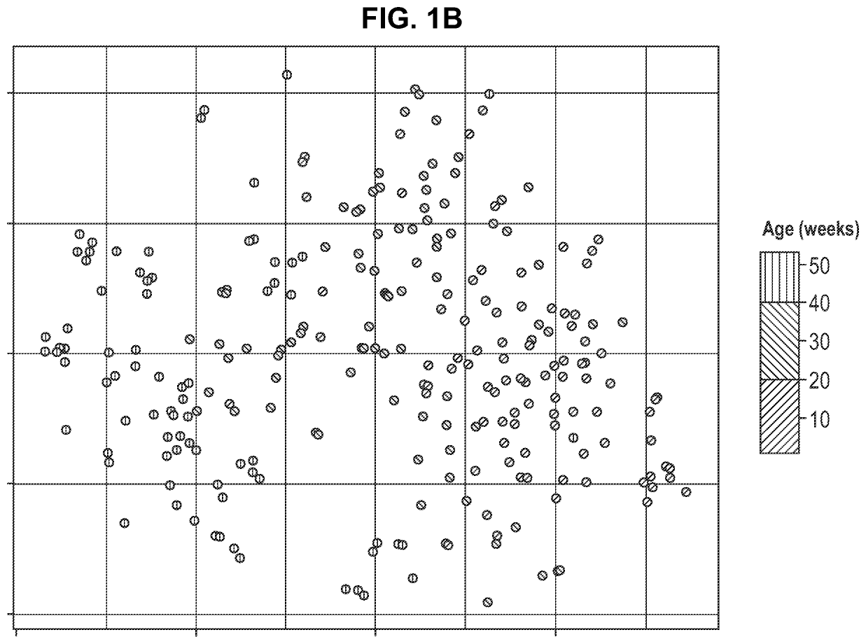

16 SrDNA was isolated from 271 samples, describing a total of 12559 OTUs before data cleaning. After identifying rares/noise, 141 OTUs remained (with the final group comprised of all rares/noise combined). The resulting OTU table is provided in Table 6. Variation in the microbial taxa (OTUs) was observed over development (time after birth) within faecal samples from the puppy cohort by multigroup principal components (PCA) and t-distributed stochastic neighbour embedding (t-SNE) data visualisation (FIG. 1).

TABLE 6
OTU Table

| OTU | Taxa |
|---|---|
| denovo46 | Bacteria; Firmicutes; Clostridia; Clostridiales; Lachnospiraceae; |
| | Lachnospiraceae_NK4A136_group; uncultured_bacterium |
| denovo115 | Bacteria; Firmicutes; Clostridia; Clostridiales; Peptostreptococcaceae; Romboutsia; |
| | uncultured_bacterium |
| denovo200 | Bacteria; Firmicutes; Bacilli; Lactobacillales; Streptococcaceae; Streptococcus; |
| | uncultured_bacterium |
| denovo275 | Bacteria; Firmicutes; Clostridia; Clostridiales; Lachnospiraceae; Tyzzerella; |
| | uncultured_bacterium |
| denovo475 | Bacteria; Proteobacteria; Gammaproteobacteria; Pasteurellales; Pasteurellaceae; |
| | Pasteurella; uncultured_bacterium |
| denovo579 | Bacteria; Firmicutes; Clostridia; Clostridiales; Peptostreptococcaceae; Paeniclostridium; |
| | uncultured_Eubacterium_sp. |
| denovo654 | Bacteria; Firmicutes; Clostridia; Clostridiales; Lachnospiraceae; Lachnoclostridium; |
| | uncultured_bacterium |
| denovo657 | Bacteria; Bacteroidetes; Bacteroidia; Bacteroidales; Bacteroidaceae; Bacteroides; |
| | uncultured_bacterium |
| denovo683 | Bacteria; Firmicutes; Bacilli; Lactobacillales; Enterococcaceae; Enterococcus; |
| | unidentified_marine_bacterioplankton |
| denovo886 | Bacteria; Proteobacteria; Gammaproteobacteria; Enterobacteriales; Enterobacteriaceae; |
| | Proteus; Proteus_mirabilis |
| denovo920 | Bacteria; Firmicutes; Clostridia; Clostridiales; Clostridiaceae_1; Clostridium_sensu_stricto _1; |
| | uncultured_bacterium |
| denovo959 | Bacteria; Firmicutes; Erysipelotrichia; Erysipelotrichales; Erysipelotrichaceae; |
| | Turicibacter; uncultured_bacterium |
| denovo989 | Bacteria; Proteobacteria; Gammaproteobacteria; Enterobacteriales; Enterobacteriaceae; |
| | Klebsiella; uncultured_bacterium |
| denovo991 | Bacteria; Firmicutes; Clostridia; Clostridiales; Clostridiaceae_1; Clostridium_sensu_stricto_1; |
| | uncultured_bacterium |
| denovo1000 | Bacteria; Firmicutes; Negativicutes; Selenomonadales; Veillonellaceae; Megamonas; |
| | uncultured_bacterium |
| denovo1022 | Bacteria; Fusobacteria; Fusobacteriia; Fusobacteriales; Fusobacteriaceae; Fusobacterium; |
| | uncultured_organism |
| denovo1074 | Bacteria; Firmicutes; Clostridia; Clostridiales; Lachnospiraceae; |
| | [Ruminococcus]_torques_group; uncultured_bacterium |
| denovo1135 | Bacteria; Firmicutes; Bacilli; Lactobacillales; Lactobacillaceae; Lactobacillus; |
| | uncultured_bacterium |
| denovo1178 | Bacteria; Actinobacteria; Actinobacteria; Bifidobacteriales; Bifidobacteriaceae; |
| | Bifido bacterium; Bifidobacterium_saeculare_DSM_6531_=_LMG_14934 |
| denovo1192 | Bacteria; Firmicutes; Negativicutes; Selenomonadales; Veillonellaceae; Megamonas; |
| | uncultured_bacterium |
| denovo1220 | Bacteria; Firmicutes; Clostridia; Clostridiales; Peptostreptococcaceae; Romboutsia; |
| | uncultured_bacterium |
| denovo1327 | Bacteria; Firmicutes; Clostridia; Clostridiales; Lachnospiraceae; uncultured; |
| | uncultured_bacterium |
| denovo1402 | Bacteria; Bacteroidetes; Bacteroidia; Bacteroidales; Bacteroidaceae; Bacteroides; |
| | uncultured_bacterium |
| denovo1404 | Bacteria; Actinobacteria; Actinobacteria; Bifidobacteriales; Bifidobacteriaceae; |
| | Bifidobacterium; uncultured_bacterium |
| denovo1476 | Bacteria; Firmicutes; Clostridia; Clostridiales; Clostridiaceae_1; Clostridium_sensu_stricto_1; |
| | uncultured_Clostridium_sp. |
| denovo1484 | Bacteria; Firmicutes; Negativicutes; Selenomonadales; Veillonellaceae; Veillonella; |
| | Veillonella_magna |
| denovo1488 | Bacteria; Firmicutes; Clostridia; Clostridiales; Family_XI; Anaerococcus; |
| | uncultured_bacterium |
| denovo1678 | Bacteria; Firmicutes; Clostridia; Clostridiales; Lachnospiraceae; Epulopiscium; |
| | uncultured_bacterium |
| denovo1696 | Bacteria; Firmicutes; Clostridia; Clostridiales; Ruminococcaceae; Flavonifractor; |
| | uncultured_bacterium |
| denovo1802 | Bacteria; Firmicutes; Clostridia; Clostridiales; Clostridiaceae_1; Candidatus_Arthromitus; |
| | uncultured_bacterium |
| denovo1830 | Bacteria; Firmicutes; Clostridia; Clostridiales; Lachnospiraceae; Lachnoclostridium; |
| | uncultured_bacterium |
| denovo1987 | Bacteria; Firmicutes; Erysipelotrichia; Erysipelotrichales; Erysipelotrichaceae; |
| | Erysipelatoclostridium; uncultured_bacterium |
| denovo2011 | Bacteria; Firmicutes; Clostridia; Clostridiales; Lachnospiraceae; Blautia; |
| | uncultured_bacterium |
| denovo2050 | Bacteria; Firmicutes; Clostridia; Clostridiales; Clostridiaceae_1; Clostridium_sensu_stricto_1; |
| | uncultured_bacterium |
| denovo2108 | Bacteria; Firmicutes; Bacilli; Lactobacillales; Streptococcaceae; Streptococcus; |
| | Streptococcus_canis |
| denovo2116 | Bacteria; Firmicutes; Clostridia; Clostridiales; Peptostreptococcaceae; Peptoclostridium; |
| | [Clostridium]_hiranonis |
| denovo2124 | Bacteria; Proteobacteria; Gammaproteobacteria; Enterobacteriales; Enterobacteriaceae; |
| | Escherichia-Shigella; uncultured_bacterium |
| denovo2203 | Bacteria; Firmicutes; Clostridia; Clostridiales; Clostridiaceae_1; Clostridium_sensu_stricto_1; |
| | uncultured_bacterium |
| denovo2226 | Bacteria; Firmicutes; Clostridia; Clostridiales; Ruminococcaceae; Anaerotruncus; |
| | uncultured_bacterium |
| denovo2292 | Bacteria; Firmicutes; Clostridia; Clostridiales; Lachnospiraceae; uncultured; |
| | uncultured_organism |
| denovo2529 | Bacteria; Firmicutes; Clostridia; Clostridiales; Peptostreptococcaceae; Romboutsia; |
| | uncultured_bacterium |
| denovo2580 | Bacteria; Firmicutes; Bacilli; Lactobacillales; Streptococcaceae; Streptococcus; |
| | uncultured_bacterium |
| denovo2584 | Bacteria; Firmicutes; Negativicutes; Selenomonadales; Veillonellaceae; Veillonella; |
| | uncultured_bacterium |
| denovo2648 | Bacteria; Firmicutes; Erysipelotrichia; Erysipelotrichales; Erysipelotrichaceae; |
| | Holdemanella; uncultured_bacterium |
| denovo2834 | Bacteria; Proteobacteria; Gammaproteobacteria; Enterobacteriales; Enterobacteriaceae; |
| | Escherichia-Shigella; uncultured_bacterium |
| denovo2910 | Bacteria; Bacteroidetes; Bacteroidia; Bacteroidales; Prevotellaceae; Alloprevotella; |
| | uncultured_bacterium |
| denovo2928 | Bacteria; Firmicutes; Negativicutes; Selenomonadales; Veillonellaceae; Megamonas; |
| | uncultured_bacterium |
| denovo2930 | Bacteria; Firmicutes; Clostridia; Clostridiales; Lachnospiraceae; Tyzzerella_3; |
| | [Clostridium]_colinum |
| denovo3055 | Bacteria; Firmicutes; Clostridia; Clostridiales; Clostridiaceae_1; Clostridium_sensu_stricto_1; |
| | uncultured_bacterium |
| denovo3073 | Bacteria; Proteobacteria; Gammaproteobacteria; Enterobacteriales; Enterobacteriaceae; |
| | Escherichia-Shigella; uncultured_bacterium |
| denovo3119 | Bacteria; Firmicutes; Clostridia; Clostridiales; Lachnospiraceae; Cellulosilyticum; |
| | uncultured_bacterium |
| denovo3179 | Bacteria; Firmicutes; Clostridia; Clostridiales; Peptostreptococcaceae; Romboutsia; |
| | uncultured_organism |
| denovo3403 | Bacteria; Proteobacteria; Epsilonproteobacteria; Campylobacterales; Campylobacteraceae; |
| | Campylobacter; uncultured_bacterium |
| denovo3694 | Bacteria; Proteobacteria; Gammaproteobacteria; Pasteurellales; Pasteurellaceae; |
| | Haemophilus; uncultured_bacterium |
| denovo3749 | Bacteria; Firmicutes; Clostridia; Clostridiales; Lachnospiraceae; Lachnoclostridium; |
| | uncultured_bacterium |
| denovo3887 | Bacteria; Firmicutes; Bacilli; Lactobacillales; Lactobacillaceae; Lactobacillus; |
| | Lactobacillus_saerimneri |
| denovo3912 | Bacteria; Bacteroidetes; Bacteroidia; Bacteroidales; Bacteroidaceae; Bacteroides; |
| | uncultured_bacterium |
| denovo4020 | Bacteria; Firmicutes; Clostridia; Clostridiales; Lachnospiraceae; uncultured; |
| | uncultured_bacterium |
| denovo4069 | Bacteria; Firmicutes; Clostridia; Clostridiales; Clostridiaceae_1; Clostridium_sensu_stricto_1; |
| | uncultured_bacterium |
| denovo4100 | Bacteria; Actinobacteria; Coriobacteriia; Coriobacteriales; Coriobacteriaceae; Eggerthella; |
| | Eggerthella_lenta |
| denovo4324 | Bacteria; Firmicutes; Clostridia; Clostridiales; Lachnospiraceae; Tyzzerella_4; |
| | uncultured_bacterium |
| denovo4476 | Bacteria; Firmicutes; Clostridia; Clostridiales; Lachnospiraceae; Lachnoclostridium; |
| | uncultured_bacterium |
| denovo4638 | Bacteria; Bacteroidetes; Bacteroidia; Bacteroidales; Prevotellaceae; |
| | Prevotellaceae_Ga6A1_group; uncultured_bacterium |
| denovo4692 | Bacteria; Proteobacteria; Epsilonproteobacteria; Campylobacterales; Helicobacteraceae; |
| | Helicobacter; Helicobacter_genosp._FL56 |
| denovo4759 | Bacteria; Firmicutes; Clostridia; Clostridiales; Lachnospiraceae; |
| | [Ruminococcus]_gnavus_group; uncultured_bacterium |
| denovo4770 | Bacteria; Firmicutes; Clostridia; Clostridiales; Lachnospiraceae; Blautia; |
| | uncultured_bacterium |
| denovo4820 | Bacteria; Firmicutes; Clostridia; Clostridiales; Lachnospiraceae; Sellimonas; |
| | uncultured_bacterium |
| denovo5010 | Bacteria; Firmicutes; Clostridia; Clostridiales; Peptostreptococcaceae; Peptoclostridium; |
| | uncultured_bacterium |
| denovo5029 | Bacteria; Firmicutes; Clostridia; Clostridiales; Peptostreptococcaceae; Intestinibacter; |
| | uncultured_bacterium |
| denovo5065 | Bacteria; Fusobacteria; Fusobacteriia; Fusobacteriales; Fusobacteriaceae; |
| | Fusobacterium; uncultured_organism |
| denovo5077 | Bacteria; Firmicutes; Bacilli; Lactobacillales; Lactobacillaceae; Lactobacillus; |
| | uncultured_bacterium |
| denovo5125 | Bacteria; Firmicutes; Clostridia; Clostridiales; Ruminococcaceae; |
| | Ruminococcaceae_UCG-014; uncultured_bacterium |
| denovo5198 | Bacteria; Firmicutes; Clostridia; Clostridiales; Lachnospiraceae; Blautia; |
| | uncultured_bacterium |
| denovo5255 | Bacteria; Firmicutes; Clostridia; Clostridiales; Clostridiaceae_1; Clostridium_sensu_stricto_1; |
| | uncultured_bacterium |
| denovo5282 | Bacteria; Proteobacteria; Betaproteobacteria; Burkholderiales; Alcaligenaceae; |
| | Parasutterella; uncultured_bacterium |
| denovo5343 | Bacteria; Firmicutes; Negativicutes; Selenomonadales; Veillonellaceae; Megamonas; |
| | uncultured_bacterium |
| denovo5401 | Bacteria; Firmicutes; Clostridia; Clostridiales; Ruminococcaceae; |
| | Ruminococcaceae_UCG-014; uncultured_bacterium |
| denovo5480 | Bacteria; Firmicutes; Negativicutes; Selenomonadales; Veillonellaceae; Veillonella; |
| | Veillonella_sp._oral_clone_VeillE3 |
| denovo5706 | Bacteria; Firmicutes; Clostridia; Clostridiales; Lachnospiraceae; Tyzzerella; |
| | uncultured_organism |
| denovo5855 | Bacteria; Firmicutes; Clostridia; Clostridiales; Peptostreptococcaceae; Romboutsia; |
| | uncultured_bacterium |
| denovo5873 | Bacteria; Firmicutes; Clostridia; Clostridiales; Lachnospiraceae; Anaerostipes; |
| | uncultured_bacterium |
| denovo6118 | Bacteria; Proteobacteria; Gammaproteobacteria; Enterobacteriales; Enterobacteriaceae; |
| | Providencia; uncultured_bacterium |
| denovo6511 | Bacteria; Firmicutes; Clostridia; Clostridiales; Lachnospiraceae; uncultured; |
| | uncultured_organism |
| denovo6738 | Bacteria; Firmicutes; Clostridia; Clostridiales; Lachnospiraceae; Roseburia; |
| | uncultured_organism |
| denovo6823 | Bacteria; Firmicutes; Clostridia; Clostridiales; Peptostreptococcaceae; uncultured; |
| | uncultured_bacterium |
| denovo6858 | Bacteria; Actinobacteria; Coriobacteriia; Coriobacteriales; Coriobacteriaceae; Collinsella; |
| | uncultured_bacterium |
| denovo6879 | Bacteria; Actinobacteria; Actinobacteria; Bifidobacteriales; Bifidobacteriaceae; |
| | Bifidobacterium; uncultured_bacterium |
| denovo7211 | Bacteria; Proteobacteria; Gammaproteobacteria; Enterobacteriales; Enterobacteriaceae; |
| | Klebsiella; Shigella_dysenteriae |
| denovo7257 | Bacteria; Proteobacteria; Gammaproteobacteria; Pasteurellales; Pasteurellaceae; |
| | Haemophilus; uncultured_bacterium |
| denovo7291 | Bacteria; Firmicutes; Bacilli; Lactobacillales; Streptococcaceae; Streptococcus; |
| | uncultured_bacterium |
| denovo7373 | Bacteria; Firmicutes; Clostridia; Clostridiales; Lachnospiraceae; uncultured; |
| | unidentified |
| denovo7504 | Bacteria; Firmicutes; Bacilli; Bacillales; Staphylococcaceae; Staphylococcus; |
| | uncultured_bacterium |
| denovo7649 | Bacteria; Firmicutes; Clostridia; Clostridiales; Family_XI; Finegoldia; |
| | uncultured_Finegoldia_sp. |
| denovo7915 | Bacteria; Firmicutes; Clostridia; Clostridiales; Family_XI; Peptoniphilus; |
| | uncultured_bacterium |
| denovo7972 | Bacteria; Actinobacteria; Actinobacteria; Bifidobacteriales; Bifidobacteriaceae; |
| | Bifidobacterium; uncultured_actinobacterium |
| denovo8295 | Bacteria; Firmicutes; Clostridia; Clostridiales; Lachnospiraceae; Roseburia; |
| | uncultured_organism |
| denovo8302 | Bacteria; Firmicutes; Bacilli; Lactobacillales; Lactobacillaceae; Lactobacillus; |
| | uncultured_bacterium |
| denovo8443 | Bacteria; Firmicutes; Clostridia; Clostridiales; Clostridiaceae_1; Clostridium_sensu_stricto_1; |
| | uncultured_bacterium |
| denovo8456 | Bacteria; Firmicutes; Negativicutes; Selenomonadales; Veillonellaceae; Veillonella; |
| | uncultured_bacterium |
| denovo8600 | Bacteria; Fusobacteria; Fusobacteriia; Fusobacteriales; Fusobacteriaceae; Fusobacterium; |
| | uncultured_bacterium |
| denovo8725 | Bacteria; Firmicutes; Clostridia; Clostridiales; Lachnospiraceae; Lachnoclostridium; |
| | uncultured_bacterium |
| denovo8737 | Bacteria; Firmicutes; Clostridia; Clostridiales; Lachnospiraceae; uncultured; unidentified |
| denovo8845 | Bacteria; Firmicutes; Clostridia; Clostridiales; Lachnospiraceae; Tyzzerella_3; |
| | uncultured_bacterium |
| denovo8862 | Bacteria; Proteobacteria; Betaproteobacteria; Burkholderiales; Alcaligenaceae; Sutterella; |
| | uncultured_bacterium |
| denovo8911 | Bacteria; Firmicutes; Negativicutes; Selenomonadales; Veillonellaceae; Veillonella; |
| | uncultured_bacterium |
| denovo8973 | Bacteria; Firmicutes; Bacilli; Lactobacillales; Streptococcaceae; Streptococcus; |
| | uncultured_bacterium |
| denovo9448 | Bacteria; Firmicutes; Bacilli; Lactobacillales; Lactobacillaceae; Pediococcus; |
| | uncultured_organism |
| denovo9465 | Bacteria; Firmicutes; Erysipelotrichia; Erysipelotrichales; Erysipelotrichaceae; Turicibacter; |
| | uncultured_bacterium |
| denovo9596 | Bacteria; Proteobacteria; Gammaproteobacteria; Pasteurellales; Pasteurellaceae; |
| | Haemophilus; Haemophilus_haemoglobinophilus |
| denovo10082 | Bacteria; Firmicutes; Negativicutes; Selenomonadales; Veillonellaceae; Megamonas; |
| | uncultured_bacterium |
| denovo10107 | Bacteria; Firmicutes; Clostridia; Clostridiales; Ruminococcaceae; Butyricicoccus; |
| | uncultured_bacterium |
| denovo10120 | Bacteria; Firmicutes; Clostridia; Clostridiales; Lachnospiraceae; |
| | [Ruminococcus]_torques_group; uncultured_bacterium |
| denovo10185 | Bacteria; Firmicutes; Negativicutes; Selenomonadales; Veillonellaceae; Veillonella; |
| | uncultured_bacterium |
| denovo10268 | Bacteria; Firmicutes; Clostridia; Clostridiales; Lachnospiraceae; Lachnoclostridium; |
| | unculturedbacterium |
| denovo10279 | Bacteria; Firmicutes; Clostridia; Clostridiales; Lachnospiraceae; Blautia; |
| | uncultured_bacterium |
| denovo10356 | Bacteria; Firmicutes; Bacilli; Lactobacillales; Enterococcaceae; Enterococcus; |
| | uncultured_bacterium |
| denovo10534 | Bacteria; Firmicutes; Bacilli; Lactobacillales; Lactobacillaceae; Lactobacillus; |
| | uncultured_bacterium |
| denovo10565 | Bacteria; Firmicutes; Bacilli; Lactobacillales; Streptococcaceae; Streptococcus; |
| | Streptococcus_sp._C8I9 |
| denovo10566 | Bacteria; Firmicutes; Clostridia; Clostridiales; Clostridiaceae_1; Clostridium_sensu_stricto_1; |
| | uncultured_bacterium |
| denovo10663 | Bacteria; Fusobacteria; Fusobacteriia; Fusobacteriales; Fusobacteriaceae; Fusobacterium; |
| | uncultured_bacterium |
| denovo10707 | Bacteria; Fusobacteria; Fusobacteriia; Fusobacteriales; Fusobacteriaceae; Fusobacterium; |
| | uncultured_Clostridium_sp. |
| denovo10831 | Bacteria; Fusobacteria; Fusobacteriia; Fusobacteriales; Fusobacteriaceae; Fusobacterium; |
| | uncultured_organism |
| denovo11006 | Bacteria; Firmicutes; Clostridia; Clostridiales; Lachnospiraceae; Blautia; |
| | uncultured_bacterium |
| denovo11009 | Bacteria; Proteobacteria; Gammaproteobacteria; Pasteurellales; Pasteurellaceae; |
| | Haemophilus; Pasteurellaceae_bacterium_canine_oral_taxon_272 |
| denovo11016 | Bacteria; Firmicutes; Clostridia; Clostridiales; Lachnospiraceae; |
| | Lachnospiraceae_NK4A136_group; uncultured_bacterium |
| denovo11369 | Bacteria; Firmicutes; Bacilli; Lactobacillales; Lactobacillaceae; Lactobacillus; |
| | uncultured_bacterium |
| denovo11380 | Bacteria; Firmicutes; Clostridia; Clostridiales; Lachnospiraceae; Blautia; |
| | uncultured_bacterium |
| denovo11572 | Bacteria; Bacteroidetes; Bacteroidia; Bacteroidales; Prevotellaceae; Prevotella_9; |
| | uncultured_bacterium |
| denovo11581 | Bacteria; Firmicutes; Clostridia; Clostridiales; Ruminococcaceae; uncultured; |
| | uncultured_bacterium |
| denovo11693 | Bacteria; Fusobacteria; Fusobacteriia; Fusobacteriales; Fusobacteriaceae; Fusobacterium; |
| | uncultured_bacterium |
| denovo11744 | Bacteria; Firmicutes; Erysipelotrichia; Erysipelotrichales; Erysipelotrichaceae; |
| | [Clostridium]_innocuum_group; uncultured_bacterium |
| denovo11790 | Bacteria; Firmicutes; Clostridia; Clostridiales; Ruminococcaceae; Faecalibacterium; |
| | uncultured_bacterium |
| denovo11942 | Bacteria; Firmicutes; Clostridia; Clostridiales; Clostridiaceae_1; |
| | Clostridium_sensu_stricto_1; uncultured_bacterium |
| denovo12042 | Bacteria; Firmicutes; Clostridia; Clostridiales; Peptostreptococcaceae; Terrisporobacter; |
| | uncultured_bacterium |
| denovo12057 | Bacteria; Firmicutes; Clostridia; Clostridiales; Lachnospiraceae; |
| | [Ruminococcus]_gauvreauii_group; uncultured_bacterium |
| denovo12145 | Bacteria; Firmicutes; Clostridia; Clostridiales; Lachnospiraceae; Blautia; |
| | uncultured_bacterium |
| denovo12176 | Bacteria; Firmicutes; Bacilli; Lactobacillales; Streptococcaceae; Streptococcus; |
| | Streptococcus_minor |
| denovo12209 | Bacteria; Firmicutes; Clostridia; Clostridiales; Lachnospiraceae; uncultured; |
| | uncultured_bacterium |
| denovo12346 | Bacteria; Proteobacteria; Gammaproteobacteria; Pasteurellales; Pasteurellaceae; |
| | Frederiksenia; uncultured_bacterium |
| denovo12377 | Bacteria; Firmicutes; Clostridia; Clostridiales; Ruminococcaceae; Butyricicoccus; |
| | uncultured_Clostridiales_bacterium |
| denovo12400 | Bacteria; Proteobacteria; Gammaproteobacteria; Oceanospirillales; Halomonadaceae; |
| | uncultured_Halomonas_sp. |

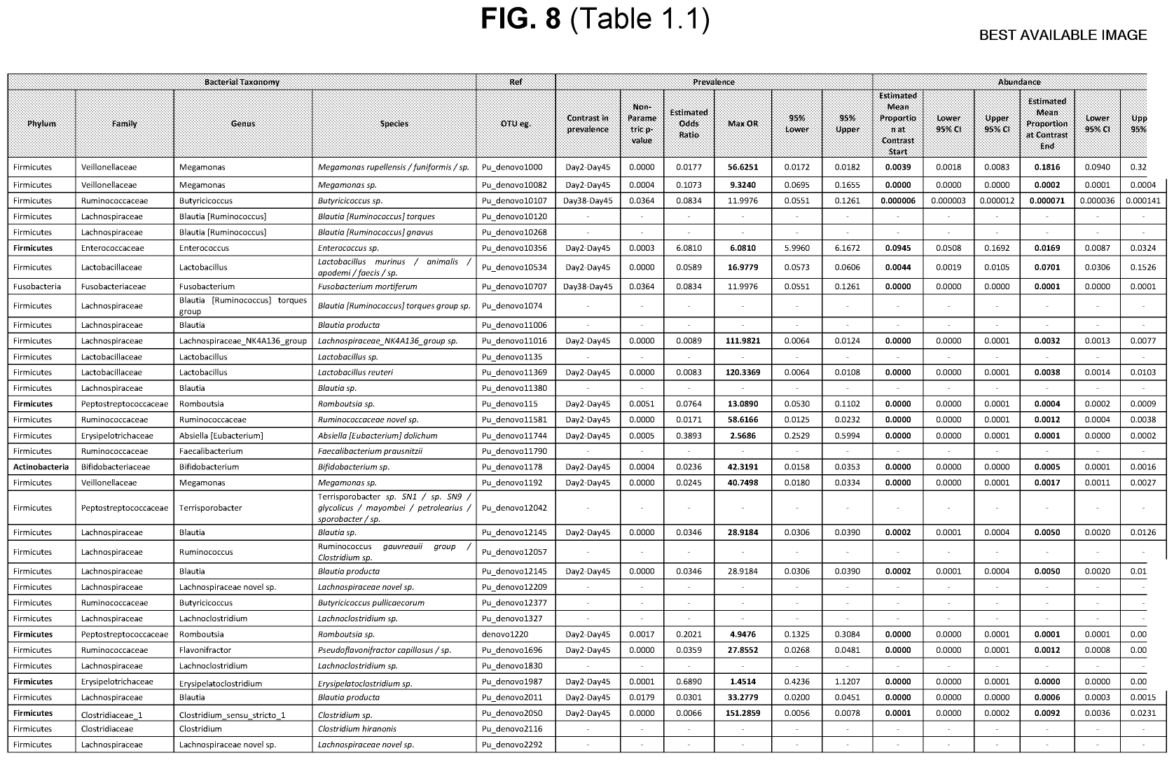

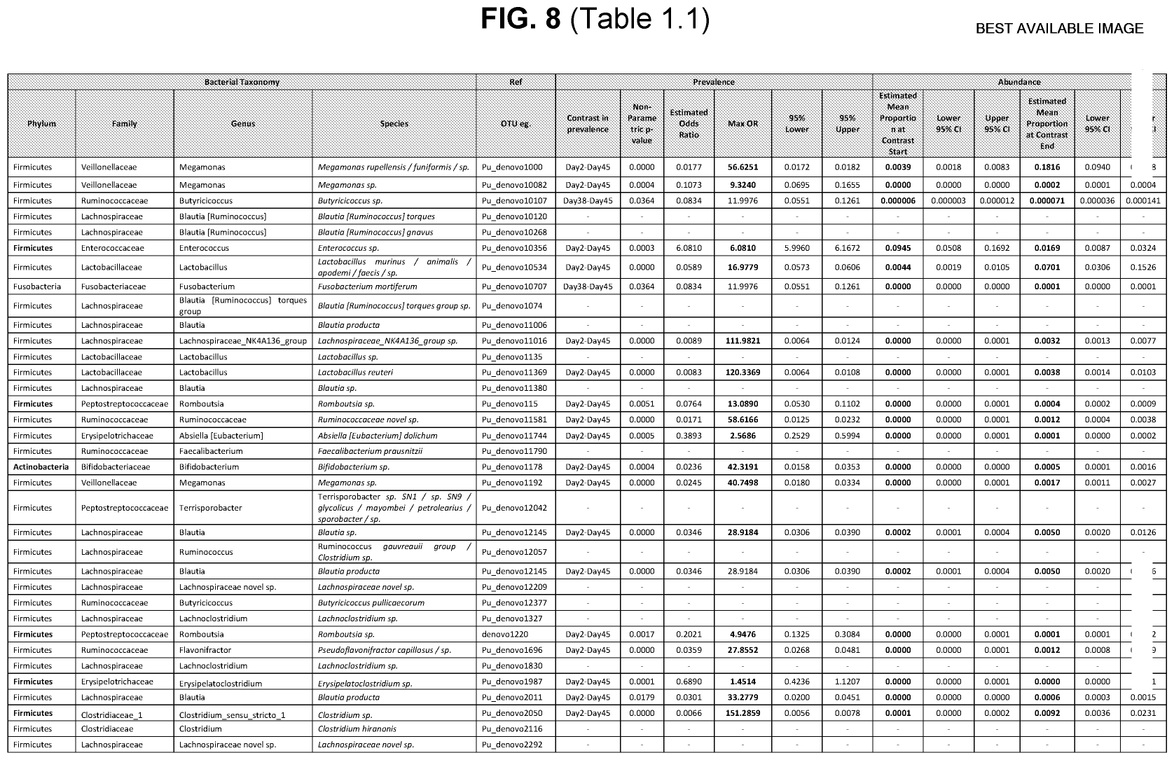

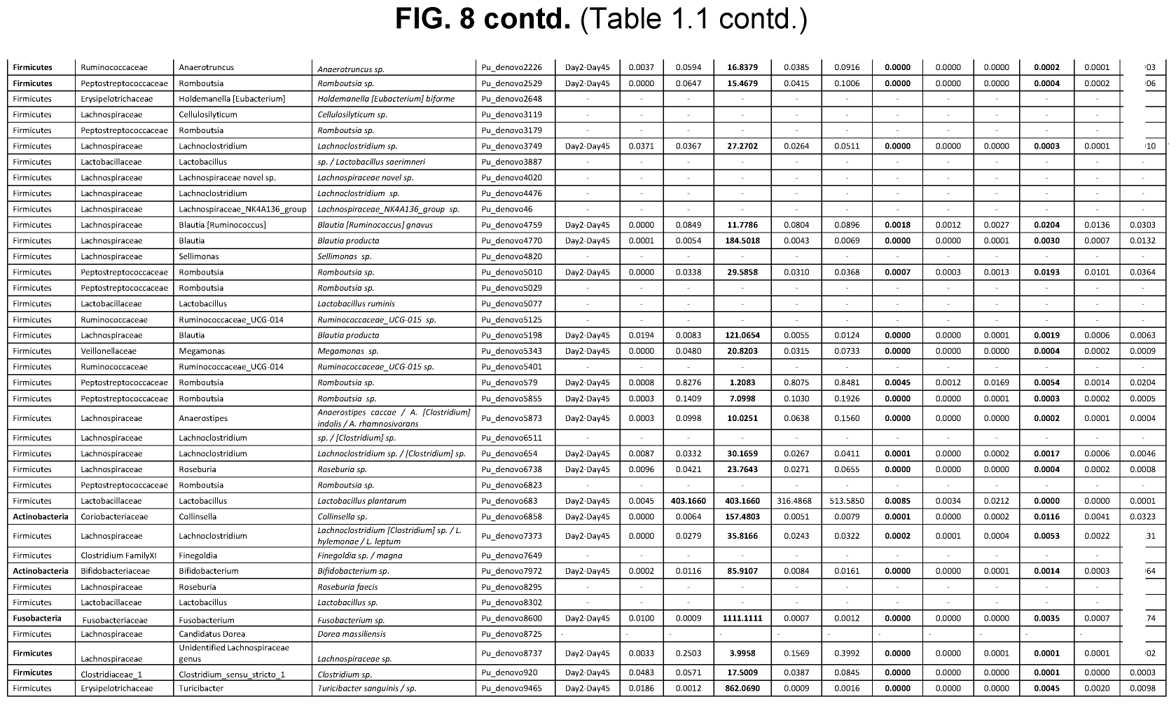

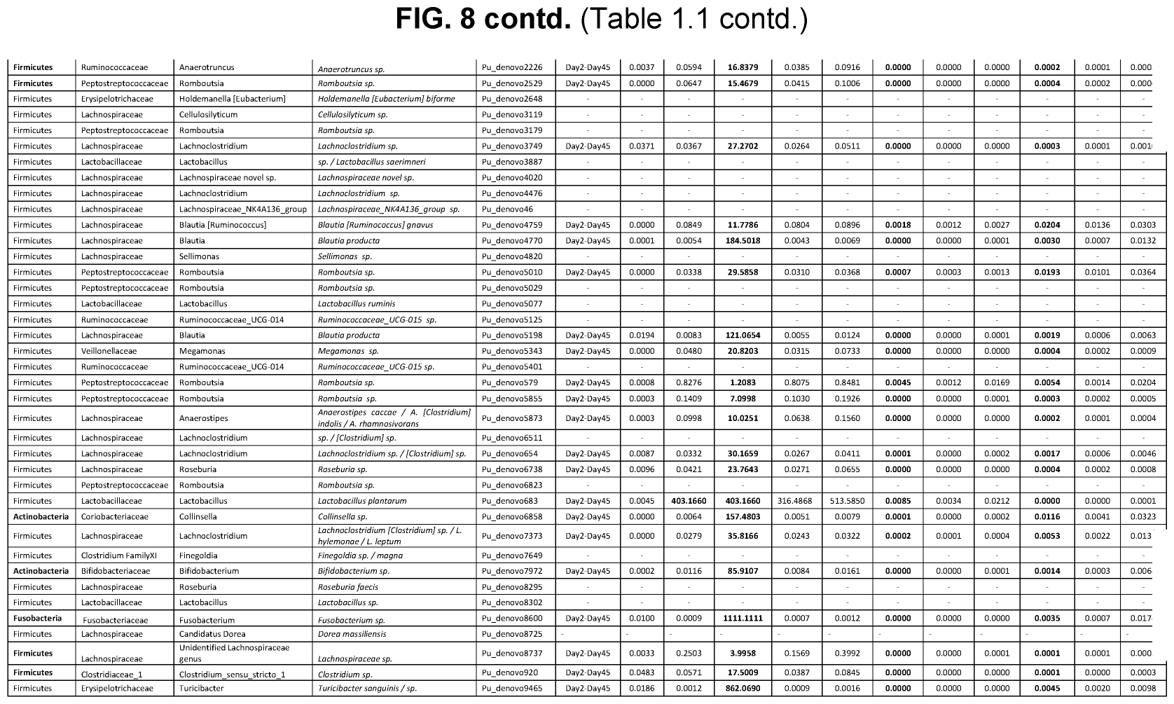

To further investigate the bacterial taxa (OTUs) detected in the puppy microbiota immediately after birth and during the receipt of colostrum and maternal milk postpartum, samples from puppies 2 days after birth were assessed for taxonomic designations in the faecal microbiota. These analyses demonstrated a high proportion of taxa in terms of species richness, that have previously been detected in healthy controls from studies of the microbiota in other mammals and hence can be considered to be associated with health in puppies (see FIG. 8 (Table 1.1) and Table 1.2 (below)). Out of a total of 141 taxa (OTUs) representing individual species, 61 (43%) were identified as bacterial species (mostly novel species) of genera associated with health in mammals or other animals.

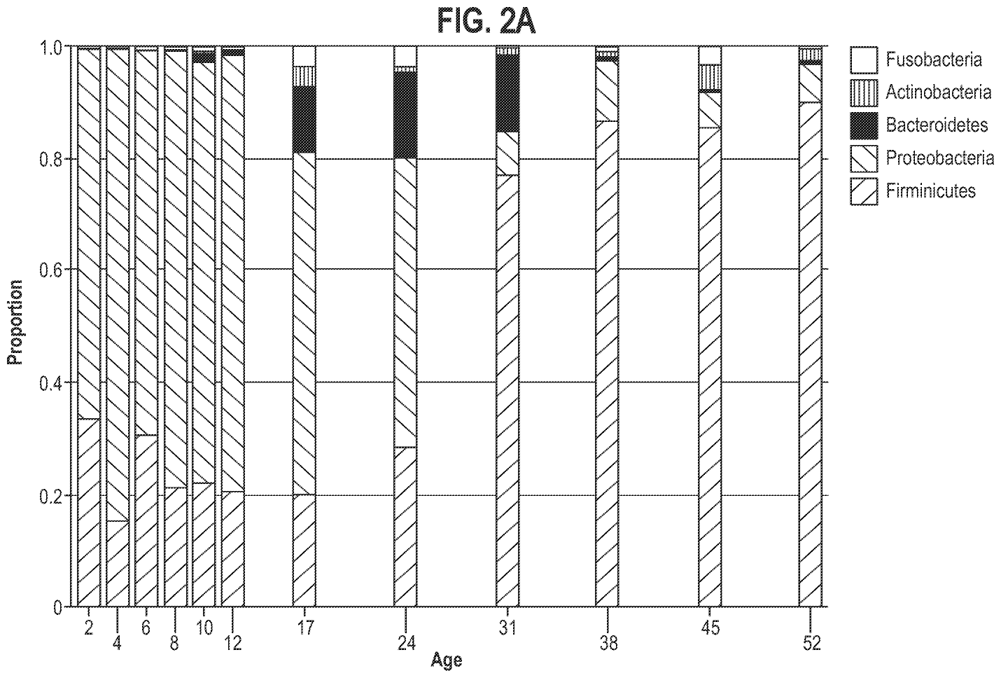

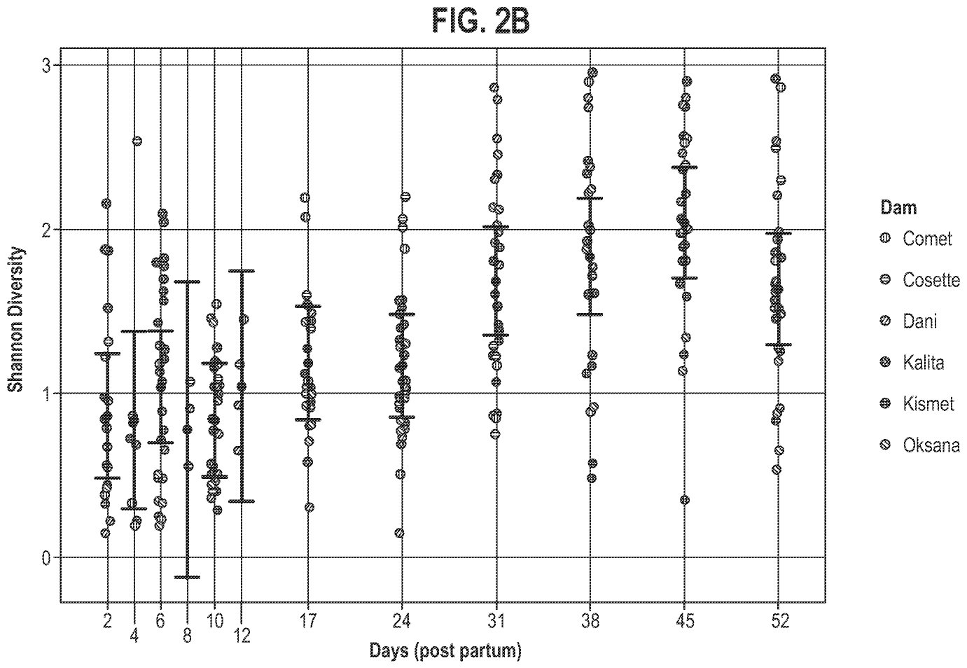

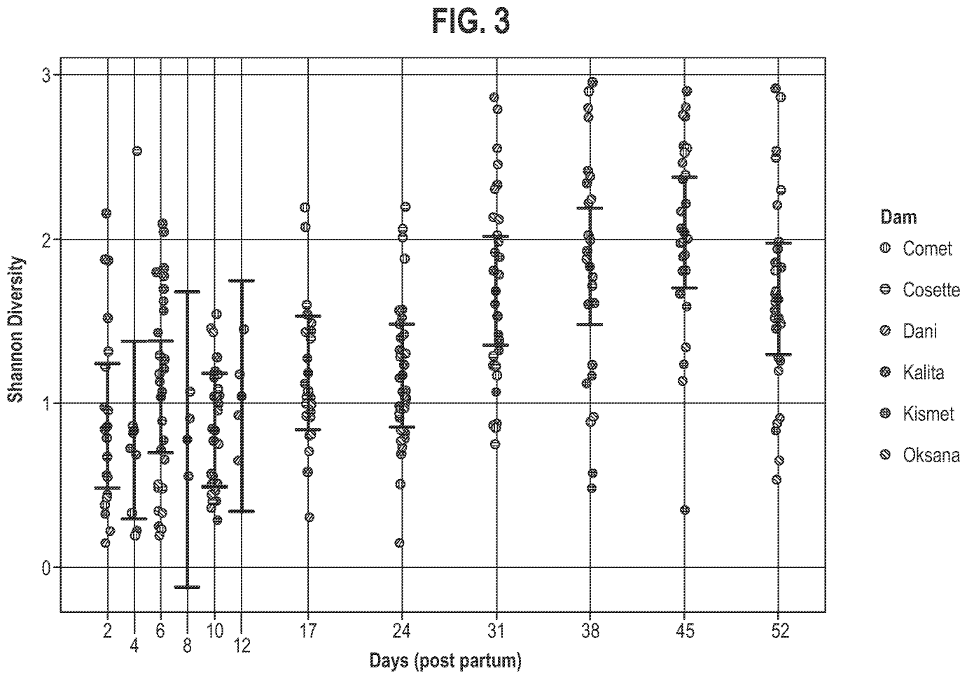

Graphical representations of the phyla represented in faeces suggested an apparent shift in the proportions of phyla detected and Shannon diversity of the faecal microbiota (FIGS. 2A and 2B). Similarly to humans the major shift in the microbiota in puppies was observed at weaning (days 19-35). The mean abundance and range of those taxa associated with health in humans and other mammals was assessed, to determine whether significant contrasts in the relative abundances were observed between day 2 (pre-weaning, earliest timepoint after birth and during receipt of colostrum/lactation) and day 45 (post weaning; see FIG. 8 (Table 1.1); see Figures). The direction of contrast and size/degree of contrasts are considered indicative of biologically relevant differences in population abundance. The magnitude of contrasts in these timepoints gives context for shifts or differences between individuals in these taxa that can have implications for health in puppies and young adult dogs.

### CONCLUSIONS

Taken together, the identification of a high proportion of taxa closely related to those associated with healthy controls across various conditions in other mammals (FIG. 8 (Table 1.1)) and additionally the observed progression of the puppy microbiota over time from birth to a more diverse community structure (FIG. 2B) are suggestive of the compositional factors associated with health in dogs. The compositional characteristics and degree of change occurring over time in the bacterial species making up the microbiome of healthy puppies, can be used in the context of health associated taxa in other mammals to inform optimal levels of both bacterial taxa and measures of diversity in puppies and in dogs post-weaning as described below in the methods of the present disclosure.

### Methods

The method involves the extraction of DNA from a freshly produced faecal sample by a means such as the QIAamp Power Faecal DNA kit (Qiagen) and subsequently the use of molecular biology techniques to assess the detection rate and abundance of the combination of the bacterial taxa or DNA sequences described below and in FIG. 8 (Table 1.1) and Table 1.2 (below) as well as biomarkers for those organisms compared to standardised healthy control samples from animals of the same (microbiome) lifestage according to the results of these studies (preweaned puppies days 2-24 post-partum or weaned puppies 24-52 days post-partum). Comparison can also be made to animals of the same ‘microbiome lifestage’ with chronic gastrointestinal enteropathy, IBD, acute diarrhoea and chronic diarrhoea. The interpretation of health status is then made based on the combination and relative abundance of the health associated organisms detected in the faeces of the dogs allow the assessment of health status of the microbiome and indicate how the health of the microbiome can be enhanced in terms of the direction and magnitude of change in the gut microbiota (See FIG. 9 (Table 1.3); see Figures).

Assessment of the microbiome components observed in the faeces of the dog can be undertaken at an individual point in time for assessment against healthy and unhealthy clinical controls in the same lifestage to receive a description of the health of the microbiome at a specific timepoint. Alternatively, the gastrointestinal health of the dog can be monitored over time by assessment of the gut microbiome periodically at intervals such as 6 monthly or one yearly tests/assessments or following particular events such as gastrointestinal upset or travel. The results of detection and relative abundance of the microbial species associated with health (or with the disease condition) can then be compared with the previous results or cumulative (averaged) results from the previous assessments of the microbiome from the individual dog. In the case of longitudinal assessment of an individual over time, adjustments must be made as the animal crosses from one microbiome lifestage to the next by additional comparisons to control cohorts such as provided within the data reported here.

After DNA extraction from freshly produced faeces and sequencing of the DNA by techniques such as 16 S rDNA amplicon, shotgun, metagenome, Illumina, nanopore or other DNA sequencing techniques, the resulting DNA sequences are clustered to species (>98% ID) level. Assessment of the relative abundance of the sequences descriptive of the organisms in table 1.1 or DNA sequences within 95% identical to those in Table 1.2 or of other DNA, RNA or protein sequences or biomarkers of those species specified in FIG. 8 (Table 1.1) and Table 1.2 is made. Briefly, sequence data obtained from the test sample is clustered into groups of sequences with from about 98% to 100% identity and a reference sequence from the clusters which represent>0.001% of the total sequences is then used to either 1) assign taxonomy or function through database homologues or to determine the nature of the biomarker through homology searches of DNA databases such as the Greengenes or Silva or the NCBI non-redundant nucleotide sequence database for comparison to known DNA sequences for species held within the databases or 2) compared to the DNA sequences given in Table 1.2.

The number and abundance of the organisms, sequences or biomarkers identified from within the bacterial combinations described in FIG. 8 (Table 1.1) and Table 1.2 are then used to compare to the same data number of organisms and abundance of the individual and total load of the health associated species described in FIG. 8 (Table 1.1) or possessing DNA sequences within 97% Identity to those in Table 1.2, according to the parameters described in FIG. 9 (Table 1.3).

### Example 2: Species for Detecting the Health of the Gut Microbiome in Adult and Senior Dogs

### Summary

The faecal microbiota was assessed in a cohort of 41 adult Beagle dogs aged between 3.8 and 15.0 years to determine the characteristics of the gut microbiota in healthy adult and mature dogs. The study cohort included 13 animals assigned to the adult group (aged 3.8-6.2 years), 20 dogs assigned to the senior group (aged 8.2-12.9 years) and 8 dogs assigned to the geriatric group (aged 14.6-15.0 years).

### BACKGROUND

The gastrointestinal (GI) microbiota is linked to the development of ‘normal’ gut histology during growth and development, whilst an altered gut histology has been reported in aging pets including in dogs and rodents. Aging is associated with an increased incidence of GI pathologies including infection, neoplasia, or other inflammatory conditions. Reported physiological alterations in digestive function associated with advancing age includes slower GI transit, altered enzymatic activity and reduced bile secretions [42]. Histological changes also occur in the gut with aging including reduced duodenal villus surface area, lower jejunal villus height, and greater colonic crypt depth [43]. Whether the full range of age-related changes in digestion and absorption of nutrients recognized in humans [44] also affects pet animals remains unclear.

Similarly to the understanding of gastrointestinal physiology in aging, human research conducted over the last decade has uncovered associations between aging and alterations in the gut microflora. More recently high-throughput sequencing (HTS) and specialised DNA array technologies have yielded further evidence of links between the microbiome and healthy longevity. The most noticeable feature in the microbiota of elderly humans is an alteration in the relative proportions of the Firmicutes and the Bacteroidetes, with the elderly having a higher proportion of Bacteroidetes while young adults have higher proportions of Firmicutes [45]. Significant decreases in bifidobacteria, Bacteroides, and Clostridium cluster XIV have also been reported to be associated with aging in humans [46]. Changes occurring in the microbiota during aging can be related to the health of the host and van Tongeren et al. (2005) [47] studied the relationship between microbial diversity and frailty scores in elderly humans.

The relationship between diet, host health, environment, and the gut microbiota in humans was studied by Claesson et al. (2012) [48] and associations were observed between microbial diversity, the functional independence measure (FIM), the Barthel index (used to evaluate performance in daily routine activities) and nutrition. Decreased microbial diversity correlated with increased frailty, decreased diet diversity and health parameters, and with increased levels of inflammatory markers. Individuals living in the community had the most diverse microbiota and were ‘healthier’ as compared to those in short- or long-term residential care. This reduced diversity associated with aging was also identified by Biagi et al., (2010) [49] in centenarians, though Bacteroidetes and Firmicutes remained the dominant phyla, with enrichment for potentially pathogenic Proteobacteria in older subjects.

The objective of this study was to determine whether differences exist in the microbiota of healthy adult, senior and geriatric dogs. The primary endpoints of interest for the analysis were microbial diversity and community composition as measured by relative taxon abundance at species level (98% 16 S rDNA sequence identity) across life stage groups.

### Methods

### Study Design

A cross-sectional study employing contrasts between groups to assess the composition of faecal bacterial populations as a marker of the gut microbiota was conducted in a cohort of 41 Beagle dogs aged between 3.8 and 15.0 years. The study was conducted at the Mars Inc. Pet Health and Nutrition Centre (PHNC, Lewisburg, Ohio, USA). Animals were assigned to one of three groups. Animal assignment to group was based on age with specific groups determined through evidence-based aging research, in which data from Banfield hospital visits and the resulting veterinary diagnoses were analysed and correlations between diagnoses and the age of the attending dogs were investigated (Salt and Saito, submitted; see also Table 5). Life stage groups were defined as adult (target age range 3-6 years), senior (target age range 9.5-12 years) and geriatric (target age range 14+ years) dogs. All Beagle dogs were fed a consistent commercial dry kibble diet (Royal Canin medium adult 7+ dog; BO189205) for a period of 30 days and freshly defaecated faecal samples were collected from each individual dog at days 21, 24 and 28 producing biological triplicate samples. Animals were housed in pen pairs overnight and were maintained in social paddock groups during the day.

### Animals

All animals received a veterinary health check to determine suitability for inclusion prior to the start of the study. The cohort of 41 adult pure-bred Beagle dogs that were assigned to the study were aged between 3.8 and 15.0 years. The study cohort included 13 animals assigned to the adult group (aged 3.8 to 6.2 years), 20 dogs assigned to the senior group (aged 8.2 to 12.9 years) and 8 dogs assigned to the geriatric group (aged 14.6 to 15.0 years). Dogs were provided with access to fresh drinking water at all times and were socialised and exercised consistently throughout the study according to standard practices for the PHNC facility.

### Diet

During the 30 days of the study all dogs received the same dry kibble commercially available mainmeal diet (Royal Canin medium adult 7+ dog; BO189205) that met AAFCO minimum standards. Additionally a 10 g bolus of wet dog food (Royal Canin;—BO188237) was fed daily to all dogs within the cohort to facilitate feeding of pills/medication in those dogs with active veterinary prescriptions. Dogs were fed at energy levels (mean energy requirements; MER) to maintain a healthy body condition score (BCS) and bodyweight. The aim was to restrict any fluctuations in bodyweight to within +/−5% throughout the study. Food portions were offered to a total of 100% of the animal's daily MER with treats offered from the main meal diet portion and with the remaining diet fed in two approximately equal (˜50% MER) portions twice a day in the morning and the afternoon.

### Wellbeing

Dogs were familiarised to study personnel and continued with their normal routine, activities and management protocols throughout the study. Animals were housed, received paddock exercise and were exercised outside of paddocks within their study cohorts. Habituation and training procedures followed the standard PHNC care package and animals were socialised with human carers for a minimum of 1 hour each day. Unsupervised meet and greets with other Beagle dogs were incorporated into activities during the whole duration of the study as standard for PHNC. Veterinary prescribed medications were given to the dogs as per standard husbandry procedures and in line with the appropriate prescription within the 10 g wet food bolus.

### Data Collection

During the study, data on the following co-variates were collected for inclusion in analyses to establish whether any contrasts existed between groups (i.e., were associated with adult, senior or geriatric life stages).
Daily and overnight faeces scores per pair*
Daily food intake
Bodyweight and body condition score
   *All collected faeces were scored using the WALTHAM 17-point faeces quality scale and incidences of poor faeces (outside of the acceptable range 1.5-3.75) were recorded.

### Faeces Sample Collection and Processing

Fresh faecal samples were collected with the samples collected frequently representing the first defaecation of the day to ensure the sample was secured. The majority of samples were freshly produced in grass paddocks. Samples were collected immediately, no more than 15 minutes after defecation. Following collection, faeces were portioned into 6 aliquots of 400 mg faeces in sterile 2 ml Lo-Bind Eppendorf tubes. Samples were stored at −80 degrees centigrade.

A 100 mg portion of the faeces was removed and DNA extraction was conducted using the QIAamp Power Faecal DNA kit (Qiagen, UK) according to the manufacturer's instructions. Following DNA extraction, DNA yields achieved per sample were determined by standard nanodrop DNA quantification methods. Faecal DNA was then diluted 1:10 prior to preparation of Illumina high throughput DNA sequencing libraries by PCR amplification of the 16 SrDNA locus (V4-6 region; Fadrosh et al., 2014) using dual indexed primers (319F: CAAGCAGAAG ACGGCATACG AGATGTGACT GGAGTTCAGA CGTGTGCTCT TCCGATCT and 806R: AATGATACGG CGACCACCGA GATCTACACT CTTTCCCTAC ACGACGCTCT TCCGATCT).

DNA sequencing of the amplified DNA libraries was conducted by Eurofins Applied Genomics Laboratory (Eurofins Genomics; Anzinger Str. 7a; 85560 Ebersberg; Germany) using a Miseq Illumina system (chemistry v.3; 2×300 bp paired end sequencing) at a depth of 160 samples/run. DNA libraries were provided in 30 ul volumes to Eurofins. Samples were quantified by Eurofins Genomics and pooled prior to loading, library pool concentrations were determined prior to processing to optimise Illumina channel loading. Data were supplied electronically.

The resulting DNA sequences were clustered into operational taxonomic units at 98% identity approximately representative of species and abundant taxa (representing>0.001 of the total sequences) were then assessed for their relative proportions. The taxonomic groups of bacteria represented by the sequences detected were determined by interrogation of the Greengenes or Silva v132 16 S rDNA databases. Comparison of the taxonomic group to organisms associated with health and disease in other mammals was utilised to highlight bacterial taxa present in the dog and representative of health of the microbiome.

Quality thresholds of a minimum of 1,000 sequence reads per sample were defined and where sequence data did not reach this level it was removed from the analysis. Sequence data was de-noised to remove chimeras and was clustered into putative taxa based on 98% sequence identity using the WALTHAM bioinformatics analysis pipeline. The resulting operational taxonomic unit (OTU) data was reduced to the non-rare portion through the removal of taxa representing<0.05% of the sequences in <2 animals from any one group. Following reduction to the non-rare portion of the population, the identification of OTUs based on a single taxon reference sequence selected as the most representative sequence of the cluster was analysed again through the WALTHAM bioinformatics analysis pipeline. Through the pipeline taxon reference sequences were used to interrogate the curated Greengenes (McDonald et al., 2012) and Silva (release 132; Yilmaz et al., 2014) databases to identify sequences in these databases with similarity criteria within 98% identity compared to the non-rare taxon reference sequences. Taxonomic assignments were then made based on sequence identity to the top database hit having first assessed the top hit against the top 10 hits resulting from database searches for each reference sequence. Greengenes taxonomic assignments were considered to be the most accurate (Personal communication Z. Lonsdale/A. Cawthrow) and hence in the case of discrepancies between searches the Greengenes assignments were used. Additionally OTU reference sequences were used to interrogate Greengenes and Silva (R 132) databases for entries with a 98% identity threshold such that only entries apparently representing the same species were returned as results.

### Statistical Methods

Preliminary exploratory analyses were performed using principal components analysis (PCA) and t-distributed stochastic neighbour embedding (t-SNE) to reduce the dimension of the data and visually represent groups. Shannon diversity was calculated for each sample and modelled using a linear mixed effects model with a fixed effect of age group and random intercept of pet. Pairwise comparisons of the life stage groups were performed with a controlled familywise error rate of 5%.

Prior to individual modelling of the bacterial OTUs which approximately represented individual species, rare OTUs were identified as those with a mean proportion of less than 0.05% and present in two or fewer samples from a single age group. After identification, rare OTUs were combined to create a single group. The relative abundance compared to the sample total for each clustered OTU, and for the combined rare group, was analysed individually using a generalised linear mixed effects model (GLMM) with a binomial distribution and logit link function. In the model, counts and total counts represented the response variables including life stage group as a fixed effect, with a random intercept of dog to account for the repeated measurements. All pairwise comparisons were performed between life stage groups using a permutation test permuting the group indicator for each pet. A familywise error rate of 5% was maintained using multiple comparisons correction. The associated primary measures were analysed with linear and generalised linear models, with random effects in the cases where repeated measures were taken per pet. A supervised dimension reduction and regression method, partial least squares discriminate analysis (PLS-DA) was used to relate these primary measures to the taxon abundance data.

### Results

Clustering of DNA sequences representative of bacterial taxa at 98% identity resulted in the identification of 10,872 species level OTUs. This total was reduced to 119 species level OTUs after removal of the rare OTUs to a pseudo group of ‘rare taxa’. Individual analysis of rare OTUs was not conducted since these taxa represented less than 0.05% of the sequences in less than two individuals from any single group.

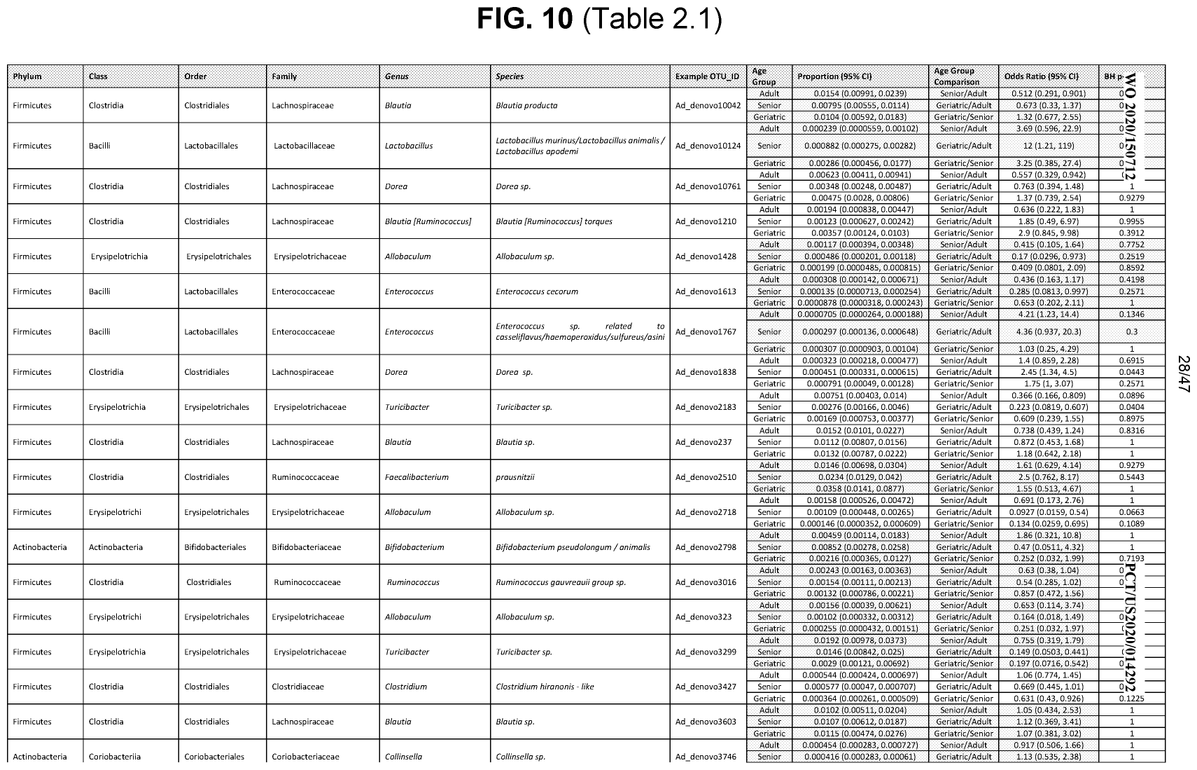

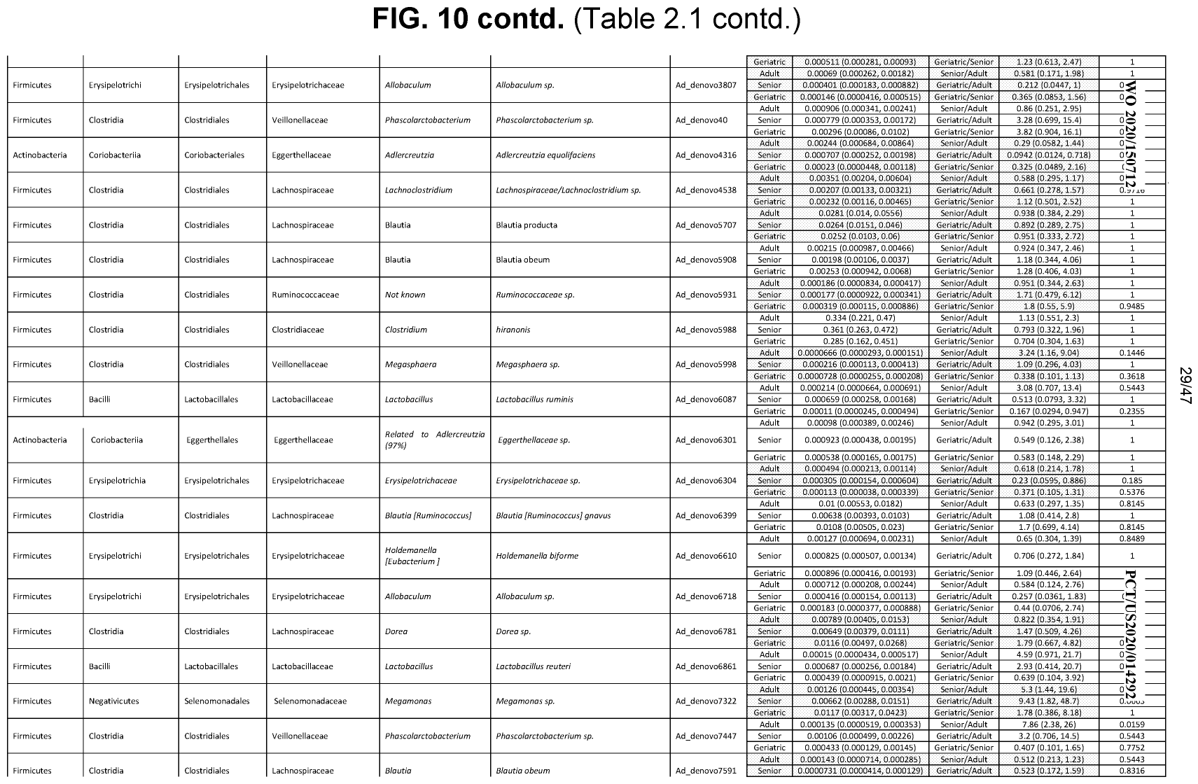

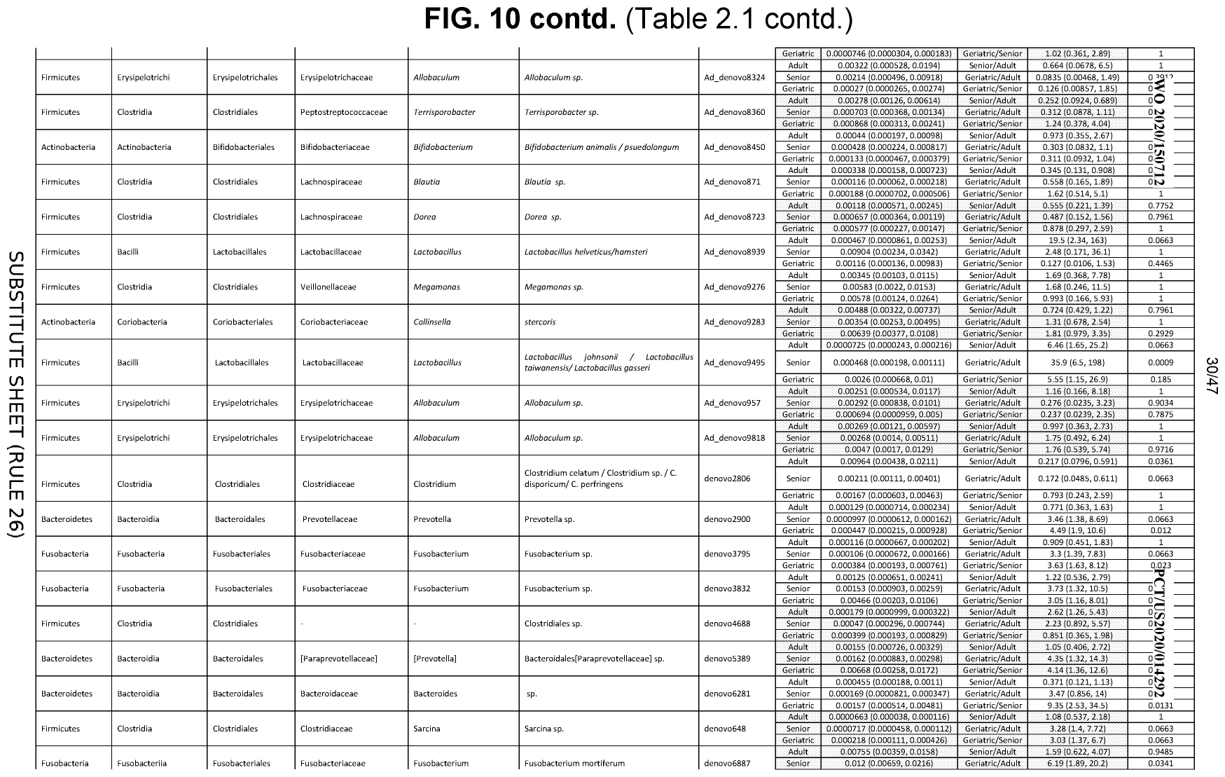

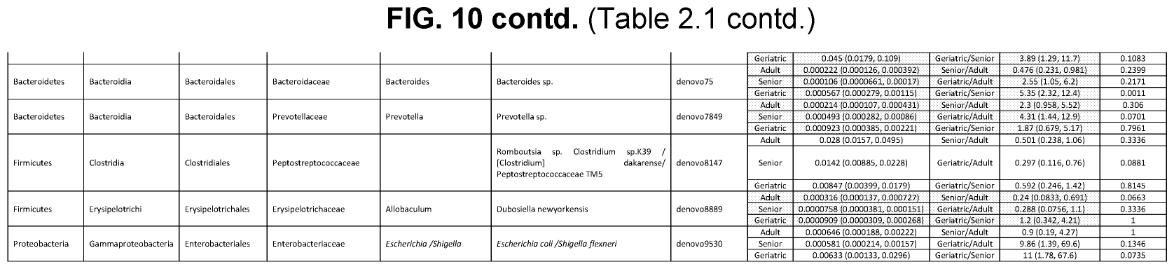

Interrogation of the Greengenes database with reference sequences representing each of the OTUs resulted in 1898 blast results and supported species assignment to 31 of the 119 common taxa (26%). For the 31 OTUs identified these were utilised as the most complete and accurate designation of taxonomy. By comparison, interrogation of the Silva database resulted in 2638 entries relevant to 70 of the 119 common taxa (58.8%). These species designations were used as secondary descriptors for the 39 species not identified by interrogation of the Greengenes database. Taxonomic designations of bacterial species (OTUs) detected in faeces from adult senior and geriatric dogs revealed microbial taxa associated with health in humans and other mammals (FIG. 10 (Table 2.1) and Table 2.2)). Out of a total of 141 taxa representing individual species, 61 (43%) were identified as bacterial species associated with health in non-canid mammals.

### Method

The method involves the extraction of DNA from a freshly produced faecal sample by a means such as the QIAamp Power Faecal DNA kit (Qiagen) and subsequently the use of molecular biology techniques to assess the detection rate and abundance of the bacterial taxa or DNA, RNA or protein sequences characteristic of those described below (FIG. 10 (Table 2.1) and Table 2.2) as well as biomarkers for those organisms compared to standardised healthy control samples and to animals with chronic gastrointestinal enteropathy, IBD, acute diarrhoea and chronic diarrhoea. The interpretation of health status is then made based on the combination and relative abundance of the health associated organisms detected in the faeces of the dogs of the same microbiome lifestage to allow the assessment of health status of the microbiome in the individual and indicate how the health of the microbiome can be enhanced.

Assessment of the microbiome components observed in the faeces or GI sample from the dog can be undertaken at an individual point in time for assessment against healthy and/or clinical controls in the same lifestage, to receive a description of the relative health of the microbiome at a specific timepoint. Alternatively, the gastrointestinal health of the dog can be monitored over time by assessment of the gut microbiome periodically at intervals such as 6 monthly or one yearly tests/assessments or following particular events such as gastrointestinal upset, or travel. The results of detection and relative abundance of the microbial species associated with health (or with the disease condition) can then be compared with the previous results or cumulative (averaged) results from the previous assessments of the microbiome from the individual dog. In the case of longitudinal assessment of an individual over time, adjustments must be made as the animal crosses from one microbiome lifestage to the next by additional comparisons to control cohorts such as provided within the data reported here.

After DNA extraction from freshly produced faeces and sequencing of the DNA by techniques such as 16 S rDNA amplicon, shotgun, metagenome, Illumina, nanopore or other DNA sequencing techniques, the resulting DNA sequences are clustered to species (>98% ID) level. Assessment of the relative abundance of the sequences descriptive of the organisms in FIG. 10 (Table 2.1) or DNA sequences within 95% identical to those in Table 2.2 or other DNA, RNA or protein sequences or biomarkers of those species specified in FIG. 10 (Table 2.1) and Table 2.2 is made. Briefly, sequence data obtained from the test sample is clustered into groups of sequences with about 98%-100% identity and a reference sequence from the clusters which represent>0.001% of the total sequences is then used to either 1) assign taxonomy or gene function through database homologues or to determine the nature of the biomarker through homology searches of DNA databases such as the Greengenes or Silva or the NCBI non-redundant nucleotide sequence database for comparison to known DNA sequences of species held within the databases or 2) compared to the DNA sequences given in Table 2.2.

The number and abundance of the organisms, sequences or biomarkers described within FIG. 10 (Table 2.1) and Table 2.2 are then used to compare to the same data number of organisms and abundance of the individual and total load of the health associated species described in FIG. 11 (Table 2.3). Should the bacterial content or abundance in the faeces or GI sample fall below the notification point listed for the organisms of all of the organisms from a genus this is indicative that the animal can benefit from an intervention to support that bacterial genus to through interventions such as dietary manipulation, supplementation, or other supportive means.

### Example 3: A Method of Detecting Health in the Canine Gut Microbiome Based on Diversity

### Background

Diversity in the gastrointestinal microbiota in humans has been associated with race/ethnicity, nutritional status, dietary diversity and with host health [50;51]. The human infant gut microbiota increases in diversity as it matures, becoming more stable over time, until the community resembles an adult-like state at around three years old [52; 22]. Bacterial species succession in human infants during development is unique to each individual, influenced by host genetics, and susceptible to the influence of multiple factors in post-natal care [22;53]. The early colonization and subsequent maturation including the development of diversity of the microbiome is reported to have long-term health implications for the human host with possible implications on immune function and allergic disease incidence impacting health in later life. The relationships are however complex with C-section birth and formula feeding reported to effect diversity compared to natural modes of birth and breast feeding [54;50;55;56]. The modes of birth and postnatal nutrition delaying the development of diversity appear to be associated with longer term health effects in humans [55].

Data in kittens fully weaned at 5 weeks of age suggest that the microbiota is compositionally similar to the adult feline as early as week 8, demonstrated by a largely stable microbiota over the period from 8 to 16 weeks [57]. Adult dogs have also been shown, similarly to humans and cats to have a highly diverse microbiota which is relatively stable over time [58;59]. The developing microbiota in puppies remains relatively undescribed and hence, research to investigate the developing microbiota in early life and throughout weaning was conducted to understand the composition and diversity of the microbiota in early development and through weaning in growing puppies.

### Methods

The same methods used in Examples 1 and 2 are followed in Example 3.

### Results

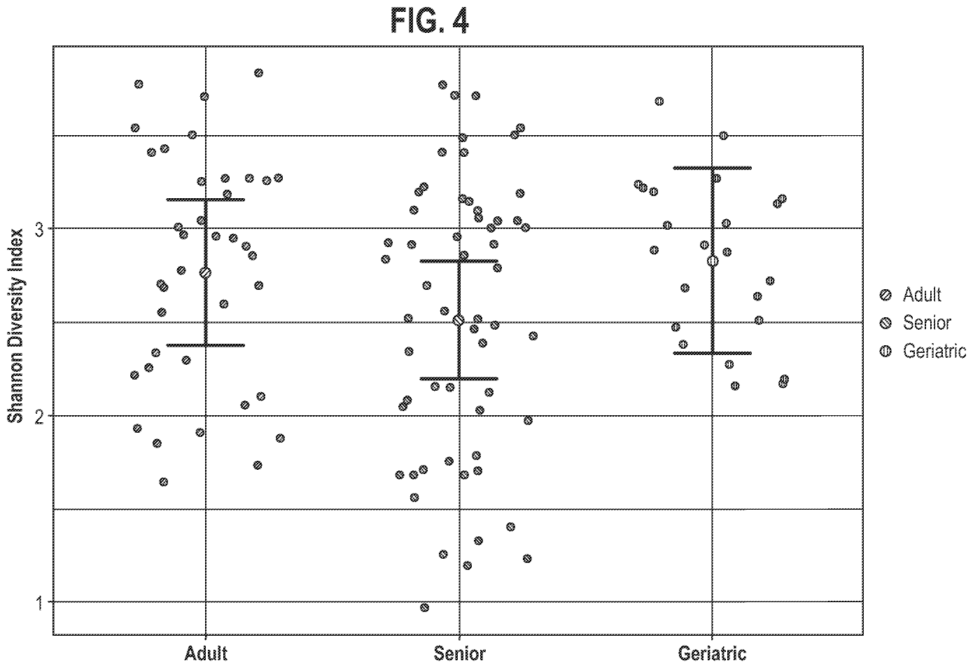

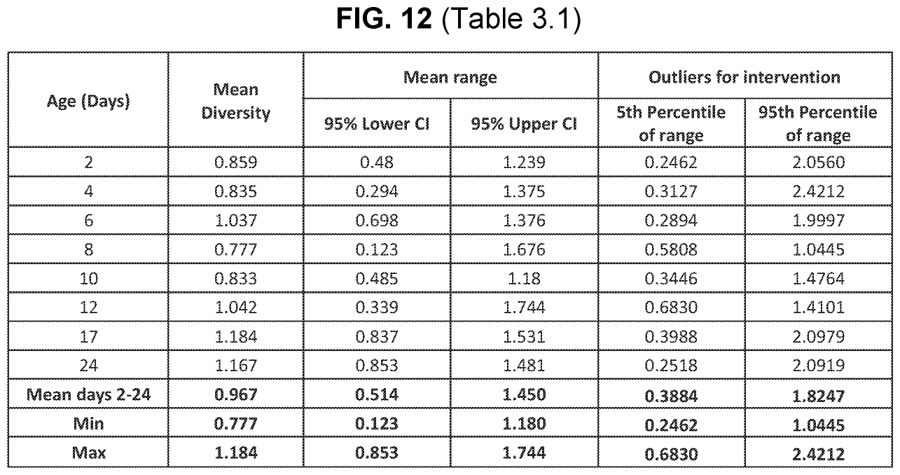

Assessment of Shannon diversity in microbiota analyses of faeces from puppies prior to and throughout the weaning period yielded diversity estimates at each time point suggested low diversity in the puppy faecal microbiota for the first 24 days with an increase after 31 days (during weaning) up to the end of the study (FIG. 4, FIG. 12 (Table 3.1), and Table 3.2). Statistically significant differences were observed between day 4 and days 31, 38, 45 and 52.

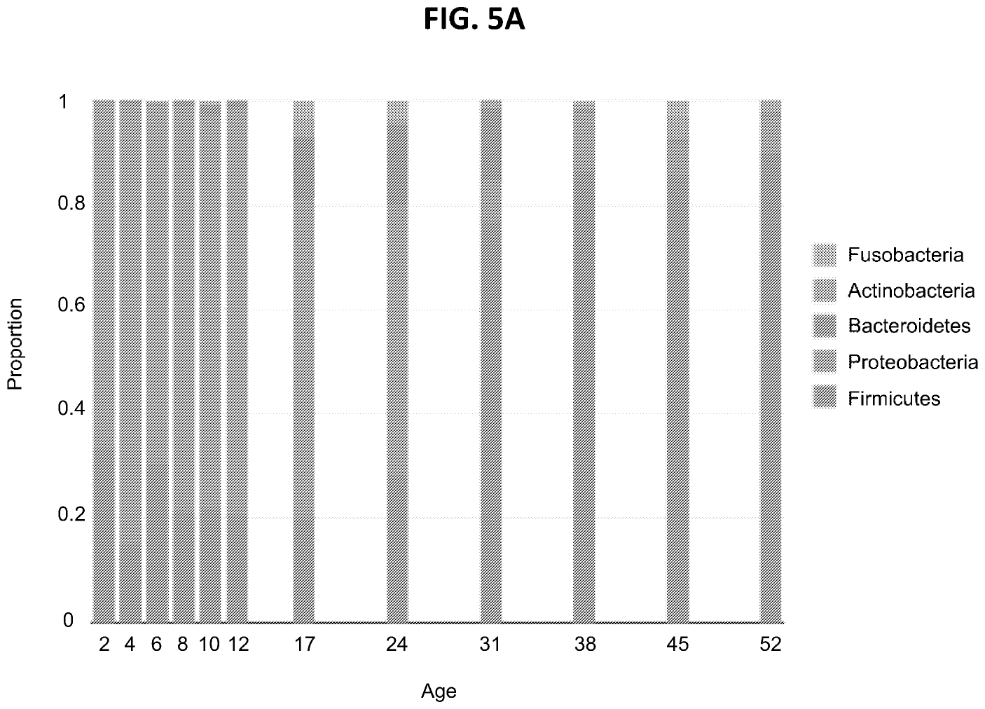

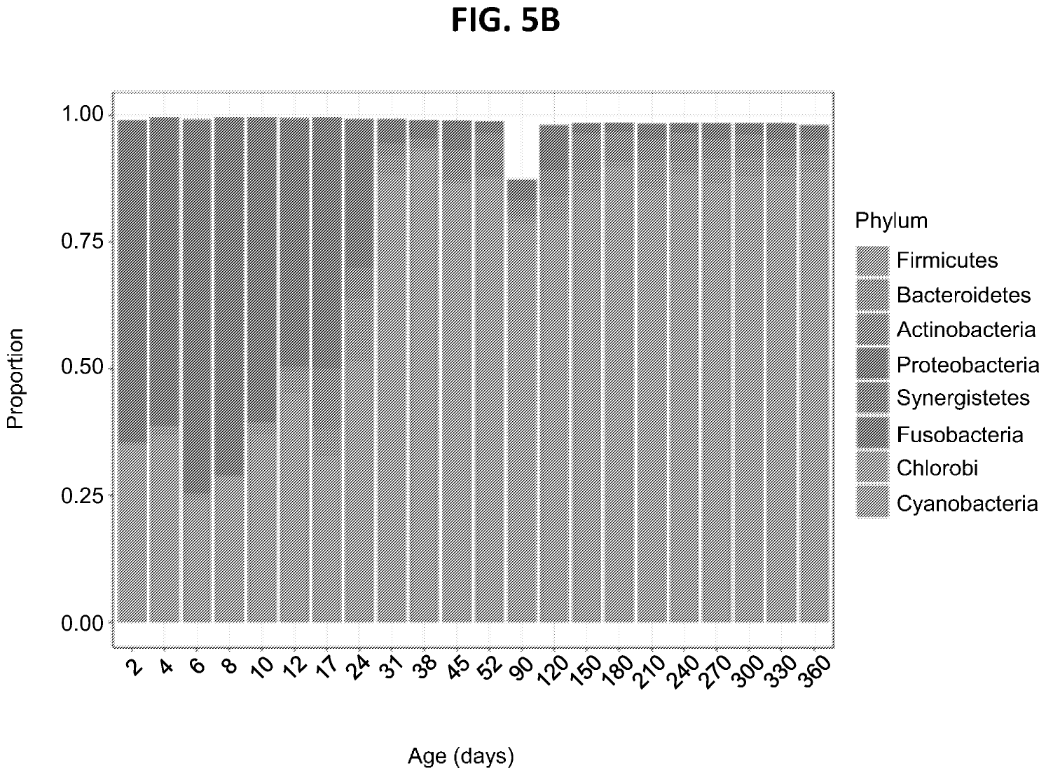

In a second study of a cohort of 48 adult Beagle dogs including 20 adult dogs and 28 in mature life stages, relative consistency was observed in the Shannon diversity of faecal bacterial content across the cohort (FIGS. 5A and 5B).

### Method

The method involves the extraction of DNA from a freshly produced faecal sample by a means such as the QIAamp Power Faecal DNA kit (Qiagen) and subsequently the use of molecular biology techniques to detect the 16 S rDNA or rRNA present or other genetic features thus determining the bacterial abundance and taxon or species richness of the microbial community in faeces or other gastrointestinal sample. After DNA extraction from freshly produced faeces and sequencing of the DNA by techniques such as 16 S rDNA amplicon, shotgun, metagenome, Illumina, nanopore or other DNA sequencing techniques, the resulting DNA sequences are clustered to species (>98% ID) level and the relative abundance of the taxa is determined for the individual OTUs as a proportion of the total sequences. The total number of OTUs and relative abundance data is used to calculate Shannon diversity which accounts for both abundance and evenness of the species detected. Shannon Diversity can be calculated by the following method:

$$Shannon Index ( H ) = - {\sum }_{i = 1}^{s} {p}_{i} ln {p}_{i}$$

After determination of the diversity of the microbiota using functions such as alpha diversity including Shannon diversity index and total OTU numbers with the sample, diversity can be compared to standardised samples from healthy control populations within the same lifestage (see FIG. 12 (Table 3.1), Table 3.2, and Table 3.3) and to animals of similar age with chronic gastrointestinal enteropathy, IBD, acute or chronic diarrhoea or other gastrointestinal symptoms.

The interpretation of health status is then made based on the level of the diversity detected in the faeces of the dog in context of the animals lifestage (puppy, adult, senior or geriatric lifestage) to allow the assessment of microbiome health and to indicate how gastrointestinal health can be enhanced in terms of the direction and magnitude of change in the gut microbial diversity.

Assessment of the microbiome components observed in the faeces of the puppy or adult or aged dog can be undertaken at an individual point in time for assessment against healthy and unhealthy clinical controls of a similar age as described above to receive a description of the health of the microbiome at a specific timepoint. Alternatively, the gastrointestinal health of an individual dog can be monitored over time by testing/assessment of the gut microbiome periodically at intervals such as 6 monthly or annual or following particular events such as gastrointestinal upset, or travel. The results of assessment of the microbial diversity can then be compared with the previous results or cumulative (averaged) results from the previous assessments of the microbiome from the individual dog.

TABLE 1.2
DNA sequences for bacterial taxa associated with health in mammals and detected in
puppies

| | SEQ | |
|---|---|---|
| OTU | ID | 16SrDNA partial DNA sequence |
| Pu_denovo1000 | 3 | CCCGTTCGCTACCCTAGCTTTCGAGCCTCAGCGTCAGTTACAGTCCAGAAAGC |
| | | CGCCTTCGCCACTGGTGTTCTTCCCAATATCTACGCATTTCACCGCTACACTGG |
| | | GAATTCCGCTTTCCTCTCCTGCACTCAAGAAAACCAGTTCGGACCCCATCACG |
| | | GGGTTGAGCCCCGCACTTTTAAGATCCGCTTAGTTTCCCGCCTGCGCTCCCTTT |
| | | ACGCCCAATAATTCCGGACAACGCTTGCCGCCTACGTATTACCGCGGCTGCTG |
| | | GCACGTAGTTAGCCGTGGCTTCCTCGTTAGGTACCGTCAAATAAAAACCTTAT |
| | | TATGATTCTTACCCTTCGTCCCTAACAACAGAACTTTACGATCCTAAGACCTTC |
| | | ATCGTTCACGCGGCGTTGCTCCGTCAGGCTTTCGCCCATTGCGGAAGATTCCCC |
| | | A |
| Pu_denovo10082 | 4 | CCTGTTTGCTCCCCACGCTTTCGAGCCTCAGCGTCAGTTACAGTCCAGAGAGTC |
| | | GCCTTCGCCACTGGTGTTCTTCCTAATCTCTACGCATTTCACCGCTACACTAGG |
| | | AATTCCACTCTCCTCTCCTGCACTCAAGAAAACCAGTTCGGACCCCATCACGG |
| | | GGTTGAGCCCCGCACTTTTAAGATCCGCTTAGTTTCCCGCCTGCGCTCCCTTTA |
| | | CGCCCAATAATTCCGGACAACGCTTGCCGCCTACGTATTACCGCGGCTGCTGG |
| | | CACGTAGTTAGCCGTGGCTTCCTCGTTAGGTACCGTCAAATAAAAACCTTATT |
| | | ATGATTCTTACCCTTCGTCCCTAACAACAGAACTTTACGATCCTAAGACCTTCA |
| | | TCGTTCACGCGGCGTTGCTCCGTCAGGCTTTCGCCCATTGCGGAAGATTCCCCA |
| Pu_denovo10107 | 5 | CCTGTTTGCTCCCCACGCTTTCGCGCCTCAGCGTCAGTTAATGTCCAGCAGGCC |
| | | GCCTTCGCCACTGGTGTTCCTCCCAATATCTACGCATTTCACCGCTACACTGGG |
| | | AATTCCGCCTGCCTCTCCATCACTCAAGACCCGCAGTTTTGAAAGCAGTTTGG |
| | | GGGTTAAGCCCCCAGATTTCACTTCCAACTTACAGGCCCGCCTACACGCCCTTT |
| | | ACACCCAGTAAATCCGGATAACGCTTGCTCCCTACGTATTACCGCGGCTGCTG |
| | | GCACGTAGTTAGCCGGAGCTTATTCTTTAGGTACCGTCATTTGTTTCGTCCCTA |
| | | ATTAAAGATTTTTACAATCCGAAGACCTTCTTCAATCACGCGGCGTTGCTGCGT |
| | | CAGGGTTGCCCCCATTGCCGAAGATTCCCTA |
| Pu_denovo10120 | 6 | CCTGTTTGCTCCCCACGCTTTCGAGCCTCAACGTCAGTTACTGTCCAGTAAGCC |
| | | GCCTTCGCCACTGGTGTTCCTCCTAATATCTACGCATTTCACCGCTACACTAGG |
| | | AATTCCACTTACCTCTCCAGCACTCTAGATGAACAGTTTCCAATGCAGTCCCGG |
| | | GGTTGAGCCCCGGGTTTTCACATCAGACTTGCCCATCCGTCTACGCTCCCTTTA |
| | | CACCCAGTAAATCCGGATAACGCTTGCACCATACGTATTACCGCGGCTGCTGG |
| | | CACGTATTTAGCCGGTGCTTCTTAGTCAGGTACCGTCATTATCTTCCCTGCTGA |
| | | TAGAGCTTTACATACCGAAATACTTCATCGCTCACGCGGCGTCGCTGCATCAG |
| | | GGTTTCCCCCATTGTGCAATATTCCCCA |
| Pu_denovo10268 | 7 | CCTGTTTGCTCCCCACGCTTTCGAGCCTCAACGTCAGTTACTGTCCAGTAAGCC |
| | | GCCTTCGCCACTGGTGTTCCTCCTAATATCTACGCATTTCACCGCTACACTAGG |
| | | AATTCCACTTACCTCTCCAGCACTCTAGCAACACAGTTTCCAAAGCAGTCCCA |
| | | GGGTTGAGCCCTGGGTTTTCACTTCAGACTTGCATCGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCTTGTTTCTTCCCTGCT |
| | | GATAGAGCTTTACATACCGAAATACTTCTTCGCTCACGCGGCGTCGCTGCATC |
| | | AGGGTTTCCCCCATTGTGCAATATTCCCCA |
| Pu_denovo10356 | 8 | CCTGTTTGCTCCCCACGCTTTCGAGCCTCAGCGTCAGTTACAGACCAGAGAGC |
| | | CGCCTTCGCCACTGGTGTTCCTCCATATATCTACGCATTTCACCGCTACACATG |
| | | GAATTCCACTCTCCTCTTCTGCACTCAAGTCTCCCAGTTTCCAATGACCCTCCC |
| | | CGGTTGAGCCGGGGGCTTTCACATCAGACTTAAGAAACCGCCTGCGCTCGCTT |
| | | TACGCCCAATAAATCCGGACAACGCTTGCCACCTACGTATTACCGCGGCTGCT |
| | | GGCACGTAGTTAGCCGTGGCTTTCTGGTTAGATACCGTCAAGGGATGAACAGT |
| | | TACTCTCATCCTTGTTCTTCTCTAACAACAGAGTTTTACGATCCGAAAACCTTC |
| | | TTCACTCACGCGGCGTTGCTCGGTCAGACTTTCGTCCATTGCCGAAGATTCCCT |
| | | A |
| Pu_denovo10534 | 9 | CCTGTTTGCTACCCACGCTTTCGAACCTCAGCGTCAGTTACAGACCAGAGAGC |
| | | CGCTTTCGCCACTGGTGTTCTTCCATATATCTACGCATTTCACCGCTACACATG |
| | | GAGTTCCACTCTCCTCTTCTGCACTCAAGTCTCCCAGTTTCCAATGCACTACTC |
| | | CGGTTAAGCCGAAGGCTTTCACATCAGACTTAAAAGACCGCCTGCGTTCCCTT |
| | | TACGCCCAATAAATCCGGATAACGCTTGCCACCTACGTATTACCGCGGCTGCT |
| | | GGCACGTAGTTAGCCGTGGCTTTCTGGTTAGATACCGTCGAAACGTGAACAGT |
| | | TACTCTCACGCACTTTCTTCTCTAACAACAGGGTTTTACGATCCGAAGACCTTC |
| | | TTCACCCACGCGGCGTTGCTCCATCAGGCTTTCGCCCATTGTGGAAGATTCCCT |
| | | A |
| Pu_denovo10707 | 10 | CCTGTTTGCTACCCACGCTTTCGCGCTTTAGCGTCAGTATCTGTCCAGTGGGCT |
| | | GGCTTCCCCATCGGCCTTCCTACAAATATCTACGAATTTCACCTCTACACTTGT |
| | | AGTTCCGCCCACCTCTCCAGTACTCTAGTTAAGCAGTTTCCAACGCAATACGG |
| | | AGTTGAGCCCCGCATTTTCACATCAGACTTACAAAACCGCCTAGACGCGCTTT |
| | | ACGCCCAATAAATCCGGATAACGCTTGCGACATACGTATTACCGCGGCTGCTG |
| | | GCACGTATTTAGCCGTCGCTTCTTCTGTTAGTACCGTCACTTACTTCGTCCCAA |
| | | CTGAAAGCACTTTACATTCCGAAAAACTTCATCGTGCACACAGAATTGCTGGA |
| | | TCAGACTTTTGGTCCATTGTCCAATATTCCCCA |
| Pu_denovo1074 | 11 | CCTGTTTGCTCCCCACGCTTTCGAGCCTCAACGTCAGTTACCGTCCAGTAAGCC |
| | | GCCTTCGCCACTGGTGTTCCTCCCAATATCTACGCATTTCACCGCTACACTGGG |
| | | AATTCCGCTTACCTCTCCGGCACTCTAGATACACAGTTTCCAAAGCAGTCCCG |
| | | GGGTTGAGCCCCGGGCTTTCACTTCAGACTTGCACATCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAGCTTTACATACCGAAATACTTCTTCGCTCACGCGGCGTCGCTGCATCA |
| | | GGGTTTCCCCCATTGTGCAATATTCCCCA |
| Pu_denovo11006 | 12 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCGAGTATCACAGTTTCCAATGCAGTCCAGG |
| | | GGTTGAGCCCCCGCCTTTCACATCAGACTTGCAACACCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAAGTTTACATACCGAGATACTTCTTCCTTCACGCGGCGTCGCTGCATCAG |
| | | GGTTTCCCCCATTGTGCAATATTCCCCA |
| Pu_denovo11016 | 13 | CCTGTTTGCTCCCCACGCTTTCGAGCCTCAACGTCAGTTACCGTCCAGTAAGCC |
| | | GCCTTCGCCACTGGTGTTCTTCCTAATATCTACGCATTTCACCGCTACACTAGG |
| | | AATTCCGCTTACCTCTCCGGCACTCTAGCTAAACAGTTTCCAAAGCAGTCCCG |
| | | GCGTTGAGCACCGGGCTTTCACTTCAGACTTGCCTTGCCGTCTACACTCCCTTT |
| | | ACACCCAGTAAATCCGGATAACGCTTGCCCCCTACGTATTACCGCGGCTGCTG |
| | | GCACGTAGTTAGCCGGGGCTTCTTATTCAGGTACCGTCACTTTCTTCCCTGCTG |
| | | ATAGAGCTTTACATACCGAAATACTTCTTCACTCACGCGGCGTCGCTGCATCA |
| | | GGGTTTCCCCCATTGTGCAATATTCCCCA |
| Pu_denovo1135 | 14 | CCTGTTTGCTACCCACGCTTTCGAACCTCAGCGTCAGTTACAGACCAGAGAGC |
| | | CGCTTTCGCCACTGGTGTTCTTCCATATATCTACGCATTCCACCGCTACACATG |
| | | GAGTTCCACTGTCCTCTTCTGCACTCAAGTCGCCCGGTTTCCGATGCACTTCTT |
| | | CGGTTAAGCCGAAGGCTTTCACATCAGACCTAAGCAACCGCCTGCGCTCGCTT |
| | | TACGCCCAATAAATCCGGATAACGCTTGCCACCTACGTATTACCGCGGCTGCT |
| | | GGCACGTAGTTAGCCGTGGCTTTCTGGTTAGATACCGTCGAAACGTGAACAGT |
| | | TACTCTCACGCACTTTCTTCTCTAACAACAGGGTTTTACGATCCGAAGACCTTC |
| | | TTCACCCACGCGGCGTTGCTCCATCAGGCTTTCGCCCATTGTGGAAGATTCCCT |
| | | A |
| Pu_denovo11369 | 15 | CCTGTTCGCTACCCATGCTTTCGAGCCTCAGCGTCAGTTGCAGACCAGACAGC |
| | | CGCCTTCGCCACTGGTGTTCTTCCATATATCTACGCATTCCACCGCTACACATG |
| | | GAGTTCCACTGTCCTCTTCTGCACTCAAGTCGCCCGGTTTCCGATGCACTTCTT |
| | | CGGTTAAGCCGAAGGCTTTCACATCAGACCTAAGCAACCGCCTGCGCTCGCTT |
| | | TACGCCCAATAAATCCGGATAACGCTTGCCACCTACGTATTACCGCGGCTGCT |
| | | GGCACGTAGTTAGCCGTGACTTTCTGGTTGGATACCGTCACTGCGTGAACAGT |
| | | TACTCTCACGCACGTTCTTCTCCAACAACAGAGCTTTACGAGCCGAAACCCTTC |
| | | TTCACTCACGCGGTGTTGCTCCATCAGGCTTGCGCCCATTGTGGAAGATTCCCT |
| | | A |
| Pu_denovo11380 | 16 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCCCTCCGGCACTCAAGCCTGGCAGTTTCCAATGCAGTCCAG |
| | | GAGTTGAGCCCCTGCCTTTCACATCAGACTTGCCATGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTATCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCCA |
| Pu_denovo115 | 17 | CCTGTTTGCTCCCCACGCTTTCGTGCCTCAGCGTCAGTTACAGTCCAGAGAGCC |
| | | GCCTTCGCAACTGGTGTTCCTCCTAATATCTACGCATTTCACCGCTACACTAGG |
| | | AATTCCACTCTCCTCTCCTGCACTCAAGTCTTACAGTTTCAAAAGCTTACTACG |
| | | GTTGAGCCGTAGCCTTTCACTTCTGACTTGAAAGACCGCCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAAACCTTCATCACTCACGCGGCGTTGCTGCATCAGG |
| | | GTTTCCCCCATTGTGCAATATTCCCCA |
| Pu_denovo11581 | 18 | CCTGTTTGCTCCCCACGCTTTCGCGCCTCAGCGTCAGTTAATGTCCAGCAGGCC |
| | | GCCTTCGCCACTGGTGTTCCTCCCAATATCTACGCATTTCACCGCTACACTGGG |
| | | AATTCCGCCTGCCTCTCCATCACTCAAGATCCGCAGTTTTGAAAGCAGTTTGGG |
| | | GGTTGAGCCCCCAGATTTCACTCCCAACTTACAGACCCGCCTACACGCCCTTTA |
| | | CACCCAGTAAATCCGGATAACGCTTGCTCCCTACGTATTACCGCGGCTGCTGG |
| | | CACGTAGTTAGCCGGAGCTTATTCTTTAGGTACCGTCATTTTTTTCGTCCCTAA |
| | | TTAAAGATTTTTACAATCCGAAGACCTTCTTCAATCACGCGGCGTTGCTGCGTC |
| | | AGGGTTGCCCCCATTGCGCAATATTCCCCA |
| Pu_denovo11744 | 19 | CCTATTTGCTCCCCACGCTTTCGTGCCTCAGTGTCAGTTACAGACCAGGCGACC |
| | | GCCTTCGCCACTGGTGTTCCTCCATATATCTACGCATTTTACCGCTACACATGG |
| | | AATTCCATCGCCCTCTTCTGCACTCTAGCATACCAGTTTCCATAGCTTACAATG |
| | | GTTGAGCCATTGCCTTTTACTACAGACTTAGTACGCCACCTACGCACCCTTTAC |
| | | GCCCAATGATTCCGGATAACGCTTGCCACCTACGTATTACCGCGGCTGCTGGC |
| | | ACGTAGTTAGCCGTGGCTTTCTGGTAAGCTACCGTCACTCCCATAGCATTTCCT |
| | | CTATGAGCCGTTCTTCACTTACAACAGAGCTTTACGATCCGAAGACCTTCTTCA |
| | | CTCACGCGGCATTGCTCGTTCAGGGTTTCCCCCATTGACGAAAATTCCCTA |
| Pu_denovo1178 | 20 | CCTGTTCGCTCCCCACGCTTTCGCTCCTCAGCGTCAGTAACGGCCCAGAGACCT |
| | | GCCTTCGCCATTGGTGTTCTTCCCGATATCTACACATTCCACCGTTACACCGGG |
| | | ATTCCAGTCTCCCCTGCCGCACTCCAGCCCGCCCGTACCCGGCGCAGATCCA |
| | | CCGTTAAGCGATGGACTTTCACACCAGACGCGACGAACCGCCTACGAGCCCTT |
| | | TACGCCCAATAAATCCGGATAACGCTTGCACCCTACGTATTACCGCGGCTGCT |
| | | GGCACGTAGTTAGCCGGTGCTTATTCAACGGGTACACTCACTCTCGCTTGCTCC |
| | | CCGATAAAAGCGGTTTACAACCCGAAGGCCTCCATCCCGCACGCGGCGTCGCT |
| | | GCGTCAGGCTTTCGCCCATTGCGCAATATTCCCCA |
| Pu_denovo11790 | 21 | CCTGTTTGCTACCCACACTTTCGAGCCTCAGCGTCAGTTGGTGCCCAGTAGGCC |
| | | GCCTTCGCCACTGGTGTTCCTCCCGATATCTACGCATTCCACCGCTACACCGGG |
| | | AATTCCGCCTACCTCTGCACTACTCAAGAAAAACAGTTTTGAAAGCAGTTTAT |
| | | GGGTTGAGCCCATAGATTTCACTTCCAACTTGTCTTCCCGCCTGCGCTCCCTTT |
| | | ACACCCAGTAATTCCGGACAACGCTTGTGACCTACGTTTTACCGCGGCTGCTG |
| | | GCACGTAGTTAGCCGTCACTTCCTTGTTGGGTACCGTCATTATCTTCCCCAACA |
| | | ACAGGAGTTTACAATCCGAAGACCTTCTTCCTCCACGCGGCGTCGCTGCATCA |
| | | GGGTTTCCCCCATTGTGCAATATTCCCCA |
| Pu_denovo1192 | 22 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTGGG |
| | | AATTCCGCTTTCCTCTCCTGCACTCAAGAAAACCAGTTCGGACCCCATCACGG |
| | | GGTTGAGCCCCGCACTTTTAAGATCCGCTTAGTTTCCCGCCTGCGCTCCCTTTA |
| | | CGCCCAATAATTCCGGACAACGCTTGCCGCCTACGTATTACCGCGGCTGCTGG |
| | | CACGTAGTTAGCCGTGGCTTCCTCGTTAGGTACCGTCAAATAAAAACCTTATT |
| | | ATGATTCTTACCCTTCGTCCCTAACAACAGAACTTTACGATCCTAAGACCTTCA |
| | | TCGTTCACGCGGCGTTGCTCCGTCAGGCTTTCGCCCATTGCGGAAGATTCCCCA |
| Pu_denovo12042 | 23 | CCTGTTTGCTCCCCACGCTTTCGTGCCTCAGTGTCAGTTACAGTCCAGAAAGCC |
| | | GCCTTCGCTACTGGTGTTCCTCCTAATATCTACGCATTTCACCGCTACACTAGG |
| | | AATTCCACTTTCCTCTCCTGCACTCAAGTTTCCCAGTTTCAAGAGCTTACTACG |
| | | GTTGAGCCGTAGCCTTTCACTCCTGACTTAAGAAACCACCTACGCACCCTTTAC |
| | | GCCCAGTAAATCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGTTTTACGACCCGAAGGCCTTCATCACTCACGCGGCGTTGCTGCATCAGG |
| | | CTTTCGCCCATTGTGCAATATTCCCCA |
| Pu_denovo12057 | 24 | CCTGTTTGCTCCCCACGCTTTCGAGCCTCAACGTCAGTTATCGTCCAGTAAGCC |
| | | GCCTTCGCCACTGGTGTTCCTCCTAATATCTACGCATTTCACCGCTACACTAGG |
| | | AATTCCACTTACCCCTCCGACACTCTAGACTGACAGTTTCCAATGCAGTCCCGG |
| | | GGTTGAGCCCCGGGTTTTCACATCAGACTTGCCAGTCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAGCTTTACATACCGAAATACTTCTTCGCTCACGCGGCGTCGCTGCATCAG |
| | | GGTTTCCCCCATTGTGCAATATTCCCCA |
| Pu_denovo12145 | 25 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCTAGAAAAACAGTTTCCAATGCAGTCCTG |
| | | GGGTTAAGCCCCAGCCTTTCACATCAGACTTGCTCTTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCCA |
| Pu_denovo12145 | 26 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCTAGAAAAACAGTTTCCAATGCAGTCCTG |
| | | GGGTTAAGCCCCAGCCTTTCACATCAGACTTGCTCTTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCCA |
| Pu_denovo1220 | 27 | CCTGTTTGCTCCCCACGCTTTCGTGCCTCAGCGTCCGTTACAGTCCAGAGAGTC |
| | | GCCTTCGCACCTGGTGTTCCTCCTAATATCTACGCATTTCACCGCTACACTAGG |
| | | AATTCCACTCTCCTCTCCTGCACTCAAGTCTTACAGTTTCAAAAGCTTACTACG |
| | | GTTGAGCCGTAGCCTTTCACTTCTGACTTGAAAGACCACCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCCCACGCGGCGTTGCTGCATCAGG |
| | | CTTTCGCCCATTGTGCAATATTCCCCA |
| Pu_denovo12209 | 28 | CCTGTTTGCTCCCCACGCTTTCGAGCCTCAACGTCAGTTACAGTCCAGTAAGCC |
| | | GCCTTCGCCACTGGTGTTCCTCCTAATATCTACGCATTTCACCGCTACACTAGG |
| | | AATTCCGCTTACCTCTCCTGCACTCCAGCAGCACAGTTTCCAAAGCAGTCCGC |
| | | GGGGTTGAGCCCCGGGCTTTCACTTCAGACTTGCACCGCCGTCTACGCTCCCTT |
| | | TACACCCAGTAAATCCGGATAACGCTTGCCCCCTACGTATTACCGCGGCTGCT |
| | | GGCACGTAGTTAGCCGGGGCTTCTTAGTCAAGTACCGTCATTTTCTTCCTTGCT |
| | | GATAGACCTTTACATACCGAAATACTTCTTCAGTCACGCGGCGTCGCTGCATC |
| | | AGGGTTTCCCCCATTGTGCAATATTCCCCA |
| Pu_denovo12377 | 29 | CCTGTTTGCTCCCCACGCTTTCGCGCCTCAGCGTCAGTTAATGTCCAGCAGGCC |
| | | GCCTTCGCCACTGGTGTTCCTCCCAATATCTACGCATTTCACCGCTACACTGGG |
| | | AATTCCGCCTGCCTCTCCATCACTCAAGACTCGCAGTTTTGAAAGCAGTTTCGG |
| | | GGTTAAGCCCCGAGATTTCACTTCCAACTTGCAAGCCCGCCTACACGCCCTTTA |
| | | CACCCAGTAAATCCGGATAACGCTTGCTCCCTACGTATTACCGCGGCTGCTGG |
| | | CACGTAGTTAGCCGGAGCTTATTCTTCAGGTACCGTCATTTTCTTCGTCCCTGA |
| | | TTAAAGATTTTTACAATCCGAAGACCTTCATCAATCACGCGGCGTTGCTGCGTC |
| | | AGGGTTGCCCCCATTGCGCAATATTCCCCA |
| Pu_denovo1327 | 30 | CCTGTTTGCTCCCCACGCTTTCGAGCCTCAACGTCAGTTACTGTCCAGTAAGCC |
| | | GCCTTCGCCACTGGTGTTCCTCCTAATATCTACGCATTTCACCGCTACACTAGG |
| | | AATTCCACTTACCTCTCCAGCACTCTAGCAAAACAGTTTCCAAAGCAGTCCCG |
| | | GGGTTAAGCCCCGGGCTTTCACTTCAGACTTGCTTCGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAGCTTTACATACCGAAATACTTCTTCACTCACGCGGCGTTGCTGCATCA |
| | | GGGTTTCCCCCATTGTGCAATATTCCCCA |
| Pu_denovo1696 | 31 | CCTGTTTGCTCCCCACGCTTTCGCGCCTCAGCGTCAGTTACTGTCCAGCAATCC |
| | | GCCTTCGCCACTGGTGTTCCTCCGTATATCTACGCATTTCACCGCTACACACGG |
| | | AATTCCGATTGCCTCTCCAGCACTCAAGAACTACAGTTTCAAATGCAGGCTGG |
| | | AGGTTGAGCCCCCAGTTTTCACATCTGACTTGCAATCCCGCCTACACGCCCTTT |
| | | ACACCCAGTAAATCCGGATAACGCTTGCCACCTACGTATTACCGCGGCTGCTG |
| | | GCACGTAGTTAGCCGTGGCTTATTCGTCAGGTACCGTCATTTGTTTCGTCCCCG |
| | | ACAAAAGAAGTTTACAACCCGAAAGCCTTCTTCCTTCACGCGGCGTTGCTGGG |
| | | TCAGGCTTGCGCCCATTGCCCAATATTCCCCA |
| Pu_denovo1830 | 32 | CCTGTTTGCTCCCCACGCTTTCGAGCCTCAACGTCAGTTATCGTCCAGTAAGCC |
| | | GCCTTCGCCACTGGTGTTCCTCCTAATATCTACGCATTTCACCGCTACACTAGG |
| | | AATTCCACTTACCTCTCCGACACTCTAGCAAAACAGTTTCCAAAGCAGTCCCA |
| | | GGGTTGAGCCCTGGGTTTTCACTTCAGACTTGCTTCGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAGCTTTACATACCGAAATACTTCTTCACTCACGCGGCGTCGCTGCATCA |
| | | GGCTTTCGCCCATTGTGCAATATTCCCCA |
| Pu_denovo1987 | 33 | CCTATTTGCTCCCCACGCTTTCGGGACTGAGCGTCAGTTGCAGGCCAGATCGTC |
| | | GCCTTCGCCACTGGTGTTCCTCCATATATCTACGCATTTCACCGCTACACATGG |
| | | AATTCCACGATCCTCTCCTGCACTCTAGCTGCCTGGTTTCTATGGCTTACTGAA |
| | | GTTAAGCTTCAGGCTTTCACCACAGACCCTTGCTGCCGCCTGCTCCCTCTTTAC |
| | | GCCCAATAATTCCGGATAACGCTTGCCACCTACGTATTACCGCGGCTGCTGGC |
| | | ACGTAGTTAGCCGTGGCTTTCTAATAAAGTACCGTCACTCGGCTACCATTTCCT |
| | | GTAGCCGCCGTTCTTCCTTTATAACAGAAGTTTACAATCCGAAAACCTTCTTCC |
| | | TTCACGCGGCGTTGCTCGGTCAGGGTTTCCCCCATTGCCGAAAATTCCCTA |
| Pu_denovo2011 | 34 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCTAGATGGACAGTTTCCAAAGCAGTCCAG |
| | | GGGTTGAGCCCCTGCCTTTCACTTCAGACTTGCCCGTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTGTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCTA |
| Pu_denovo2011 | 35 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCTAGATGGACAGTTTCCAAAGCAGTCCAG |
| | | GGGTTGAGCCCCTGCCTTTCACTTCAGACTTGCCCGTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTGTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCTA |
| Pu_denovo2050 | 36 | CCTGTTTGCTCCCCACGCTTTCGAGCCTCAGCGTCAGTTACAGTCCAGAGAGTC |
| | | GCCTTCGCCACTGGTGTTCTTCCTAATCTCTACGCATTTCACCGCTACACTAGG |
| | | AATTCCACTCTCCTCTCCTGCACTCTAGATAATCAGTTTGGAATGCAGCCCCCA |
| | | GGTTGAGCCTGAGTATTTCACATCCCACTTAATTATCCGCCTACGCTCCCTTTA |
| | | CGCCCAATAATTCCGGATAACGCTCGCCACCTACGTATTACCGCGGCTGCTGG |
| | | CACGTAGTTAGCCGTGGCTTCCTCCTTGGGTACCGTCATTATCGTCCCCAAAGA |
| | | CAGAGCTTTACAATCCGAAGACCGTCATCACTCACGCGGCGTTGCTGCATCAG |
| | | GGTTTCCCCCATTGTGCAATATTCCCCA |
| Pu_denovo2116 | 37 | CCTGTTTGCTCCCCACGCTTTCGTGCCTCAGCGTCAGTTACAGTCCAGAAAGCC |
| | | GCCTTCGCTACTGGTGTTCCTCCCAATATCTACGCATTTCACCGCTACACTGGG |
| | | AATTCCGCTTTCCTCTCCTGCACTCAAGTCAGACAGTATCAGGAGCTTACTACG |
| | | GTTGAGCCGTAGCCTTTAACTCCTGACTTGAAAGACCGCCTACGCACCCTTTAC |
| | | GCCCAGTAAATCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTGCGCCCATTGTGCAATATTCCCCA |
| Pu_denovo2226 | 38 | CCTGTTTGCTCCCCACGCTTTCGAGCCTCAGCGTCAGTTAAAGCCCAGCAGGC |
| | | CGCCTTCGCCACTGGTGTTCCTCCTAATATCTACGCATTTCACCGCTACACTAG |
| | | GAATTCCGCCTGCCTCTACTTCACTCAAGAACGGCAGTTTAGAACGCAGCCAC |
| | | CGGTTGAGCCGATGGATTTAACATTCTACTTGCCATCCCGCCTACGCTCCCTTT |
| | | ACACCCAGTAATTCCGGACAACGCTTGCTCCCTACGTATTACCGCGGCTGCTG |
| | | GCACGTAGTTAGCCGGAGCTTCCTCTTTGGGTACCGTCATTTTCTTCCCCAAAG |
| | | ACAGAGGTTTACAATCCGAAGACCGTCTTCCCTCACGCGGCGTCGCTGCATCA |
| | | GGCTTTCGCCCATTGTGCAATATCCCCCA |
| Pu_denovo2292 | 39 | CCTGTTCGCTCCCCACGCTTTCGAGCCTCAGCGTCAGTTTCAGTCCAGAAAGCC |
| | | GCCTTCGCCACCGGTGTTCTTCCTAATATCTACGCATTTCACCGCTACACTAGG |
| | | AATTCCGCTTTCCTCTCCTGTACTCTAGCTTGATAGTTTAAAATGCAATCCTCG |
| | | GGTTAAGCCCAAGGCTTTCACATCTTACTTACCATGCCGCCTACGCTCCCTTTA |
| | | CACCCAGTAATTCCGGATAACGCTTGCCCCCTACGTATTACCGCGGCTGCTGG |
| | | CACGTAGTTAGCCGGGGCTTCTTATTTGGGTACCGTCATTCTTTTCTTCCCCAT |
| | | CGATAGAAGTTTACAATCCGAAAACCGTCTTCCTTCACGCGGCGTTGCTGCAT |
| | | CAGGGTTTCCCCCATTGTGCAATATTCCCCA |
| Pu_denovo2529 | 40 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCACTCTCCTCTCCTGCACTCAAGTCTTACAGTTTCAAAAGCTTACTACG |
| | | GTTGAGCCGTAGCCTTTCACTTCTGACTTGAAAGACCGCCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTTCGCCCATTGTGCAATATTCCCCA |
| Pu_denovo2648 | 41 | CCTATTTGCTCCCCACGCTTTCGTGCTTCAGTGTCAGAATCCAGACCAGACGGC |
| | | CGCCTTCGCCACCGGTGTTCTTCCATATATCTACGCATTTTACCGCTACACATG |
| | | GAGTTCCGCCGTCCTCTTCTGTTCTCTAGCTGATCAGTTTCCAGAGCAAGTACG |
| | | GGTTGAGCCCATACCTTTTACTCCAGACTTGATCTGCCACCTACGCACCCTTTA |
| | | CGCCCAATCATTCCGGATAACGCTCGCCACCTACGTATTACCGCGGCTGCTGG |
| | | CACGTAGTTAGCCGTGACTTTCTGGTAAGATACCATCACTCACTCATCATTCCC |
| | | TATGAGTGCCGTTTTTCTCTTACAACAGAGCTTTACGATCCGAAGACCTTCCTC |
| | | ACTCACGCGGCATTGCTCGTTCAGGGTTCCCCCCATTGACGAAAATTCCCTA |
| Pu_denovo3119 | 42 | CCTGTTTGCTCCCCACGCTTTCGTGCCTCAGCGTCAGTTATGGTCCAGAAAGCC |
| | | GCCTTCGCTACTGGTGTTCCTTTGAATCTCTACGCATTTCACCGCTACACTCAA |
| | | AGTTCCACTTTCCTCTCCCACACTCTAGCCTCTCAGTTTCGGTAGCAGCTCCGG |
| | | GGTTGAGCCCCGAAATTTCACTTCCGACTTAAAAGGCCGCCTACGCACCCTTT |
| | | ACACCCAGTAAATCCGGATAACGCTTGCTCCCTACGTATTACCGCGGCTGCTG |
| | | GCACGTAGTTAGCCGGAGCTTCTTAGTCAGGTACCGTCATTTCTTCTTCCCTGC |
| | | TGATAGAAGTTTACAATCCGAAGACCTTCTTCCTTCACGCGGCGTTGCTGCATC |
| | | AGGCTTTCGCCCATTGTGCAATATTCCCCA |
| Pu_denovo3179 | 43 | CCTGTTTGCTCCCCACGCTTTCGTGCCTCAGTGTCAGTTACAGTCCAGAGAGCC |
| | | GCCTTCGCAACTGGTGTTCCTCCTAATATCTACGCATTTCACCGCTACACTAGG |
| | | AATTCCACTCTCCTCTCCTGCACTCAAGTTTATCAGTTTCAAAAGCTTACTATG |
| | | GTTAAGCCATAGCCTTTCACTTCTGACTTGATAAACCACCTACGCACCCTTTAC |
| | | GCCCAGTAATTCCGGATAACGCTTGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGTTTTACGACCCTAAGGCCTTCTTCACTCACGCGGCGTTGCTGCATCAGG |
| | | CTTTCGCCCATTGTGCAATATTCCCCA |
| Pu_denovo3749 | 44 | CCTGTTTGCTCCCCACGCTTTCGAGCCTCAACGTCAGTTATCGTCCAGTAAGCC |
| | | GCCTTCGCCACTGATGTTCCTCCTAATATCTACGCATTTCACCGCTACACTAGG |
| | | AATTCCGCTTACCTCTCCGACACTCTAGAAGCACAGTTTCCAAAGCAGTCACG |
| | | GGGTTGAGCCCCGGGCTTTCACTTCAGACTTGCACTTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAAATACTTCTTCCTTCACGCGGCGTCGCTGCATCA |
| | | GGGTTTCCCCCATTGTGCAATATTCCCCA |
| Pu_denovo3749 | 45 | CCTGTTTGCTCCCCACGCTTTCGAGCCTCAACGTCAGTTATCGTCCAGTAAGCC |
| | | GCCTTCGCCACTGATGTTCCTCCTAATATCTACGCATTTCACCGCTACACTAGG |
| | | AATTCCGCTTACCTCTCCGACACTCTAGAAGCACAGTTTCCAAAGCAGTCACG |
| | | GGGTTGAGCCCCGGGCTTTCACTTCAGACTTGCACTTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAAATACTTCTTCCTTCACGCGGCGTCGCTGCATCA |
| | | GGGTTTCCCCCATTGTGCAATATTCCCCA |
| Pu_denovo3887 | 46 | CCTGTTCGCTACCCACGCTTTCGAACCTCAGCGTCAGTTACAGACCAGAGAGC |
| | | CGCTTTCGCCACTGGTGTTCTTCCATATATCTACGCATTTCACCGCTACACATG |
| | | GAGTTCCACTCTCCTCTTCTGCACTCAAGTTCCCCAGTTTCCAATGCACATCTT |
| | | CGGTTAAGCCGAAGGCTTTCACATCAGACTTAAAGAACCGCCTGCGTTCCCTT |
| | | TACGCCCAATAAATCCGGATAACGCTTGCCACCTACGTATTACCGCGGCTGCT |
| | | GGCACGTAGTTAGCCGTGACTTGCTGGTTAGATACCGTCAACAGGTGAACAGT |
| | | TACTCTCACCCGTGTTCTTCTCTAACAACAGAGTTTTACGATCCGAAGACCTTC |
| | | TTCGCTCACGCGGCGTTGCTCCATCAGACTTGCGTCCATTGTGGAAGATTCCCT |
| | | A |
| Pu_denovo4020 | 47 | CCTGTTTGCTCCCCACGCTTTCGAGCCTCAACGTCAGTTACTGTCCAGCAAGCC |
| | | GCCTTCGCCACTGGTGTTCCTCCTAATATCTACGCATTTCACCGCTACACTAGG |
| | | AATTCCGCTTGCCTCTCCAGCACTCTAGCCCAACAGTTTCCAAAGCAGTTCCCG |
| | | GGTTGAGCCCGGGGATTTCACTTCAGACTTGCTGTGCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCACTTTCTTCCCTGCTGA |
| | | TAGAGCTTTACATACCGAAATACTTCTTCACTCACGCGGCGTTGCTGCATCAG |
| | | GGTTTCCCCCATTGTGCAATATTCCCCA |
| Pu_denovo4476 | 48 | CCTGTTTGCTCCCCACGCTTTCGAGCCTCAACGTCAGTCATCGTCCAGTAAGCC |
| | | GCCTTCGCCACTGGTGTTCCTCCTAATATCTACGCATTTCACCGCTACACTAGG |
| | | AATTCCACTTACCTCTCCGACACTCTAGAAAAACAGTTTCCAATGCAGTCCCG |
| | | GGGTTGAGCCCCGGGTTTTCACATCAGACTTGCCTCTCCGTCTACGCTCCCTTT |
| | | ACACCCAGTAAATCCGGATAACGCTTGCACCATACGTATTACCGCGGCTGCTG |
| | | GCACGTATTTAGCCGGTGCTTCTTAGTCAGGTACCGTCATTTTCTTCCCTGCTG |
| | | ATAGAGCTTTACATACCGAAATACTTCATCGCTCACGCGGCGTCGCTGCATCA |
| | | GGGTTTCCCCCATTGTGCAATATTCCCCA |
| Pu_denovo46 | 49 | CCTGTTTGCTCCCCACGCTTTCGAGCCTCAACGTCAGTTACCGTCCAGTAAGCC |
| | | GCCTTCGCCACTGGTGTTCTTCCTAATATCTACGCATTTCACCGCTACACTAGG |
| | | AATTCCGCTTACCTCTCCGGCACTCTAGCTAAACAGTTTCCAAAGCAGTCCCG |
| | | GCGTTGAGCACCGGGCTTTCACTTCAGACTTGCCTTGCCGTCTACACTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GAGTTTCCTCCATTGTGCAATATTCCCCA |
| Pu_denovo4759 | 50 | CCTGTTTGCTCCCCACGCTTTCGAGCCTCAACGTCAGTCATCGTCCAGAAAGCC |
| | | GCCTTCGCCACTGGTGTTCCTCCTAATATCTACGCATTTCACCGCTACACTAGG |
| | | AATTCCGCTTTCCTCTCCGACACTCTAGCCTGACAGTTCCAAATGCAGTCCCGG |
| | | GGTTGAGCCCCGGGCTTTCACATCTGGCTTGCCATGCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAGCTTTACATACCGAAATACTTCATCGCTCACGCGGCGTCGCTGCATCAG |
| | | GGTTTCCCCCATTGTGCAATATTCCCCA |
| Pu_denovo4770 | 51 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCAAGATGGACAGTTTCCAATGCAGTCCCG |
| | | GGGTTAAGCCCCGGGCTTTCACATCAGACTTGCCCGTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GAGTTTCCTCCATTGTGCAATATTCCCCA |
| Pu_denovo4820 | 52 | CCTGTTTGCTCCCCACGCTTTCGAGCCTCAACGTCAGTCACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCCAGATCTGCAGTTTCCAAAGCAGTCCCAG |
| | | GGTTGAGCCCTGGGTTTTCACTCCAGACTTGCCTATCCGTCTACGCTCCCTTTA |
| | | CACCCAGTAAATCCGGATAACGCTTGCACCATACGTATTACCGCGGCTGCTGG |
| | | CACGTATTTAGCCGGTGCTTCTTACTCAGGTACCGTCATCTTCTTCCCTGCTGA |
| | | TAGAAGTTTACATACCGAAATACTTCTTCCTTCACGCGGCGTCGCTGCATCAG |
| | | GGTTTCCCCCATTGTGCAATATTCCCCA |
| Pu_denovo5010 | 53 | CCTGTTTGCTCCCCACGCTTTCGTGCCTCAGCGTCAGTTACAGTCCAGAGAGCC |
| | | GCCTTCGCAACTGGTGTTCCTCCTAATATCTACGCATTTCACCGCTACACTAGG |
| | | AATTCCACTCTCCTCTCCTGCACTCAAGTCTCCCAGTTTCAAGAGCTTACTACG |
| | | GTTGAGCCGTAGCCTTTCACTCCTGACTTGAAAGACCGCCTACGCACCCTTTAC |
| | | GCCCAGTAAATCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGTTTTACGACCCGAAGGCCTTCATCACTCACGCGGCGTTGCTGCATCAGG |
| | | CTTTCGCCCATTGTGCAATATTCCCCA |
| Pu_denovo5010 | 54 | CCTGTTTGCTCCCCACGCTTTCGTGCCTCAGCGTCAGTTACAGTCCAGAGAGCC |
| | | GCCTTCGCAACTGGTGTTCCTCCTAATATCTACGCATTTCACCGCTACACTAGG |
| | | AATTCCACTCTCCTCTCCTGCACTCAAGTCTCCCAGTTTCAAGAGCTTACTACG |
| | | GTTGAGCCGTAGCCTTTCACTCCTGACTTGAAAGACCGCCTACGCACCCTTTAC |
| | | GCCCAGTAAATCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGTTTTACGACCCGAAGGCCTTCATCACTCACGCGGCGTTGCTGCATCAGG |
| | | CTTTCGCCCATTGTGCAATATTCCCCA |
| Pu_denovo5029 | 55 | CCTGTTTGCTCCCCACGCTTTCGTGCCTCAGTGTCAGTTACAGTCCAGAGAGCC |
| | | GCCTTCGCAACTGGTATTCCTCCTAATATCTACGCATTTCACCGCTACACTAGG |
| | | AATTCTACTCTCCTCTCCTGCACTCAAGTTTCTCAGTTTCAAAGGCTTACTACG |
| | | GTTGAGCCGTAGCCTTTCACCTCTGACTTAAGAAACCACCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTTCGCCCATTGTGCAATATTCCCCA |
| Pu_denovo507756 | | CCTGTTTGCTACCCACGCTTTCGAACCTCAGCGTCAGTTACAGACCAGAGAGC |
| | | CGCTTTCGCCACTGGTGTTCTTCCATATATCTACGCATTTCACCGCTACACATG |
| | | GAGTTCCACTCTCCTCTTCTGCACTCAAGTCTTCCAGTTTCCAATGCACTACTT |
| | | CGGTTAAGCCGAAGGCTTTCACATCAGACTTAAAAGACCGCCTGCGTTCCCTT |
| | | TACGCCCAATAAATCCGGACAACGCTCGCCACCTACGTATTACCGCGGCTGCT |
| | | GGCACGTAGTTAGCCGTGGCTTTCTGGTCAGATACCGTCAATACGTGAACAGT |
| | | TACTCTCACGCACGTTCTTCTCTGACAACAGAATTTTACGACCCGAAGGCCTTC |
| | | TTCATTCACGCGGCGTTGCTCCATCAGACTTTCGTCCATTGTGGAAGATTCCCT |
| | | A |
| Pu_denovo5125 | 57 | CCTGTTTGCTCCCCACACTTTCGTGCCTCAACGTCAGTTACTGTCCAGAAAGTC |
| | | GCCTTCGCCACTGGTGTTCCTCCTAATATCTACGCATTTCACCGCTACACTAGG |
| | | AATTCCACTTTCCTCTCCAGCACTCAAGAAATATAGTTTTGGTTGCAATTCCTC |
| | | GGTTGAGCCGAGGGATTTCACAACCAACTTGCATTCCCGTCTACGCACCCTTT |
| | | ACACCCAATAAATCCGGATAACGCTTGCTCCCTACGTATTACCGCGGCTGCTG |
| | | GCACGTAGTTAGCCGGAGCTTATTCTACAGGTACTGTCTTGTTTCTTCCCTGTC |
| | | TAAAGCAGTTTACAATCCGAAAACCTTCTTCCTGCACGCGGCGTCACTGCGTC |
| | | AGAGTTTCCTCCATTGCGCAATATTCCCGA |
| Pu_denovo5198 | 58 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGTACTCAAGATCAACAGTTTCCAATGCAGTCCAG |
| | | GGGTTGAGCCCCTGCCTTTCACATCAGACTTGCTGCTCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCCA |
| Pu_denovo5343 | 59 | CCTGTTTGCTCCCCACGCTTTCGAGCCTCAGCGTCAGTTACAGACCAGAGAGC |
| | | CGCTTTCGCCACCGGTGTTCCTCCATATATCTACGCATTTCACCGCTACACTGG |
| | | GAATTCCGCTTTCCTCTCCTGCACTCAAGAAAACCAGTTCGGACCCCATCACG |
| | | GGGTTGAGCCCCGCACTTTTAAGATCCGCTTAGTTTCCCGCCTGCGCTCCCTTT |
| | | ACGCCCAATAATTCCGGACAACGCTTGCCGCCTACGTATTACCGCGGCTGCTG |
| | | GCACGTAGTTAGCCGTGGCTTCCTCGTTAGGTACCGTCAAATAAAAACCTTAT |
| | | TATGATTCTTACCCTTCGTCCCTAACAACAGAACTTTACGATCCTAAGACCTTC |
| | | ATCGTTCACGCGGCGTTGCTCCGTCAGGCTTTCGCCCATTGCGGAAGATTCCCC |
| | | A |
| Pu_denovo5343 | 60 | CCTGTTTGCTCCCCACGCTTTCGAGCCTCAGCGTCAGTTACAGACCAGAGAGC |
| | | CGCTTTCGCCACCGGTGTTCCTCCATATATCTACGCATTTCACCGCTACACTGG |
| | | GAATTCCGCTTTCCTCTCCTGCACTCAAGAAAACCAGTTCGGACCCCATCACG |
| | | GGGTTGAGCCCCGCACTTTTAAGATCCGCTTAGTTTCCCGCCTGCGCTCCCTTT |
| | | ACGCCCAATAATTCCGGACAACGCTTGCCGCCTACGTATTACCGCGGCTGCTG |
| | | GCACGTAGTTAGCCGTGGCTTCCTCGTTAGGTACCGTCAAATAAAAACCTTAT |
| | | TATGATTCTTACCCTTCGTCCCTAACAACAGAACTTTACGATCCTAAGACCTTC |
| | | ATCGTTCACGCGGCGTTGCTCCGTCAGGCTTTCGCCCATTGCGGAAGATTCCCC |
| | | A |
| Pu_denovo5401 | 61 | CCTGTTTGCTCCCCACACTTTCGTGCCTCAACGTCAGTTGCTGTCCAGAAAGTC |
| | | GCCTTCGCCACTGGTGTTCCTCCTAATATCTACGCATTTCACCGCTACACTAGG |
| | | AATTCCACTTTCCTCTCCAGCACTCAAGAAAAGCAGTTTTAGTCGCAGTTCCTC |
| | | AGTTGAGCCGAGGGATTTCACAACTAACTTACCTTCCCGTCTACGCACCCTTTA |
| | | CACCCAATAAATCCGGATAACGCTTGCTCCCTACGTATTACCGCGGCTGCTGG |
| | | CACGTAGTTAGCCGGAGCTTATTCTACAAGTACTGTCTTGTTTCTTCCTTGTCT |
| | | AAAATGGTTTACAATCCGAAAACCTTCTTCCCATACGCGGCGTCACTGCGTCA |
| | | GAGTTTCCTCCATTGCGCAATATTCCCGA |
| Pu_denovo579 | 62 | CCTGTTTGCTCCCCACGCTTTCGTGCCTCAGCGTCAGTTACAGTCCAGAGAGCC |
| | | GCCTTCGCTACTGGTGTTCCTCCTAATATCTACGCATTTCACCGCTACACTAGG |
| | | AATTCCACTCTCCTCTCCTGCACTCAAGTCCTACAGTTCCAAAAGCTTACTACG |
| | | GTTGAGCCGTAGCCTTTCACTTCTGGCTTGAAAGACCGCCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTTCGCCCATTGTGCAATATTCCCCA |
| Pu_denovo579 | 63 | CCTGTTTGCTCCCCACGCTTTCGTGCCTCAGCGTCAGTTACAGTCCAGAGAGCC |
| | | GCCTTCGCTACTGGTGTTCCTCCTAATATCTACGCATTTCACCGCTACACTAGG |
| | | AATTCCACTCTCCTCTCCTGCACTCAAGTCCTACAGTTCCAAAAGCTTACTACG |
| | | GTTGAGCCGTAGCCTTTCACTTCTGGCTTGAAAGACCGCCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTTCGCCCATTGTGCAATATTCCCCA |
| Pu_denovo5855 | 64 | CCTGTTTGCTCCCCACGCTTTCGAGCCTCAGCGTCAGTTACAGACCAGAGAGC |
| | | CGCTTTCGCCACCGGTGTTCCTCCATATATCTACGCATTTCACCGCTACACTAG |
| | | GAATTCCACTCTCCTCTCCTGCACTCAAGTCTTACAGTTTCAAAAGCTTACTAC |
| | | GGTTGAGCCGTAGCCTTTCACTTCTGACTTGAAAGACCGCCTACGCACCCTTTA |
| | | CGCCCAGTAATTCCGGATAACGCTAGCCCCCTACGTATTACCGCGGCTGCTGG |
| | | CACGTAGTTAGCCGGGGCTTCCTCCTCAAGTACCGTCATTATCTTCCTTGAGGA |
| | | CAGAGCTTTACGACCCGAAGGCCTTCATCGCTCACGCGGCGTTGCTGCATCAG |
| | | GCTTTCGCCCATTGTGCAATATTCCCCA |
| Pu_denovo5855 | 65 | CCTGTTTGCTCCCCACGCTTTCGAGCCTCAGCGTCAGTTACAGACCAGAGAGC |
| | | CGCTTTCGCCACCGGTGTTCCTCCATATATCTACGCATTTCACCGCTACACTAG |
| | | GAATTCCACTCTCCTCTCCTGCACTCAAGTCTTACAGTTTCAAAAGCTTACTAC |
| | | GGTTGAGCCGTAGCCTTTCACTTCTGACTTGAAAGACCGCCTACGCACCCTTTA |
| | | CGCCCAGTAATTCCGGATAACGCTAGCCCCCTACGTATTACCGCGGCTGCTGG |
| | | CACGTAGTTAGCCGGGGCTTCCTCCTCAAGTACCGTCATTATCTTCCTTGAGGA |
| | | CAGAGCTTTACGACCCGAAGGCCTTCATCGCTCACGCGGCGTTGCTGCATCAG |
| | | GCTTTCGCCCATTGTGCAATATTCCCCA |
| Pu_denovo5873 | 66 | CCTGTTTGCTCCCCACGCTTTCGTGCATCAGTGTCAGTGACAGTCCAGTAAGCC |
| | | GCCTTCGCCACTGGTGTTCCTCCTAATATCTACGCATTTCACCGCTACACTAGG |
| | | AATTCCGCTTACCTCTCCTGCACTCCAGCATGACAGTTTCAAAAGCAGTCCCG |
| | | GGGTTAAGCCCCGGGCTTTCACTTCTGACTTACCATGCCACCTACGCACCCTTT |
| | | ACACCCAGTAATTCCGGATAACGCTTGCCCCCTACGTATTACCGCGGCTGCTG |
| | | GCACGTAGTTAGCCGGGGCTTCTTAGTCAGGTACCGTCTGTTTTCTTCCCTGCT |
| | | GATAGAGCTTTACATACCGAAATACTTCTTCACTCACGCGGCGTCGCTGCATC |
| | | AGGGTTTCCCCCATTGTGCAATATTCCCCA |
| Pu_denovo6511 | 67 | CCTGTTTGCTCCCCACGCTTTCGAGCCTCAACGTCAGTTACTGTCCAGTAAGCC |
| | | GCCTTCGCCACTGGTGTTCCTCCTAATATCTACGCATTTCACCGCTACACTAGG |
| | | AATTCCACTTACCTCTCCAGCACTCTAGCAGAACAGTTTCCAAAGCAGTCCCG |
| | | GGGTTGAGCCCCGGGCTTTCACTTCAGACTTGCTCCGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAGATACTTCTTCCTTCACGCGGCGTCGCTGCATCA |
| | | GGGTTTCCCCCATTGTGCAATATTCCCCA |
| Pu_denovo654 | 68 | CCTGTTTGCTCCCCACGCTTTCGAGCCTCAACGTCAGTTACTGTCCAGTAAGCC |
| | | GCCTTCGCCACTGGTGTTCCTCCTAATATCTACGCATTTCACCGCTACACTAGG |
| | | AATTCCGCTTACCTCTCCAGCACTCCAGCTTAACAGTTTCCAAAGCAGTCCCGG |
| | | GGTTGAGCCCCGGGCTTTCACTTCAGACTTGCTAAGCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTCTCTTCCCTGCTGA |
| | | TAGAGCTTTACATACCGAAATACTTCTTCACTCACGCGGCGTCGCTGCATCAG |
| | | GGTTTCCCCCATTGTGCAATATTCCCCA |
| Pu_denovo6738 | 69 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCCAGGCCCGCAGTTTCCAATGCACTCCCGG |
| | | GGTTGGGCCCCGGGTTTTCACATCAGACTTGCTGGCCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAAGTTTACGTACCGAAATACTTCATCCTTCACGCGGCGTCGCTGCATCAG |
| | | GGTTCCCCCCATTGTGCAATATTCCCCA |
| Pu_denovo6738 | 70 | CCTGTTTGCTCCCCACGCTTTCGAGCCTCAACGTCAGTTACCGTCCAGTAAGCC |
| | | GCCTTCGCCACTGGTGTTCCTCCTAATATCTACGCATTTCACCGCTACACTAGG |
| | | AATTCCGCTTACCTCTCCGGCACTCCAGGCCCGCAGTTTCCAATGCACTCCCGG |
| | | GGTTGGGCCCCGGGTTTTCACATCAGACTTGCTGGCCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAAGTTTACGTACCGAAATACTTCATCCTTCACGCGGCGTCGCTGCATCAG |
| | | GGTTCCCCCCATTGTGCAATATTCCCCA |
| Pu_denovo6823 | 71 | CCTGTTTGCTCCCCACGCTTTCGTGCCTCAGTGTCAGTTACAGTCCAGAGAGCC |
| | | GCCTTCGCAACTGGTGTTCCTCCTAATATCTACGCATTTCACCGCTACACTAGG |
| | | AATTCCACTCTCCTCTCCTGCACTCAAGTTTATCAGTTTCAAAAGCTTACTATG |
| | | GTTGAGCCGTAGCCTTTCACTTCTGACTTGAAAGACCGCCTACGCACCCTTTAC |
| | | GCCCAGTAATTCCGGATAACGCTAGCCCCCTACGTATTACCGCGGCTGCTGGC |
| | | ACGTAGTTAGCCGGGGCTTCCTCCTCAAGTACCGTCATTATCTTCCTTGAGGAC |
| | | AGAGCTTTACGACCCGAAGGCCTTCATCGCTCACGCGGCGTTGCTGCATCAGG |
| | | CTTTCGCCCATTGTGCAATATTCCCCA |
| Pu_denovo683 | 72 | CCTGTTTGCTCCCCACGCTTTCGCACCTGAGCGTCAGTTACAGACCAGAGAGC |
| | | CGCCTTCGCCACTGGTGTTCCTCCATATATCTACGCATTTCACCGCTACACATG |
| | | GAATTCCACTCTCCTCTTCTGCACTCAAGTCTCCCAGTTTCCAATGACCCTCCC |
| | | CGGTTGAGCCGGGGGCTTTCACATCAGACTTAAGAAACCGCCTGCGCTCGCTT |
| | | TACGCCCAATAAATCCGGACAACGCTTGCCACCTACGTATTACCGCGGCTGCT |
| | | GGCACGTAGTTAGCCGTGGCTTTCTGGTTAGATACCGTCAGGGGACGTTCAGT |
| | | TACTAACGTCCTTGTTCTTCTCTAACAACAGAGTTTTACGATCCGAAAACCTTC |
| | | TTCACTCACGCGGCGTTGCTCGGTCAGACTTTCGTCCATTGCCGAAGATTCCCT |
| | | A |
| Pu_denovo6858 | 73 | CCTGTTCGCTCCCCCAGCTTTCGCGCCTCAGCGTCGGTCTCGGCCCAGAGGGCC |
| | | GCCTTCGCCACCGGTGTTCCACCCGATATCTGCGCATTCCACCGCTACACCGG |
| | | GTGTTCCACCCTCCCCTACCGGACCCGAGCCCGGCGGTTCAGGGGGCGGGACG |
| | | GGGTTGAGCCCCGCCATTTGACCCCCTGCCTGCCGGGCCGCCTACGCGCGCTT |
| | | TACGCCCAATGAATCCGGATAACGCTCGCCCCCTACGTATTACCGCGGCTGCT |
| | | GGCACGTAGTTAGCCGGGGCTTCTTCTGCAGGTACCGTCTTGTCTCATCCCTGC |
| | | TGAAAGCGGTTTACGACCCGAGGGCCTTCGTCCCGCACGCGGCGTCGCTGCGT |
| | | CAGGGTTCCCCCCATTGCGCAAGATTCCCCA |
| Pu_denovo7373 | 74 | CCTGTTTGCTCCCCACGCTTTCGAGCCTCAACGTCAGTTATCGTCCAGCAAGCC |
| | | GCCTTCGCCACTGGTGTTCCTCCTAATATCTACGCATTTCACCGCTACACTAGG |
| | | AATTCCGCTTGCCTCTCCGACACTCCAGCTGCACAGTTTCCAAAGCAGTCCCG |
| | | GGGTTGAGCCCCGGGCTTTCACTTCAGACTTGCACTGCCGTCTACGCTCCCTTT |
| | | ACACCCAGTAAATCCGGATAACGCTTGCCCCCTACGTATTACCGCGGCTGCTG |
| | | GCACGTAGTTAGCCGGGGCTTCTTAGTCAGGTACCGTCATTTTCTTCCCTGCTG |
| | | ATAGAAGTTTACATACCGAAATACTTCATCCTTCACGCGGCGTCGCTGCATCA |
| | | GGGTTTCCCCCATTGTGCAATATTCCCCA |
| Pu_denovo7649 | 75 | CCTGTTTGCTCCCCACGCTTTCGTACCTCAGCGTCAGTTTGTGTCCAGAAAGTC |
| | | GCCTTCGCTACTGGTATTCCTCCTAATATCTACGTATTTCACCACTACACTAGG |
| | | AATTCCACTTTCCTCTCCACTACTCAAGTTTATCAGTTTCCAATGCTTTACGGG |
| | | GTTGAGCCCCGATCTTTAACATTCGACTTATTAAACCGCCTGCGTACCCTTTAC |
| | | GCCCAATAATTCCGGACAACGCTCGCTCCATACGTATTACCGCGGCTGCTGGC |
| | | ACGTATTTAGCCGGAGCTTTCTTCTATGGTACTGTCATTATCTTCCCATAGGAC |
| | | AGAACTTTACGATACGAATACCTTCTTCGTTCACGCGGCGTCGCTGCATCAGG |
| | | GTTTCCCCCATTGTGCAATATTCCCCA |
| Pu_denovo7972 | 76 | CCTGTTCGCTCCCCACGCTTTCGCTCCTCAGCGTCAGTAACGGCCCAGAGACCT |
| | | GCCTTCGCCATTGGTGTTCTTCCCGATATCTACACATTCCACCGTTACACCGGG |
| | | AATTCCAGTCTCCCCTACCGCACTCAAGCCCGCCCGTACCCGGCGCGGATCCA |
| | | CCGTTAAGCGATGGACTTTCACACCGGACGCGACGAACCGCCTACGAGCCCTT |
| | | TACGCCCAATAATTCCGGATAACGCTTGCACCCTACGTATTACCGCGGCTGCT |
| | | GGCACGTAGTTAGCCGGTGCTTATTCAACGGGTAAACTCACTCTCGCTTGCTCC |
| | | CCGATAAAAGAGGTTTACAACCCGAAGGCCTCCATCCCTCACGCGGCGTCGCT |
| | | GCATCAGGCTTGCGCCCATTGTGCAATATTCCCCA |
| Pu_denovo8295 | 77 | CCTGTTTGCTCCCCACGCTTTCGAGCCTCAGCGTCAGTCATCGTCCAGTAAGCC |
| | | GCCTTCGCCACTGGTGTTCCTCCTAATATCTACGCATTTCACCGCTACACTAGG |
| | | AATTCCACTTACCCCTCCGACACTCTAGTCCGACAGTTTCCAATGCAGTACCGG |
| | | GGTTGAGCCCCGGGCTTTCACATCAGACTTGCCGTACCGCCTGCGCTCCCTTTA |
| | | CACCCAGTAAATCCGGATAACGCTTGCACCATACGTATTACCGCGGCTGCTGG |
| | | CACGTATTTAGCCGGTGCTTCTTAGTCAGGTACCGTCATTTCTTCTTCCCTGCT |
| | | GATAGAGCTTTACATACCGAAATACTTCTTCGCTCACGCGGCGTCGCTGCATC |
| | | AGGGTTTCCCCCATTGTGCAATATTCCCCA |
| Pu_denovo8302 | 78 | CCTGTTTGCTCCCCACGCTTTCGCGCCTCAGTGTCAGTTACAGACCAGGAAGCC |
| | | GCCTTCGCCACTGGTGTTCCTCCATATCTCTACGCATTTCACCGCTACACATGG |
| | | AGTTCCACTCTCCTCTTCTGCACTCAAGTCTCCCAGTTTCCAATGCACTACTCC |
| | | GGTTAAGCCGAAGGCTTTCACATCAGACTTAAAAGACCGCCTGCGTTCCCTTT |
| | | ACGCCCAATAAATCCGGATAACGCTTGCCACCTACGTATTACCGCGGCTGCTG |
| | | GCACGTAGTTAGCCGTGGCTTTCTGGTTAGATACCGTCGAAACGTGAACAGTT |
| | | ACTCTCACGCACTTTCTTCTCTAACAACAGGGTTTTACGATCCGAAGACCTTCT |
| | | TCACCCACGCGGCGTTGCTCCATCAGGCTTTCGCCCATTGTGGAAGATTCCCTA |
| Pu_denovo8600 | 79 | CCTGTTTGCTACCCACGCTTTCGCGCTTTAGCGTCAGTATCTGTCCAGTAGGCT |
| | | GGCTTCCCCATCGGCATTCCTACAAATATCTACGAATTTCACCTCTACACTTGT |
| | | AGTTCCGCCTACCTCTCCAGTACTCTAGTTTGGCAGTTTCCAACGCAATACGGA |
| | | GTTGAGCCCCGCATTTTCACATCAGACTTACCAAACCGCCTAGACGCGCTTTA |
| | | CGCCCAATAAATCCGGATAACGCTTGCGACATACGTATTACCGCGGCTGCTGG |
| | | CACGTATTTAGCCGTCGCTTCTTCTGTTGGTACCGTCACTTTCTTCTTCCCAACT |
| | | GAAAGCACTTTACATTCCGAAAAACTTCATCGTGCACACAGAATTGCTGGATC |
| | | AGACTTTTGGTCCATTGTCCAATATTCCCCA |
| Pu_denovo8725 | 80 | CCTGTTTGCTCCCCACGCTTTCGAGCCTCAACGTCAGTCACTGTCCAGTAAGCC |
| | | GCCTTCGCCACTGGTGTTCCTCCTAATATCTACGCATTTCACCGCTACACTAGG |
| | | AATTCCACTTACCTCTCCAGCACTCTAGCTATACAGTTTCCAAAGCAGTCCCGG |
| | | GGTTGAGCCCCGGGCTTTCACTTCAGACTTGCACAGCCGTCTACGCTCCCTTTA |
| | | CACCCAGTAAATCCGGATAACGCTTGCCCCCTACGTATTACCGCGGCTGCTGG |
| | | CACGTAGTTAGCCGGGGCTTCTTAGTCAGGTACCGTCATTTTCTTCCCTGCTGA |
| | | TAGAAGTTTACATACCGAAATACTTCATCCTTCACGCGGCGTCGCTGCATCAG |
| | | GCTTTCGCCCATTGTGCAATATTCCCCA |
| Pu_denovo8737 | 81 | CCTGTTTGCTCCCCACGCTTTCGAGCCTCAGCGTCAGTTACAGACCAGAGAGC |
| | | CGCTTTCGCCACCGGTGTTCCTCCATATATCTACGCATTTCACCGCTACACTAG |
| | | GAATTCCGCTTGCCTCTCCGACACTCCAGCTGCACAGTTTCCAAAGCAGTCCC |
| | | GGGGTTGAGCCCCGGGCTTTCACTTCAGACTTGCACTGCCGTCTACGCTCCCTT |
| | | TACACCCAGTAAATCCGGATAACGCTTGCCCCCTACGTATTACCGCGGCTGCT |
| | | GGCACGTAGTTAGCCGGGGCTTCTTAGTCAGGTACCGTCATTTTCTTCCCTGCT |
| | | GATAGAAGTTTACATACCGAAATACTTCATCCTTCACGCGGCGTCGCTGCATC |
| | | AGGGTTTCCCCCATTGTGCAATATTCCCCA |
| Pu_denovo920 | 82 | CCTGTTTGCTCCCCACGCTTTCGAGCCTCAGCGTCAGTTACAGTCCAGAAAGG |
| | | CGCCTTCGCCACTGGTATTCTTCCTAATCTCTACGCATTTCACCGCTACACTAG |
| | | GAATTCTCCTTTCCTCTCCTGCACTCTAGATATCCAGTTTGGAATGCAGCACTC |
| | | AAGTTGAGCCCGAGTATTTCACATCCCACTTAAACATCCGCCTACGCTCCCTTT |
| | | ACGCCCAGTAAATCCGGACAACGCTTGCCACCTACGTATTACCGCGGCTGCTG |
| | | GCACGTAGTTAGCCGTGGCTTCCTCCTCAGGTACCGTCATTATCGTCCCTGAAG |
| | | ACAGAGTTTTACAACCCGAAGGCCGTCATCACTCACGCGGCGTTGCTGCATCA |
| | | GGCTTTCGCCCATTGTGCAATATTCCCCA |
| Pu_denovo9465 | 83 | CCTGTTTGCTCCCCACGCTTTCGCGCCTCAGTGTCAGTTACAGACCAGGAAGCC |
| | | GCCTTCGCCACTGGTGTTCCTCCATATCTCTACGCATTTCACCGCTACACATGG |
| | | AATTCCACTTCCCTCTTCTGCACTCAAGTCGACCAGTTTCCAATGACCCTCCAC |
| | | GGTTAAGCCGTGGGCTTTCACATCAGACTTAATCAACCACCTGCGCGCTCTTTA |
| | | CGCCCAATAATTCCGGATAACGCTCGCCACCTACGTATTACCGCGGCTGCTGG |
| | | CACGTAGTTAGCCGTGGCTTTCTCATAAGGTACCGTCACACTCTAGCCATTTCC |
| | | TACTAAAGTCGTTCTTCCCTTATAACAGAATTTTACAACCCGAAGGCCTTCATC |
| | | ATTCACGCGGCGTTGCTCGGTCAGGCTTTCGCCCATTGCCGAAGATTCCCTA |
| Pu_denovo9465 | 84 | CCTGTTTGCTCCCCACGCTTTCGCGCCTCAGTGTCAGTTACAGACCAGGAAGCC |
| | | GCCTTCGCCACTGGTGTTCCTCCATATCTCTACGCATTTCACCGCTACACATGG |
| | | AATTCCACTTCCCTCTTCTGCACTCAAGTCGACCAGTTTCCAATGACCCTCCAC |
| | | GGTTAAGCCGTGGGCTTTCACATCAGACTTAATCAACCACCTGCGCGCTCTTTA |
| | | CGCCCAATAATTCCGGATAACGCTCGCCACCTACGTATTACCGCGGCTGCTGG |
| | | CACGTAGTTAGCCGTGGCTTTCTCATAAGGTACCGTCACACTCTAGCCATTTCC |
| | | TACTAAAGTCGTTCTTCCCTTATAACAGAATTTTACAACCCGAAGGCCTTCATC |
| | | ATTCACGCGGCGTTGCTCGGTCAGGCTTTCGCCCATTGCCGAAGATTCCCTA |
| Pu_denovo959 | 85 | CCTGTTTGCTCCCCACGCTTTCGCGCCTCAGTGTCAGTTGCAGACCAGGAAGCC |
| | | GCCTTCGCCACTGGTGTTCCTCCATATCTCTACGCATTTCACCGCTACACATGG |
| | | AATTCCACTTCCCTCTTCTGCACTCAAGTCAACCAGTTTCCAATGACCCTCCAC |
| | | GGTTAAGCCGTGGGCTTTCACATCAGACTTAATTAACCACCTGCGCGCTCTTTA |
| | | CGCCCAATAATTCCGGATAACGCTTGCCACCTACGTATTACCGCGGCTGCTGG |
| | | CACGTAGTTAGCCGTGGCTTTCTCATAAGGTACCGTCAATTGATAGTCATTTCC |
| | | TCCTATCACCTTTCTTCCCTTATAACAGAATTTTACAACCCGAAGGCCTTCTTC |
| | | ATTCACGCGGCGTTGCTCGGTCAGGCTTTCGCCCATTGCCGAAGATTCCCTA |

TABLE 2.2
DNA sequences for bacterial taxa associated with health in mammals and detected in
adult and mature dogs

| OTU_ID | SEQ ID | 16SrDNA partial DNA sequence |
|---|---|---|
| Ad_denovo40 | 86 | CCCGTTCGCTACCCTGGCTTTCGCATCTCAGCGTCAGACACAGTCCAGAAAGGCG |
| | | CCTTCGCCACTGGTGTTCCTCCCAATATCTACGCATTTCACCGCTACACTGGGAAT |
| | | TCCCCTTTCCTCTCCTGCACTCAAGTCTCCCAGTATCCAGAGCCATACGGGGTTGA |
| | | GCCCCGCATTTTCACTCCAGACTTAAGAAACCGCCTACATGCTCTTTACGCCCAAT |
| | | AATTCCGGACAACGCTCGCCACCTACGTATTACCGCGGCTGCTGGCACGTAGTTA |
| | | GCCGTGGCTTATTCGTTTACTACCGTCATTACAAATAATTATTCACAATCTGCACA |
| | | TTCGTCATAAACAAAAGAGTTTTACGGAACGAATTCCTTCATCACTCACGCGGCA |
| | | TTGCTCCGTCAGGCTTTCGCCCATTGCGGAAGATTCCCCA |
| Ad_denovo75 | 87 | CCTGTTTGATACCCGCACTTTCGAGCATCAGCGTCAGTTACGGTCCAGCAAGCTG |
| | | CCTTCGCAATCGGAGTTCTTCGTGATATCTAAGCATTTCACCGCTACACCACGAAT |
| | | TCCGCCTGCCTTTACCGCACTCAAGAACTCCAGTATCAACTGCAATTTTACGGTTG |
| | | AGCCGCAAACTTTCACAACTGACTTAAAATTCCGCCTACGCTCCCTTTAAACCCA |
| | | ATAAATCCGGATAACGCTCGGATCCTCCGTATTACCGCGGCTGCTGGCACGGAGT |
| | | TAGCCGATCCTTATTCATACGGTACATACAAAAAGCCACACGTGGCTAACTTTAT |
| | | TCCCGTATAAAAGAAGTTTACAACCCATAGGGCAGTCATCCTTCACGCTACTTGG |
| | | CTGGTTCAGACTCTCGTCCATTGACCAATATTCCTCA |
| Ad_denovo237 | 88 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCTCTCCGGCACTCGAGTATCACAGTTTCCAATGCAGTCCAGGGGTTG |
| | | AGCCCCCGCCTTTCACATCAGACTTGCAACACCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCCA |
| Ad_denovo323 | 89 | CCTATTTGCTCCCCACGCTTTCGTGCCTCAGCGTCAGGTACAGGCCAGGCGGCCG |
| | | CCTTCGCCGCTGGTGTTCTTCCACATCTCTACGCATTTTACCGCTACACATGGAGT |
| | | TCCACCGCCCTCTCCTGTCCTCAAGCCGTGCAGTTTCCAAAGCCTGAACCGGTTGA |
| | | GCCGGTCCCTTTTACTTCAGACTTGCACAGCCGCCTGCGCACCCTTTACGCCCAAT |
| | | CATTCCGGATAACGCTTGCCACCTACGTATTACCGCGGCTGCTGGCACGTAGTTA |
| | | GCCGTGGCTTTCTGGCCGGGTACCATCAAACAGTCAGCTTTCCACTCTGGCTGTCC |
| | | TTTGTCCCCGGCAACAGGGCTTTACAATCCGAAGACCGTCTTCACCCACGCGGCA |
| | | TTGCTCGTTCAGGCTTGCGCCCATTGACGAAAATTCCCTA |
| Ad_denovo648 | 90 | CCTGTTTGCTCCCCACGCTTTCGAGCCTCAGCGTCAGTTACAGTCCAGAGAATCGC |
| | | CTTCGCCACTGGTGTTCTTCCTAATCTCTACGCATTTCACCGCTACACTAGGAATT |
| | | CCATTCTCCTCTCCTGCACTCTAGATACCCAGTTTGGAATGCAGCTCCCAGGTTAA |
| | | GCCCAGGTATTTCACATCCCACTTAAGTATCCGCCTACGCTCCCTTTACGCCCAGT |
| | | AAATCCGGATAACGCTTGCCACCTACGTATTACCGCGGCTGCTGGCACGTAGTTA |
| | | GCCGTGGCTTCCTCCTTAGGTACCGTCATTATCGTCCCTAAAGACAGAGCTTTACA |
| | | ATCCGAAGACCTTCATCACTCACGCGGCGTTGCTGCATCAGGGTTTCCCCCATTGT |
| | | GCAATATTCCCCA |
| Ad_denovo871 | 91 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCCCTCCGGCACTCAAGCCTGGCAGTTTCCAATGCAGTCCAGGAGTTG |
| | | AGCCCCTGCCTTTCACATCAGACTTGCCATGCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTATCTTCCCTGCTGATAGAAGTTTAC |
| | | ATACCGAAATACTTCATCGCTCACGCGGCGTCGCTGCATCAGGGTTTCCCCCATTG |
| | | TGCAATATTCCCCA |
| Ad_denovo957 | 92 | CCTATTTGCTCCCCACGCTTTCGTGCCTCAGCGTCAGTCACAGGCCAGGCGGCCGC |
| | | CTTCGCCACTGGTGTTCTTCCATATCTCTGCGCATTTTACCGCTACACATGGAGTT |
| | | CCACCGCCCTCTCCTGTCCTCAAGTCTGCCAGTTTCTGAATCATGAATGAGTTGAG |
| | | CTCATCCCTTTGCCTTCAGACTTAACAGACCGCCTGCGCACCCTTTACGCCCAATC |
| | | ATTCCGGATAACGCTCGCCACCTACGTATTACCGCGGCTGCTGGCACGTAGTTAG |
| | | CCGTGGCTTTCTGGCCGGGTACCATCCATGAAATGCCATTTCCTGCATTCCCTCTT |
| | | TTTCCCCGGCAACAGAGCTTTACAATCCGAAGACCTTCTTCACTCACGCGGCATTG |
| | | CTCGTTCAGGCTTGCGCCCATTGACGAAAATTCCCTA |
| Ad_denovo1210 | 93 | CCTGTTTGCTCCCCACGCTTTCGAGCCTCAACGTCAGTTACTGTCCAGTAAGCCGC |
| | | CTTCGCCACTGGTGTTCCTCCTAATATCTACGCATTTCACCGCTACACTAGGAATT |
| | | CCACTTACCTCTCCAGCACTCTAGATGAACAGTTTCCAATGCAGTCCCGGGGTTG |
| | | AGCCCCGGGTTTTCACATCAGACTTGCCCATCCGTCTACGCTCCCTTTACACCCAG |
| | | TAAATCCGGATAACGCTTGCACCATACGTATTACCGCGGCTGCTGGCACGTATTT |
| | | AGCCGGTGCTTCTTAGTCAGGTACCGTCATTATCTTCCCTGCTGATAGAGCTTTAC |
| | | ATACCGAAATACTTCATCGCTCACGCGGCGTCGCTGCATCAGGGTTTCCCCCATTG |
| | | TGCAATATTCCCCA |
| Ad_denovo1428 | 94 | CCTATTTGCTCCCCACGCTTTCGTGCCTCAGCGTCAGTAACAGTCCAGGCGGCCGC |
| | | CTTCGCAACTGGTGTTCTTCCATATATCTACGCATTTTACCGCTACACATGGAGTT |
| | | CCACCGCCCTCTCCTGTCCTCGAGCGCGCCAGTTTCCAAAGCCTGCACAGGTTGA |
| | | GCCTGTACCTTTTACTTCAGACTTGACGCGCCGCCTGCGCACCCTTTACGCCCAAT |
| | | CATTCCGGATAACGCTCGCCACCTACGTATTACCGCGGCTGCTGGCACGTAGTTA |
| | | GCCGTGACTTTCTGGCGGGGTACCATCAAAAGCAGACCATTTCCTTTCTGCTTCCT |
| | | TTTTCCCCCGCAACAGAGCTTTACGATCCGAAGACCTTCCTCACTCACGCGGCATT |
| | | GCTCGTTCAGGCTTGCGCCCATTGACGAAAATTCCCTA |
| Ad_denovo1613 | 95 | CCTGTTTGCTCCCCACGCTTTCGAGCCTCAGCGTCAGTTACAGACCAGAAAGCCG |
| | | CCTTCGCCACTGGTGTTCCTCCATATATCTACGCATTTCACCGCTACACATGGAAT |
| | | TCCGCTTTCCTCTTCTGCACTCAAGTCTCCCAGTTTCCAATGACCCTCCCCGGTTA |
| | | AGCCGGGGGCTTTCACATCAGACTTAAAAGACCGCCTGCGCTCGCTTTACGCCCA |
| | | ATAAATCCGGACAACGCTTGCCACCTACGTATTACCGCGGCTGCTGGCACGTAGT |
| | | TAGCCGTGGCTTTCTGGTTAGATACCGTCAAGGGATGAACTTTCCACTCTCATCCT |
| | | TGTTCTTCTCTAACAACAGAGTTTTACGATCCGAAAACCTTCTTCACTCACGCGGC |
| | | GTTGCTCGGTCAGACTTGCGTCCATTGCCGAAGATTCCCTA |
| Ad_denovo1767 | 96 | CCTGTTTGCTCCCCACGCTTTCGAGCCTCAGCGTCAGTTACAGACCAGAGAGCCG |
| | | CCTTCGCCACTGGTGTTCCTCCATATATCTACGCATTTCACCGCTACACATGGAAT |
| | | TCCACTCTCCTCTTCTGCACTCAAGTCTCCCAGTTTCCAATGACCCTCCCCGGTTG |
| | | AGCCGGGGGCTTTCACATCAGACTTAAGAAACCGCCTGCGCTCGCTTTACGCCCA |
| | | ATAAATCCGGACAACGCTTGCCACCTACGTATTACCGCGGCTGCTGGCACGTAGT |
| | | TAGCCGTGGCTTTCTGGTTAGATACCGTCAAGGGATGAACAGTTACTCTCATCCTT |
| | | GTTCTTCTCTAACAACAGAGTTTTACGATCCGAAAACCTTCTTCACTCACGCGGCG |
| | | TTGCTCGGTCAGACTTTCGTCCATTGCCGAAGATTCCCTA |
| Ad_denovo1838 | 97 | CCTGTTTGCTACCCACGCTTTCGAGCCTCAGCGTCAGTTAAAGCCCAGCAGGCCG |
| | | CCTTCGCCACTGGTGTTCCTCCTAATATCTACGCATTTCACCGCTACACTAGGAAT |
| | | TCCGCCTGCCTCTACTTCACTCAAGAAAAACAGTTTTGAAAGCAGCTCATGGGTT |
| | | GAGCCCATGCATTTCACTTCCAACTTGCTTTCCCGCCTACGCTCCCTTTACACCCA |
| | | GTAATTCCGGACAACGCTCGCTCCCTACGTATTACCGCGGCTGCTGGCACGTAGT |
| | | TAGCCGGAGCTTCCTTCTTCGGTACCGTCACTTTCTTCGTCCCGAATGACAGAGGT |
| | | TTACAACCCGAAGGCCGTCTTCCCTCACGCGGCGTCGCTGCATCAGGCTTTCGCCC |
| | | ATTGTGCAATATCCCCCA |
| Ad_denovo2183 | 98 | CCTGTTTGCTCCCCACGCTTTCGCGCCTCAGTGTCAGTTACAGACCAGGAAGCCGC |
| | | CTTCGCCACTGGTGTTCCTCCATATCTCTACGCATTTCACCGCTACACATGGAATT |
| | | CCACTTCCCTCTTCTGCACTCAAGTCGACCAGTTTCCAATGACCCTCCACGGTTAA |
| | | GCCGTGGGCTTTCACATCAGACTTAATCAACCACCTGCGCGCTCTTTACGCCCAAT |
| | | AATTCCGGATAACGCTCGCCACCTACGTATTACCGCGGCTGCTGGCACGTAGTTA |
| | | GCCGTGGCTTTCTCATAAGGTACCGTCACACTCTAGCCATTTCCTACTAAAGTCGT |
| | | TCTTCCCTTATAACAGAATTTTACAACCCGAAGGCCTTCATCATTCACGCGGCGTT |
| | | GCTCGGTCAGGCTTTCGCCCATTGCCGAAGATTCCCTA |
| Ad_denovo2510 | 99 | CCTGTTTGCTACCCACACTTTCGAGCCTCAGCGTCAGTTGGTGCCCAGTAGGCCGC |
| | | CTTCGCCACTGGTGTTCCTCCCGATATCTACGCATTCCACCGCTACACCGGGAATT |
| | | CCGCCTACCTCTGCACTACTCAAGAAAAACAGTTTTGAAAGCAGTTTATGGGTTG |
| | | AGCCCATAGATTTCACTTCCAACTTGTCTTCCCGCCTGCGCTCCCTTTACACCCAG |
| | | TAATTCCGGACAACGCTTGTGACCTACGTTTTACCGCGGCTGCTGGCACGTAGTTA |
| | | GCCGTCACTTCCTTGTTGGGTACCGTCATTATCTTCCCCAACAACAGGAGTTTACA |
| | | ATCCGAAGACCTTCTTCCTCCACGCGGCGTCGCTGCATCAGGGTTTCCCCCATTGT |
| | | GCAATATTCCCCA |
| Ad_denovo2718 | 100 | CCTATTTGCTCCCCACGCTTTCGTGCCTCAGCGTCAGTAACAGGCCAGGCGGCCG |
| | | CCTTCGCCACTGGTGTTCTTCCATATATCTACGCATTTTACCGCTACACATGGAGT |
| | | TCCACCGCCCTCTCCTGTCCTCCAGCCTGCCAGTTTCCAAAGCCTGTACCGGTTGA |
| | | GCCGGTACCTTTCACTTCAGACTTAACAGGCCGCCTACGCACCCTTTACGCCCAAT |
| | | CATTCCGGATAACGCTCGCCACCTACGTATTACCGCGGCTGCTGGCACGTAGTTA |
| | | GCCGTGACTTCCTCGAAAGGTAATATCACTTCACCAGCATTTCCTCTGGTGTTCCT |
| | | TTTTCCCTCTCAACAGAACTTTACGATCCGAAGACCTTCCTCGTTCACGCGGCATT |
| | | GCTCGTTCAGGCTTGCGCCCATTGACGAAAATTCCCTA |
| Ad_denovo2798 | 101 | CCTGTTCGCTCCCCACGCTTTCGCTCCTCAGCGTCAGTAACGGCCCAGAGACCTGC |
| | | CTTCGCCATTGGTGTTCTTCCCGATATCTACACATTCCACCGTTACACCGGGAATT |
| | | CCAGTCTCCCCTACCGCACTCAAGCCCGCCCGTACCCGGCGCGGATCCACCGTTA |
| | | AGCGATGGACTTTCACACCGGACGCGACGAACCGCCTACGAGCCCTTTACGCCCA |
| | | ATAAATCCGGATAACGCTTGCACCCTACGTATTACCGCGGCTGCTGGCACGTAGT |
| | | TAGCCGGTGCTTATTCGAACAATCCACTCAACACGGCCAAAGACCGTGCCTTGCC |
| | | CTTGAACAAAAGCGGTTTACAACCCGAAGGCCTCCATCCCGCACGCGGCGTCGCT |
| | | GCATCAGGCTTGCGCCCATTGTGCAATATTCCCCA |
| Ad_denovo2806 | 102 | CCTGTTTGCTCCCCACGCTTTCGAGCCTCAGCGTCAGTTACAGTCCAGAGAATCGC |
| | | CTTCGCCACTGGTGTTCTTCCTAATCTCTACGCATTTCACCGCTACACTAGGAATT |
| | | CCATTCTCCTCTCCTGCACTCTAGACTTCCAGTTTGAAATGCAGCACCCAAGTTGA |
| | | GCCCGGGTATTTCACATCTCACTTAAAAGTCCGCCTACGCTCCCTTTACGCCCAGT |
| | | AAATCCGGACAACGCTCGCCACCTACGTATTACCGCGGCTGCTGGCACGTAGTTA |
| | | GCCGTGGCTTCCTCCTCAGGTACCGTCATTATCGTCCCTGAAGACAGAGCTTTACA |
| | | ACCCGAAGGCCGTCATCACTCACGCGGCGTTGCTGCATCAGGGTTTCCCCCATTG |
| | | TGCAATATTCCCCA |
| Ad_denovo2900 | 103 | CCTGTTTGATACCCGCACTTTCGAGCCTCAGCGTCAGTTACACTCCAGATACCTGC |
| | | CTTCGCGATCGGAGTTCCTCATGATATCTGAGCATTTCACCGCTACACCATGAATT |
| | | CCAGTATCTCTGCGTGTACTCAAGACTCCCAGTATCAACTGCAGTCCGACGGTTG |
| | | AGCCGCCGTATTTCACAACTGACTTAAGAGTCCGCCTGCGCTCCCTTTAAACCCA |
| | | ATAAATCCGGATAACGCCTGGACCTTCCGTATTACCGCGGCTGCTGGCACGGAAT |
| | | TAGCCGGTCCTTTTTCTGATGGTACATACAAAACGGTACACGTACCGCACTTTATT |
| | | CCCAACTAAAAGCAGTTTACAACCCAGAGGGCCGTCATCCTGCACGCTACTTGGC |
| | | TGGTTCAGACTTCCGTCCATTGACCAATATTCCTCA |
| Ad_denovo3016 | 104 | CCTGTTTGCTCCCCACGCTTTCGAGCCTCAACGTCAGTTATCGTCCAGTAAGCCGC |
| | | CTTCGCCACTGGTGTTCCTCCTAATATCTACGCATTTCACCGCTACACTAGGAATT |
| | | CCACTTACCCCTCCGACACTCTAGACTGACAGTTTCCAATGCAGTCCCGGGGTTG |
| | | AGCCCCGGGTTTTCACATCAGACTTGCCAGTCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAGCTTTAC |
| | | ATACCGAAATACTTCTTCGCTCACGCGGCGTCGCTGCATCAGGGTTTCCCCCATTG |
| | | TGCAATATTCCCCA |
| Ad_denovo3299 | 105 | CCTGTTTGCTCCCCACGCTTTCGCGCCTCAGTGTCAGTTGCAGACCAGGAAGCCGC |
| | | CTTCGCCACTGGTGTTCCTCCATATCTCTACGCATTTCACCGCTACACATGGAATT |
| | | CCACTTCCCTCTTCTGCACTCAAGTCAACCAGTTTCCAATGACCCTCCACGGTTAA |
| | | GCCGTGGGCTTTCACATCAGACTTAATTAACCACCTGCGCGCTCTTTACGCCCAAT |
| | | AATTCCGGATAACGCTTGCCACCTACGTATTACCGCGGCTGCTGGCACGTAGTTA |
| | | GCCGTGGCTTTCTCATAAGGTACCGTCAATTGATAGTCATTTCCTCCTATCACCTT |
| | | TCTTCCCTTATAACAGAATTTTACAACCCGAAGGCCTTCTTCATTCACGCGGCGTT |
| | | GCTCGGTCAGGCTTTCGCCCATTGCCGAAGATTCCCTA |
| Ad_denovo3427 | 106 | CCTGTTTGCTCCCCACGCTTTCGTGCCTCAGCGTCAGTTACAGTCCAGAAAGCCGC |
| | | CTTCGCTACTGGTGTTCCTCCCAATATCTACGCATTTCACCGCTACACTGGGAATT |
| | | CCGCTTTCCTCTCCTGCACTCAAGTCAGACAGTATCAGGAGCTTACTACGGTTGAG |
| | | CCGTAGCCTTTAACTCCTGACTTGAAAGACCGCCTACGCACCCTTTACGCCCAGTA |
| | | AATCCGGATAACGCTAGCCCCCTACGTATTACCGCGGCTGCTGGCACGTAGTTAG |
| | | CCGGGGCTTCCTCCTCAAGTACCGTCATTATCTTCCTTGAGGACAGAGCTTTACGA |
| | | CCCGAAGGCCTTCATCGCTCACGCGGCATTGCTCGTTCAGGCTTGCGCCCATTGAC |
| | | GAAAATTCCCTA |
| Ad_denovo3603 | 107 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCTCTCCGGTACTCAAGATCAACAGTTTCCAATGCAGTCCGGGGGTTG |
| | | AGCCCCCGCCTTTCACATCAGACTTGCTGCTCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCCA |
| Ad_denovo3746 | 108 | CCTGTTCGCTCCCCCAGCTTTCGCGCCTCAGCGTCGGTCTCGGCCCAGAGGGCCGC |
| | | CTTCGCCACCGGTGTTCCTCCCGATATCTGCGCATTCCACCGCTACACCGGGAATT |
| | | CCACCCTCCCCTACCGGACCCGAGCCGCGGGGTTCGGGGGGCGGACCGGGGTTG |
| | | AGCCCCGGGATTTGACCCCCCGCCTACGCGGCCGCCTACGCGCGCTTTACGCCCA |
| | | ATGAATCCGGATAACGCTCGCCCCCTACGTATTACCGCGGCTGCTGGCACGTAGT |
| | | TAGCCGGGGCTTCTTCTGCAGGTACCGTCTTGACTCGTCCCTGCTGAAAGCGGTTT |
| | | ACGACCCGAAGGCCTTCGTCCCGCACGCGGCGTCGCTGCGTCAGGGTTGCCCCCA |
| | | TTGCGCAAGATTCCCCA |
| Ad_denovo3795 | 109 | CCTGTTTGCTCCCCATGCTTTCGCGCTTCAGCGTCAGTATCTGTCCAGTGAGCTGA |
| | | CTTCTCTATCGGCATTCCTACAAATATCTACGAATTTCACCTCTACACTTGTAGTT |
| | | CCGCCCACCTCTCCAGTACTCTAGTCTGGCAGTTTCCAACGCAATACGGAGTTGA |
| | | GCCCCGCATTTCCACATCAGACTTACCAGACCGCCTAGACGCGCTTTACGCCCAA |
| | | TAAATCCGGATAACGCTTGCGACATACGTATTACCGCGGCTGCTGGCACGTATTT |
| | | AGCCGTCGCTTCTTCTGTTGGTACCGTCACTTTCTTCTTCCCAACTGAAAGCACTTT |
| | | ACATTCCGAAAAACTTCATCGTGCACACAGAATTGCTGGATCAGACTTTTGGTCC |
| | | ATTGTCCAATATTCCCCA |
| Ad_denovo3807 | 110 | CCTATTTGCTCCCCACGCTTTCGTGCCTCAGCGTCAGCAACAGGCCAGGCGGCCG |
| | | CCTTCGCCACTGGTGTTCTTCCATATATCTACGCATTTTACCGCTACACATGGAGT |
| | | TCCACCGCCCTCTCCTGCTCTCCAGTCCTCCAGTTTCCAAAGCCATGCATGAGTTG |
| | | AGCTCATGCGTTTCACTCCAGACTTGCAGGACCGCCTGCGCACCCTTTACGCCCA |
| | | ATCATTCCGGATAACGCTCGCCACCTACGTATTACCGCGGCTGCTGGCACGTAGT |
| | | TAGCCGTGACTTTCTGGCGGGGCACCATCAGTCAGCACCCATTTCCTGATGCTGCC |
| | | TTTTTTCCCCCGCAACAGAGCTTTACGACCCGAAGGCCTTCTTCACTCACGCGGCA |
| | | TCGCTCGTTCAGGCTTGCGCCCATTGACGAAAATTCCCTA |
| Ad_denovo3832 | 111 | CCTGTTTGCTCCCCATGCTTTCGCGCTTCAGCGTCAGTATCTGTCCAGTGAGCTGA |
| | | CTTCTCTATCGGCATTCCTACAAATATCTACGAATTTCACCTCTACACTTGTAGTT |
| | | CCGCCCACCTCTCCAGTACTCTAGTCTGGCAGTTTCCAACGCAATACGGAGTTGA |
| | | GCCCCGCATTTCCACATCAGACTTACCAGACCGCCTAGACGCGCTTTACGCCCAA |
| | | TAAATCCGGATAACGCTTGCGACATACGTATTACCGCGGCTGCTGGCACGTATTT |
| | | AGCCGTCGCTTCTTCTATCGGTACCATCACTTTCTTTTTCCCGATTGAAAGCACTTT |
| | | ACAATCCTAAGACCGTCATCGTGCACACAGAATTGCTGGATCAGACTTTTGGTCC |
| | | ATTGTCCAATATTCCCCA |
| Ad_denovo4316 | 112 | CCTGTTCGCTCCCCCAGCTTTCGCGCCTCAGCGTCAGTGGCGGCCCAGCAGGCTG |
| | | CCTTCGCCATCGGTGTTCTTCCCGATATCTGCGCATTCCACCGCTACACCGGGAAT |
| | | TCCGCCTGCCTCTACCGCACTCGAGCCGCCCAGTCCGGAACCCGGCCCGAGGTTG |
| | | AGCCCCGGGATTAGAGGTTCCGCTTAGGCGGCCGCCTACGCGCGCTTTACGCCCA |
| | | ATGAATCCGGATAACGCTCGCCCCCTACGTATTACCGCGGCTGCTGGCACGTAGT |
| | | TAGCCGGAGCTTCTTCTGCAGGTACCGTCTATGTCTTCCCTGCTGAAAGCGGTTTA |
| | | CAACCCGAAGGCCTTCGTCCCGCACGCGGCGTTGCTGCGTCAGGGTTTCCCCCAT |
| | | TGCGCAAAATTCCCCA |
| Ad_denovo4538 | 113 | CCTGTTTGCTCCCCACGCTTTCGAGCCTCAACGTCAGTCATCGTCCAGTAAGCCGC |
| | | CTTCGCCACTGGTGTTCCTCCTAATATCTACGCATTTCACCGCTACACTAGGAATT |
| | | CCACTTACCTCTCCGACACTCTAGAAAAACAGTTTCCAATGCAGTCCCGGGGTTG |
| | | AGCCCCGGGTTTTCACATCAGACTTGCCTCTCCGTCTACGCTCCCTTTACACCCAG |
| | | TAAATCCGGATAACGCTTGCACCATACGTATTACCGCGGCTGCTGGCACGTATTT |
| | | AGCCGGTGCTTCTTAGTCAGGTACCGTCATTTTCTTCCCTGCTGATAGAGCTTTAC |
| | | ATACCGAAATACTTCATCGCTCACGCGGCGTCGCTGCATCAGGGTTTCCCCCATTG |
| | | TGCAATATTCCCCA |
| Ad_denovo4688 | 114 | CCTGTTTGCTCCCCACGCTTTCGCGCCTCAGCGTCAGTTGTCGTCTAGAAAGTCGC |
| | | CTTCGCCACCGGTGTTCTTCCTAATCTCTACGCATTTCACCGCTACACTAGGAATT |
| | | CCACTTTCCCCTCCGACACTCCAGCCCTGCAGTTTCCATCCCCTCATGGGGTTAAG |
| | | CCCCACGCTTTTAAGATGGACTTGCACGGCCGCCTGCGCGCGCTTTACGCCCAAT |
| | | AATTCCGGACAACGCTTGCCACCTACGTATTACCGCGGCTGCTGGCACGTAGTTA |
| | | GCCGTGGCTTGCTTTTCCGGTACCGTCAACATCAATCAATGTTCTCAATCAATGCC |
| | | TTCGTCCCGGATCACAGAACTTTACAATCCGAAGACCTTCATCGTTCACGCGGCG |
| | | TTGCTCCGTCAGACTTTCGTCCATTGCGGAAGATTCCCCA |
| Ad_denovo5389 | 115 | CCTGTTTGATACCCGCACTTTCGAGCCTCAGCGTCAGTTGCACCCCGGATACCTGC |
| | | CTTCGCGATCGGAGTTCTTCATGATATCTGAGCATTTCACCGCTACACCATGAATT |
| | | CCAGCATCCCTGTGTGCACTCAAGACTCCCAGTATCAACTGCAGTCCGACGGTTG |
| | | AGCCGCCGTATTTCACAACTGACTTAAGAGTCCGCCTGCGCTCCCTTTAAACCCA |
| | | ATAAATCCGGATAACGCCTGGACCTTCCGTATTACCGCGGCTGCTGGCACGGAAT |
| | | TAGCCGGTCCTTTTTCTGATGGTACATACAAAACAGCTCACGAGCTGCACTTTATT |
| | | CCCAACTAAAAGCAGTTTACAACCCGGAGGGCCGTCATCCTGCACGCTACTTGGC |
| | | TGGTTCAGACTTGCGTCCATTGACCAATATTCCTCA |
| Ad_denovo5707 | 116 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCTCTCCGGCACTCTAGAAAAACAGTTTCCAATGCAGTCCTGGGGTTA |
| | | AGCCCCAGCCTTTCACATCAGACTTGCTCTTCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCCA |
| Ad_denovo5908 | 117 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCCCTCCGGTACTCAAGACTGACAGTTTCCAATGCAGTCCAGGGGTTG |
| | | AGCCCCTGCCTTTCACATCAGACTTGCCATTCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCCA |
| Ad_denovo5931 | 118 | CCTGTTTGCTACCCACGCTTTCGAGCCTCAGCGTCAGTTACAGCCCAGTAGGCCGC |
| | | CTTCGCCACTGGTGTTCCTCCTAATATCTACGCATTTCACCGCTACACTAGGAATT |
| | | CCGCCTACCTCTACTGCACTCAAGAGTGGCAGTTTTGAACGCGACTATCAGTTGA |
| | | GCCGATAGTTTAGACATTCAACTTGCCTCCCCGCCTACGCTCCCTTTACACCCAGT |
| | | AATTCCGGACAACGCTTGCCACCTACGTATTACCGCGGCTGCTGGCACGTAGTTA |
| | | GCCGTGGCTTCCTCCTCGGGTACCGTCATTTATTCGTCCCCGAAGACAGAGGTTTA |
| | | CAACCCGAAGGCCGTCTTCCCTCACGCGGCGTCGCTGCATCAGGGTTTCCCCCATT |
| | | GTGCAATATCCCCCA |
| Ad_denovo5988 | 119 | CCTGTTTGCTCCCCACGCTTTCGTGCCTCAGCGTCAGTTACAGTCCAGAAAGCCGC |
| | | CTTCGCTACTGGTGTTCCTCCCAATATCTACGCATTTCACCGCTACACTGGGAATT |
| | | CCGCTTTCCTCTCCTGCACTCAAGTCAGACAGTATCAGGAGCTTACTACGGTTGAG |
| | | CCGTAGCCTTTAACTCCTGACTTGAAAGACCGCCTACGCACCCTTTACGCCCAGTA |
| | | AATCCGGATAACGCTAGCCCCCTACGTATTACCGCGGCTGCTGGCACGTAGTTAG |
| | | CCGGGGCTTCCTCCTCAAGTACCGTCATTATCTTCCTTGAGGACAGAGCTTTACGA |
| | | CCCGAAGGCCTTCATCGCTCACGCGGCGTTGCTGCATCAGGCTTGCGCCCATTGT |
| | | GCAATATTCCCCA |
| Ad_denovo5998 | 120 | CCCGTTTGCTCCCCTGGCTTTCGCGCCTCAGCGTCAGTTGTCGTCCAGAAAGCCGC |
| | | TTTCGCCACTGGTGTTCCTCCTAATATCTACGCATTTCACCGCTACACTAGGAATT |
| | | CCGCTTTCCTCTCCGATACTCTAGCATCGCAGTTTCGGTCCCCTCACGGGGTTAAG |
| | | CCCCGCACTTTTAAGACCGACTTACGACGCCGCCTGCGCGCCCTTTACGCCCAAT |
| | | AATTCCGGACAACGCTTGCCACCTACGTATTACCGCGGCTGCTGGCACGTAGTTA |
| | | GCCGTGGCTTTCTCTTACGGTACCGTCACTCGTAACGGGTATTGACCGCTACGCCA |
| | | TTCGTCCCGTATAACAGAACTTTACAACCCGAAGGCCGTCATCGTTCACGCGGCG |
| | | TTGCTCCGTCAGACTTTCGTCCATTGCGGAAGATTCCCCA |
| Ad_denovo6087 | 121 | CCTGTTTGCTACCCACGCTTTCGAACCTCAGCGTCAGTTACAGACCAGAGAGCCG |
| | | CTTTCGCCACTGGTGTTCTTCCATATATCTACGCATTTCACCGCTACACATGGAGT |
| | | TCCACTCTCCTCTTCTGCACTCAAGTCTTCCAGTTTCCAATGCACTACTTCGGTTAA |
| | | GCCGAAGGCTTTCACATCAGACTTAAAAGACCGCCTGCGTTCCCTTTACGCCCAA |
| | | TAAATCCGGACAACGCTTGCCACCTACGTATTACCGCGGCTGCTGGCACGTAGTT |
| | | AGCCGTGGCTTTCTGGTCAGATACCGTCAATACGTGAACAGTTACTCTCACGCAC |
| | | GTTCTTCTCTGACAACAGAATTTTACGACCCGAAGGCCTTCTTCATTCACGCGGCG |
| | | TTGCTCCATCAGACTTTCGTCCATTGTGGAAGATTCCCTA |
| Ad_denovo6281 | 122 | CCTGTTTGATACCCGCACTTTCGAGCATCAGCGTCAGTTACGGTCCAGTAAGCTGC |
| | | CTTCGCAATCGGAGTTCTTCGTGATATCTAAGCATTTCACCGCTACACCACGAATT |
| | | CCGCCTACCTATACCGCACTCAAGAAATCCAGTATCAACTGCAATTTTACGGTTG |
| | | AGCCGCAAACTTTCACAACTGACTTAAACTTCCGCCTACGCTCCCTTTAAACCCAA |
| | | TAAATCCGGATAACGCTCGGATCCTCCGTATTACCGCGGCTGCTGGCACGGAGTT |
| | | AGCCGATCCTTATTCATACGGTACATACAAAAAAGCACACGTGCTTCACTTTATTC |
| | | CCGTATAAAAGAAGTTTACAACCCATAGGGCAGTCATCCTTCACGCTACTTGGCT |
| | | GGTTCAGACTCTCGTCCATTGACCAATATTCCTCA |
| Ad_denovo6301 | 123 | CCTGTTCGCTCCCCCGGCTTTCGCGCCTCAGCGTCAGTGTCGGCCCAGCAGGCTGC |
| | | CTTCGCCATCGGTGTTCCTCCCGATATCTGCGCATTCCACCGCTACACCGGGAATT |
| | | CCACCTGCCCCTACCGCACTCGAGCCGCCCAGTTCGGAACCCGGCCGCGGGGTTG |
| | | AGCCCCGGGATTAGAGGTTCCGCTTAGGCGGCCGCCTACGCGCGCTTTACGCCCA |
| | | ATGAATCCGGATAACGCTCGCCCCCTACGTATTACCGCGGCTGCTGGCACGTAGT |
| | | TAGCCGGGGCTTCTTCTGCAGGTACCGTCTCGGTTCTTCCCTGCTGAAAGCGGTTT |
| | | ACAACCCGAAGGCCTTCGTCCCGCACGCGGCGTTGCTGCGTCAGGGTTGCCCCCA |
| | | TTGCGCAAAATTCCCCA |
| Ad_denovo6304 | 124 | CCTATTTGCTCCCCACGCTTTCGGGACTGAGCGTCAGTTATGCGCCAGATCGTCGC |
| | | CTTCGCCACTGGTGTTCCTCCATATATCTACGCATTTCACCGCTACACATGGAATT |
| | | CCACGATCCTCTCACACACTCTAGCTCTACGGTTTCCATGGCTTACCGAAGTTAAG |
| | | CTTCGATCTTTCACCACAGACCCTTAGTGCCGCCTGCTCCCTCTTTACGCCCAATA |
| | | ATTCCGGATAACGCTCGCCACCTACGTATTACCGCGGCTGCTGGCACGTAGTTAG |
| | | CCGTGGCTTTCTTATAGAGTACCGTCACTTGGATATCATTCCCTATATCCACCGTT |
| | | CTTCCTCTATGACAGAAGTTTACATAACGAATTACTTCTTCCTTCACGCGGCGTTG |
| | | CTCGGTCAGGGTTTCCCCCATTGCCGAAAATTCCCTA |
| Ad_denovo6399 | 125 | CCTGTTTGCTCCCCACGCTTTCGAGCCTCAACGTCAGTCATCGTCCAGAAAGCCGC |
| | | CTTCGCCACTGGTGTTCCTCCTAATATCTACGCATTTCACCGCTACACTAGGAATT |
| | | CCGCTTTCCTCTCCGACACTCTAGCCTGACAGTTCCAAATGCAGTCCCGGGGTTGA |
| | | GCCCCGGGCTTTCACATCTGGCTTGCCATGCCGTCTACGCTCCCTTTACACCCAGT |
| | | AAATCCGGATAACGCTTGCCCCCTACGTATTACCGCGGCTGCTGGCACGTAGTTA |
| | | GCCGGGGCTTCTTAGTCAGGTACCGTCATTTTCTTCCCTGCTGATAGAGCTTTACA |
| | | TACCGAAATACTTCATCGCTCACGCGGCGTCGCTGCATCAGGGTTTCCCCCATTGT |
| | | GCAATATTCCCCA |
| Ad_denovo6610 | 126 | CCTATTTGCTCCCCACGCTTTCGTGCTTCAGTGTCAGAATCCAGACCAGACGGCCG |
| | | CCTTCGCCACCGGTGTTCTTCCATATATCTACGCATTTTACCGCTACACATGGAGT |
| | | TCCGCCGTCCTCTTCTGTTCTCTAGCTGATCAGTTTCCAGAGCAAGTACGGGTTGA |
| | | GCCCATACCTTTTACTCCAGACTTGATCTGCCACCTACGCACCCTTTACGCCCAAT |
| | | CATTCCGGATAACGCTCGCCACCTACGTATTACCGCGGCTGCTGGCACGTAGTTA |
| | | GCCGTGACTTTCTGGTAAGATACCATCACTCACTCATCATTCCCTATGAGTGCCGT |
| | | TTTTCTCTTACAACAGAGCTTTACGATCCGAAGACCTTCCTCACTCACGCGGCATT |
| | | GCTCGTTCAGGGTTCCCCCCATTGACGAAAATTCCCTA |
| Ad_denovo6718 | 127 | CCTATTTGCTCCCCACGCTTTCGTGCCTGAGCGTCAGTAACAGTCCAGGCGGCCGC |
| | | CTTCGCCACTGGTGTTCTTCCATATATCTACGCATTTTACCGCTACACATGGAGTT |
| | | CCACCGCCCTCTCCTGTCCTCGAGCGTGACAGTTTCCAAAGCCTGTACAGGTTGA |
| | | GCCCGTACCTTTCACTTCAGACTTGCCACGCCGCCTGCGCACCCTTTACGCCCAAT |
| | | CATTCCGGATAACGCTTGCCACCTACGTATTACCGCGGCTGCTGGCACGTAGTTA |
| | | GCCGTGACTTTCTGGCAGGTTACTATCAAAAGAAAAGCATTTCCTCTTCCCTTCTT |
| | | TTCTGACCTGCAACAGAGCTTTACGATCCGAAGACCTTCCTCACTCACGCGGCATT |
| | | GCTCGTTCAGGCTTGCGCCCATTGACGAAAATTCCCTA |
| Ad_denovo6781 | 128 | CCTGTTTGCTCCCCACGCTTTCGAGCCTCAACGTCAGTCATCGTCCAGCAAGCCGC |
| | | CTTCGCCACTGGTGTTCCTCCTAATATCTACGCATTTCACCGCTACACTAGGAATT |
| | | CCGCTTGCCTCTCCGACACTCTAGCTGCACAGTTTCCAAAGCAGTCCCGGGGTTG |
| | | AGCCCCGGGCTTTCACTTCAGACTTGCACTGCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAAGTTTAC |
| | | ATACCGAAATACTTCATCCTTCACGCGGCGTCGCTGCATCAGGGTTTCCCCCATTG |
| | | TGCAATATTCCCCA |
| Ad_denovo6861 | 129 | CCTGTTCGCTACCCATGCTTTCGAGCCTCAGCGTCAGTTGCAGACCAGACAGCCG |
| | | CCTTCGCCACTGGTGTTCTTCCATATATCTACGCATTCCACCGCTACACATGGAGT |
| | | TCCACTGTCCTCTTCTGCACTCAAGTCGCCCGGTTTCCGATGCACTTCTTCGGTTA |
| | | AGCCGAAGGCTTTCACATCAGACCTAAGCAACCGCCTGCGCTCGCTTTACGCCCA |
| | | ATAAATCCGGATAACGCTTGCCACCTACGTATTACCGCGGCTGCTGGCACGTAGT |
| | | TAGCCGTGACTTTCTGGTTGGATACCGTCACTGCGTGAACAGTTACTCTCACGCAC |
| | | GTTCTTCTCCAACAACAGAGCTTTACGAGCCGAAACCCTTCTTCACTCACGCGGTG |
| | | TTGCTCCATCAGGCTTGCGCCCATTGTGGAAGATTCCCTA |
| Ad_denovo6887 | 130 | CCTGTTTGCTACCCACGCTTTCGCGCTTTAGCGTCAGTATCTGTCCAGTAGGCTGG |
| | | CTTCCCCATCGGCATTCCTACAAATATCTACGAATTTCACCTCTACACTTGTAGTT |
| | | CCGCCTACCTCTCCAGTACTCTAGTTTGGCAGTTTCCAACGCAATACGGAGTTGAG |
| | | CCCCGCATTTTCACATCAGACTTACCAAACCGCCTAGACGCGCTTTACGCCCAAT |
| | | AAATCCGGATAACGCTTGCGACATACGTATTACCGCGGCTGCTGGCACGTATTTA |
| | | GCCGTCGCTTCTTCTGTTGGTACCGTCACTTTCTTCTTCCCAACTGAAAGCACTTTA |
| | | CATTCCGAAAAACTTCATCGTGCACACAGAATTGCTGGATCAGACTTTTGGTCCA |
| | | TTGTCCAATATTCCCCA |
| Ad_denovo7322 | 131 | CCCGTTCGCTACCCTAGCTTTCGAGCCTCAGCGTCAGTTACAGTCCAGAAAGCCG |
| | | CCTTCGCCACTGGTGTTCTTCCCAATATCTACGCATTTCACCGCTACACTGGGAAT |
| | | TCCGCTTTCCTCTCCTGCACTCAAGAAAATCAGTTCGGACCCCCTCACGAGGTTGA |
| | | GCCCCGCACTTTTAAGATCCGCTTAATTTCCCGCCTGCGCTCCCTTTACGCCCAAT |
| | | AATTCCGGACAACGCTTGCCACCTACGTATTACCGCGGCTGCTGGCACGTAGTTA |
| | | GCCGTGGCTTCCTCGTTAAGTACCGTCAAATACTTACTGTATTATAATAAGCATCC |
| | | TTCGCCCTTAACAACAGAACTTTACGATCCGAAGACCTTCTTCGTTCACGCGGCGT |
| | | TGCTCCGTCAGGCTTTCGCCCATTGCGGAAGATTCCCCA |
| Ad_denovo7447 | 132 | CCCGTTCGCTACCCTGGCTTTCGCATCTCAGCGTCAGACACAGTCCAGAAAGGCG |
| | | CCTTCGCCACTGGTGTTCCTCCCAATATCTACGCATTTCACCGCTACACTGGGAAT |
| | | TCCCCTTTCCTCTCCTGCACTCAAGTCTTCCAGTATCCAGAGCCATACGGGGTTGA |
| | | GCCCCGCATTTTCACTCCAGACTTAAAAAACCGCCTACATGCTCTTTACGCCCAAT |
| | | AATTCCGGACAACGCTCGCCACCTACGTATTACCGCGGCTGCTGGCACGTAGTTA |
| | | GCCGTGGCTTCCTCGTCTACTACCGTCATTACACGTCATTGTTCACAACATGCACA |
| | | TTCGTCATAGACAACAGAGCTTTACGGGACGAATCCCTTCATCACTCACGCGGCA |
| | | TTGCTCCGTCAGGCTTTCGCCCATTGCGGAAGATTCCCCA |
| Ad_denovo7591 | 133 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCCCTCCGGCACTCAAGTATGACAGTTTCCAATGCAGTCCACAGGTTG |
| | | AGCCCATGCCTTTCACATCAGACTTGCCACACCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCACTATCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCCA |
| Ad_denovo7849 | 134 | CCTGTTCGATACCCGCACTTTCGAGCTTCAGCGTCAGTTGCGCTCCAGTGAGCTGC |
| | | CTTCGCAATCGGAGTTCTTCGTGATATCTAAGCATTTCACCGCTACACCACGAATT |
| | | CCGCCCACTTTGTGCGTACTCAAGGAAACCAGTTCGCGCTGCAGTGCAGACGTTG |
| | | AGCGTCTACATTTCACAACACGCTTAATCTCCGGCCTACGCTCCCTTTAAACCCAA |
| | | TAAATCCGGATAACGCCCGGACCTTCCGTATTACCGCGGCTGCTGGCACGGAATT |
| | | AGCCGGTCCTTATTCATAAGGTACATGCAAAAAGAGTCACGACTCCCACTTTATT |
| | | CCCTTATAAAAGCAGTTTACAACCCATAGGGCCGTCATCCTGCACGCTACTTGGC |
| | | TGGTTCAGACTCTCGTCCATTGACCAATATTCCTCA |
| Ad_denovo8147 | 135 | CCTGTTTGCTCCCCACGCTTTCGTGCCTCAGTGTCAGTTACAGTCCAGAGAGCCGC |
| | | CTTCGCAACTGGTATTCCTCCTAATATCTACGCATTTCACCGCTACACTAGGAATT |
| | | CTACTCTCCTCTCCTGCACTCAAGTTTCTCAGTTTCAAAGGCTTACTACGGTTGAG |
| | | CCGTAGCCTTTCACCTCTGACTTAAGAAACCACCTACGCACCCTTTACGCCCAGTA |
| | | ATTCCGGATAACGCTAGCCCCCTACGTATTACCGCGGCTGCTGGCACGTAGTTAG |
| | | CCGGGGCTTCCTCCTCAAGTACCGTCATTATCTTCCTTGAGGACAGAGCTTTACGA |
| | | CCCGAAGGCCTTCATCGCTCACGCGGCGTTGCTGCATCAGGCTTTCGCCCATTGTG |
| | | CAATATTCCCCA |
| Ad_denovo8324 | 136 | CCTATTTGCTCCCCACGCTTTCGTGCCTGAGCGTCAGCAACAGTCCAGGCGGCCG |
| | | CCTTCGCCACTGGTGTTCTTCCATATATCTACGCATTTTACCGCTACACATGGAGT |
| | | TCCACCGCCCTCTCCTGTCCTCGAGCGCGCCAGTTTCCAAAGCCTGTACAGGTTGA |
| | | GCCTGTACCTTTCACTTCAGACTTGACGCGCCGCCTGCGCACCCTTTACGCCCAAT |
| | | CATTCCGGATAACGCTTGCCACCTACGTATTACCGCGGCTGCTGGCACGTAGTTA |
| | | GCCGTGACTTTCTGGCGGGGTACCATCAAGAAGAAATCATTTCCTCTTCCTTCCCT |
| | | TTTTCCCCCGCAACAGAGCTTTACGATCCGAAGACCTTCCTCACTCACGCGGCATT |
| | | GCTCGTTCAGGCTTGCGCCCATTGACGAAAATTCCCTA |
| Ad_denovo8360 | 137 | CCTGTTTGCTCCCCACGCTTTCGTGCCTCAGTGTCAGTTACAGTCCAGAAAGCCGC |
| | | CTTCGCTACTGGTGTTCCTCCTAATATCTACGCATTTCACCGCTACACTAGGAATT |
| | | CCACTTTCCTCTCCTGCACTCAAGTTTCCCAGTTTCAAGAGCTTACTACGGTTAAG |
| | | CCGTAGCCTTTCACTCCTGACTTAAGAAACCACCTACGCACCCTTTACGCCCAGTA |
| | | AATCCGGATAACGCTAGCCCCCTACGTATTACCGCGGCTGCTGGCACGTAGTTAG |
| | | CCGGGGCTTCCTCCTCAAGTACCGTCATTATCTTCCTTGAGGACAGAGTTTTACGA |
| | | CCCGAAGGCCTTCATCACTCACGCGGCGTTGCTGCATCAGGCTTTCGCCCATTGTG |
| | | CAATATTCCCCA |
| Ad_denovo8450 | 138 | CCTGTTCGCTCCCCACGCTTTCGCTCCTCAGCGTCAGTGACGGCCCAGAGACCTGC |
| | | CTTCGCCATTGGTGTTCTTCCCGATATCTACACATTCCACCGTTACACCGGGAATT |
| | | CCAGTCTCCCCTACCGCACTCCAGCCCGCCCGTACCCGGCGCAGATCCACCGTTA |
| | | GGCGATGGACTTTCACACCGGACGCGACGAACCGCCTACGAGCCCTTTACGCCCA |
| | | ATAAATCCGGATAACGCTCGCACCCTACGTATTACCGCGGCTGCTGGCACGTAGT |
| | | TAGCCGGTGCTTATTCGAACAATCCACTCAACACGGCCCGAAGCCGTGCCTTGCC |
| | | CTTGAACAAAAGCGGTTTACAACCCGAAGGCCTCCATCCCGCACGCGGCGTCGCT |
| | | GCATCAGGCTTGCGCCCATTGTGCAATATTCCCCA |
| Ad_denovo8723 | 139 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCTCTCCGGCACTCTAGAAGCACAGTTTCCAATGCAGTCCCGTGGTTGA |
| | | GCCTCGGGTTTTCACATCAGACTTGCACTTCCGTCTACGCTCCCTTTACACCCAGT |
| | | AAATCCGGATAACGCTTGCCCCCTACGTATTACCGCGGCTGCTGGCACGTAGTTA |
| | | GCCGGGGCTTCTTACTCAGGTACCGTCATTTTCTTCCCTGCTGATAGAAGTTTACA |
| | | TACCGAAATACTTCATCCTTCACGCGGCGTCGCTGCATCAGGGTTTCCCCCATTGT |
| | | GCAATATTCCCCA |
| Ad_denovo8889 | 140 | CCTATTTGCTCCCCACGCTTTCGTGCCTCAGCGTCAGATGCAGGCCAGACGGCCG |
| | | CCTTCGCCACCGGTGTTCTTCCATATATCTACGCATTTTACCGCTACACATGGAGT |
| | | TCCACCGTCCTCTCCTGCTCTCCAGCCGGACAGTTTCCGCAGCCCCTGAAGGTTGA |
| | | GCCTCCAGTTTTTACTGCGGACTTGCCCGGCCGCCTGCGCACCCTTTACGCCCAAT |
| | | CATTCCGGATAACGCTCGCCACCTACGTATTACCGCGGCTGCTGGCACGTAGTTA |
| | | GCCGTGACTTCCTCGCCGGGTACCATCACTCAGAAAGCTTTCCACTCTTTCTGCCT |
| | | TTTGTCCCCGGCAACAGAGCTTTACGATCCGAAGACCTTCCTCGCTCACGCGGCA |
| | | TTGCTCGTTCAGGGTTCCCCCCATTGACGAAAATTCCCTA |
| Ad_denovo8939 | 141 | CCTGTTCGCTACCCATGCTTTCGAGCCTCAGCGTCAGTTGCAGACCAGAGAGCCG |
| | | CCTTCGCCACTGGTGTTCTTCCATATATCTACGCATTCCACCGCTACACATGGAGT |
| | | TCCACTCTCCTCTTCTGCACTCAAGAAAAACAGTTTCCGATGCAGTTCCTCGGTTA |
| | | AGCCGAGGGCTTTCACATCAGACTTATTCTTCCGCCTGCGCTCGCTTTACGCCCAA |
| | | TAAATCCGGACAACGCTTGCCACCTACGTATTACCGCGGCTGCTGGCACGTAGTT |
| | | AGCCGTGACTTTCTGGTTGATTACCGTCAAATAAAGGCCAGTTACTACCTCTATCC |
| | | TTCTTCACCAACAACAGAGCTTTACGATCCGAAAACCTTCTTCACTCACGCGGCGT |
| | | TGCTCCATCAGACTTTCGTCCATTGTGGAAGATTCCCTA |
| Ad_denovo9276 | 142 | CCCGTTCGCTACCCTAGCTTTCGAGCCTCAGCGTCAGTTACAGTCCAGAAAGCCG |
| | | CCTTCGCCACTGGTGTTCTTCCCAATATCTACGCATTTCACCGCTACACTGGGAAT |
| | | TCCGCTTTCCTCTCCTGCACTCAAGAAAACCAGTTCGGACCCCATCACGGGGTTG |
| | | AGCCCCGCACTTTTAAGATCCGCTTAGTTTCCCGCCTGCGCTCCCTTTACGCCCAA |
| | | TAATTCCGGACAACGCTTGCCGCCTACGTATTACCGCGGCTGCTGGCACGTAGTT |
| | | AGCCGTGGCTTCCTCGTTAGGTACCGTCAAATAAAAACCTTATTATGATTCTTACC |
| | | CTTCGTCCCTAACAACAGAACTTTACGATCCTAAGACCTTCATCGTTCACGCGGCG |
| | | TTGCTCCGTCAGGCTTTCGCCCATTGCGGAAGATTCCCCA |
| Ad_denovo9283 | 143 | CCTGTTCGCTCCCCCAGCTTTCGCGCCTCAGCGTCGGTCTCGGCCCAGAGGGCCGC |
| | | CTTCGCCACCGGTGTTCCACCCGATATCTGCGCATTCCACCGCTACACCGGGTGTT |
| | | CCACCCTCCCCTACCGGACCCGAGCCCGGCGGTTCAGGGGGCGGGACGGGGTTG |
| | | AGCCCCGCCATTTGACCCCCTGCCTGCCGGGCCGCCTACGCGCGCTTTACGCCCA |
| | | ATGAATCCGGATAACGCTCGCCCCCTACGTATTACCGCGGCTGCTGGCACGTAGT |
| | | TAGCCGGGGCTTCTTCTGCAGGTACCGTCTTGTCTCATCCCTGCTGAAAGCGGTTT |
| | | ACGACCCGAGGGCCTTCGTCCCGCACGCGGCGTCGCTGCGTCAGGGTTCCCCCCA |
| | | TTGCGCAAGATTCCCCA |
| Ad_denovo9495 | 144 | CCTGTTCGCTACCCATGCTTTCGAGCCTCAGCGTCAGTTGCAGACCAGAGAGCCG |
| | | CCTTCGCCACTGGTGTTCTTCCATATATCTACGCATTCCACCGCTACACATGGAGT |
| | | TCCACTCTCCTCTTCTGCACTCAAGTTCAACAGTTTCTGATGCAATTCTCCGGTTG |
| | | AGCCGAAGGCTTTCACATCAGACTTATTGAACCGCCTGCACTCGCTTTACGCCCA |
| | | ATAAATCCGGACAACGCTTGCCACCTACGTATTACCGCGGCTGCTGGCACGTAGT |
| | | TAGCCGTGACTTTCTAAGTAATTACCGTCAAATAAAGGCCAGTTACTACCTCTATC |
| | | TTTCTTCACTACCAACAGAGCTTTACGAGCCGAAACCCTTCTTCACTCACGCGGCG |
| | | TTGCTCCATCAGACTTTCGTCCATTGTGGAAGATTCCCTA |
| Ad_denovo9530 | 145 | CCTGTTTGCTCCCCACGCTTTCGCACCTGAGCGTCAGTCTTCGTCCAGGGGGCCGC |
| | | CTTCGCCACCGGTATTCCTCCAGATCTCTACGCATTTCACCGCTACACCTGGAATT |
| | | CTACCCCCCTCTACGAGACTCAAGCTTGCCAGTATCAGATGCAGTTCCCAGGTTG |
| | | AGCCCGGGGATTTCACATCTGACTTAACAAACCGCCTGCGTGCGCTTTACGCCCA |
| | | GTAATTCCGATTAACGCTTGCACCCTCCGTATTACCGCGGCTGCTGGCACGGAGTT |
| | | AGCCGGTGCTTCTTCTGCGGGTAACGTCAATGAGCAAAGGTATTAACTTTACTCC |
| | | CTTCCTCCCCGCTGAAAGTACTTTACAACCCGAAGGCCTTCTTCATACACGCGGCA |
| | | TGGCTGCATCAGGCTTGCGCCCATTGTGCAATATTCCCCA |
| Ad_denovo9818 | 146 | CCTATTTGCTCCCCACGCTTTCGTGCCTGAGCGTCAGTTACAGGCCAGGCGGCCGC |
| | | CTTCGCCACTGGTGTTCTTCCACATCTCTACGCATTTTACCGCTACACGTGGAGTT |
| | | CCACCGCCCTCTCCTGTCCTCGAGCATACCAGTTTCCAAAGCCTGTACAGGTTGAG |
| | | CCCGTACCTTTCACTTCAGACTTGATATGCCGCCTGCGCACCCTTTACGCCCAATC |
| | | ATTCCGGATAACGCTCGCCACCTACGTATTACCGCGGCTGCTGGCACGTAGTTAG |
| | | CCGTGACTTTCTGGCGAGGTACCATCAAAAGAGAATCATTCCCTCTTCTCTTCCTT |
| | | TTTCCCTCGCAACAGAGCTTTACGATCCGAAGACCTTCTTCACCCACGCGGCATTG |
| | | CTCGTTCAGGCTTGCGCCCATTGACGAAAATTCCCTA |
| Ad_denovo10042 | 147 | CCTGTTTGCTCCCCACGCTTTCGAGCCTCAACGTCAGTTACCGTCCAGTAAGCCGC |
| | | CTTCGCCACTGGTGTTCCTCCTAATATCTACGCATTTCACCGCTACACTAGGAATT |
| | | CCGCTTACCTCTCCGGCACTCTAGATGGACAGTTTCCAAAGCAGTCCAGGGGTTG |
| | | AGCCCCTGCCTTTCACTTCAGACTTGCCCGTCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTGTCTTCCCTGCTGATAGAAGTTTAC |
| | | ATACCGAGATACTTCTTCCTTCACGCGGCGTCGCTGCATCAGGGTTTCCCCCATTG |
| | | TGCAATATTCCCTA |
| Ad_denovo10124 | 148 | CCTGTTTGCTACCCACGCTTTCGAACCTCAGCGTCAGTTACAGACCAGAGAGCCG |
| | | CTTTCGCCACTGGTGTTCTTCCATATATCTACGCATTTCACCGCTACACATGGAGT |
| | | TCCACTCTCCTCTTCTGCACTCAAGTCTCCCAGTTTCCAATGCACTACTCCGGTTA |
| | | AGCCGAAGGCTTTCACATCAGACTTAAAAGACCGCCTGCGTTCCCTTTACGCCCA |
| | | ATAAATCCGGATAACGCTTGCCACCTACGTATTACCGCGGCTGCTGGCACGTAGT |
| | | TAGCCGTGGCTTTCTGGTTAGATACCGTCGAAACGTGAACAGTTACTCTCACGCA |
| | | CTTTCTTCTCTAACAACAGGGTTTTACGATCCGAAGACCTTCTTCACCCACGCGGC |
| | | GTTGCTCCATCAGGCTTTCGCCCATTGTGGAAGATTCCCTA |
| Ad_denovo10761 | 149 | CCTGTTTGCTCCCCACGCTTTCGAGCCTCAACGTCAGTTACCGTCCAGTAAGCCGC |
| | | CTTCGCCACTGGTGTTCCTCCCAATATCTACGCATTTCACCGCTACACTGGGAATT |
| | | CCGCTTACCTCTCCGGCACTCTAGATACACAGTTTCCAAAGCAGTCCCGGGGTTG |
| | | AGCCCCGGGCTTTCACTTCAGACTTGCACATCCGTCTACGCTCCCTTTACACCCAG |
| | | TAAATCCGGATAACGCTTGCCCCCTACGTATTACCGCGGCTGCTGGCACGTAGTT |
| | | AGCCGGGGCTTCTTAGTCAGGTACCGTCATTTTCTTCCCTGCTGATAGAGCTTTAC |
| | | ATACCGAAATACTTCTTCGCTCACGCGGCGTCGCTGCATCAGGGTTTCCCCCATTG |
| | | TGCAATATTCCCCA |
| Prevotella sp. | 150 | GAGTTTGATC CTGGCTCAGG ATGAACGCTA GCTACAGGCT TAACACATGC |
| canine oral | | AAGTCGAGGGGCATCATGCA GGTTGCTTGC GATCTGTGAT GGCGACCGGC |
| taxon 195 | | GCACGGGTGA GTAACGCGTATCCAACCTAC CTTCGGCAGG GGCATAACCC |
| | | GGTGAAAGCC GGCCTAATTC CCCATGGTCCCCGTTGATGT CATCTGATTC |
| | | GGGGTAAAGG TGTTTTTTCC GGCCGTTGAT GGGGATGCGTCCGATTAGTT |
| | | AGTTGGCGGG GTAAAGGCCC ACCAAGACAG TGATCGGTAG |
| | | GGGTTCTGAGAGGAAGATCC CCCACATTGG GACTGAGACA CGGCCCAAAC |
| | | TCCTACGGGA GGCAGCAGTGAGGAATATTG GTCAATGGGC GTAAGCCTGA |
| | | ACCAGCCAAG TAGCGTGGAG GACGACCGCCCTATGGGTTG TAAACTCCTT |
| | | TTATGCGGGA ATAAATTTCG GGACGCGTTC CCGTTTTGCATGTACCGCAT |
| | | GAATAAGGAC CGGCTAATTC CGTGCCAGCA GCCGCGGTAA |
| | | TACGGAAGGTTCCGGTGTTA TCCGGATTTA TTGGGTTTAA AGGGAGCGCG |
| | | GACTGCTTGT CAAGCGTGCAGTGAAACGCC GCGGCTCAAC CGCGGTCCTG |
| | | CTGCGCGAAC TGGCTTGCTT GAGTGGGCTGTAGGTACGCG GAATTCGTGG |
| | | TGTAGCGGTG AAATGCTTAG ATATCACGAG GAACTCCGATTGCGAAGGCA |
| | | GCGTACCATA TCCCGACTGA CGTTTATGCT CGAAGGTGCG |
| | | GGTATCAAACAGGATTAGAT ACCCTGGTAG TCCGCACTGT AAACGATGGA |
| | | TGCTCGCTGT CGGCGACATATTGCCGGTGG CCCAGCGAAA GCGTTAAGCA |
| | | TCCCACCTGG GGAGTACGCC GGCAACGGTGAAACTCAAAG GAATTGACGG |
| | | GGGCCCGCAC AAGCGGAGGA ACATGTGGTT TAATTCGATGATACGCGAGG |
| | | AACCTTACCC GGGCTTGAAT TGCAGGAGAA CGATACAGAG |
| | | ATGTTGAGGCCTTTCGGGGC TCCTGTGAAG GTGCTGCATG GTTGTCGTCA |
| | | GCTCGTGCCG TGAGGTGTCGGCTTAAGTGC CATAACGAGC GCAACCCCTT |
| | | TGCGTAGTTG CCATCGGGTG ATGCCGGGCACTCTTCGCAT ACTGCCACCG |
| | | CAAGGTGTGA GGAAGGTGGG GATGACGTCA AATCAGCACGGCCCTTACGT |
| | | CCGGGGCTAC ACACGTGTTA CAATGGTGGG TACAGAGTGT |
| | | TGTTCGTGCGCAAGCACGTT CCAATCACAA AATCCCTCCT CAGTTCGGAC |
| | | TGGGGTCTGC AACCCGACCCCACGAAGCTG GATTCGCTAG TAATCGCGCA |
| | | TCAGCCATGG CGCGGTGAAT ACGTTCCCGGGCCTTGTACA CACCGCCCGT |
| | | CAAGCCATGA AAGCCGGGGG CGCCTGAAGT CCGTGACCGCGAGGGTCGGC |
| | | CTAGGGCGAA ACCGGTGATT GGGGCTAAGT CGTAACAAGG T |
| Prevotella sp. | 151 | GAGTTTGATC CTGGCTCAGG ATGAACGCTA GCTACAGGCT TAACACATGC |
| canine oral | | AAGTCGAGGG GCAGCATGAA GTCAGCTTGC TGACTTTGAT GGCGACCGGC |
| taxon 226 | | GCACGGGTGC GTAACGCGTA TCAAACCTGC CGCATACTCG GGGATAGCCT |
| | | TGCGAAAGTA AGATTAATAC CCGATGTTAT TATGCCCTCG CATGAGGGTA |
| | | TAATCAAAGA TTTTATCGGT ATGCGATGGT GATGCGTCTG ATTAGGTAGT |
| | | AGGCGGGGTA ACGGCCCACC TAGCCAACGA TCAGTAGGGG TTCTGAGAGG |
| | | AAGGTCCCCC ACACTGGAAC TGAGACACGG TCCAGACTCC TACGGGAGGC |
| | | AGCAGTGAGG AATATTGGTC AATGGACGGA AGTCTGAACC AGCCAAGTAG |
| | | CGTGCAGGAT GACGGCCCTC CGGGTTGTAA ACTGCTTTTA GTTGGGAATA |
| | | ACGGCGGGGA CGCGTCCCCG AAAGGAATGT ACCATCAGAA AAAGGACCGG |
| | | CTAATTCCGT GCCAGCAGCC GCGGTAATAC GGAAGGTCCA GGCGTTATCC |
| | | GGATTTATTG GGTTTAAAGG GAGCGTAGGC GGGCTATTAA GTCAGCGGTT |
| | | AAAGCGTGTG GCTCAACCAT ACATTGCCGT TGAAACTGGT GGTCTTGAGT |
| | | GCACACAGGG ATGCTGGAAC TCGTGGTGTA GCGGTGAAAT GCTTAGATAT |
| | | CACGATGAAC TCCGATCGCG AAGGCAGGTG TCCGGGGTGC TACTGACGCT |
| | | GAGGCTCGAA AGTGTGGGTA TCAAACAGGA TTAGATACCC TGGTAGTCCA |
| | | CACAGTAAAC GATGTATACT CGTAGTTTGC GATAGATTGT AAGCTACCAA |
| | | GCGAAAGCAT TAAGTATACC ACCTGGGGAG TACGCCGGCA ACGGTGAAAC |
| | | TCAAAGGAAT TGACGGGGGC CCGCACAAGC GGAGGAACAT GTGGTTTAAT |
| | | TCGATGATAC GCGAGGAACC TTACCCGGGC TTGAACTAGA CAGGACTTAC |
| | | CAAGAGATTG GTATTTCTTC GGACCTGTTT AGAGGTGCTG CATGGTTGTC |
| | | GTCAGCTCGT GCCGTGAGGT GTCGGCTTAA GTGCCATAAC GAGCGCAACC |
| | | CTTCTCCTCG GTTGCCATCA GGTGATGCTG GGCACTCCGT GGACACTGCC |
| | | ATCGTAAGAT GTGAGGAAGG TGGGGATGAC GTCAAATCAG CACGGCCCTT |
| | | ACGTCCGGGG CTACACACGT GTTACAATGG GGGGTACAGA GGGTCGCTAC |
| | | CTGGTGACAG GATGCTAATC TCGAAAACCT CTCTCAGTTC GGATTGGAGT |
| | | CTGCAACCCG ACTCCATGAA GCTGGATTCG CTAGTAATCG CGCATCAGCC |
| | | ATGGCGCGGT GAATACGTTC CCGGGCCTTG TACACACCGC CCGTCAAGCC |
| | | ATGAAAGCCG GGGGTGCCTG AAAGCCGTGA CCGCAAGGGT CGGCCTAGGG |
| | | TAAAACTGGT GATTGGGGCT AAGTCGTAAC AAGGTAGCCG TACCGGAAGG |
| | | TGCGGCTGGA TCACCTCCTT |
| Blautia sp. | 152 | GAGTTTGATC CTGGCTCAGG ATGAACGCTG GCGGCGTGCT TAACACATGC |
| canine taxon | | AAGTCGAACG AAGCACTTGA ATGGAATTCT TCGGAAGGAA GCCCAAGTGA |
| 143 | | CTGAGTGGCG GACGGGTGAG TAACGCGTGG GTAACCTGCC TCATACAGGG |
| | | GGATAACAGT TAGAAATGAC TGCTAATACC GCATAAGCAC ACGTGATCGC |
| | | ATGATCGAGT GTGAAAAACT CCGGTGGTAT GAGATGGACC CGCGTCTGAT |
| | | TAGCTAGTTG GTGGGGTAAT GGCCCACCAA GGCGACGATC AGTAGCCGGC |
| | | CTGAGAGGGT GAACGGCCAC ATTGGGACTG AGACACGGCC CAAACTCCTA |
| | | CGGGAGGCAG CAGTGGGGAA TATTGCACAA TGGGGGAAAC CCTGATGCAG |
| | | CGACGCCGCG TGAAGGATGA AGTATTTCGG TATGTAAACT TCTATCAGCA |
| | | GGGAAGAAAA TGACGGTACC TGACTAAGAA GCCCCGGCTA ACTACGTGCC |
| | | AGCAGCCGCG GTAATACGTA GGGGGCAAGC GTTATCCGGA TTTACTGGGT |
| | | GTAAAGGGAG CGTAGACGGC AGTGCAAGTC TGAAGTGAAA GCCCGGGGCT |
| | | CAACCCCGGG ACTGCTTTGG AAACTGTGCA GCTAGAGTGT CGGAGAGGCA |
| | | AGCGGAATTC CTAGTGTAGC GGTGAAATGC GTAGATATTA GGAGGAACAC |
| | | CAGTGGCGAA GGCGGCTTGC TGGACGATGA CTGACGTTGA GGCTCGAAAG |
| | | CGTGGGGAGC AAACAGGATT AGATACCCTG GTAGTCCACG CCGTAAACGA |
| | | TGACTACTAG GTGTCGGGGA GCAAAGCTCT TCGGTGCCGC AGCCAACGCA |
| | | ATAAGTAGTC CACCTGGGGA GTACGTTCGC AAGAATGAAA CTCAAAGGAA |
| | | TTGACGGGGA CCCGCACAAG CGGTGGAGCA TGTGGTTTAA TTCGAAGCAA |
| | | CGCGAAGAAC CTTACCTGCT CTTGACATCC CTCTGACCGC TCTTTAATCG |
| | | GAGCTTTCCT TCGGGACAGA GGAGACAGGT GGTGCATGGT TGTCGTCAGC |
| | | TCGTGTCGTG AGATGTTGGG TTAAGTCCCG CAACGAGCGC AACCCCTATC |
| | | TTCAGTAGCC AGCGGTAAGG CCGGGCACTC TGGAGAGACT GCCAGGGATA |
| | | ACCTGGAGGA AGGTGGGGAT GACGTCAAAT CATCATGCCC CTTATGAGCA |
| | | GGGCTACACA CGTGCTACAA TGGCGTAAAC AAAGGGAAGC AGAGTCGTGA |
| | | GGCCGAGCAA ATCCCAAAAA TAACGTCTCA GTTCGGATTG TAGTCTGCAA |
| | | CTCGACTACA TGAAGCTGGA ATCGCTAGTA ATCGCGAATC AGAATGTCGC |
| | | GGTGAATACG TTCCCGGGTC TTGTACACAC CGCCCGTCAC ACCATGGGAG |
| | | TCAGTAACGC CCGAAGTCAG TGACCCAACC GCAAGGAGGG AGCTGCCGAA |
| | | GGTGGGACCG ATAACTGGGG TGAAGTCGTA ACAAGGTAGC CGTATCGGAA |
| | | GGTGCGGCTG GATCACCTCC TT |
| Blautia sp. | 153 | GAGTTTGATC CTGGCTCAGG ATGAACGCTG GCGGCGTGCT TAACACATGC |
| canine taxon | | AAGTCGAACG AAGCACTGGA AACGGAATTC TTCGGAAGGA AGTATTTAGT |
| 337 | | GACTGAGTGG CGGACGGGTG AGTAACGCGT GGGTAACCTG CCTCATACAG |
| | | GGGGATAACA GTTAGAAATA GCTGCTAATA CCGCATAAGA CCACAGAGTC |
| | | GCATGACTCA GTGGGAAAAA CTCCGGTGGT ATGAGATGGA CCCGCGTCTG |
| | | ATTAGCTAGT TGGTAAGGTA ACGGCTTACC AAGGCGACGA TCAGTAGCCG |
| | | ACCTGAGAGG GTGACCGGCC ACATTGGGAC TGAGACACGG CCCAAACTCC |
| | | TACGGGAGGC AGCAGTGGGG AATATTGCAC AATGGGGGAA ACCCTGATGC |
| | | AGCGACGCCG CGTGAGTGAT GAAGTATTTC GGTATGTAAA GCTCTATCAG |
| | | CAGGGAAGAA AATGACGGTA CCTGACTAAG AAGCCCCGGC TAACTACGTG |
| | | CCAGCAGCCG CGGTAATACG TAGGGGGCAA GCGTTATCCG GATTTACTGG |
| | | GTGTAAAGGG AGTGTAGACG GTGATGTAAG TCTGATGTGA AAATTTGGGG |
| | | CTCAACCCCA AAACTGCATT GGAAACTATG TCACTAGAGT GTCGGAGAGG |
| | | TAAGTGGAAT TCCTAGTGTA GCGGTGAAAT GCGTAGATAT TAGGAGGAAC |
| | | ACCAGTGGCG AAGGCGGCTT ACTGGACGAT GACTGACGTT GAGGCTCGAA |
| | | AGCGTGGGGA GCAAACAGGA TTAGATACCC TGGTAGTCCA CGCCGTAAAC |
| | | GATGAATACT AGGTGTCGGG TGGCAAAGCC ATTCGGTGCC GTCGCAAACG |
| | | CAATAAGTAT TCCACCTGGG GAGTACGTTC GCAAGAATGA AACTCAAAGG |
| | | AATTGACGGG GACCCGCACA AGCGGTGGAG CATGTGGTTT AATTCGAAGC |
| | | AACGCGAAGA ACCTTACCTG GTCTTGACAT CCCCTTGACA GAGTATGTAA |
| | | TGTACTTTTC CTTCGGGACA AGGGAGACAG GTGGTGCATG GTTGTCGTCA |
| | | GCTCGTGTCG TGAGATGTTG GGTTAAGTCC CGCAACGAGC GCAACCCCTA |
| | | TCTTTAGTAG CCAGCATATG AGGTGGGCAC TCTAGAGAGA CTGCCAGGGA |
| | | TAACCTGGAG GAAGGTGGGG ATGACGTCAA ATCATCATGC CCCTTATGAT |
| | | CAGGGCTACA CACGTGCTAC AATGGCGTAA ACAAAGGGAA GCGACCCTGT |
| | | GAAGGCAAGC AAATCTCAAA AATAACGTCT CAGTTCGGAT TGTAGTCTGC |
| | | AACTCGACTA CATGAAGCTG GAATCGCTAG TAATCGCGAA TCAGAATGTC |
| | | GCGGTGAATA CGTTCCCGGG TCTTGTACAC ACCGCCCGTC ACACCATGGG |
| | | AGTCAGTAAC GCCCGAAGTC AGTGACCCAA CCGAAAGGAG GGAGCTGCCG |
| | | AAGGTGGAAC CGATAACTGG GGTGAAGTCG TAACAAGGTA |
| Allobaculum | 154 | GATGAACGCT GGCGGCATGC CTAATACATG CAAGTCGAAC GAGCTACTTC |
| stercoricanis | | GGTAGCTAGT GGCGAACGGG TGAGTAACAC GTAGATAACC TGCCCATACC |
| DSM 13633 | | CGGGGGATAC GCTTTGGAAA CGAAGTCTAA AACCCCATAG GAAGATTTAA |
| | | GGCATCTTAA ATTTTTGAAA TAAGCTTTGG CTTAGGGGAT GGATGGATCT |
| | | GCGGTGCATT AGCTAGTTGG TGAGGTAACA GCTCACCAAG GCGATGATGC |
| | | ATAGCCGGCC TGAGAGGGCG ATCGGCCACA CTGGGACTGA GACACGGCCC |
| | | AGACTCCTAC GGGAGGCAGC AGTAGGGAAT TTTCGTCAAT GGGCGCAAGC |
| | | CTGAACGAGC AATGCCGCGT GGGTGAAGAA GGTCTTCGGA TCGTAAAGCT |
| | | CTGTTGCGAG GGAAAAAGGA AGAGAAGAGG GAATGATTCT CTTTTGATGG |
| | | TACCTCGCCA GAAAGTCACG GCTAACTACG TGCCAGCAGC CGCGGTAATA |
| | | CGTAGGTGGC GAGCGTTATC CGGAATGATT GGGCGTAAAG GGTGCGCAGG |
| | | CGGCATATCA AGTCTGAAGT GAAAGGTACG GGCTCAACCT GTACAGGCTT |
| | | TGGAAACTGG TATGCTCGAG GACAGGAGAG GGCGGTGGAA CTCCACGTGT |
| | | AGCGGTAAAA TGCGTAGAGA TGTGGAAGAA CACCAGTGGC GAAGGCGGCC |
| | | GCCTGGCCTG TAACTGACGC TCAGGCACGA AAGCGTGGGG AGCAAATAGG |
| | | ATTAGATACC CTAGTAGTCC ACGCCCTAAA CGATGAGGAG CAGGTGTCGG |
| | | GGGTAGTACC TCGGTGCCGA AGCTAACGCA ATGACTCCTC CGCCTGGGGA |
| | | GTATGCACGC AAGTGTGAAA CTCAAAGGAA TTGACGGGGG CCCGCACAAG |
| | | CGGTGGAGTA TGTGGTTTAA TTCGAAGCAA CGCGAAGAAC CTTACCAGGC |
| | | CTTGACATCC CGAGCAAAGA CATAGAGATA TGTTAGAGGT TATCTCGGTG |
| | | ACAGGTGGTG CATGGTTGTC GTCAGCTCGT GTCGTGAGAT GTTCAGTTAA |
| | | GTCTGGCAAC GAGCGCAACC CTCGTGATGT GTTACTACCA TTCAGTTGAG |
| | | GACTCACATC AGACTGCCGG TGACAAACCG GAGGAAGGCG GGGATGACGT |
| | | CAAATCATCA TGCCCCTTAT GGCCTGGGCT ACACACGTAC TACAATGGCA |
| | | TCTACAGACG GAAGCGAACC TGTGAAGGCA AGCCAATCCG AGAAAAGATG |
| | | TCCCAGTTCG GATTGAAGTC TGCAACTCGA CTTCATGAAG TTGGAATCGC |
| | | TAGTAATCGC GGATCAGCAT GCCGCGGTGA ATACGTTCCC GGGCCTTGTA |
| | | CACACCGCCC GTCAAACCAT GGAAGCCGGT AACGCCCGAA GCCGATGGCA |
| | | TAACCCGTAA GGGAGTGAGT CGTCGAAGGC GGGACCGATG ACTGGGGTTA |
| | | AGTCGTAACA AGGTATCCCT ACGGGAACG |
| Clostridium | 155 | GAGTTTGATC CTGGCTCAGG ATGAACGCTG GCGGCGTGCC TAACACATGC |
| hiranonis | | AAGTCGAGCG ATTCTCTTCG GAGAAGAGCG GCGGACGGGT GAGTAACGCG |
| | | TGGGTAACCT GCCCTGTACA CACGGATAAC ATACCGAAAG GTATGCTAAT |
| | | ACGGGATAAT ATATAAGAGT CGCATGACTT TTATATCAAA GATTTTTCGG |
| | | TACAGGATGG ACCCGCGTCT GATTAGCTTG TTGGCGGGGT AACGGCCCAC |
| | | CAAGGCGACG ATCAGTAGCC GACCTGAGAG GGTGATCGGC CACATTGGAA |
| | | CTGAGACACG GTCCAAACTC CTACGGGAGG CAGCAGTGGG GAATATTGCA |
| | | CAATGGGCGC AAGCCTGATG CAGCAACGCC GCGTGAGCGA TGAAGGCCTT |
| | | CGGGTCGTAA AGCTCTGTCC TCAAGGAAGA TAATGACGGT ACTTGAGGAG |
| | | GAAGCCCCGG CTAACTACGT GCCAGCAGCC GCGGTAATAC GTAGGGGGCT |
| | | AGCGTTATCC GGATTTACTG GGCGTAAAGG GTGCGTAGGC GGTCTTTCAA |
| | | GTCAGGAGTT AAAGGCTACG GCTCAACCGT AGTAAGCTCC TGATACTGTC |
| | | TGACTTGAGT GCAGGAGAGG AAAGCGGAAT TCCCAGTGTA GCGGTGAAAT |
| | | GCGTAGATAT TGGGAGGAAC ACCAGTAGCG AAGGCGGCTT TCTGGACTGT |
| | | AACTGACGCT GAGGCACGAA AGCGTGGGGA GCAAACAGGA TTAGATACCC |
| | | TGGTAGTCCA CGCTGTAAAC GATGAGTACT AGGTGTCGGA GGTTACCCCC |
| | | TTCGGTGCCG CAGCTAACGC ATTAAGTACT CCGCCTGGGG AGTACGCACG |
| | | CAAGTGTGAA ACTCAAAGGA ATTGACGGGG ACCCGCACAA GTAGCGGAGC |
| | | ATGTGGTTTA ATTCGAAGCA ACGCGAAGAA CCTTACCTAG GCTTGACATC |
| | | CTTCTGACCG AGGACTAATC TCCTCTTTCC CTCCGGGGAC AGAAGTGACA |
| | | GGTGGTGCAT GGTTGTCGTC AGCTCGTGTC GTGAGATGTT GGGTTAAGTC |
| | | CCGCAACGAG CGCAACCCTT GTCTTTAGTT GCCATCATTA AGTTGGGCAC |
| | | TCTAGAGAGA CTGCCAGGGA TAACCTGGAG GAAGGTGGGG ATGACGTCAA |
| | | ATCATCATGC CCCTTATGCC TAGGGCTACA CACGTGCTAC AATGGGTGGT |
| | | ACAGAGGGCA GCCAAACCGT GAGGTGGAGC AAATCCCTTA AAGCCATTCT |
| | | CAGTTCGGAT TGTAGGCTGA AACTCGCCTA CATGAAGCTG GAGTTACTAG |
| | | TAATCGCAGA TCAGAATGCT GCGGTGAATG CGTTCCCGGG TCTTGTACAC |
| | | ACCGCCCGTC ACACCATGGG AGTTGGAGAC ACCCGAAGCC GACTATCTAA |
| | | CCTTTTGGGA GAAGTCGTCG AAGGTGGAAT CAATAACTGG GGTGAAGTCG |
| | | TAACAAGGTA GCCGTATCGG AAGGTGCGGC TGGATCACCT CCTT |
| Pepto- | 156 | GAGTTTGATC CTGGCTCAGG ATGAACGCTG GCGGCGTGCC TAACACATGC |
| streptococcus | | AAGTCGAGCG CGGTTGTGCT TAGTATTGAG TGTTTTATTG ATATAAAACA |
| sp. canine | | TTGAATTCTA TGCACAACTG AGCGGCGGAC GGGTGAGTAA CGCGTGGGTA |
| oral taxon 033 | | ACCTGCCCTA TACACATGGA TAACATACTG AAAAGTTTAC TAATACATGA |
| | | TAAAATAGTT TTTCGGCATC GAAGAATTAT CAAAGTGTTT GCGGTATAGG |
| | | ATGGACCCGC GTCTGATTAG CTAGTTGGTG AGATAACTGC CCACCAAGGC |
| | | GACGATCAGT AGCCGACCTG AGAGGGTGAT CGGCCACATT GGAACTGAGA |
| | | CACGGTCCAA ACTCCTACGG GAGGCAGCAG TGGGGAATAT TGCACAATGG |
| | | GCGCAAGCCT GATGCAGCAA CGCCGCGTGA ACGATGAAGG TCTTCGGATC |
| | | GTAAAGTTCT GTTGCAGGGG AAGATAATGA CGGTACCCTG TGAGGAAGCC |
| | | CCGGCTAACT ACGTGCCAGC AGCCGCGGTA ATACGTAGGG GGCTAGCGTT |
| | | ATCCGGATTT ACTGGGCGTA AAGGGTGCGT AGGTGGTCTT TCAAGTCGGT |
| | | GGTTAAAGGC TACGGCTCAA CCGTAGTAAG CCTCCGAAAC GGTTAGACTT |
| | | GAGTGCAGGA GAGGAAAGTG GAATTCCCAG TGTAGCGGTG AAATGCGTAG |
| | | ATATTGGGAG GAACACCAGT AGCGAAGGCG GCTTTCTGGA CTGCAACTGA |
| | | CACTGAGGCA CGAAAGCGTG GGTAGCAAAC AGGATTAGAT ACCCTGGTAG |
| | | TCCACGCCGT AAACGATGAG TACTAGGTGT CGGGGGTTAC CCCCCTCGGT |
| | | GCCGCAGCTA ACGCATTAAG TACTCCGCCT GGGGAGTACG CACGCAAGTG |
| | | TGAAACTCAA AGGAATTGAC GGGGACCCGC ACAGGTAGCG GAGCATGTGG |
| | | TTTAATTCGA AGCAACGCGA AGAACCTTAC CTAAGCTTGA CATCCCTCGG |
| | | ACCGGTGTTT AATCACACCT TTCCTTCGGG ACTGAGGAGA CAGGTGGTGC |
| | | ATGGTTGTCG TCAGCTCGTG TCGTGAGATG TTGGGTTAAG TCCCGCAACG |
| | | AGCGCAACCC TTGTCTTTAG TTGCCATCAT TAAGTTGGGC ACTCTAGAGA |
| | | GACTGCCAGG GACAACCTGG AGGAAGGTGG GGATGACGTC AAATCATCAT |
| | | GCCCCTTATG CTTAGGGCTA CACACGTGCT ACAATGGGTG GTACAGAGGG |
| | | TTGCCAAACC GTGAGGTGGA GCCAATCCCT TAAAGCCACT CTCAGTTCGG |
| | | ATTGTAGGCT GAAACTCGCC TACATGAAGC TGGAGTTACT AGTAATCGCA |
| | | GATCAGAATG CTGCGGTGAA TGCGTTCCCG GGTCTTGTAC ACACCGCCCG |
| | | TCACACCATG GGAGTCGGAA GCACCCGAAG CCGATTATCT AACCGCAAGG |
| | | AGGAGATCGT CGAAGGTGGC GTCGATAACT GGGGTGAAGT CGTAACAAGG |
| | | TAGCCGTATC GGAAGGTGCG GCTGGATCAC CTCCTT |
| Pepto- | 157 | GAGTTTGATC CTGGCTCAGG ATGAACGCTG GCGGCGTGCC TAACACATGC |
| streptococcus | | AAGTCGAGCG CGACTGATTT GATGCTTGCA TCGATGAAAG TTGAGCGGCG |
| sp. canine | | GACGGGTGAG TAACGCGTGG GCAACCTGCC CTGTACACAT GGATAACATA |
| oral taxon 227 | | CTGAAAAGTT TACTAATACA TGATAATATA GTTTTTCGGC ATCGAAGAAT |
| | | TATCAAAGTG TTAGCGGTAC AGGATGGGCC CGCGTCTGAT TAGCTAGTTG |
| | | GTGAGATAAC TGCCCACCAA GGCGACGATC AGTAGCCGAC CTGAGAGGGT |
| | | GATCGGCCAC ATTGGAACTG AGACACGGTC CAAACTCCTA CGGGAGGCAG |
| | | CAGTGGGGAA TATTGCACAA TGGGCGCAAG CCTGATGCAG CAACGCCGCG |
| | | TGAACGATGA AGGTCTTCGG ATCGTAAAGT TCTGTTGCAG GGGAAGACAA |
| | | TGACGGTACC CTGTGAGGAA GCCCCGGCTA ACTACGTGCC AGCAGCCGCG |
| | | GTAATACGTA GGGGGCTAGC GTTATCCGGA TTTACTGGGC GTAAAGGGTG |
| | | CGTAGGTGGT CCTTCAAGTC GGTGGTTAAA GGCTACGGCT CAACCGTAGT |
| | | AAGCCTCCGA AACTGTTGGA CTTGAGTGCA GGAGAGGAAA GTGGAATTCC |
| | | CAGTGTAGCG GTGAAATGCG TAGATATTGG GAGGAACACC AGTAGCGAAG |
| | | GCGGCTTTCT GGACTGCAAC TGACACTGAG GCACGAAAGC GTGGGTAGCA |
| | | AACAGGATTA GATACCCTGG TAGTCCACGC TGTAAACGAT GAGTACTAGG |
| | | TGTCGGGGGT TACCCCCCTC GGTGCCGCAG CTAACGCATT AAGTACTCCG |
| | | CCTGGGGAGT ACGCACGCAA GTGTGAAACT CAAAGGAATT GACGGGGACC |
| | | CGCACAAGTA GCGGAGCATG TGGTTTAATT CGAAGCAACG CGAAGAACCT |
| | | TACCTAAGCT TGACATCCCT CGGACCGGTG TTTAATCACA CCTTTCCTTC |
| | | GGGACTGAGG TGACAGGTGG TGCATGGTTG TCGTCAGCTC GTGTCGTGAG |
| | | ATGTTGGGTT AAGTCCCGCA ACGAGCGCAA CCCTTGTCTT TAGTTGCCAT |
| | | CATTAAGTTG GGCACTCTAG AGAGACTGCC AGGGATAACC TGGAGGAAGG |
| | | TGGGGATGAC GTCAAATCAT CATGCCCCTT ATGCTTAGGG CTACACACGT |
| | | GCTACAATGG GTGGTACAGA GGGTTGCCAA ACCGTGAGGT GGAGCTAATC |
| | | CCTTAAAGCC ATTCTCAGTT CGGATTGTAG GCTGAAACTC GCCTACATGA |
| | | AGCTGGAGTT ACTAGTAATC GCAGATCGGA ATGCTGCGGT GAATGCGTTC |
| | | CCGGGTCTTG TACACACCGC CCGTCACACC ATGGGAGTCG GAAACACCCG |
| | | AAGCCGATTA TCTAACCGCA AGGAGGAAGT CGTCGAAGGT GGCGTCGATA |
| | | ACTGGGGTGA AGTCGTAACA AGGTAGCCGT ATCGGAAGGT GCGGCTGGAT |
| | | CACCTCCTT |
| Pepto- | 158 | GAGTTTGATC CTGGCTCAGG AGGAACGCTG GCGGCGTGCC TAACACATGC |
| streptococcaceae | | AAGTCGAGCG AGAAATAAAG AAACGGAGAA TTCGTTCAAA GATTCTTTAT |
| bacterium | | GGAAAGCGGC GGACGGGTGA GTAACGCGTA GGCAACCTGC CTCATACAAA |
| canine taxon | | GGGATAGCCT CGGGAAACCG GGATTAAAAC CTTATAAAAC CGAAGGAGCA |
| 066 | | CATGCTTCAT CGGTCAAAGA TTTATCGGTA TGAGATGGGC CTGCGTCTGA |
| | | TTAGCTGGTT GGTGAGGTAA CGGCTCACCA AGGCGACGAT CAGTAGCCGA |
| | | CCTGAGAGGG TAAACGGCCA CATTGGAACT GAGACCCGGT CCAAACTCCT |
| | | ACGGGAGGCA GCAGTGGGGA ATATTGCACA ATGGGCGAAA GCCTGATGCA |
| | | GCAACGCCGC GTGAGTGATG AAGGCCCTTG GGTCGTAAAA CTCTGTTGAG |
| | | AGGGAAGAAA AAAATGACGG TACCTCTTGA GGAAGCCCCG GCTAACTACG |
| | | TGCCAGCAGC CGCGGTAATA CGTAGGGGGC GAGCGTTATC CGGAATTATT |
| | | GGGCGTAAAG AGTGCGTAGG TGGTTATCTA AGCGTGGGGT GAAAGGCAGT |
| | | GGCTTAACCA TTGTAAGCCT TGCGAACTGG ATAGCTTGAG TGCAGGAGGG |
| | | GAAAGTGGAA TTCCTAGTGT AGCGGTGAAA TGCGTAGATA TTAGGAGGAA |
| | | CACCGGTGGC GAAGGCGGCT TTCTGGACTG TAACTGACAC TGAGGCACGA |
| | | AAGCGTGGGT AGCAAACAGG ATTAGATACC CTGGTAGTCC ACGCCGTAAA |
| | | CGATGAGCAC TAGGTGTTGG GGGGAGAACT CTCAGTGCCG CAGTCAACGC |
| | | AATAAGTGCT CCGCCTGGGG AGTACGCACG CAAGTGTAAA ACTCAAAGGA |
| | | ATTGACGGGG ACCCGCACAA GCAGCGGAGC ATGTGGTTTA ATTCGAAGCA |
| | | ACGCGAAGAA CCTTACCGGG ACTTGACATC CGCCTGACGT CTCCTTAACC |
| | | GGAGATTTCT TCGGACAGGC AAGACAGGTG GTGCATGGTT GTCGTCAGCT |
| | | CGTGTCGTGA GATGTTGGGT TAAGTCCCGC AACGAGCGCA ACCCTTGTCA |
| | | ATAGTTGCCA GCAGTAAGAT GGGCACTCTA TTGAGACTGC CGTGGATAAC |
| | | ACGGAGGAAG GTGGGGATGA CGTCAAATCA TCATGCCCCT TATGTTCCGG |
| | | GCTACACACG TGCTACAATG GCCGGTACAA CGAGAAGCAA GACCGCAAGG |
| | | TGGAGCAAAT CTTAAAAAGC CGGTCCCAGT TCGGATTGTA GGCTGCAACT |
| | | CGCCTACATG AAGATGGAGT TGCTAGTAAT CGCAGATCAG AATGCTGCGG |
| | | TGAATGCGTT CCCGGGTCTT GTACACACCG CCCGTCACAC CATGGAAGTT |
| | | GGGGGTGCCC GAAGTCGGTT AGAAAATAGG CTGCCGAAGG CAAAACCAAT |
| | | GACTGGGGTG AAGTCGTAAC AAGGTAGCCG TATCGGAAGG TGCGGCTGGA |
| | | TCACCTCCTT |
| Pepto- | 159 | GAGTTTGATC CTGGCTCAGG ATGAACGCTG GCGGCGTGCC TAACACATGC |
| streptococcaceae | | AAGTCGAGCG AAAAATCCAT AATCGAATCT TCGGACAAGA GAGTGGATGG |
| bacterium | | AAAGCGGCGG ACGGGTGAGT AACGCGTAGG TAACCTGCCC TGTACAGAGG |
| canine taxon | | GATAGCCACC GGAAACGGTG ATTAATACCT CATAACACCG AAAGTTCACA |
| 139 | | TGGACAGTCG GTCAAAGATT TATCGGTACA GGATGGACCT GCGTCTGATT |
| | | AGTTAGTTGG TGAGGTAACG GCTCACCAAG GCGACGATCA GTAGCCGACC |
| | | TGAGAGGGTG ATCGGCCACA TTGGAACTGA GACACGGTCC AAACTCCTAC |
| | | GGGAGGCAGC AGTGGGGAAT ATTGCACAAT GGGGGAAACC CTGATGCAGC |
| | | AACGCCGCGT GAATGAAGAA GGCCTTTGGG TTGTAAAATT CTGTTCTGAG |
| | | GGAAGAAGAA AGTGACGGTA CCTCAGGAGA AAGCCCCGGC TAACTACGTG |
| | | CCAGCAGCCG CGGTAATACG TAGGGGGCAA GCGTTGTCCG GAATCATTGG |
| | | GCGTAAAGAG TACGTAGGCG GTTTGGCAAG CGTAAGGTTT AAGGCAACAG |
| | | CTCAACTGTT GTTCGCCTTG TGAACTGTCA AACTTGAGTG CGGGAGAGGA |
| | | AAGCGGAATT CCTGGTGTAG CGGTGAAATG CGTAGATATC AGGAGGAATA |
| | | CCGGTGGCGA AGGCGGCTTT CTGGACCGTA ACTGACGCTG AGGTACGAAA |
| | | GCGTGGGGAG CAAACAGGAT TAGATACCCT GGTAGTCCAC GCCGTAAACG |
| | | ATGAGCACTA GGTGTCGGGG CTTTAGAGCT TCGGTGCCGC AGTTAACGCA |
| | | ATAAGTGCTC CGCCTGGGGA GTACGCACGC AAGTGTGAAA CTCAAAGGAA |
| | | TTGACGGGGA CCCGCACAAG CAGCGGAGCA TGTGGTTTAA TTCGAAGCAA |
| | | CGCGAAGAAC CTTACCAGGG CTTGACATCC TTCTGACGTA TCCTTAATCG |
| | | GATATTTCTA CGGACAGAAG AGACAGGTGG TGCATGGTTG TCGTCAGCTC |
| | | GTGTCGTGAG ATGTTGGGTT AAGTCCCGCA ACGAGCGCAA CCCTTGTCAT |
| | | TAGTTACTAA CGATATAAGT CGAGGACTTT AATGAGACTG CCGGGGAGAA |
| | | CTCGGAGGAA GGTGGGGATG ACGTCAAATC ATCATGCCCC TTATGTTCTG |
| | | GGCTACACAC GTGCTACAAT GGTCGGTACA AAGAGAAGCG AGACTGTGAA |
| | | GTGGAGCAAA ACTCAAAAGC CGATCCCAGT TCGGACTGTA GGCTGAAACC |
| | | CGCCTACACG AAGTCGGAGT TGCTAGTAAT CGTGGATCAG AATGCCACGG |
| | | TGAATGCGTT CCCGGGTCTT GTACACACCG CCCGTCACAC CATGGAAGTT |
| | | GGGGGCGCCC GAAGTCGGTC GAAAAATAGA CTGCCTAAGG TGAAACCAAT |
| | | GACTGGGGTG AAGTCGTAAC AAGGTAGCCG TATCGGAAGG TGCGGCTGGA |
| | | TCACCTCCTT |

TABLE 3.2
Estimated Shannon index diversity post-weaning in puppies
expressed as group means with 95% confidence intervals.

| | | Normal range | | Outliers for intervention | |
|---|---|---|---|---|---|
| | | 95% | 95% | 5th | 95th |
| Age | Mean | Lower | Upper | Percentile | Percentile |
| (Days) | Diversity | CI | CI | of range | of range |
| 31 | 1.682 | 1.351 | 2.012 | 0.8595 | 2.6721 |
| 38 | 1.831 | 1.473 | 2.188 | 0.6351 | 2.8786 |
| 45 | 2.038 | 1.699 | 2.377 | 1.1738 | 2.7788 |
| 52 | 1.632 | 1.294 | 1.97 | 0.7218 | 2.7338 |
| Mean | 1.796 | 1.454 | 2.137 | 0.8476 | 2.7658 |
| Days | | | | | |
| 31-52 | | | | | |
| Min | 1.632 | 1.294 | 1.970 | 0.6351 | 2.6721 |
| Max | 2.038 | 1.699 | 2.377 | 1.1738 | 2.8786 |

TABLE 3.3
Estimated Shannon index diversity expressed as group means with
95% confidence intervals in adult senior and geriatric dogs.

| | | | | | | Outliers for | |
|---|---|---|---|---|---|---|---|
| | | | | | | intervention | |
| | | | | Mean range | | 5th | 95th |
| | Age (years) | | Mean | 95% | 95% | Percentile | Percentile |
| Lifestage | Mean | Range | Diversity | Lower CI | Upper CI | of range | of range |
| Adult | 5.2 | 3.8-6.2 | 2.7644 | 2.3755 | 3.1534 | 1.83 | 3.72 |
| Senior | 10.0 | 8.2-12.9 | 2.5117 | 2.1971 | 2.8263 | 1.24 | 3.55 |
| Geriatric | 14.8 | 14.6-15.0 | 2.8306 | 2.3339 | 3.3273 | 2.16 | 3.47 |

| | | | | | |
|---|---|---|---|---|---|
| Mean diversity | 2.7022 | 2.3022 | 3.1023 | 1.74 | 3.58 |

### Example 4: Puppy Microbiota Blautia spp., Clostridium hiranonsis and Megamonas

A recent study of the puppy faecal microbiota described changes in the bacterial communities detected within the faeces of healthy puppies during the first year of life. The microbiota detected within the faeces of healthy puppies during the first year of life demonstrated that in the period before weaning, the most common types of bacteria belong to the Phylum Proteobacteria (FIG. 5). After weaning bacteria from the phylum Firmicutes were the most abundant detected. The diversity of the bacterial community also increased after weaning.

The initial elements of the puppy microbiota are likely from a maternal source and include Staphylococcus aureus and Bifidobacterium longum, which is known to be able to exploit the oligosaccharides present in the maternal milk, and a Clostridium sensu stricto 1 sp., amongst others. The presence of these taxa suggests that they are able to exploit the environment of the neonatal gut, given the availability of a source of nutrients from maternal milk, and the tolerance of various environmental stressors such as an unfavourable pH.

Following weaning, a number of species become more prevalent in the neonatal gut. The most notable examples are Megamonas sp. and Blautia sp. both of which are prolific fermenters of complex carbohydrates and producers of short chain fatty acids. In general, these species are associated with a healthy gut microbiome due to their production of short chain fatty acids, and their decreased abundance in dogs with diarrhoea [60, 61] and canine IBD [62]. Following this a large and sustained increase in Clostridium hiranonsis was observed, such that this becomes the most abundant taxa at all sampling points from Day 52 onwards until the final sampling point at Day 360. This species is also associated with a healthy gut microbiota, being involved in deconjugation of bile acids and decreased in cases of canine chronic enteropathy [22] and having a reported ability to inhibit the pathogen Clostridium difficile via secondary bile acids [23]. Overall, the increased abundance of Blautia spp., Clostridium hiranonsis and Megamonas spp. post-weaning indicate a healthy microbiota in puppies and adult dogs.

### Example 5: Allobaculum, Peptostreptococcus and Core Bifidobacterium, Lactobacillus, and Enterococcus

In a study of 22 dogs receiving a course of metronidazole prophylaxis for clinical signs of gastrointestinal dysbiosis, the faecal microbiota was assessed prior to, during, and following treatment. The study aimed to assess the extent, variability, and longevity of metronidazole treatment on the faecal microbiota in dogs. Metronidazole treatment was associated with a reduction in diarrhoea within the cohort. Assessment of the faecal microbiota by 16 S rRNA gene amplicon sequencing revealed reduced Shannon diversity and altered community composition during and immediately following treatment. While the animals received metronidazole, a core microbiota, dominated by OTU (sequence type) assigned to the lactic acid bacteria (Bifidobacterium, Lactobacillus, and Enterococcus) was observed across the cohort. This core microbiota representative of the organisms associated with metronidazole treatment was enriched for operational taxonomic units assigned to the genera Bifidobacterium, Lactobacillus, and Enterococcus. Diversity and species richness of the faecal microbiota increased to a post-treatment plateau around 4 weeks following the cessation of treatment. The increase in microbial diversity was associated with an apparent evolution within the microbial community composition of individuals, characterised by consistent signatures at both the OTU and genus taxonomic levels. Metronidazole treatment was associated with reduced microbial diversity, establishment of a core microbiota, and conserved features indicative of a consistent hierarchy in the evolution of gut microbiota community composition during the re-establishment of microbial diversity across individuals. The core microbiota associated with metronidazole treatment was enriched for sequences assigned to the lactic acid bacteria suggestive of innate resistance and the capability to perform activities essential to gut microbiome function.

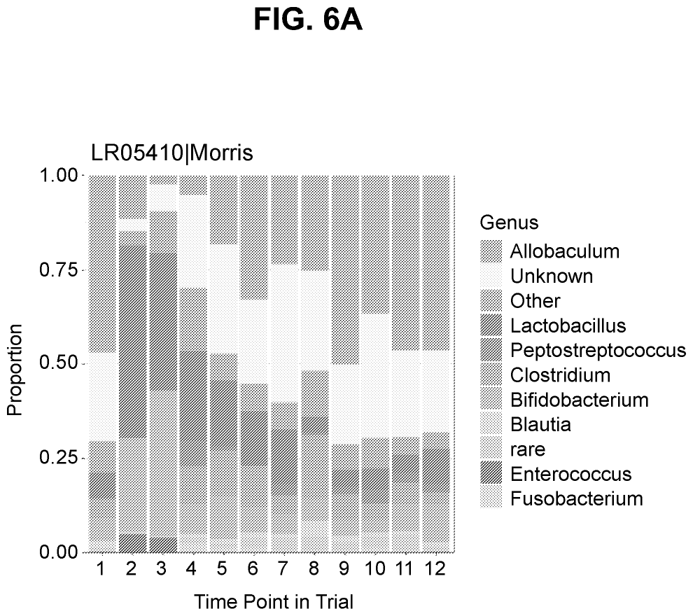

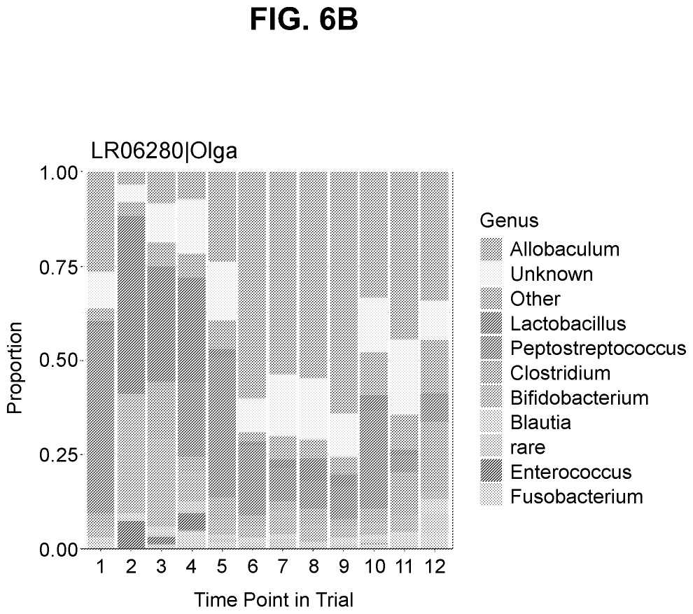

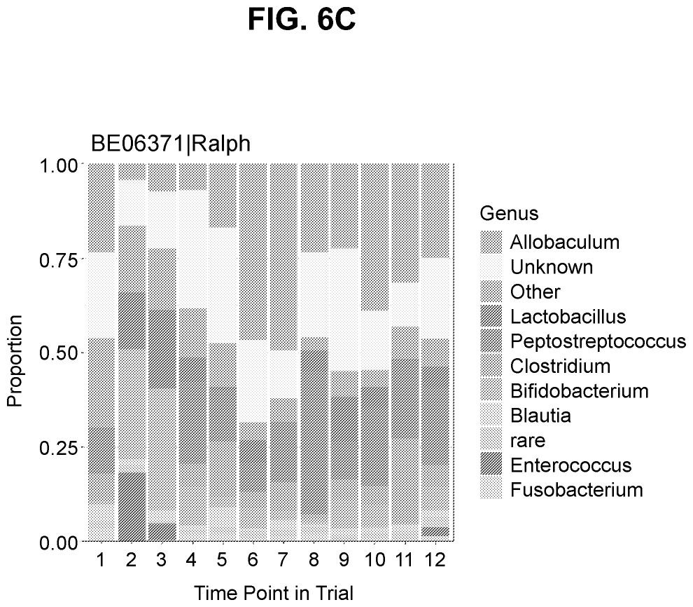

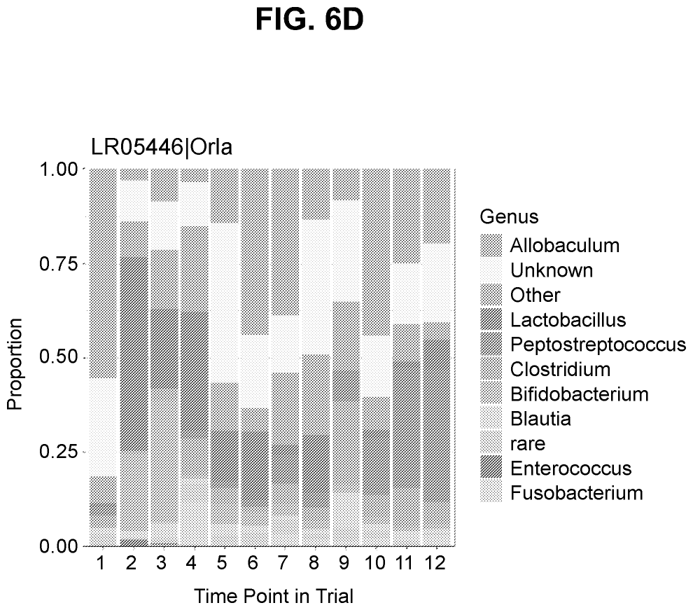

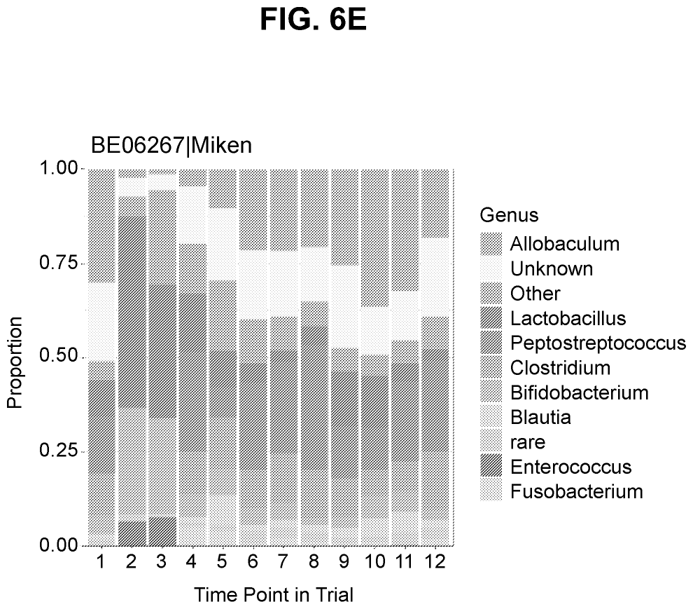

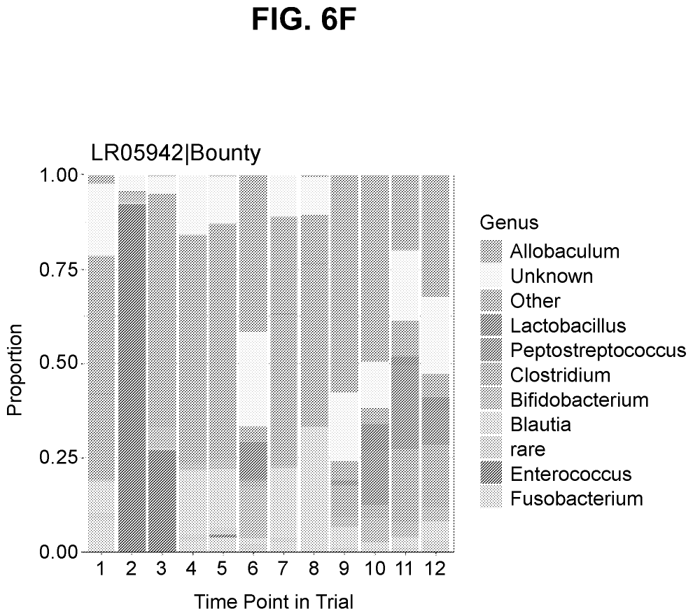

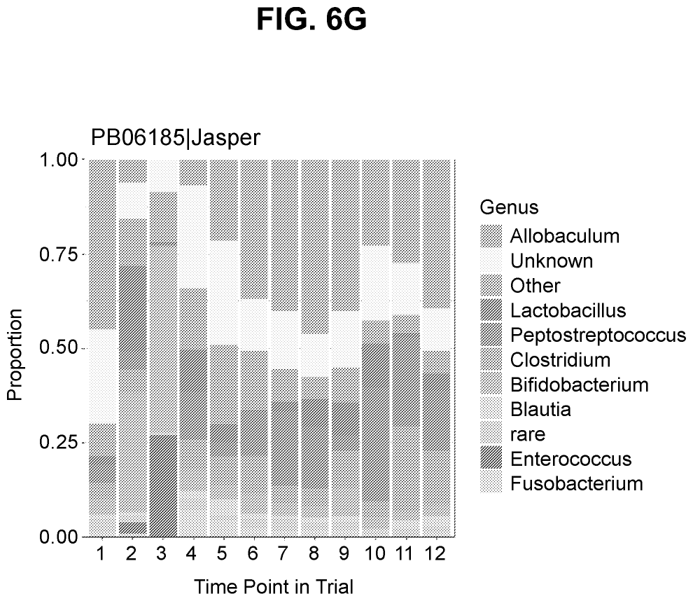

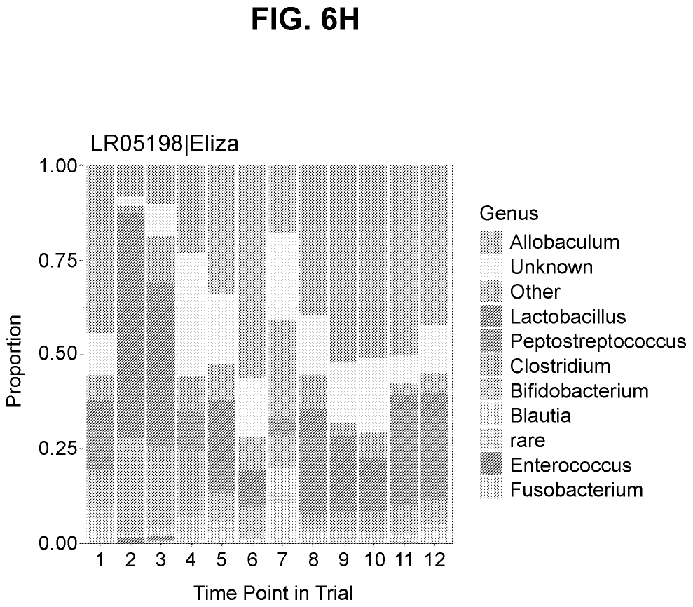

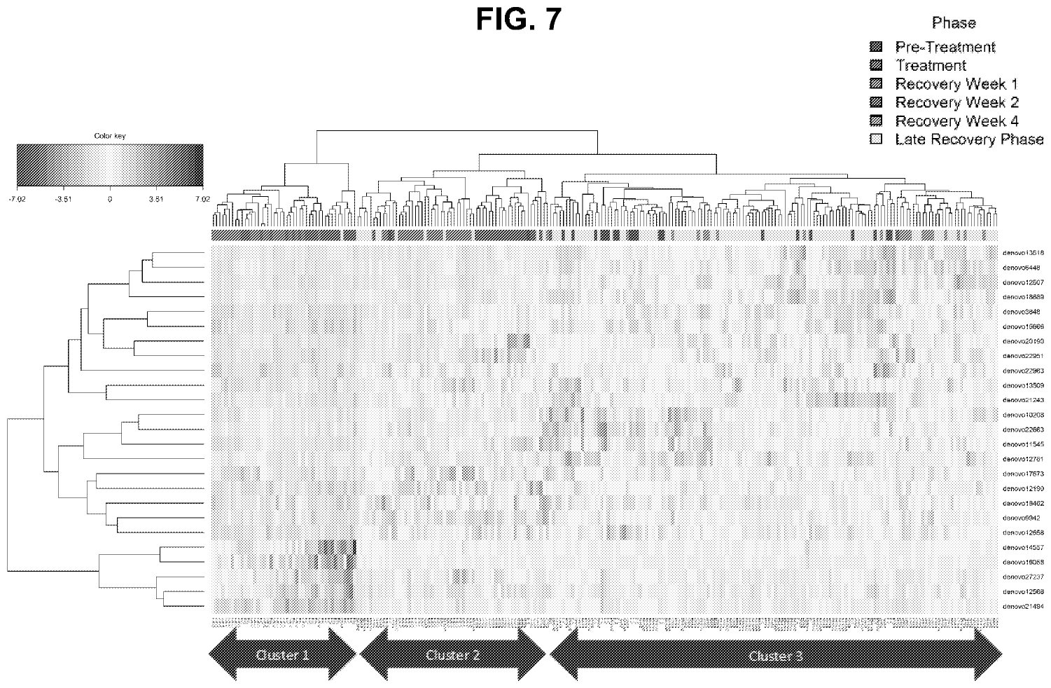

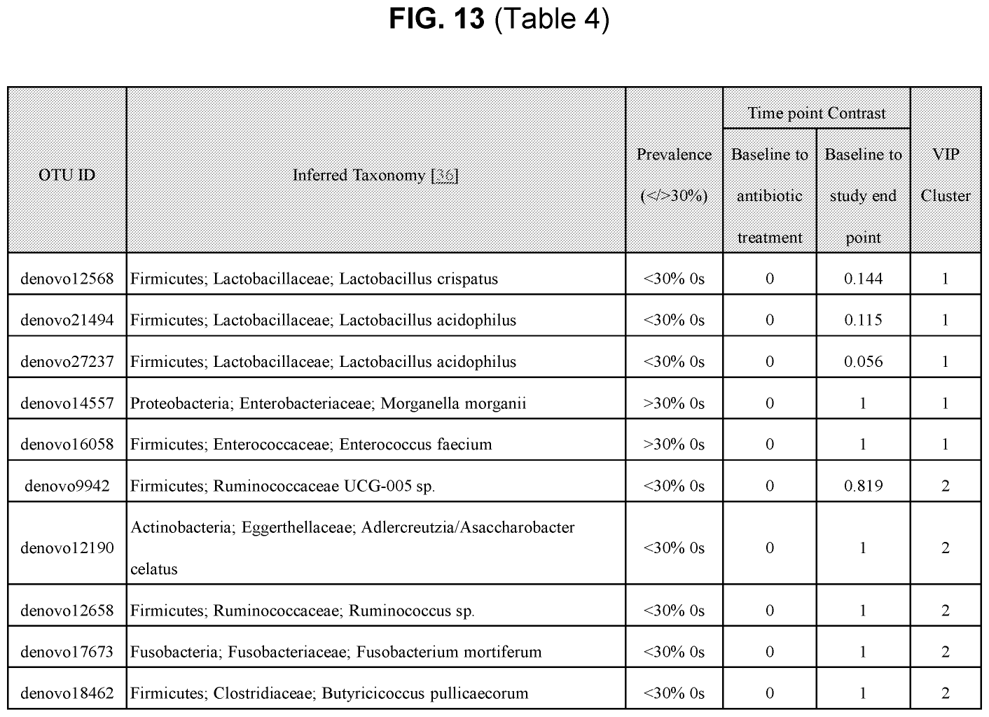

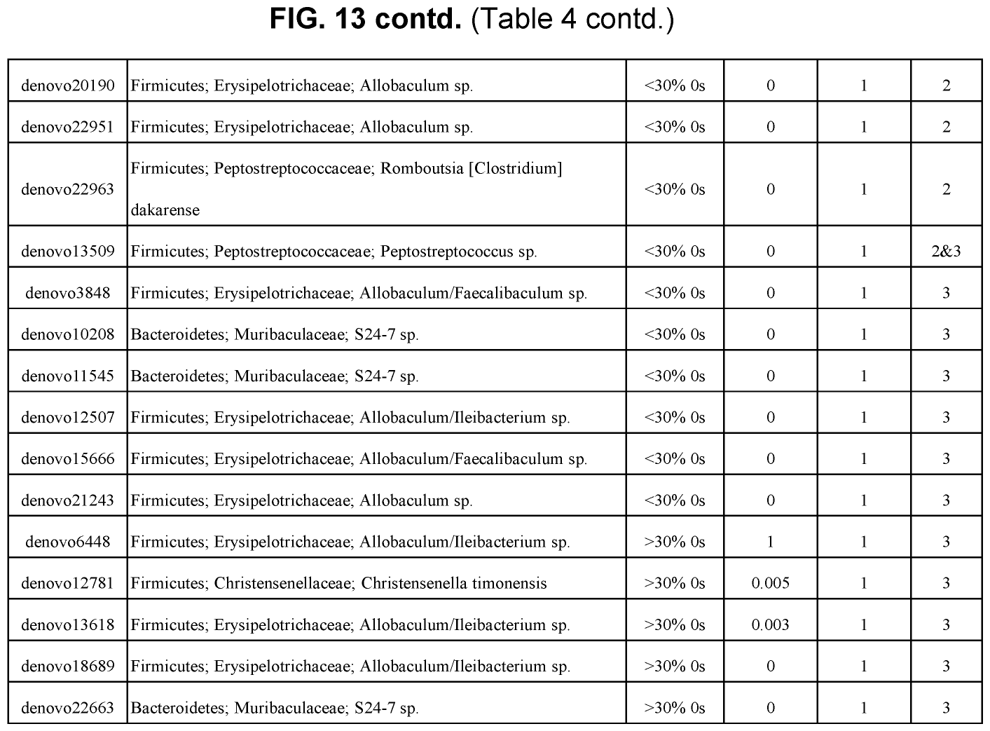

The composition of the microbiota during and immediately following treatment was dominated by lactic acid bacteria from the genera Lactobacillus, Bifidobacterium, and Enterococcus. The enhanced relative abundance of these genera, considered to be associated with gastrointestinal health in humans, is therefore likely to be responsible for the clinical resolution of dysbiosis and, by inference from their consistent representation across the cohort, can represent a healthy core microbiota naturally resistant to metronidazole and capable of performing the functions of the microbiome and restoring the gut microbiota and physiology. In the 1-2 weeks following the completion of antibiotic prophylaxis, a change in the genera represented was apparent with sequence types assigned to Allobaculum, Clostridium, and Peptostreptococcus spp. increasing in abundance as well as OTUs assigned to the genera Blautia. These bacteria therefore form the first organisms to recolonise the gut. By visual assessment of stacked plots, the treatment and first recovery time points (t=2 and t=3) dominated by lactic acid bacteria were followed in subsequent time points by the completion of treatment. Peptostreptococcus and Allobaculum genera, before return to a complexity similar to that observed during the baseline phase (FIGS. 6A-6H). This subset of OTUs best describes those most influential in driving the separation of samples into clusters associated with treatment phase based on their relative abundance profiles in faeces samples from the cohort. The subset comprised 9 OTUs assigned to the genus Allobaculum, 3 assigned to Lactobacillus, 3 to S24-7, and individual OTUs from the genera Christensenella, Peptostreptococcus, Romboutsia, Morganella, Adlercreutzia/Asaccharobacter, Enterococcus, and Butyricicoccus as well as 2 OTUs assigned to the family Ruminococcaceae (FIG. 7 and FIG. 13 (Table 4)). During and immediately following metronidazole treatment the relative abundance of three predominant OTUs were influential in the clustering, these were all assigned to the genus Lactobacillus. Additionally, two more minor OTUs detected in less than 30% of samples also influenced the clustering of samples into antibiotic and first sampling 2-3 days post-antibiotic therapy based on VIP score. These OTUs were assigned to the genera Enterococcus and Morganella (Enterobacteriaceae family). All OTUs in the second cluster influential in the early recovery phase during the first two weeks after treatment were prevalent, being detected in greater than 30% of the population. Taxonomic assignment of those OTUs driving the clustering of samples in this early recovery phase following the completion of antibiotic therapy described 2 OTUs from the family Ruminococcaceae and genus Allobaculum and one each from the family Eggerthellaceae and the genera Butyricicoccus, Fusobacterium, Romboutsia, and Peptostreptococcus. Finally, a third cluster defined by PLSDA VIP scores contained samples from the baseline and post-treatment phase 28 days after the completion of antibiotic treatment. OTUs represented at increased abundance within this cluster and prevalent being represented in more than 30% of the sample set included 4 Erysipelotrichaceae sp. assigned to the genus Allobaculum and 2 OTUs from the group S24-7, likely Muribaculaceae sp. A further group of lower prevalence OTUs were detected in less than 30% of the samples and included 3 OTUs assigned to the Erysipelotrichaceae genus Allobaculum/Ileibacterium, and one OTU each assigned to the S24-7 group and Christensenella genus. Clusters 1 and 2 (FIG. 13 (Table 4)) therefore represent basic core microbiota with health associated species associated with the restoration of clinical health.

### Detailed Description of FIG. 6A-6H

Stacked bar plots from eight representative dogs within the cohort demonstrating the distribution in the abundant taxonomic groups at each sampling point. Phylogenetic assignment to the genus level is shown as determined by DNA sequence of the 16 S rRNA gene v4 region. Abundance is expressed as a proportion relative to the total sequences for the sample. Sequences not assigned a nearest hit in the Green genes database (version 12_10) were collated into the ‘Unknown’ group; sequences of low abundance for visualisation and those representing less than 0.001% of sequences within the sample were assigned to Other and Rare groups respectively. Pre-treatment phase: t=1—Baseline; Treatment phase: t=2—Antibiotic administered; Recovery phase: t=3—Early week 1; t=4—Late week 1; t=5—week 2; t=6—week 4; t=7—week 6; t=8—week 8; t=9—month 3; t=10—month 4; t=11—month 5; t=12—month 6 Genera designations for the eight taxa that were most abundant throughout the study. Taxa that were observed that were not able to be expressed due to low level abundance were classified as Other; those that are not found as commonly were termed Rare; unclassified genera were Unknown.

### Detailed Description of FIG. 7

Partial least Square discriminate analysis (PLS-DA) correlation plot based on likeness in bacterial abundance data for the 25 OTUs displaying the greatest influence on clustering of the samples (variable importance in projection scores>1). Sample and OTU descriptors have been replaced for ease of visualisation with a colour guide (see key for details). Faeces samples are represented in vertical rows while bacterial OTUs are represented by horizontal rows within the heat plot. The heat map results are read in a similar manner to correlations although values are not constrained to (−1, 1). Dark red or blue sections on the heatmap indicate positively and negatively correlated groups of measurements respectively.

TABLE 7
Bacterial species associated with dysbiosis

| | SEQ | |
|---|---|---|
| Organism | ID NO | Sequence |
| Prevotella copri DSM | 160 | AGAGTTTGAT CCTGGCTCAG GATGAACGCT AGCTACAGGC |
| 18205 | | TTAACACATG CAAGTCGAGG GGAAACGACA TCGAAAGCTT |
| | | GCTTTTGATG GGCGTCGACC GGCGCACGGG TGAGTAACGC |
| | | GTATCCAACC TGCCCACCAC TTGGGGATAA CCTTGCGAAA |
| | | GTAAGACTAA TACCCAATGA TATCTCTAGA AGACATCTGA |
| | | AAGAGATTAA AGATTTATCG GTGATGGATG GGGATGCGTC |
| | | TGATTAGCTT GTTGGCGGGG TAACGGCCCA CCAAGGCGAC |
| | | GATCAGTAGG GGTTCTGAGA GGAAGGTCCC CCACATTGGA |
| | | ACTGAGACAC GGTCCAAACT CCTACGGGAG GCAGCAGTGA |
| | | GGAATATTGG TCAATGGGCG AGAGCCTGAA CCAGCCAAGT |
| | | AGCGTGCAGG ATGACGGCCC TATGGGTTGT AAACTGCTTT |
| | | TATAAGGGAA TAAAGTGAGC CTCGTGAGGC TTTTTGCATG |
| | | TACCTTATGA ATAAGGACCG GCTAATTCCG TGCCAGCAGC |
| | | CGCGGTAATA CGGAAGGTCC GGGCGTTATC CGGATTTATT |
| | | GGGTTTAAAG GGAGCGTAGG CCGGAGATTA AGCGTGTTGT |
| | | GAAATGTAGA CGCTCAACGT CTGCACTGCA GCGCGAACTG |
| | | GTTTCCTTGA GTACGCACAA AGTGGGCGGA ATTCGTGGTG |
| | | TAGCGGTGAA ATGCTTAGAT ATCACGAAGA ACTCCGATTG |
| | | CGAAGGCAGC TCACTGGAGC GCAACTGACG CTGAAGCTCG |
| | | AAAGTGCGGG TATCGAACAG GATTAGATAC CCTGGTAGTC |
| | | CGCACGGTAA ACGATGGATG CCCGCTGTTG GTCTGAACAG |
| | | GTCAGCGGCC AAGCGAAAGC ATTAAGCATC CCACCTGGGG |
| | | AGTACGCCGG CAACGGTGAA ACTCAAAGGA ATTGACGGGG |
| | | GCCCGCACAA GCGGAGGAAC ATGTGGTTTA ATTCGATGAT |
| | | ACGCGAGGAA CCTTACCCGG GCTTGAATTG CAGAGGAAGG |
| | | ATTTGGAGAC AATGACGCCC TTCGGGGCCT CTGTGAAGGT |
| | | GCTGCATGGT TGTCGTCAGC TCGTGCCGTG AGGTGTCGGC |
| | | TTAAGTGCCA TAACGAGCGC AACCCCTCTC CTTAGTTGCC |
| | | ATCAGGTTAA GCTGGGCACT CTGGGGACAC TGCCACCGTA |
| | | AGGTGTGAGG AAGGTGGGGA TGACGTCAAA TCAGCACGGC |
| | | CCTTACGTCC GGGGCTACAC ACGTGTTACA ATGGCAGGTA |
| | | CAGAGAGACG GTYSTATGYA AATASGATCA AATCCTTAAA |
| | | GCCTGTCTCA GTTCGGACTG GGGTCTGCAA CCCGACCCCA |
| | | CGAAGCTGGA TTCGCTAGTA ATCGCGCATC AGCCATGGCG |
| | | CGGTGAATAC GTTCCCGGGC CTTGTACACA CCGCCCGTCA |
| | | AGCCATGAAA GCCGGGGGCG CCTAAAGTCC GTGACCGTAA |
| | | GGAGCGGCCT AGGGCGAAAC TGGTAATTGG GGCTAAGTCG |
| | | TAACAAGGTA ACC |
| Mogibacterium COT112 | 161 | GAGTTTGATC CTGGCTCAGG ATGAACGCTG GCGGCGTGCC |
| | | TAATACATGC AAGTCGAGCG AGATGTTAGT GCATGAACCT |
| | | TCGGGGGATT ATACTAACGG ACAGCGGCGG ACGGGTGAGT |
| | | AACGCGTAGG CAACCTGCCC CTGACAGAGG GATAGCCATT |
| | | GGAAACGATG ATTAAAACCT CATGACACCG TAGAAGCACA |
| | | TGCTTCATCG GTCAAAGATT TATCGGTCGG GGATGGGCCT |
| | | GCGTCTGATT AACTAGTTGG TGAGGTAACG GCTCACCAAG |
| | | GTGACGATCA GTAGCCGACC TGAGAGGGTG ATCGGCCACA |
| | | TTGGAACTGA GACACGGTCC AAACTTCTAC GGAAGGCAGC |
| | | AGTAGGGAAT CTTGCACAAT GGGCGAAAGC CTGATGCAGC |
| | | AACGCCGCGT GAAGGATGAA GGCCTTCGGG TTGTAAACTT |
| | | CTGTTCTAAG GGAAGAAAGA AATGACGGTA CCTTAGGAGC |
| | | AAGCCCCGGC TAACTACGTG CCAGCAGCCG CGGTAATACG |
| | | TAGGGGGCAA GCGTTATCCG GAATTATTGG GCGTAAAGAG |
| | | TGCGTAGGTG GTTACCTAAG CGCAAGGTTT AATTTAGAGG |
| | | CTCAACCTCT ACTTGCCTTG CGAACTGGGC TACTTGAGTG |
| | | CAGGAGGGGA AAGCGGAATT CCTAGTGTAG CGGTGAAATG |
| | | CGTAGATATT AGGAGGAACA CCAGCGGCGA AGGCGGCTTT |
| | | CTGGACTGTA ACTGACACTG AGGCACGAAA GCGTGGGTAG |
| | | CAAACAGGAT TAGATACCCT GGTAGTCCAC GCCGTAAACG |
| | | ATGAGCACTA GGTGTTGGGT CCGTTAGGAC TCAGTGCCGC |
| | | AGTTAACGCA ATAAGTGCTC CGCCTGGGGA GTACGCTCGC |
| | | AAGAGTAAAA CTCAAAGGAA TTGACGGGGA CCCGCACAAG |
| | | CAGCGGAGCA TGTGGTTTAA TTCGAAGCAA CGCGAAGAAC |
| | | CTTACCAGGG CTTGACATCC TGCTGACAGG ACTTTAACAG |
| | | GTTCCTTCTT CGGACAGCAG AGACAGGTGG TGCATGGTTG |
| | | TCGTCAGCTC GTGTCGTGAG ATGTTGGGTT AAGTCCCGCA |
| | | ACGAGCGCAA CCCTTGTCGC TAGTTACTAA CATTCAGTTG |
| | | AGGACTCTAG CGAGACTGCC GAGGTCAACT CGGAGGAAGG |
| | | TGGGGATGAC GTCAAATCAT CATGCCCCTT ATGTTCTGGG |
| | | CTACACACGT GCTACAATGG TCGGTACAAT GAGAGGCAAT |
| | | ACTGCGAAGT GGAGCGAATC ACCAAAACCG ATCCCAGTTC |
| | | GGATTGTAGG CTGCAACTCG CCTACATGAA GTTGGAGTTG |
| | | CTAGTAATCG CAGATCAGAA TGCTGCGGTG AATGCGTTCC |
| | | CGGGTCTTGT ACACACCGCC CGTCACACCA TGGAAGTTGG |
| | | GGGTGCCCAA AGTCGGTTAA TTAATCTATC GCCTAAGGCA |
| | | AAACCAATGA CTGGGGTGAA GTCGTAACAA GGTAG |
| Mogibacterium COT343 | 162 | GAGTTTGATC CTGGCTCAGG ATGAACGCTG GCGGCGTGCC |
| (timidum) | | TAATACATGC AAGTCGAGCG AGAAGCTTGG AAATGACGCT |
| | | TCGGTTGATT TTCCAAGCGG ACAGCGGCGG ACGGGTGAGT |
| | | AACGCGTAGG CAACCTGCCC CTGACAGAGG GATAGCCATT |
| | | GGAAACGATG ATTAAAACCT CATGACACCG TAGTAGCACA |
| | | TGCTACATCG GTCAAAGATT TATCGGTCAG GGATGGGCCT |
| | | GCGTCTGATT AACTGGTTGG TGAGGTAACG GCTCACCAAG |
| | | GTGACGATCA GTAGCCGACC TGAGAGGGTG ATCGGCCACA |
| | | TTGGAACTGA GACACGGTCC AAACTTCTAC GGAAGGCAGC |
| | | AGTAGGGAAT CTTGCACAAT GGGCGAAAGC CTGATGCAGC |
| | | AACGCCGCGT GAAGGATGAA GGCCTTCGGG TTGTAAACTT |
| | | CTGTTCTAAG GGAAGAAAGA AATGACGGTA CCTTAGGAGC |
| | | AAGCCCCGGC TAACTACGTG CCAGCAGCCG CGGTAATACG |
| | | TAGGGGGCAA GCGTTATCCG GAATTATTGG GCGTAAAGAG |
| | | TGCGTAGGTG GTTACCTAAG CGCAAGGTTT AAATTAGAGG |
| | | CTCAACCTCT ACATGCCTTG CGAACTGGGC TACTTGAGTG |
| | | CAGGAGGGGA AAGCGGAATT CCTAGTGTAG CGGTGAAATG |
| | | CGTAGATATT AGGAGGAACA CCGGCGGCGA AGGCGGCTTT |
| | | CTGGACTGTA ACTGACACTG AGGCACGAAA GCGTGGGTAG |
| | | CAAACAGGAT TAGATACCCT GGTAGTCCAC GCCGTAAACG |
| | | ATGAGCACTA GGTGTTGGGT CCGTTAGGAC TCAGTGCCGC |
| | | AGTTAACGCA ATAAGTGCTC CGCCTGGGGA GTACGCTCGC |
| | | AAGAGTAAAA CTCAAAGGAA TTGACGGGGA CCCGCACAAG |
| | | CAGCGGAGCA TGTGGTTTAA TTCGAAGCAA CGCGAAGAAC |
| | | CTTACCAGGG CTTGACATCC TGCTGACAGA ACCTTAATCG |
| | | GCTTTTTCTT CGGACAGCAG AGACAGGTGG TGCATGGTTG |
| | | TCGTCAGCTC GTGTCGTGAG ATGTTGGGTT AAGTCCCGCA |
| | | ACGAGCGCAA CCCTTGTCGC TAGTTACTAA CATTCAGTTG |
| | | AGGACTCTAG CGAGACTGCC GAGGTCAACT CGGAGGAAGG |
| | | TGGGGATGAC GTCAGATCAT CATGCCCCTT ATGTTCTGGG |
| | | CTACACACGT GCTACAATGG TCGGTACAAT GAGATGCAAT |
| | | ACTGCGAAGT GGAGCGAAAC ACCAAAACCG ATCCCAGTTC |
| | | GGATTGTAGG CTGCAACTCG CCTACATGAA GTCGGAGTTG |
| | | CTAGTAATCG CAGATCAGAA TGCTGCGGTG AATGCGTTCC |
| | | CGGGTCTTGT ACACACCGCC CGTCACACCA TGGAAGTTGG |
| | | GGGTGCCCAA AGTCGGTTAA TTAATCTATC GCCTAAGGCA |
| | | AAACCAATGA CTGGGGTGAA GTCGTAACAA GGTAGCCGTT |
| | | CGAGAACGAG CGGCTGGATC ACCTCCTT |
| Fusobacterium | 163 | GAGTTTGATC CTGGCTCAGG ATGAACGCTG ACAGAATGCT |
| canifelinum | | TAACACATGC AAGTCTACTT GAATTTGGGT TTTTTAACTT |
| | | CGATTTGGGT GGCGGACGGG TGAGTAACGC GTAAAGAACT |
| | | TGCCTCACAG CTAGGGACAA CATTTGGAAA CGAATGCTAA |
| | | TACCTGATAT TATGATTTTA GGGCATCCTA GAATTATGAA |
| | | AGCTATATGC GCTGTGAGAG AGCTTTGCGT CCCATTAGCT |
| | | AGTTGGAGAG GTAACGGCTC ACCAAGGCGA TGATGGGTAG |
| | | CCGGCCTGAG AGGGTGATCG GCCACAAGGG GACTGAGACA |
| | | CGGCCCTTAC TCCTACGGGA GGCAGCAGTG GGGAATATTG |
| | | GACAATGGAC CAAGAGTCTG ATCCAGCAAT TCCGTGTGCA |
| | | CGATGAAGTT TTTCGGAATG TAAAGTGCTT TCAGTTGGGA |
| | | AGAAAAAAAT GACGGTACCA ACAGAAGAAG TGACGGCTAA |
| | | ATACGTGCCA GCAGCCGCGG TAATACGTAT GTCACAAGCG |
| | | TTATCCGGAT TTATTGGGCG TAAAGCGCGT CTAGGTGGTT |
| | | ATGTAAGTCT GATGTGAAAA TGCAGGGCTC AACTCTGTAT |
| | | TGCGTTGGAA ACTGTGTAAC TAGAGTACTG GAGAGGTAAG |
| | | CGGAACTACA AGTGTAGAGG TGAAATTCGT AGATATTTGT |
| | | AGGAATGCCG ATGGGGAAGC CAGCTTACTG GACAGATACT |
| | | GACGCTGAAG CGCGAAAGCG TGGGTAGCAA ACAGGATTAG |
| | | ATACCCTGGT AGTCCACGCC GTAAACGATG ATTACTAGGT |
| | | GTTGGGGGTC GAACCTCAGC GCCCAAGCAA ACGCGATAAG |
| | | TAATCCGCCT GGGGAGTACG TACGCAAGTA TGAAACTCAA |
| | | AGGAATTGAC GGGGACCCGC ACAAGCGGTG GAGCATGTGG |
| | | TTTAATTCGA CGCAACGCGA GGAACCTTAC CAGCGTTTGA |
| | | CATCTTAGGA ATGAGACAGA GATGTTTCAG TGTCCCTTCG |
| | | GGGAAACCTA AAGACAGGTG GTGCATGGCT GTCGTCAGCT |
| | | CGTGTCGTGA GATGTTGGGT TAAGTCCCGC AACGAGCGCA |
| | | ACCCCTTTCG TATGTTACCA TCATTAAGTT GGGGACTCAT |
| | | GCGATACTGC CTACGATGAG TAGGAGGAAG GTGGGGATGA |
| | | CGTCAAGTCA TCATGCCCCT TATACGCTGG GCTACACACG |
| | | TGCTACAATG GGTAGTACAG AGAGTCGCAA AGCCGTGAGG |
| | | TAGAGCTAAT CTCAGAAAAC TATTCTTAGT TCGGATTGTA |
| | | CTCTGCAACT CGAGTACATG AAGTTGGAAT CGCTAGTAAT |
| | | CGCGAATCAG CAATGTCGCG GTGAATACGT TCTCGGGTCT |
| | | TGTACACACC GCCCGTCACA CCACGAGAGT TGGTTGCACC |
| | | TGAAGTAGCA GGCCTAACCG CAAGGAGGGA TGCTCCGAGG |
| | | GTGTGATTAG CGATTGGGGT GAAGTCGTAA CAAGGT |
| Fusobacterium | 164 | GAGTTTGATC CTGGCTCAGG ATGAACGCTG ACAGAATGCT |
| necrophorum | | TAACACATGC AAGTCGACTC GAGTCTTCGG ACTTGGGTGG |
| | | CGCACGGGTG AGTAACGCGT AAAGAACTTG CCTCTTAGAC |
| | | CGGGACAACA TCTGGAAACG GATGCTAATA CCGGATATTA |
| | | TGGTTTTTTC GCATGGAGGA ATCATGAAAG CTAGATGCGC |
| | | TAAGAGAGAG CTTTGCGTCC CATTAGCTAG TTGGTGAGGT |
| | | AACGGCCCAC CAAGGCAATG ATGGGTAGCC GGCCTGAGAG |
| | | GGTGAACGGC CACAAGGGGA CTGAGACACG GCCCTTACTC |
| | | CTACGGGAGG CAGCAGTGGG GAATATTGGA CAATGGACCA |
| | | CAAGTCTGAT CCAGCAATTC TGTGTGCACG ATGACGTTTT |
| | | TCGGAATGTA AAGTGCTTTC AGTCGGGAAG AAGTCAGTGA |
| | | CGGTACCGAC AGAAGAAGCG ACGGCTAAAT ACGTGCCAGC |
| | | AGCCGCGGTA ATACGTATGT CGCAAGCGTT ATCCGGATTT |
| | | ATTGGGCGTA AAGCGCGTCT AGGCGGCAAG GAAAGTCTGA |
| | | TGTGAAAATG CGGAGCTCAA CTCCGTATGG CGTTGGAAAC |
| | | TGCCTTACTA GAGTACTGGA GAGGTAGGCG GAACTACAAG |
| | | TGTAGAGGTG AAATTCTTAG ATATTTGTAG GAATGCCGAT |
| | | GGGGAAGCCA GCCTACTGGA CAGATACTGA CGCTAAAGCG |
| | | CGAAAGCGTG GGTAGCAAAC AGGATTAGAT ACCCTGGTAG |
| | | TCCACGCTGT AAACGATGAT TACTAGGTGT TGGGGGTCAA |
| | | ACCTCAGCGC CCAAGCTAAC GCGATAAGTA ATCCGCCTGG |
| | | GGAGTACGTA CGCAAGTATG AAACTCAAAG GAATTGACGG |
| | | GGACCCGCAC AAGCGGTGGA GCATGTGGTT TAATTCGACG |
| | | CAACGCGAGG AACCTTACCA GCGTTTGACA TCCTACGAAC |
| | | GGAGCAGAGA TGCGCCGGTG CCCTTTCGGG GGAACGTAGT |
| | | GACAGGTGGT GCATGGCTGT CGTCAGCTCG TGTCGTGAGA |
| | | TGTTGGGTTA AGTCCCGCAA CGAGCGCAAC CCCTATCGTA |
| | | TGTTACCATC CTTCAGTTGG GGACTCATGC GGTACTGCCT |
| | | GCGACGAGCA GGAGGAAGGT GGGGATGACG TCAAGTCATC |
| | | ATGCCCCTTA TACGCTGGGC TACACACGTG CTACAATGGG |
| | | TAGTACAGAG AGCAGCAAAC CCGCGAGGGG GAGCAAATCT |
| | | CAGAAAACTA TTCTTAGTTC GGATTGTACT CTGCGACTCG |
| | | AGTACATGAA GTTGGAATCG CTAGTAATCG CAAATCAGCA |
| | | ATGTTGCGGT GAATACGTTC TCGGGTCTTG TACACACCGC |
| | | CCGTCACACC ACGAGAGTTG GTTGCACCTG AAGTAGCAGG |
| | | CCTAACCTTA GGGAAGGATG CTCCGAGGGT GTGGTTAGCG |
| | | ATTGGGGTGA AGTCGTAACA AGGT |
| Fusobacterium | 165 | GAGTTTGATC CTGGCTCAGG ATGAACGCTG ACAGAATGCT |
| nucleatum subsp. | | TAACACATGC AAGTCAACTT GAATTTGGGT TTTTAACTTA |
| Animalis | | GATTTGGGTG GCGGACGGGT GAGTAACGCG TAAAGAACTT |
| | | GCCTCACAGC TAGGGACAAC ATTTAGAAAT GAATGCTAAT |
| | | ACCTGATATT ATGATTTTAA GGCATCTTAG AATTATGAAA |
| | | GCTATAAGCA CTGTGAGAGA GCTTTGCGTC CCATTAGCTA |
| | | GTTGGAGAGG TAACAGCTCA CCAAGGCGAT GATGGGTAGC |
| | | CGGCCTGAGA GGGTGAACGG CCACAAGGGG ACTGAGACAC |
| | | GGCCCTTACT CCTACGGGAG GCAGCAGTGG GGAATATTGG |
| | | ACAATGGACC GAGAGTCTGA TCCAGCAATT CTGTGTGCAC |
| | | GATGAAGTTT TTCGGAATGT AAAGTGCTTT CAGTTGGGAA |
| | | GAAATAAATG ACGGTACCAA CAGAAGAAGT GACGGCTAAA |
| | | TACGTGCCAG CAGCCGCGGT AATACGTATG TCACGAGCGT |
| | | TATCCGGATT TATTGGGCGT AAAGCGCGTC TAGGTGGTTA |
| | | TGTAAGTCTG ATGTGAAAAT GCAGGGCTCA ACTCTGTATT |
| | | GCGTTGGAAA CTGTGTAACT AGAGTACTGG AGAGGTAAGC |
| | | GGAACTACAA GTGTAGAGGT GAAATTCGTA GATATTTGTA |
| | | GGAATGCCGA TGGGGAAGCC AGCTTACTGG ACAGATACTG |
| | | ACGCTAAAGC GCGAAAGCGT GGGTAGCAAA CAGGATTAGA |
| | | TACCCTGGTA GTCCACGCTG TAAACGATGA TTACTAGGTG |
| | | TTGGGGGTCG AACCTCAGCG CCCAAGCAAA CGCGATAAGT |
| | | AATCCGCCTG GGGAGTACGT ACGCAAGTAT GAAACTCAAA |
| | | GGAATTGACG GGGACCCGCA CAAGCGGTGG AGCATGTGGT |
| | | TTAATTCGAC GCAACGCGAG GAACCTTACC AGCGTTTGAC |
| | | ATCTTAGGAA TGAGATAGAG ATATTTCAGT GTCCCTTCGG |
| | | GGAAACCTAA AGACAGGTGG TGCATGGCTG TCGTCAGCTC |
| | | GTGTCGTGAG ATGTTGGGTT AAGTCCCGCA ACGAGCGCAA |
| | | CCCCTTTCGT ATGTTACCAT CATTAAGTTG GGGACTCATG |
| | | CGATACTGCC TACGATGAGT AGGAGGAAGG TGGGGATGAC |
| | | GTCAAGTCAT CATGCCCCTT ATACGCTGGG CTACACACGT |
| | | GCTACAATGG GTAGAACAGA GAGTTGCAAA GCCGTGAGGT |
| | | GAAGCTAATC TCAGAAAACT ATTCTTAGTT CGGATTGTAC |
| | | TCTGCAACTC GAGTACATGA AGTTGGAATC GCTAGTAATC |
| | | GCGAATCAGC AATGTCGCGG TGAATACGTT CTCGGGTCTT |
| | | GTACACACCG CCCGTCACAC CACGAGAGTT GGTTGCACCT |
| | | GAAGTAGCAG GCCTAACCGT AAGGAGGGAT GCTCCGAGGG |
| | | TGTGATTAGC GATTGGGGTG AAGTCGTAAC AAGGT |
| Escherichia/Shigella | 166 | GAGTTTGATC ATGGCTCAGA TTGAACGCTG GCGGCAGGCC |
| COT277 | | TAACACATGC AAGTCGAACG GTAACAGGAA GAAGCTTGCT |
| | | TCTTTGCTGA CGAGTGGCGG ACGGGTGAGT AATGTCTGGG |
| | | AAACTGCCTG ATGGAGGGGG ATAACTACTG GAAACGGTAG |
| | | CTAATACCGC ATAACGTCGC AAGACCAAAG AGGGGGACCT |
| | | TCGGGCCTCT TGCCATCGGA TGTGCCCAGA TGGGATTAGC |
| | | TAGTAGGTGG GGTAACGGCT CACCTAGGCG ACGATCCCTA |
| | | GCTGGTCTGA GAGGATGACC AGCCACACTG GAACTGAGAC |
| | | ACGGTCCAGA CTCCTACGGG AGGCAGCAGT GGGGAATATT |
| | | GCACAATGGG CGCAAGCCAG ATGCAGCCAT GCCGCGTGTA |
| | | TGAAGAAGGC CTTCGGGTTG TAAAGTACTT TCAGCGGGGA |
| | | GGAAGGGAGT AAAGTTAATA CCTTTGCTCA TTGACGTTAC |
| | | CCGCAGAAGA AGCACCGGCT AACTCCGTGC CAGCAGCCGC |
| | | GGTAATACGG AGGGTGCAAG CGTTAATCGG AATTACTGGG |
| | | CGTAAAGCGC ACGCAGGCGG TTTGTTAAGT CAGATGTGAA |
| | | ATCCCCGGGC TCAACCTGGG AACTGCATCT GATACTGGCA |
| | | AGCTTGAGTC TCGTAGAGGG GGGTAGAATT CCAGGTGTAG |
| | | CGGTGAAATG CGTAGAGATC TGGAGGAATA CCGGTGGCGA |
| | | AGGCGGCCCC CTGGACGAAG ACTGACGCTC AGGTGCGAAA |
| | | GCGTGGGGAG CAAACAGGAT TAGATACCCT GGTAGTCCAC |
| | | GCCGTAAACG ATGTCGACTT GGAGGTTGTG CCCTTGAGGC |
| | | GTGGCTTCCG GAGCTAACGC GTTAAGTCGA CCGCCTGGGG |
| | | AGTACGGCCG CAAGGTTAAA ACTCAAATGA ATTGACGGGG |
| | | GCCCGCACAA GCGGTGGAGC ATGTGGTTTA ATTCGATGCA |
| | | ACGCGAAGAA CCTTACCTGG TCTTGACATC CACAGAACTT |
| | | CCCAGAGATG GATTGGTGCC TTCGGGAACT GTGAGACAGG |
| | | TGCTGCATGG CTGTCGTCAG CTCGTGTTGT GAAATGTTGG |
| | | GTTAAGTCCC GCAACGAGCG CAACCCTTAT CCTTTGTTGC |
| | | CAGCGGTCCG GCCGGGAACT CAAAGGAGAC TGCCAGTGAT |
| | | AAACTGGAGG AAGGTGGGGA TGACGTCAAG TCATCATGGC |
| | | CCTTACGACC AGGGCTACAC ACGTGCTACA ATGGCGCATA |
| | | CAAAGAGAAG CGACCTCGCG AGAGCAAGCG GACCTCATAA |
| | | AGTGCGTCGT AGTCCGGATT GGAGTCTGCA ACTCGACTCC |
| | | ATGAAGTCGG AATCGCTAGT AATCGTGGAT CAGAATGCCA |
| | | CGGTGAATAC GTTCCCGGGC CTTGTACACA CCGCCCGTCA |
| | | CACCATGGGA GTGGGTTGCA AAAGAAGTAG GTAGCTTAAC |
| | | CTTCGGGAGG GCGCTTACCA CTTTGTGATT CATGACTGGG |
| | | GTGAAGTCGT AACAAGGTAA CCGTAGGGGA ACCTGCGGTT |
| | | GGATCACCTC CTT |

TABLE 8
Relevant to ranges of bacteria in Examples 1, 2, 4 & 5:

| | | | | | | Sequence type reference | |
|---|---|---|---|---|---|---|---|
| | | Minimum | Maximum | Upper | Lower | numbers | SEQ |
| | | Range in | Range in | 5% | 5% | (OTU and Sequence ID) | ID |
| Genus | n= | Abundance | Abundance | range | range | Greengenes V13.5 | NO |
| Allobaculum | 8102 | 5.62E−06 | 0.816219869 | 0.04135299 | 0.0504309 | 1105860, 386788, | 154 |
| | | | | | | 4379961, 4310326, | |
| | | | | | | 135952, 130091, 428953, | |
| | | | | | | 1108699, 134101, 277143 | |
| Bifidobacterium | 4354 | 4.84E−06 | 0.629647871 | 0.02062964 | 0.0268991 | 4426298, 359098, 72820, | |
| | | | | | | 4413347, 822770, | |
| | | | | | | 471180, 681370, 519290, | |
| | | | | | | 1142029, 559527 | |
| Blautia | 29338 | 1.97E−05 | 0.6373751 | 0.07062645 | 0.0779575 | 364048, 583089, 532203, | 152 |
| | | | | | | 365455, 367456, 567715, | & |
| | | | | | | 326865, 364824, 328628, | 153 |
| | | | | | | 292633, 344488, 580087, | |
| | | | | | | 351163, 362108, 589313, | |
| | | | | | | 696563, 436032, | |
| | | | | | | 4429536, 192326, | |
| | | | | | | 363507, 191601, 362037, | |
| | | | | | | 194384, 357168, 199243, | |
| | | | | | | 311712, 297970, 348362, | |
| | | | | | | 404292, 525378, 354095, | |
| | | | | | | 338626, 293543, 304309, | |
| | | | | | | 554160, 302090, 333024, | |
| | | | | | | 297529, 587269, 325848, | |
| | | | | | | 2035344 | |
| Clostridium | 7358 | 1.35E−05 | 0.844050631 | 0.18917637 | 0.20662887 | 582379, 309107, 356403, | 155 |
| hiranonis | | | | | | 351084, 352473, | |
| | | | | | | 4414489, 347131 | |
| Dorea | 12104 | 3.26E−05 | 0.760047249 | 0.02715946 | 0.03003408 | 320468, 305313, 293869, | |
| | | | | | | 181990, 3172379, | |
| | | | | | | 195999, 3409363, | |
| | | | | | | 367535, 363232, 357020, | |
| | | | | | | 2555599, 317646, | |
| | | | | | | 320430, 592933, | |
| | | | | | | 1760821, 187338, | |
| | | | | | | 342110, 1105552 | |
| Enterobacteriaceae | 9177 | 2.90E−06 | 0.986857172 | 0.07881779 | 0.09660931 | 4425571, 114510, | 166 |
| | | | | | | 566243, 4111715, | |
| | | | | | | 782953, 588216, 470879, | |
| | | | | | | 345362, 299267, 295053, | |
| | | | | | | 797229, 1111294, | |
| | | | | | | 169182, 304641, | |
| | | | | | | 1890229, 813217, | |
| | | | | | | 525841, 284672, 331697 | |
| Enterococcus | 4230 | 1.67E−06 | 0.995548705 | 0.02141405 | 0.03115899 | 624891, 584241, 295146, | |
| | | | | | | 1135616, 3697034, | |
| | | | | | | 1111582, 766768, | |
| | | | | | | 226338, 590982, 1085646 | |
| Fusobacterium | 10983 | 2.32E−06 | 0.737701238 | 0.06439895 | 0.07305165 | 809380, 572889, | 163 |
| | | | | | | 1654477, 345114, | & |
| | | | | | | 342025, 1592748, | 164 |
| | | | | | | 4439398, 4254313, | & |
| | | | | | | 351979, 444857, 828676, | 165 |
| | | | | | | 4333154, 2825358 | |
| Lachnospiraceae | 87865 | 2.20E−05 | 0.88446714 | 0.1696669 | 0.18127336 | 370098, 298247, 364048, | |
| | | | | | | 360015, 546876, 320468, | |
| | | | | | | 583089, 305313, 383971, | |
| | | | | | | 532203, 3579831, | |
| | | | | | | 300716, 518438, 156357, | |
| | | | | | | 573110, 184729, 293869, | |
| | | | | | | 331150, 360703, 365455, | |
| | | | | | | 845273, 586271, 367456, | |
| | | | | | | 514272, 567715, 304206, | |
| | | | | | | 299858, 578511, 392887, | |
| | | | | | | 570049, 326865, 181990, | |
| | | | | | | 189817, 364824, | |
| | | | | | | 3172379, 328628, | |
| | | | | | | 363400, 153965, 196047, | |
| | | | | | | 300418, 259772, 195999, | |
| | | | | | | 367909, 192291, 528266, | |
| | | | | | | 3409363, 759751, | |
| | | | | | | 292633, 537661, 344488, | |
| | | | | | | 580087, 351163, 295023, | |
| | | | | | | 362108, 589313, 696563, | |
| | | | | | | 436032, 183288, 367535, | |
| | | | | | | 4429536, 185937, | |
| | | | | | | 196990, 364034, 136518, | |
| | | | | | | 192326, 177930, 363507, | |
| | | | | | | 192364, 342397, 363232, | |
| | | | | | | 191601, 563803, 362037, | |
| | | | | | | 357020, 194384, 352529, | |
| | | | | | | 357168, 594227, 199243, | |
| | | | | | | 300297, 2555599, | |
| | | | | | | 302321, 317646, 190653, | |
| | | | | | | 311712, 293330, 297970, | |
| | | | | | | 348362, 579452, 404292, | |
| | | | | | | 339417, 1105328, | |
| | | | | | | 1111191, 360329, | |
| | | | | | | 320430, 525378, 510286, | |
| | | | | | | 454156, 354095, | |
| | | | | | | 1146349, 183045, | |
| | | | | | | 571081, 592933, 530973, | |
| | | | | | | 338626, 293543, 296516, | |
| | | | | | | 3409154, 1760821, | |
| | | | | | | 265503, 298536, 304309, | |
| | | | | | | 369486, 554160, 187338, | |
| | | | | | | 294352, 211354, 342110, | |
| | | | | | | 290852, 302090, 333024, | |
| | | | | | | 297529, 4415649, | |
| | | | | | | 564849, 587269, 325848, | |
| | | | | | | 190908, 198251, 183925, | |
| | | | | | | 2035344, 1105552, | |
| | | | | | | 4379141, 752354 | |
| Lactobacillus | 10064 | 1.59E−06 | 0.984613836 | 0.05844216 | 0.07242688 | 536754, 178213, | |
| | | | | | | 4321285, 807795, | |
| | | | | | | 298954, 484444, 84709, | |
| | | | | | | 318764, 333178, 588197, | |
| | | | | | | 593376, 1144153, | |
| | | | | | | 622013, 292057, 851733, | |
| | | | | | | 549756, 1107027, | |
| | | | | | | 354256, 538223, 549991, | |
| | | | | | | 338852, 315189, 255367, | |
| | | | | | | 716286, 553352, 703741, | |
| | | | | | | 134726, 1019465 | |
| Megamonas | 4031 | 2.14E−06 | 0.890949358 | 0.03239651 | 0.04267114 | 2530636, 349065, | |
| | | | | | | 223773, 325808, 422878, | |
| | | | | | | 219064 | |
| Mogibacterium | 2239 | 2.05E−06 | 0.106604882 | 0.00314802 | 0.003875 | 337327, 207340, 316342, | 161 |
| | | | | | | 326850, 197505, 331417 | & |
| | | | | | | | 162 |
| Paraprevotella | 7784 | 2.66E−06 | 0.570824486 | 0.03513149 | 0.04167489 | 323303, 130336, 423264, | |
| | | | | | | 4410807, 4371344, | |
| | | | | | | 1106254, 332968, | |
| | | | | | | 1136390, 4385760, | |
| | | | | | | 4468464 | |
| Prevotella | 18386 | 2.24E−06 | 0.88007735 | 0.05720068 | 0.06828211 | 840914, 326482, 568118, | 150 |
| | | | | | | 292921, 323303, 130336, | & |
| | | | | | | 588929, 545061, 346938, | 151 |
| | | | | | | 329650, 423264, 527941, | |
| | | | | | | 2075910, 4436552, | |
| | | | | | | 4371344, 558839, | |
| | | | | | | 332968, 1136390, | |
| | | | | | | 336372, 519836, | |
| | | | | | | 4385760, 293843, | |
| | | | | | | 321743, 4468464, | |
| | | | | | | 524891, 513003, 509109, | |
| | | | | | | 4370491, 2280817, | |
| | | | | | | 525264, 589329, 925131 | |
| Prevotella | 12327 | 2.38E−06 | 0.834618017 | 0.04030409 | 0.05127829 | 840914, 326482, 568118, | 160 |
| copri | | | | | | 292921, 588929, 545061, | |
| | | | | | | 346938, 329650, 527941, | |
| | | | | | | 2075910, 4436552, | |
| | | | | | | 558839, 336372, 293843, | |
| | | | | | | 321743, 524891, 513003, | |
| | | | | | | 509109, 2280817, | |
| | | | | | | 589329, 925131 | |
| Ruminococcaceae | 28332 | 5.54E−06 | 0.286589102 | 0.01773615 | 0.02003801 | 3390534, 173942, | |
| | | | | | | 523140, 183207, 40798, | |
| | | | | | | 325558, 334215, 228199, | |
| | | | | | | 361722, 581079, 584978, | |
| | | | | | | 1132942, 510295, | |
| | | | | | | 367213, 189899, 851865, | |
| | | | | | | 332430, 180121, 186881, | |
| | | | | | | 304973, 351768, 181035, | |
| | | | | | | 537452, 576712, | |
| | | | | | | 2781880, 297111, | |
| | | | | | | 351095, 535399, 539820, | |
| | | | | | | 230421, 291270, 334044, | |
| | | | | | | 180235, 591635, 523244, | |
| | | | | | | 227565, 574122, | |
| | | | | | | 4314258, 363997, | |
| | | | | | | 189091, 306315, 186956, | |
| | | | | | | 195350, 547114, 165924, | |
| | | | | | | 311961, 350373, 738351, | |
| | | | | | | 579541, 716984, 192566, | |
| | | | | | | 212686, 366143, 208739, | |
| | | | | | | 197250, 554303, 199182, | |
| | | | | | | 181575, 205241, 591734, | |
| | | | | | | 187223, 106786, 298163 | |
| Turicibacter | 4136 | 2.49E−06 | 0.476346943 | 0.00486555 | 0.00660719 | 368490, 214919, 347529 | |

### REFERENCES

[1] Frank et al. (2007) Proc. Natl. Acad. Sci. USA 104, 13780-13785.
[2] Gevers et al. (2014) Cell Host Microbe 15, 382-392.
[3] Ni et al. (2017) Sci. Transl. Med. 9, eaah6888.
[4] Kostic et al. (2013) Cell Host Microbe 14, 207-215.
[5] Johnson and Foster (2018) Nature Reviews Microbiology, October; 16(10):647-655
[6] Kirchoff et al. (2018) PeerJ Preprints 6:e26990v1
[7] Sharon et al. (2013) Genome research, 23(1), pp. 111-120.
[8] Hart et al. (2015) PLoS One. November 24; 10(11):e0143334
[9] WO2018/006080
[10] Gennaro (2000) Remington: The Science and Practice of Pharmacy. 20th edition, ISBN: 0683306472.
[11] Molecular Biology Techniques: An Intensive Laboratory Course, (Ream et al., eds., 1998, Academic Press).
[12] Methods In Enzymology (S. Colowick and N. Kaplan, eds., Academic Press, Inc.)
[13] Handbook of Experimental Immunology, Vols. I-IV (D. M. Weir and C. C. Blackwell, eds, 1986, Blackwell Scientific Publications)
[14] Sambrook et al. (2001) Molecular Cloning: A Laboratory Manual, 3rd edition (Cold Spring Harbor Laboratory Press).
[15] Handbook of Surface and Colloidal Chemistry (Birdi, K. S. ed., CRC Press, 1997)
[16] Ausubel et al. (eds) (2002) Short protocols in molecular biology, 5th edition (Current Protocols).
[17] PCR (Introduction to Biotechniques Series), 2nd ed. (Newton & Graham eds., 1997, Springer Verlag)
[18] Current Protocols in Molecular Biology (F. M. Ausubel et al., eds., 1987) Supplement 30
[19] Smith & Waterman (1981) Adv. Appl. Math. 2: 482-489.
[20] Koenig, J. E., et al., Succession of microbial consortia in the developing infant gut microbiome. Proc Natl Acad Sci USA, 2011. 108 Suppl 1: p. 4578-85.
[21] Palmer, C., et al., Development of the human infant intestinal microbiota. PLoS Biol, 2007. 5(7): p. e177.
[22] Dominguez-Bello, M. G., et al., Delivery mode shapes the acquisition and structure of the initial microbiota across multiple body habitats in newborns. Proc Natl Acad Sci USA, 2010. 107(26): p. 11971-5.
[23] Gritz, E. C. and V. Bhandari, The human neonatal gut microbiome: a brief review. Front Pediatr, 2015. 3: p. 17.
[24] Schulfer, A. and M. J. Blaser, Risks of Antibiotic Exposures Early in Life on the Developing Microbiome. PLoS Pathog, 2015. 11(7): p. e1004903.
[25] Gibson, M. K., T. S. Crofts, and G. Dantas, Antibiotics and the developing infant gut microbiota and resistome. Curr Opin Microbiol, 2015. 27: p. 51-6.
[26] Martinez, I., C. E. Muller, and J. Walter, Long-term temporal analysis of the human fecal microbiota revealed a stable core of dominant bacterial species. PLoS One, 2013. 8(7): p. e69621.
[27] Stinson, L. F., Payne, M. S., & Keelan, J. A. (2017). Planting the seed: Origins, composition, and postnatal health significance of the fetal gastrointestinal microbiota. Crit Rev Microbiol, 43(3), 352-369.
[28] DiGiulio, D. B., Romero, R., Amogan, H. P., Kusanovic, J. P., Bik, E. M., Gotsch, F, Relman, D. A. (2008). Microbial prevalence, diversity and abundance in amniotic fluid during preterm labor: a molecular and culture-based investigation. PLoS One, 3(8), e3056. doi:10.1371/journal.pone.0003056
[29] Ardissone, A. N., de la Cruz, D. M., Davis-Richardson, A. G., Rechcigl, K. T., Li, N., Drew, J. C, Neu, J. (2014). Meconium microbiome analysis identifies bacteria correlated with premature birth. PLoS One, 9(3)
[30] Wampach, L., Heintz-Buschart, A., Hogan, A., Muller, E. E. L., Narayanasamy, S., Laczny, C. C., Wilmes, P. (2017). Colonization and Succession within the Human Gut Microbiome by Archaea, Bacteria, and Microeukaryotes during the First Year of Life. Front Microbiol, 8, 738
[31] Wilczynska, P., Skarzynska, E., & Lisowska-Myjak, B. (2018). Meconium microbiome as a new source of information about long-term health and disease: questions and answers. J Matern Fetal Neonatal Med, 1-6.
[32] Chong, C. Y. L., Bloomfield, F. H., & O'Sullivan, J. M. (2018). Factors Affecting Gastrointestinal Microbiome Development in Neonates. Nutrients, 10(3).
[33] Fernandez, L., Langa, S., Martin, V., Maldonado, A., Jimenez, E., Martin, R., & Rodriguez, J. M. (2013). The human milk microbiota: origin and potential roles in health and disease. Pharmacol Res, 69(1), 1-10.
[34] McGuire, M. K., & McGuire, M. A. (2015). Human milk: mother nature's prototypical probiotic food? Adv Nutr, 6(1), 112-123.
[35] Pannaraj, P. S., Li, F., Cerini, C., Bender, J. M., Yang, S., Rollie, A, Aldrovandi, G. M. (2017). Association Between Breast Milk Bacterial Communities and Establishment and Development of the Infant Gut Microbiome. JAMA Pediatr, 171(7), 647-654.
[36] Mackie, R. I., A. Sghir, and H. R. Gaskins, Developmental microbial ecology of the neonatal gastrointestinal tract. Am J Clin Nutr, 1999. 69(5): p. 10355-10455.
[37] Rodriguez, J. M. (2014). The origin of human milk bacteria: is there a bacterial entero-mammary pathway during late pregnancy and lactation? Adv Nutr, 5(6), 779-784
[38] Houghteling, P. D. and W. A. Walker, Why is initial bacterial colonization of the intestine important to infants' and children's health? J Pediatr Gastroenterol Nutr, 2015. 60(3): p. 294-307.
[39] Sela, D. A., et al., The genome sequence of Bifidobacterium longum subsp. infantis reveals adaptations for milk utilization within the infant microbiome. Proc Natl Acad Sci USA, 2008. 105(48): p. 18964-9.
[40] Underwood, M. A., et al., Bifidobacterium longum subspecies infantis: champion colonizer of the infant gut. Pediatr Res, 2015. 77(1-2): p. 229-35.
[41] Marcobal, A. and J. L. Sonnenburg, Human milk oligosaccharide consumption by intestinal microbiota. Clin Microbiol Infect, 2012. 18 Suppl 4: p. 12-5.
[42] Laflamme, D. and Gunn-Moore, D., 2014. Nutrition of aging cats. Veterinary Clinics: Small Animal Practice, 44(4), pp. 761-774.
[43] Kuzmuk, K. N., Swanson, K. S., Tappenden, K. A., Schook, L. B. and Fahey Jr, G. C., 2005. Diet and age affect intestinal morphology and large bowel fermentative end-product concentrations in senior and young adult dogs. The Journal of nutrition, 135(8), pp. 1940-1945.
[44] Woudstra, T. and Thomson, A. B., 2002. Nutrient absorption and intestinal adaptation with ageing. Best Practice & Research Clinical Gastroenterology, 16(1), pp. 1-15.
[45] Mariat D, Firmesse O, Levenez F, Guimaraes V, Sokol H, Dore J, Corthier G, Furet J P. The Firmicutes/Bacteroidetes ratio of the human microbiota changes with age. BMC Microbiol. 2009 Jun. 9; 9:123.
[46] Zwielehner J, Liszt K, Handschur M, Lassl C, Lapin A, Haslberger A G. Combined PCR-DGGE fingerprinting and quantitative-PCR indicates shifts in fecal population sizes and diversity of Bacteroides, bifidobacteria and Clostridium cluster IV in institutionalized elderly. Exp Gerontol. 2009 June-July; 44(6-7):440-6.
[47] van Tongeren S P, Slaets J P, Harmsen H J, Welling G W. Fecal microbiota composition and frailty. Appl Environ Microbiol. 2005 October; 71(10):6438-42 [48] Claesson et al., Gut microbiota composition correlates with diet and health in the elderly. Nature. 2012 Aug. 9; 488(7410):178-84.
[49] Biagi et al. Through Ageing, and Beyond: Gut Microbiota and Inflammatory Status in Seniors and Centenarians. PLoS One. 2010; 5(5): e10667.
[50] Sordillo, et al. (2017). Factors influencing the infant gut microbiome at age 3-6 months: findings from the ethnically diverse Vitamin D Antenatal Asthma Reduction Trial (VDAART). Journal of Allergy and Clinical Immunology, 139(2), pp. 482-491.
[51] Clarke, et al. (2014) Exercise and associated dietary extremes impact on gut microbial diversity. Gut, pp. gutjnl-2013.
[52] Yatsunenko, T., et al., Human gut microbiome viewed across age and geography. Nature, 2012. 486(7402): p. 222-7
[53] Goodrich, J. K., et al., Human genetics shape the gut microbiome. Cell, 2014. 159(4): p. 789-99.
[54] Jakobsson, et al. 2014. Decreased gut microbiota diversity, delayed Bacteroidetes colonisation and reduced Th1 responses in infants delivered by caesarean section. Gut, 63(4), pp. 559-566.
[55] Abrahamsson, et al. 2014. Low gut microbiota diversity in early infancy precedes asthma at school age. Clinical & Experimental Allergy, 44(6), pp. 842-850.
[56] Mueller, et al. 2015. The infant microbiome development: mom matters. Trends in molecular medicine, 21(2), pp. 109-117.
[57] Deusch, O., et al., Deep Illumina-based shotgun sequencing reveals dietary effects on the structure and function of the fecal microbiome of growing kittens. PLoS One, 2014. 9(7): p. e101021.
[58] Hand et al. Pyrosequencing the canine faecal microbiota: breadth and depth of biodiversity. PLoS One. 2013; 8:
[59] Handl et al. Massive parallel 16 S rRNA gene pyrosequencing reveals highly diverse fecal bacterial and fungal communities in healthy dogs and cats. FEMS Microbiol Ecol. 2011; 76:301-310.
[60] Guard, B. C., et al., Characterization of microbial dysbiosis and metabolomic changes in dogs with acute diarrhea. PLoS One, 2015. 10(5): p. e0127259.
[61] Bresciani, F., et al., Effect of an extruded animal protein-free diet on fecal microbiota of dogs with food-responsive enteropathy. J Vet Intern Med, 2018. 32(6): p. 1903-1910.
[62] Minamoto, Y., et al., Alteration of the fecal microbiota and serum metabolite profiles in dogs with idiopathic inflammatory bowel disease. Gut Microbes, 2015. 6(1): p. 33-47.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A method of determining the health of a canid's microbiome, comprising detecting at least four bacterial taxa in a sample obtained from the canid; wherein the presence of the at least four bacterial taxa is indicative of a healthy microbiome.

2. The method of claim 1, wherein the bacterial taxa are bacterial species from genera selected from the group consisting of Blautia, Lactobacillus, Faecalibacterium, Terrisporobacter, Lachnospiraceae novel sp., Butyricicoccus, Lachnoclostridium, Clostridium, Holdemanella, Cellulosilyticum, Romboutsia, Lachnospiraceae_NK4A136_group, Peptostreptococcus, Sellimonas, Ruminococcaceae_UCG-014, Finegoldia, and Candidatus Dorea.

3. (canceled)

4. The method of claim 2, wherein the bacterial taxa have a 16 S rDNA with at least about 95% identity to the sequence of any one of SEQ ID Nos: 6, 7, 11, 12, 14, 16, 21, 23, 24, 28, 29, 30, 32, 37, 39, 41-43, 46-49, 52, 55-57, 61, 67, 71, 75, 77, 78 and 80.

5. A method of determining the health of a canid's microbiome, comprising quantitating four or more bacterial species in a sample obtained from the canid to determine their abundance; and comparing the abundance to the abundance of the same species in a control data set; wherein an increase or decrease in the abundance of the four or more bacterial species relative to the control data set is indicative of an unhealthy microbiome.

6. The method of claim 5, wherein the bacterial species are from genera selected from the group consisting ofAbsiella [Eubacterium], Anaerostipes, Anaerotruncus, Bifidobacterium, Blautia, Blautia [Ruminococcus] torques group, Butyricicoccus, Candidatus, Dorea, Cellulosilyticum, Clostridium, Clostridium_sensu_stricto_1, Collinsella, Enterococcus, Erysipelatoclostridium, Faecalibacterium, Finegoldia, Flavonifractor, Fusobacterium, Holdemanella [Eubacterium], Lachnoclostridium, Lachnospiraceae novel sp., Lachnospiraceae_NK4A136_group, Lactobacillus, Megamonas, Peptostreptococcus, Romboutsia, Roseburia, Ruminococcaceae, Ruminococcaceae_UCG-014, Ruminococcus, Sellimonas, Terrisporobacter, Turicibacter, and Lachnospiraceae.

7. (canceled)

8. The method of claim 6, wherein a decrease in abundance relative to the control data set is indicative of an unhealthy microbiome.

9. The method of claim 5, wherein the bacterial species is Fusobacterium mortiferum, and an increase in abundance relative to the control data set is indicative of an unhealthy microbiome.

10. (canceled)

11. The method of claim 5, wherein the bacterial taxa have a 16 S rDNA sequence selected from the group consisting of SEQ ID Nos: 3-85.

12. The method of claim 5, wherein the control data set comprises microbiome data of a canid at the same life stage.

13. (canceled)

14. The method of claim 5, wherein the bacterial taxa are species from the genera selected from the group consisting of Ruminococcus, Clostridiales sp., Paraprevotella, Adlercreutzia, Allobaculum, Allobaculum/Dubosiella, Bacteroides, Bifidobacterium, Blautia, Clostridales, Clostridium, Collinsella, Dorea, Enterococcus, Erysipelotrichaceae, Faecalibacterium, Fusobacterium, Holdemanella [Eubacterium], Lachnoclostridium, Lactobacillus, Megamonas, Megasphaera, Peptostreptococcus, Phascolarctobacterium, Prevotella, Sarcina, Terrisporobacter, and Turicibacter.

15. The method of claim 14, wherein the bacterial taxa have a 16 s rDNA with at least about 95% identity to the sequence of any one of SEQ ID NOs: 86-166.

16. (canceled)

17. The method of claim 1, further comprising a step of changing the microbiome composition of the canid.

18. The method of claim 17, wherein the method comprises a step of changing the diet of the canid and/or administering a pharmaceutical composition or a nutraceutical composition to the canid.

19. A method of determining the health of a canid's microbiome, comprising calculating the diversity index for the species within the canid's microbiome and comparing the diversity index to the diversity index of a control data set.

20. The method of claim 19, wherein the canid is a pre-weaned puppy and the microbiome is considered healthy if the diversity index falls in the range of about 0.123 to about 1.744.

21. The method of claim 19, wherein the canid is a post-weaned puppy and the microbiome is considered healthy if the diversity index falls in the range of about 1.294 to about 2.377.

22. The method of claim 19, wherein the canid is an adult and the microbiome is considered healthy if the diversity index falls in the range of about 1.83 to about 3.72.

23. The method of claim 19, wherein the canid is a senior and the microbiome is considered healthy if the diversity index falls in the range of about 1.24 to about 3.55.

24. The method of claim 19, wherein the canid is geriatric and the microbiome is considered healthy if the diversity index falls in the range of about 2.16 to about 3.47.

25. (canceled)

26. (canceled)

27. The method of claim 1, wherein the sample is from the gastrointestinal tract.

28. (canceled)

29. (canceled)

30. (canceled)

31. (canceled)

32. (canceled)

33. (canceled)

34. (canceled)

35. (canceled)


SEQUENCE LISTING (166 sequences)

SEQ ID NO: 1 - DNA, 58 bp, Artificial Sequence
  Description of Artificial Sequence Synthetic primer
caagcagaagacggcatacgagatgtgactggagttcagacgtgtgctcttccgatct

SEQ ID NO: 2 - DNA, 58 bp, Artificial Sequence
  Description of Artificial Sequence Synthetic primer
aatgatacggcgaccaccgagatctacactctttccctacacgacgctcttccgatct

SEQ ID NO: 3 - DNA, 429 bp, Megamonas sp.
cccgttcgctaccctagctttcgagcctcagcgtcagttacagtccagaaagccgccttcgccactggtgttcttcccaatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 4 - DNA, 429 bp, Megamonas sp.
cctgtttgctccccacgctttcgagcctcagcgtcagttacagtccagagagtcgccttcgccactggtgttcttcctaatctctacgcatttcaccgctacactaggaattccactctcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 5 - DNA, 407 bp, Butyricicoccus sp.
cctgtttgctccccacgctttcgcgcctcagcgtcagttaatgtccagcaggccgccttcgccactggtgttcctcccaatatctacgcatttcaccgctacactgggaattccgcctgcctctccatcactcaagacccgcagttttgaaagcagtttgggggttaagcccccagatttcacttccaacttacaggcccgcctacacgccctttacacccagtaaatccggataacgcttgctccctacgtattaccgcggctgctggcacgtagttagccggagcttattctttaggtaccgtcatttgtttcgtccctaattaaagatttttacaatccgaagaccttcttcaatcacgcggcgttgctgcgtcagggttgcccccattgccgaagattcccta

SEQ ID NO: 6 - DNA, 404 bp, Ruminococcus torques
cctgtttgctccccacgctttcgagcctcaacgtcagttactgtccagtaagccgccttcgccactggtgttcctcctaatatctacgcatttcaccgctacactaggaattccacttacctctccagcactctagatgaacagtttccaatgcagtcccggggttgagccccgggttttcacatcagacttgcccatccgtctacgctccctttacacccagtaaatccggataacgcttgcaccatacgtattaccgcggctgctggcacgtatttagccggtgcttcttagtcaggtaccgtcattatcttccctgctgatagagctttacataccgaaatacttcatcgctcacgcggcgtcgctgcatcagggtttcccccattgtgcaatattcccca

SEQ ID NO: 7 - DNA, 405 bp, Ruminococcus gnavus
cctgtttgctccccacgctttcgagcctcaacgtcagttactgtccagtaagccgccttcgccactggtgttcctcctaatatctacgcatttcaccgctacactaggaattccacttacctctccagcactctagcaacacagtttccaaagcagtcccagggttgagccctgggttttcacttcagacttgcatcgccgtctacgctccctttacacccagtaaatccggataacgcttgccccctacgtattaccgcggctgctggcacgtagttagccggggcttcttagtcaggtaccgtcttgtttcttccctgctgatagagctttacataccgaaatacttcttcgctcacgcggcgtcgctgcatcagggtttcccccattgtgcaatattcccca

SEQ ID NO: 8 - DNA, 429 bp, Enterococcus sp.
cctgtttgctccccacgctttcgagcctcagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaagggatgaacagttactctcatccttgttcttctctaacaacagagttttacgatccgaaaaccttcttcactcacgcggcgttgctcggtcagactttcgtccattgccgaagattcccta

SEQ ID NO: 9 - DNA, 429 bp, Lactobacillus sp.
cctgtttgctacccacgctttcgaacctcagcgtcagttacagaccagagagccgctttcgccactggtgttcttccatatatctacgcatttcaccgctacacatggagttccactctcctcttctgcactcaagtctcccagtttccaatgcactactccggttaagccgaaggctttcacatcagacttaaaagaccgcctgcgttccctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattcccta

SEQ ID NO: 10 - DNA, 407 bp, Fusobacterium mortiferum
cctgtttgctacccacgctttcgcgctttagcgtcagtatctgtccagtgggctggcttccccatcggccttcctacaaatatctacgaatttcacctctacacttgtagttccgcccacctctccagtactctagttaagcagtttccaacgcaatacggagttgagccccgcattttcacatcagacttacaaaaccgcctagacgcgctttacgcccaataaatccggataacgcttgcgacatacgtattaccgcggctgctggcacgtatttagccgtcgcttcttctgttagtaccgtcacttacttcgtcccaactgaaagcactttacattccgaaaaacttcatcgtgcacacagaattgctggatcagacttttggtccattgtccaatattcccca

SEQ ID NO: 11 - DNA, 404 bp, Ruminococcus sp.
cctgtttgctccccacgctttcgagcctcaacgtcagttaccgtccagtaagccgccttcgccactggtgttcctcccaatatctacgcatttcaccgctacactgggaattccgcttacctctccggcactctagatacacagtttccaaagcagtcccggggttgagccccgggctttcacttcagacttgcacatccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcgctcacgcggcgtcgctgcatcagggtttcccccattgtgcaatattcccca

SEQ ID NO: 12 - DNA, 404 bp, Blautia producta
cctgtttgctccccacgctttcgagcctcaacgtcagttaccgtccagtaagccgccttcgccactggtgttcctcctaatatctacgcatttcaccgctacactaggaattccgcttacctctccggcactcgagtatcacagtttccaatgcagtccaggggttgagcccccgcctttcacatcagacttgcaacaccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 13 - DNA, 404 bp, Unknown
  Description of Unknown Lachnospiraceae family NK4A136 bacterium sequence
cctgtttgctccccacgctttcgagcctcaacgtcagttaccgtccagtaagccgccttcgccactggtgttcttcctaatatctacgcatttcaccgctacactaggaattccgcttacctctccggcactctagctaaacagtttccaaagcagtcccggcgttgagcaccgggctttcacttcagacttgccttgccgtctacactccctttacacccagtaaatccggataacgcttgccccctacgtattaccgcggctgctggcacgtagttagccggggcttcttattcaggtaccgtcactttcttccctgctgatagagctttacataccgaaatacttcttcactcacgcggcgtcgctgcatcagggtttcccccattgtgcaatattcccca

SEQ ID NO: 14 - DNA, 429 bp, Lactobacillus sp.
cctgtttgctacccacgctttcgaacctcagcgtcagttacagaccagagagccgctttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattcccta

SEQ ID NO: 15 - DNA, 429 bp, Lactobacillus reuteri
cctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattcccta

SEQ ID NO: 16 - DNA, 404 bp, Blautia sp.
cctgtttgctccccacgctttcgagcctcaacgtcagttaccgtccagtaagccgccttcgccactggtgttcctcctaatatctacgcatttcaccgctacactaggaattccgcttacccctccggcactcaagcctggcagtttccaatgcagtccaggagttgagcccctgcctttcacatcagacttgccatgccgtctacgctccctttacacccagtaaatccggataacgcttgccccctacgtattaccgcggctgctggcacgtagttagccggggcttcttagtcaggtaccgtcattatcttccctgctgatagaagtttacataccgagatacttcttccttcacgcggcgtcgctgcatcagggtttcccccattgtgcaatattcccca

SEQ ID NO: 17 - DNA, 403 bp, Romboutsia sp.
cctgtttgctccccacgctttcgtgcctcagcgtcagttacagtccagagagccgccttcgcaactggtgttcctcctaatatctacgcatttcaccgctacactaggaattccactctcctctcctgcactcaagtcttacagtttcaaaagcttactacggttgagccgtagcctttcacttctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaaaccttcatcactcacgcggcgttgctgcatcagggtttcccccattgtgcaatattcccca

SEQ ID NO: 18 - DNA, 407 bp, Unknown
  Description of Unknown Ruminococcaceae family novel bacterium sequence
cctgtttgctccccacgctttcgcgcctcagcgtcagttaatgtccagcaggccgccttcgccactggtgttcctcccaatatctacgcatttcaccgctacactgggaattccgcctgcctctccatcactcaagatccgcagttttgaaagcagtttgggggttgagcccccagatttcactcccaacttacagacccgcctacacgccctttacacccagtaaatccggataacgcttgctccctacgtattaccgcggctgctggcacgtagttagccggagcttattctttaggtaccgtcatttttttcgtccctaattaaagatttttacaatccgaagaccttcttcaatcacgcggcgttgctgcgtcagggttgcccccattgcgcaatattcccca

SEQ ID NO: 19 - DNA, 428 bp, Absiella dolichum
cctatttgctccccacgctttcgtgcctcagtgtcagttacagaccaggcgaccgccttcgccactggtgttcctccatatatctacgcattttaccgctacacatggaattccatcgccctcttctgcactctagcataccagtttccatagcttacaatggttgagccattgccttttactacagacttagtacgccacctacgcaccctttacgcccaatgattccggataacgcttgccacctacgtattaccgcggctgctggcacgtagttagccgtggctttctggtaagctaccgtcactcccatagcatttcctctatgagccgttcttcacttacaacagagctttacgatccgaagaccttcttcactcacgcggcattgctcgttcagggtttcccccattgacgaaaattcccta

SEQ ID NO: 20 - DNA, 409 bp, Bifidobacterium sp.
cctgttcgctccccacgctttcgctcctcagcgtcagtaacggcccagagacctgccttcgccattggtgttcttcccgatatctacacattccaccgttacaccgggaattccagtctcccctgccgcactccagcccgcccgtacccggcgcagatccaccgttaagcgatggactttcacaccagacgcgacgaaccgcctacgagccctttacgcccaataaatccggataacgcttgcaccctacgtattaccgcggctgctggcacgtagttagccggtgcttattcaacgggtacactcactctcgcttgctccccgataaaagcggtttacaacccgaaggcctccatcccgcacgcggcgtcgctgcgtcaggctttcgcccattgcgcaatattcccca

SEQ ID NO: 21 - DNA, 404 bp, Faecalibacterium prausnitzii
cctgtttgctacccacactttcgagcctcagcgtcagttggtgcccagtaggccgccttcgccactggtgttcctcccgatatctacgcattccaccgctacaccgggaattccgcctacctctgcactactcaagaaaaacagttttgaaagcagtttatgggttgagcccatagatttcacttccaacttgtcttcccgcctgcgctccctttacacccagtaattccggacaacgcttgtgacctacgttttaccgcggctgctggcacgtagttagccgtcacttccttgttgggtaccgtcattatcttccccaacaacaggagtttacaatccgaagaccttcttcctccacgcggcgtcgctgcatcagggtttcccccattgtgcaatattcccca

SEQ ID NO: 22 - DNA, 429 bp, Megamonas sp.
cctgtttgctccccacgctttcgagcctcaacgtcagttaccgtccagtaagccgccttcgccactggtgttcctcctaatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 23 - DNA, 403 bp, Terrisporobacter sp.
cctgtttgctccccacgctttcgtgcctcagtgtcagttacagtccagaaagccgccttcgctactggtgttcctcctaatatctacgcatttcaccgctacactaggaattccactttcctctcctgcactcaagtttcccagtttcaagagcttactacggttgagccgtagcctttcactcctgacttaagaaaccacctacgcaccctttacgcccagtaaatccggataacgctagccccctacgtattaccgcggctgctggcacgtagttagccggggcttcctcctcaagtaccgtcattatcttccttgaggacagagttttacgacccgaaggccttcatcactcacgcggcgttgctgcatcaggctttcgcccattgtgcaatattcccca

SEQ ID NO: 24 - DNA, 404 bp, Unknown
  Description of Unknown Ruminococcus gauvreauii group/Clostridium sp. bacterium sequence
cctgtttgctccccacgctttcgagcctcaacgtcagttatcgtccagtaagccgccttcgccactggtgttcctcctaatatctacgcatttcaccgctacactaggaattccacttacccctccgacactctagactgacagtttccaatgcagtcccggggttgagccccgggttttcacatcagacttgccagtccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcgctcacgcggcgtcgctgcatcagggtttcccccattgtgcaatattcccca

SEQ ID NO: 25 - DNA, 404 bp, Blautia sp.
cctgtttgctccccacgctttcgagcctcaacgtcagttaccgtccagtaagccgccttcgccactggtgttcctcctaatatctacgcatttcaccgctacactaggaattccgcttacctctccggcactctagaaaaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 26 - DNA, 404 bp, Blautia sp.; identical to SEQ ID NO: 25

SEQ ID NO: 27 - DNA, 403 bp, Romboutsia sp.
cctgtttgctccccacgctttcgtgcctcagcgtccgttacagtccagagagtcgccttcgcacctggtgttcctcctaatatctacgcatttcaccgctacactaggaattccactctcctctcctgcactcaagtcttacagtttcaaaagcttactacggttgagccgtagcctttcacttctgacttgaaagaccacctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgcccacgcggcgttgctgcatcaggctttcgcccattgtgcaatattcccca

SEQ ID NO: 28 - DNA, 405 bp, Unknown
  Description of Unknown Lachnospiraceae family novel bacterium sequence
cctgtttgctccccacgctttcgagcctcaacgtcagttacagtccagtaagccgccttcgccactggtgttcctcctaatatctacgcatttcaccgctacactaggaattccgcttacctctcctgcactccagcagcacagtttccaaagcagtccgcggggttgagccccgggctttcacttcagacttgcaccgccgtctacgctccctttacacccagtaaatccggataacgcttgccccctacgtattaccgcggctgctggcacgtagttagccggggcttcttagtcaagtaccgtcattttcttccttgctgatagacctttacataccgaaatacttcttcagtcacgcggcgtcgctgcatcagggtttcccccattgtgcaatattcccca

SEQ ID NO: 29 - DNA, 407 bp, Butyricicoccus pullicaecorum
cctgtttgctccccacgctttcgcgcctcagcgtcagttaatgtccagcaggccgccttcgccactggtgttcctcccaatatctacgcatttcaccgctacactgggaattccgcctgcctctccatcactcaagactcgcagttttgaaagcagtttcggggttaagccccgagatttcacttccaacttgcaagcccgcctacacgccctttacacccagtaaatccggataacgcttgctccctacgtattaccgcggctgctggcacgtagttagccggagcttattcttcaggtaccgtcattttcttcgtccctgattaaagatttttacaatccgaagaccttcatcaatcacgcggcgttgctgcgtcagggttgcccccattgcgcaatattcccca

SEQ ID NO: 30 - DNA, 404 bp, Lachnoclostridium sp.
cctgtttgctccccacgctttcgagcctcaacgtcagttactgtccagtaagccgccttcgccactggtgttcctcctaatatctacgcatttcaccgctacactaggaattccacttacctctccagcactctagcaaaacagtttccaaagcagtcccggggttaagccccgggctttcacttcagacttgcttcgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgttgctgcatcagggtttcccccattgtgcaatattcccca

SEQ ID NO: 31 - DNA, 407 bp, Pseudoflavonifractor sp.
cctgtttgctccccacgctttcgcgcctcagcgtcagttactgtccagcaatccgccttcgccactggtgttcctccgtatatctacgcatttcaccgctacacacggaattccgattgcctctccagcactcaagaactacagtttcaaatgcaggctggaggttgagcccccagttttcacatctgacttgcaatcccgcctacacgccctttacacccagtaaatccggataacgcttgccacctacgtattaccgcggctgctggcacgtagttagccgtggcttattcgtcaggtaccgtcatttgtttcgtccccgacaaaagaagtttacaacccgaaagccttcttccttcacgcggcgttgctgggtcaggcttgcgcccattgcccaatattcccca

SEQ ID NO: 32 - DNA, 404 bp, Lachnoclostridium sp.
cctgtttgctccccacgctttcgagcctcaacgtcagttatcgtccagtaagccgccttcgccactggtgttcctcctaatatctacgcatttcaccgctacactaggaattccacttacctctccgacactctagcaaaacagtttccaaagcagtcccagggttgagccctgggttttcacttcagacttgcttcgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcaggctttcgcccattgtgcaatattcccca

SEQ ID NO: 33 - DNA, 428 bp, Erysipelatoclostridium sp.
cctatttgctccccacgctttcgggactgagcgtcagttgcaggccagatcgtcgccttcgccactggtgttcctccatatatctacgcatttcaccgctacacatggaattccacgatcctctcctgcactctagctgcctggtttctatggcttactgaagttaagcttcaggctttcaccacagacccttgctgccgcctgctccctctttacgcccaataattccggataacgcttgccacctacgtattaccgcggctgctggcacgtagttagccgtggctttctaataaagtaccgtcactcggctaccatttcctgtagccgccgttcttcctttataacagaagtttacaatccgaaaaccttcttccttcacgcggcgttgctcggtcagggtttcccccattgccgaaaattcccta

SEQ ID NO: 34 - DNA, 404 bp, Blautia producta
cctgtttgctccccacgctttcgagcctcaacgtcagttaccgtccagtaagccgccttcgccactggtgttcctcctaatatctacgcatttcaccgctacactaggaattccgcttacctctccggcactctagatggacagtttccaaagcagtccaggggttgagcccctgcctttcacttcagacttgcccgtccgtctacgctccctttacacccagtaaatccggataacgcttgccccctacgtattaccgcggctgctggcacgtagttagccggggcttcttagtcaggtaccgtcattgtcttccctgctgatagaagtttacataccgagatacttcttccttcacgcggcgtcgctgcatcagggtttcccccattgtgcaatattcccta

SEQ ID NO: 35 - DNA, 404 bp, Blautia producta; identical to SEQ ID NO: 34

SEQ ID NO: 36 - DNA, 404 bp, Clostridium sp.
cctgtttgctccccacgctttcgagcctcagcgtcagttacagtccagagagtcgccttcgccactggtgttcttcctaatctctacgcatttcaccgctacactaggaattccactctcctctcctgcactctagataatcagtttggaatgcagcccccaggttgagcctgagtatttcacatcccacttaattatccgcctacgctccctttacgcccaataattccggataacgctcgccacctacgtattaccgcggctgctggcacgtagttagccgtggcttcctccttgggtaccgtcattatcgtccccaaagacagagctttacaatccgaagaccgtcatcactcacgcggcgttgctgcatcagggtttcccccattgtgcaatattcccca

SEQ ID NO: 37 - DNA, 403 bp, Clostridium hiranonis
cctgtttgctccccacgctttcgtgcctcagcgtcagttacagtccagaaagccgccttcgctactggtgttcctcccaatatctacgcatttcaccgctacactgggaattccgctttcctctcctgcactcaagtcagacagtatcaggagcttactacggttgagccgtagcctttaactcctgacttgaaagaccgcctacgcaccctttacgcccagtaaatccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggcttgcgcccattgtgcaatattcccca

SEQ ID NO: 38 - DNA, 403 bp, Anaerotruncus sp.
cctgtttgctccccacgctttcgagcctcagcgtcagttaaagcccagcaggccgccttcgccactggtgttcctcctaatatctacgcatttcaccgctacactaggaattccgcctgcctctacttcactcaagaacggcagtttagaacgcagccaccggttgagccgatggatttaacattctacttgccatcccgcctacgctccctttacacccagtaattccggacaacgcttgctccctacgtattaccgcggctgctggcacgtagttagccggagcttcctctttgggtaccgtcattttcttccccaaagacagaggtttacaatccgaagaccgtcttccctcacgcggcgtcgctgcatcaggctttcgcccattgtgcaatatccccca

SEQ ID NO: 39 - DNA, 407 bp, Unknown
  Description of Unknown Lachnospiraceae family novel bacterium sequence
cctgttcgctccccacgctttcgagcctcagcgtcagtttcagtccagaaagccgccttcgccaccggtgttcttcctaatatctacgcatttcaccgctacactaggaattccgctttcctctcctgtactctagcttgatagtttaaaatgcaatcctcgggttaagcccaaggctttcacatcttacttaccatgccgcctacgctccctttacacccagtaattccggataacgcttgccccctacgtattaccgcggctgctggcacgtagttagccggggcttcttatttgggtaccgtcattcttttcttccccatcgatagaagtttacaatccgaaaaccgtcttccttcacgcggcgttgctgcatcagggtttcccccattgtgcaatattcccca

SEQ ID NO: 40 - DNA, 403 bp, Romboutsia sp.
cctgtttgctccccacgctttcgagcctcaacgtcagttaccgtccagtaagccgccttcgccactggtgttcctcctaatatctacgcatttcaccgctacactaggaattccactctcctctcctgcactcaagtcttacagtttcaaaagcttactacggttgagccgtagcctttcacttctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 41 - DNA, 429 bp, Eubacterium biforme
cctatttgctccccacgctttcgtgcttcagtgtcagaatccagaccagacggccgccttcgccaccggtgttcttccatatatctacgcattttaccgctacacatggagttccgccgtcctcttctgttctctagctgatcagtttccagagcaagtacgggttgagcccataccttttactccagacttgatctgccacctacgcaccctttacgcccaatcattccggataacgctcgccacctacgtattaccgcggctgctggcacgtagttagccgtgactttctggtaagataccatcactcactcatcattccctatgagtgccgtttttctcttacaacagagctttacgatccgaagaccttcctcactcacgcggcattgctcgttcagggttccccccattgacgaaaattcccta

SEQ ID NO: 42 - DNA, 406 bp, Cellulosilyticum sp.
cctgtttgctccccacgctttcgtgcctcagcgtcagttatggtccagaaagccgccttcgctactggtgttcctttgaatctctacgcatttcaccgctacactcaaagttccactttcctctcccacactctagcctctcagtttcggtagcagctccggggttgagccccgaaatttcacttccgacttaaaaggccgcctacgcaccctttacacccagtaaatccggataacgcttgctccctacgtattaccgcggctgctggcacgtagttagccggagcttcttagtcaggtaccgtcatttcttcttccctgctgatagaagtttacaatccgaagaccttcttccttcacgcggcgttgctgcatcaggctttcgcccattgtgcaatattcccca

SEQ ID NO: 43 - DNA, 403 bp, Romboutsia sp.
cctgtttgctccccacgctttcgtgcctcagtgtcagttacagtccagagagccgccttcgcaactggtgttcctcctaatatctacgcatttcaccgctacactaggaattccactctcctctcctgcactcaagtttatcagtttcaaaagcttactatggttaagccatagcctttcacttctgacttgataaaccacctacgcaccctttacgcccagtaattccggataacgcttgccccctacgtattaccgcggctgctggcacgtagttagccggggcttcctcctcaagtaccgtcattatcttccttgaggacagagttttacgaccctaaggccttcttcactcacgcggcgttgctgcatcaggctttcgcccattgtgcaatattcccca

SEQ ID NO: 44 - DNA, 404 bp, Lachnoclostridium sp.
cctgtttgctccccacgctttcgagcctcaacgtcagttatcgtccagtaagccgccttcgccactgatgttcctcctaatatctacgcatttcaccgctacactaggaattccgcttacctctccgacactctagaagcacagtttccaaagcagtcacggggttgagccccgggctttcacttcagacttgcacttccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 45 - DNA, 404 bp, Lachnoclostridium sp.; identical to SEQ ID NO: 44

SEQ ID NO: 46 - DNA, 429 bp, Lactobacillus sp.
cctgttcgctacccacgctttcgaacctcagcgtcagttacagaccagagagccgctttcgccactggtgttcttccatatatctacgcatttcaccgctacacatggagttccactctcctcttctgcactcaagttccccagtttccaatgcacatcttcggttaagccgaaggctttcacatcagacttaaagaaccgcctgcgttccctttacgcccaataaatccggataacgcttgccacctacgtattaccgcggctgctggcacgtagttagccgtgacttgctggttagataccgtcaacaggtgaacagttactctcacccgtgttcttctctaacaacagagttttacgatccgaagaccttcttcgctcacgcggcgttgctccatcagacttgcgtccattgtggaagattcccta

SEQ ID NO: 47 - DNA, 404 bp, Unknown
  Description of Unknown Lachnospiraceae family novel bacterium sequence
cctgtttgctccccacgctttcgagcctcaacgtcagttactgtccagcaagccgccttcgccactggtgttcctcctaatatctacgcatttcaccgctacactaggaattccgcttgcctctccagcactctagcccaacagtttccaaagcagttcccgggttgagcccggggatttcacttcagacttgctgtgccgtctacgctccctttacacccagtaaatccggataacgcttgccccctacgtattaccgcggctgctggcacgtagttagccggggcttcttagtcaggtaccgtcactttcttccctgctgatagagctttacataccgaaatacttcttcactcacgcggcgttgctgcatcagggtttcccccattgtgcaatattcccca

SEQ ID NO: 48 - DNA, 404 bp, Lachnoclostridium sp.
cctgtttgctccccacgctttcgagcctcaacgtcagtcatcgtccagtaagccgccttcgccactggtgttcctcctaatatctacgcatttcaccgctacactaggaattccacttacctctccgacactctagaaaaacagtttccaatgcagtcccggggttgagccccgggttttcacatcagacttgcctctccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcatcgctcacgcggcgtcgctgcatcagggtttcccccattgtgcaatattcccca

SEQ ID NO: 49 - DNA, 404 bp, Unknown
  Description of Unknown Lachnospiraceae family NK4A136 bacterium sequence
cctgtttgctccccacgctttcgagcctcaacgtcagttaccgtccagtaagccgccttcgccactggtgttcttcctaatatctacgcatttcaccgctacactaggaattccgcttacctctccggcactctagctaaacagtttccaaagcagtcccggcgttgagcaccgggctttcacttcagacttgccttgccgtctacactccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagagtttcctccattgtgcaatattcccca

SEQ ID NO: 50 - DNA, 404 bp, Ruminococcus gnavus
cctgtttgctccccacgctttcgagcctcaacgtcagtcatcgtccagaaagccgccttcgccactggtgttcctcctaatatctacgcatttcaccgctacactaggaattccgctttcctctccgacactctagcctgacagttccaaatgcagtcccggggttgagccccgggctttcacatctggcttgccatgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcatcgctcacgcggcgtcgctgcatcagggtttcccccattgtgcaatattcccca

SEQ ID NO: 51 - DNA, 404 bp, Blautia producta
cctgtttgctccccacgctttcgagcctcaacgtcagttaccgtccagtaagccgccttcgccactggtgttcctcctaatatctacgcatttcaccgctacactaggaattccgcttacctctccggcactcaagatggacagtttccaatgcagtcccggggttaagccccgggctttcacatcagacttgcccgtccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagagtttcctccattgtgcaatattcccca

SEQ ID NO: 52 - DNA, 404 bp, Sellimonas sp.
cctgtttgctccccacgctttcgagcctcaacgtcagtcaccgtccagtaagccgccttcgccactggtgttcctcctaatatctacgcatttcaccgctacactaggaattccgcttacctctccggcactccagatctgcagtttccaaagcagtcccagggttgagccctgggttttcactccagacttgcctatccgtctacgctccctttacacccagtaaatccggataacgcttgcaccatacgtattaccgcggctgctggcacgtatttagccggtgcttcttactcaggtaccgtcatcttcttccctgctgatagaagtttacataccgaaatacttcttccttcacgcggcgtcgctgcatcagggtttcccccattgtgcaatattcccca

SEQ ID NO: 53 - DNA, 403 bp, Romboutsia sp.
cctgtttgctccccacgctttcgtgcctcagcgtcagttacagtccagagagccgccttcgcaactggtgttcctcctaatatctacgcatttcaccgctacactaggaattccactctcctctcctgcactcaagtctcccagtttcaagagcttactacggttgagccgtagcctttcactcctgacttgaaagaccgcctacgcaccctttacgcccagtaaatccggataacgctagccccctacgtattaccgcggctgctggcacgtagttagccggggcttcctcctcaagtaccgtcattatcttccttgaggacagagttttacgacccgaaggccttcatcactcacgcggcgttgctgcatcaggctttcgcccattgtgcaatattcccca

SEQ ID NO: 54 - DNA, 403 bp, Romboutsia sp.; identical to SEQ ID NO: 53

SEQ ID NO: 55 - DNA, 403 bp, Romboutsia sp.
cctgtttgctccccacgctttcgtgcctcagtgtcagttacagtccagagagccgccttcgcaactggtattcctcctaatatctacgcatttcaccgctacactaggaattctactctcctctcctgcactcaagtttctcagtttcaaaggcttactacggttgagccgtagcctttcacctctgacttaagaaaccacctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 56 - DNA, 429 bp, Lactobacillus ruminis
cctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactacttcggttaagccgaaggctttcacatcagacttaaaagaccgcctgcgttccctttacgcccaataaatccggacaacgctcgccacctacgtattaccgcggctgctggcacgtagttagccgtggctttctggtcagataccgtcaatacgtgaacagttactctcacgcacgttcttctctgacaacagaattttacgacccgaaggccttcttcattcacgcggcgttgctccatcagactttcgtccattgtggaagattcccta

SEQ ID NO: 57 - DNA, 405 bp, Unknown
  Description of Unknown Ruminococcaceae family UCG-015 bacterium sequence
cctgtttgctccccacactttcgtgcctcaacgtcagttactgtccagaaagtcgccttcgccactggtgttcctcctaatatctacgcatttcaccgctacactaggaattccactttcctctccagcactcaagaaatatagttttggttgcaattcctcggttgagccgagggatttcacaaccaacttgcattcccgtctacgcaccctttacacccaataaatccggataacgcttgctccctacgtattaccgcggctgctggcacgtagttagccggagcttattctacaggtactgtcttgtttcttccctgtctaaagcagtttacaatccgaaaaccttcttcctgcacgcggcgtcactgcgtcagagtttcctccattgcgcaatattcccga

SEQ ID NO: 58 - DNA, 404 bp, Blautia producta
cctgtttgctccccacgctttcgagcctcaacgtcagttaccgtccagtaagccgccttcgccactggtgttcctcctaatatctacgcatttcaccgctacactaggaattccgcttacctctccggtactcaagatcaacagtttccaatgcagtccaggggttgagcccctgcctttcacatcagacttgctgctccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 59 - DNA, 429 bp, Megamonas sp.
cctgtttgctccccacgctttcgagcctcagcgtcagttacagaccagagagccgctttcgccaccggtgttcctccatatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 60 - DNA, 429 bp, Megamonas sp.; identical to SEQ ID NO: 59

SEQ ID NO: 61 - DNA, 405 bp, Unknown
  Description of Unknown Ruminococcaceae family UCG-015 bacterium sequence
cctgtttgctccccacactttcgtgcctcaacgtcagttgctgtccagaaagtcgccttcgccactggtgttcctcctaatatctacgcatttcaccgctacactaggaattccactttcctctccagcactcaagaaaagcagttttagtcgcagttcctcagttgagccgagggatttcacaactaacttaccttcccgtctacgcaccctttacacccaataaatccggataacgcttgctccctacgtattaccgcggctgctggcacgtagttagccggagcttattctacaagtactgtcttgtttcttccttgtctaaaatggtttacaatccgaaaaccttcttcccatacgcggcgtcactgcgtcagagtttcctccattgcgcaatattcccga

SEQ ID NO: 62 - DNA, 403 bp, Romboutsia sp.
cctgtttgctccccacgctttcgtgcctcagcgtcagttacagtccagagagccgccttcgctactggtgttcctcctaatatctacgcatttcaccgctacactaggaattccactctcctctcctgcactcaagtcctacagttccaaaagcttactacggttgagccgtagcctttcacttctggcttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 63 - DNA, 403 bp, Romboutsia sp.; identical to SEQ ID NO: 62

SEQ ID NO: 64 - DNA, 403 bp, Romboutsia sp.
cctgtttgctccccacgctttcgagcctcagcgtcagttacagaccagagagccgctttcgccaccggtgttcctccatatatctacgcatttcaccgctacactaggaattccactctcctctcctgcactcaagtcttacagtttcaaaagcttactacggttgagccgtagcctttcacttctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 65 - DNA, 403 bp, Romboutsia sp.; identical to SEQ ID NO: 64

SEQ ID NO: 66 - DNA, 405 bp, Unknown
  Description of Unknown Anaerostipes caccae/Clostridium indolis/Anaerostipes rhamnosivorans bacterium sequence
cctgtttgctccccacgctttcgtgcatcagtgtcagtgacagtccagtaagccgccttcgccactggtgttcctcctaatatctacgcatttcaccgctacactaggaattccgcttacctctcctgcactccagcatgacagtttcaaaagcagtcccggggttaagccccgggctttcacttctgacttaccatgccacctacgcaccctttacacccagtaattccggataacgcttgccccctacgtattaccgcggctgctggcacgtagttagccggggcttcttagtcaggtaccgtctgttttcttccctgctgatagagctttacataccgaaatacttcttcactcacgcggcgtcgctgcatcagggtttcccccattgtgcaatattcccca

SEQ ID NO: 67 - DNA, 404 bp, Lachnoclostridium sp.
cctgtttgctccccacgctttcgagcctcaacgtcagttactgtccagtaagccgccttcgccactggtgttcctcctaatatctacgcatttcaccgctacactaggaattccacttacctctccagcactctagcagaacagtttccaaagcagtcccggggttgagccccgggctttcacttcagacttgctccgccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 68 - DNA, 404 bp, Lachnoclostridium sp.
cctgtttgctccccacgctttcgagcctcaacgtcagttactgtccagtaagccgccttcgccactggtgttcctcctaatatctacgcatttcaccgctacactaggaattccgcttacctctccagcactccagcttaacagtttccaaagcagtcccggggttgagccccgggctttcacttcagacttgctaagccgtctacgctccctttacacccagtaaatccggataacgcttgccccctacgtattaccgcggctgctggcacgtagttagccggggcttcttagtcaggtaccgtcattctcttccctgctgatagagctttacataccgaaatacttcttcactcacgcggcgtcgctgcatcagggtttcccccattgtgcaatattcccca

SEQ ID NO: 69 - DNA, 404 bp, Roseburia sp.
cctgtttgctccccacgctttcgagcctcaacgtcagttaccgtccagtaagccgccttcgccactggtgttcctcctaatatctacgcatttcaccgctacactaggaattccgcttacctctccggcactccaggcccgcagtttccaatgcactcccggggttgggccccgggttttcacatcagacttgctggcccgtctacgctccctttacacccagtaaatccggataacgcttgccccctacgtattaccgcggctgctggcacgtagttagccggggcttcttagtcaggtaccgtcattttcttccctgctgatagaagtttacgtaccgaaatacttcatccttcacgcggcgtcgctgcatcagggttccccccattgtgcaatattcccca

SEQ ID NO: 70 - DNA, 404 bp, Roseburia sp.; identical to SEQ ID NO: 69

SEQ ID NO: 71 - DNA, 403 bp, Roseburia sp.
cctgtttgctccccacgctttcgtgcctcagtgtcagttacagtccagagagccgccttcgcaactggtgttcctcctaatatctacgcatttcaccgctacactaggaattccactctcctctcctgcactcaagtttatcagtttcaaaagcttactatggttgagccgtagcctttcacttctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 72 - DNA, 429 bp, Lactobacillus plantarum
cctgtttgctccccacgctttcgcacctgagcgtcagttacagaccagagagccgccttcgccactggtgttcctccatatatctacgcatttcaccgctacacatggaattccactctcctcttctgcactcaagtctcccagtttccaatgaccctccccggttgagccgggggctttcacatcagacttaagaaaccgcctgcgctcgctttacgcccaataaatccggacaacgcttgccacctacgtattaccgcggctgctggcacgtagttagccgtggctttctggttagataccgtcaggggacgttcagttactaacgtccttgttcttctctaacaacagagttttacgatccgaaaaccttcttcactcacgcggcgttgctcggtcagactttcgtccattgccgaagattcccta

SEQ ID NO: 73 - DNA, 404 bp, Collinsella sp.
cctgttcgctcccccagctttcgcgcctcagcgtcggtctcggcccagagggccgccttcgccaccggtgttccacccgatatctgcgcattccaccgctacaccgggtgttccaccctcccctaccggacccgagcccggcggttcagggggcgggacggggttgagccccgccatttgaccccctgcctgccgggccgcctacgcgcgctttacgcccaatgaatccggataacgctcgccccctacgtattaccgcggctgctggcacgtagttagccggggcttcttctgcaggtaccgtcttgtctcatccctgctgaaagcggtttacgacccgagggccttcgtcccgcacgcggcgtcgctgcgtcagggttccccccattgcgcaagattcccca

SEQ ID NO: 74 - DNA, 404 bp, Lachnoclostridium sp.
cctgtttgctccccacgctttcgagcctcaacgtcagttatcgtccagcaagccgccttcgccactggtgttcctcctaatatctacgcatttcaccgctacactaggaattccgcttgcctctccgacactccagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 75 - DNA, 403 bp, Finegoldia sp.
cctgtttgctccccacgctttcgtacctcagcgtcagtttgtgtccagaaagtcgccttcgctactggtattcctcctaatatctacgtatttcaccactacactaggaattccactttcctctccactactcaagtttatcagtttccaatgctttacggggttgagccccgatctttaacattcgacttattaaaccgcctgcgtaccctttacgcccaataattccggacaacgctcgctccatacgtattaccgcggctgctggcacgtatttagccggagctttcttctatggtactgtcattatcttcccataggacagaactttacgatacgaataccttcttcgttcacgcggcgtcgctgcatcagggtttcccccattgtgcaatattcccca

SEQ ID NO: 76 - DNA, 409 bp, Bifidobacterium sp.
cctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataattccggataacgcttgcaccctacgtattaccgcggctgctggcacgtagttagccggtgcttattcaacgggtaaactcactctcgcttgctccccgataaaagaggtttacaacccgaaggcctccatccctcacgcggcgtcgctgcatcaggcttgcgcccattgtgcaatattcccca

SEQ ID NO: 77 - DNA, 406 bp, Roseburia faecis
cctgtttgctccccacgctttcgagcctcagcgtcagtcatcgtccagtaagccgccttcgccactggtgttcctcctaatatctacgcatttcaccgctacactaggaattccacttacccctccgacactctagtccgacagtttccaatgcagtaccggggttgagccccgggctttcacatcagacttgccgtaccgcctgcgctccctttacacccagtaaatccggataacgcttgcaccatacgtattaccgcggctgctggcacgtatttagccggtgcttcttagtcaggtaccgtcatttcttcttccctgctgatagagctttacataccgaaatacttcttcgctcacgcggcgtcgctgcatcagggtttcccccattgtgcaatattcccca

SEQ ID NO: 78 - DNA, 429 bp, Lactobacillus sp.
cctgtttgctccccacgctttcgcgcctcagtgtcagttacagaccaggaagccgccttcgccactggtgttcctccatatctctacgcatttcaccgctacacatggagttccactctcctcttctgcactcaagtctcccagtttccaatgcactactccggttaagccgaaggctttcacatcagacttaaaagaccgcctgcgttccctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattcccta

SEQ ID NO: 79 - DNA, 407 bp, Fusobacterium sp.
cctgtttgctacccacgctttcgcgctttagcgtcagtatctgtccagtaggctggcttccccatcggcattcctacaaatatctacgaatttcacctctacacttgtagttccgcctacctctccagtactctagtttggcagtttccaacgcaatacggagttgagccccgcattttcacatcagacttaccaaaccgcctagacgcgctttacgcccaataaatccggataacgcttgcgacatacgtattaccgcggctgctggcacgtatttagccgtcgcttcttctgttggtaccgtcactttcttcttcccaactgaaagcactttacattccgaaaaacttcatcgtgcacacagaattgctggatcagacttttggtccattgtccaatattcccca

SEQ ID NO: 80 - DNA, 404 bp, Dorea massiliensis
cctgtttgctccccacgctttcgagcctcaacgtcagtcactgtccagtaagccgccttcgccactggtgttcctcctaatatctacgcatttcaccgctacactaggaattccacttacctctccagcactctagctatacagtttccaaagcagtcccggggttgagccccgggctttcacttcagacttgcacagccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcaggctttcgcccattgtgcaatattcccca

SEQ ID NO: 81 - DNA, 404 bp, Unknown
  Description of Unknown Lachnospiraceae family bacterium sequence
cctgtttgctccccacgctttcgagcctcagcgtcagttacagaccagagagccgctttcgccaccggtgttcctccatatatctacgcatttcaccgctacactaggaattccgcttgcctctccgacactccagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 82 - DNA, 404 bp, Clostridium sp.
cctgtttgctccccacgctttcgagcctcagcgtcagttacagtccagaaaggcgccttcgccactggtattcttcctaatctctacgcatttcaccgctacactaggaattctcctttcctctcctgcactctagatatccagtttggaatgcagcactcaagttgagcccgagtatttcacatcccacttaaacatccgcctacgctccctttacgcccagtaaatccggacaacgcttgccacctacgtattaccgcggctgctggcacgtagttagccgtggcttcctcctcaggtaccgtcattatcgtccctgaagacagagttttacaacccgaaggccgtcatcactcacgcggcgttgctgcatcaggctttcgcccattgtgcaatattcccca

SEQ ID NO: 83 - DNA, 429 bp, Turicibacter sp.
cctgtttgctccccacgctttcgcgcctcagtgtcagttacagaccaggaagccgccttcgccactggtgttcctccatatctctacgcatttcaccgctacacatggaattccacttccctcttctgcactcaagtcgaccagtttccaatgaccctccacggttaagccgtgggctttcacatcagacttaatcaaccacctgcgcgctctttacgcccaataattccggataacgctcgccacctacgtattaccgcggctgctggcacgtagttagccgtggctttctcataaggtaccgtcacactctagccatttcctactaaagtcgttcttcccttataacagaattttacaacccgaaggccttcatcattcacgcggcgttgctcggtcaggctttcgcccattgccgaagattcccta

SEQ ID NO: 84 - DNA, 429 bp, Turicibacter sp.; identical to SEQ ID NO: 83

SEQ ID NO: 85 - DNA, 429 bp, Turicibacter sp.
cctgtttgctccccacgctttcgcgcctcagtgtcagttgcagaccaggaagccgccttcgccactggtgttcctccatatctctacgcatttcaccgctacacatggaattccacttccctcttctgcactcaagtcaaccagtttccaatgaccctccacggttaagccgtgggctttcacatcagacttaattaaccacctgcgcgctctttacgcccaataattccggataacgcttgccacctacgtattaccgcggctgctggcacgtagttagccgtggctttctcataaggtaccgtcaattgatagtcatttcctcctatcacctttcttcccttataacagaattttacaacccgaaggccttcttcattcacgcggcgttgctcggtcaggctttcgcccattgccgaagattcccta

SEQ ID NO: 86 - DNA, 429 bp, Phascolarctobacterium sp.
cccgttcgctaccctggctttcgcatctcagcgtcagacacagtccagaaaggcgccttcgccactggtgttcctcccaatatctacgcatttcaccgctacactgggaattcccctttcctctcctgcactcaagtctcccagtatccagagccatacggggttgagccccgcattttcactccagacttaagaaaccgcctacatgctctttacgcccaataattccggacaacgctcgccacctacgtattaccgcggctgctggcacgtagttagccgtggcttattcgtttactaccgtcattacaaataattattcacaatctgcacattcgtcataaacaaaagagttttacggaacgaattccttcatcactcacgcggcattgctccgtcaggctttcgcccattgcggaagattcccca

SEQ ID NO: 87 - DNA, 424 bp, Bacteroides sp.
cctgtttgatacccgcactttcgagcatcagcgtcagttacggtccagcaagctgccttcgcaatcggagttcttcgtgatatctaagcatttcaccgctacaccacgaattccgcctgcctttaccgcactcaagaactccagtatcaactgcaattttacggttgagccgcaaactttcacaactgacttaaaattccgcctacgctccctttaaacccaataaatccggataacgctcggatcctccgtattaccgcggctgctggcacggagttagccgatccttattcatacggtacatacaaaaagccacacgtggctaactttattcccgtataaaagaagtttacaacccatagggcagtcatccttcacgctacttggctggttcagactctcgtccattgaccaatattcctca

SEQ ID NO: 88 - DNA, 404 bp, Blautia sp.; identical to SEQ ID NO: 12

SEQ ID NO: 89 - DNA, 429 bp, Allobaculum sp.
cctatttgctccccacgctttcgtgcctcagcgtcaggtacaggccaggcggccgccttcgccgctggtgttcttccacatctctacgcattttaccgctacacatggagttccaccgccctctcctgtcctcaagccgtgcagtttccaaagcctgaaccggttgagccggtcccttttacttcagacttgcacagccgcctgcgcaccctttacgcccaatcattccggataacgcttgccacctacgtattaccgcggctgctggcacgtagttagccgtggctttctggccgggtaccatcaaacagtcagctttccactctggctgtcctttgtccccggcaacagggctttacaatccgaagaccgtcttcacccacgcggcattgctcgttcaggcttgcgcccattgacgaaaattcccta

SEQ ID NO: 90 - DNA, 404 bp, Sarcina sp.
cctgtttgctccccacgctttcgagcctcagcgtcagttacagtccagagaatcgccttcgccactggtgttcttcctaatctctacgcatttcaccgctacactaggaattccattctcctctcctgcactctagatacccagtttggaatgcagctcccaggttaagcccaggtatttcacatcccacttaagtatccgcctacgctccctttacgcccagtaaatccggataacgcttgccacctacgtattaccgcggctgctggcacgtagttagccgtggcttcctccttaggtaccgtcattatcgtccctaaagacagagctttacaatccgaagaccttcatcactcacgcggcgttgctgcatcagggtttcccccattgtgcaatattcccca

SEQ ID NO: 91 - DNA, 404 bp, Blautia sp.
cctgtttgctccccacgctttcgagcctcaacgtcagttaccgtccagtaagccgccttcgccactggtgttcctcctaatatctacgcatttcaccgctacactaggaattccgcttacccctccggcactcaagcctggcagtttccaatgcagtccaggagttgagcccctgcctttcacatcagacttgccatgccgtctacgctccctttacacccagtaaatccggataacgcttgccccctacgtattaccgcggctgctggcacgtagttagccggggcttcttagtcaggtaccgtcattatcttccctgctgatagaagtttacataccgaaatacttcatcgctcacgcggcgtcgctgcatcagggtttcccccattgtgcaatattcccca

SEQ ID NO: 92 - DNA, 428 bp, Allobaculum sp.
cctatttgctccccacgctttcgtgcctcagcgtcagtcacaggccaggcggccgccttcgccactggtgttcttccatatctctgcgcattttaccgctacacatggagttccaccgccctctcctgtcctcaagtctgccagtttctgaatcatgaatgagttgagctcatccctttgccttcagacttaacagaccgcctgcgcaccctttacgcccaatcattccggataacgctcgccacctacgtattaccgcggctgctggcacgtagttagccgtggctttctggccgggtaccatccatgaaatgccatttcctgcattccctctttttccccggcaacagagctttacaatccgaagaccttcttcactcacgcggcattgctcgttcaggcttgcgcccattgacgaaaattcccta

SEQ ID NO: 93 - DNA, 404 bp, Ruminococcus torques; identical to SEQ ID NO: 6

SEQ ID NO: 94 - DNA, 428 bp, Allobaculum sp.
cctatttgctccccacgctttcgtgcctcagcgtcagtaacagtccaggcggccgccttcgcaactggtgttcttccatatatctacgcattttaccgctacacatggagttccaccgccctctcctgtcctcgagcgcgccagtttccaaagcctgcacaggttgagcctgtaccttttacttcagacttgacgcgccgcctgcgcaccctttacgcccaatcattccggataacgctcgccacctacgtattaccgcggctgctggcacgtagttagccgtgactttctggcggggtaccatcaaaagcagaccatttcctttctgcttcctttttcccccgcaacagagctttacgatccgaagaccttcctcactcacgcggcattgctcgttcaggcttgcgcccattgacgaaaattcccta

SEQ ID NO: 95 - DNA, 430 bp, Enterococcus cecorum
cctgtttgctccccacgctttcgagcctcagcgtcagttacagaccagaaagccgccttcgccactggtgttcctccatatatctacgcatttcaccgctacacatggaattccgctttcctcttctgcactcaagtctcccagtttccaatgaccctccccggttaagccgggggctttcacatcagacttaaaagaccgcctgcgctcgctttacgcccaataaatccggacaacgcttgccacctacgtattaccgcggctgctggcacgtagttagccgtggctttctggttagataccgtcaagggatgaactttccactctcatccttgttcttctctaacaacagagttttacgatccgaaaaccttcttcactcacgcggcgttgctcggtcagacttgcgtccattgccgaagattcccta

SEQ ID NO: 96 - DNA, 429 bp, Enterococcus sp.; identical to SEQ ID NO: 8

SEQ ID NO: 97 - DNA, 407 bp, Dorea sp.
cctgtttgctacccacgctttcgagcctcagcgtcagttaaagcccagcaggccgccttcgccactggtgttcctcctaatatctacgcatttcaccgctacactaggaattccgcctgcctctacttcactcaagaaaaacagttttgaaagcagctcatgggttgagcccatgcatttcacttccaacttgctttcccgcctacgctccctttacacccagtaattccggacaacgctcgctccctacgtattaccgcggctgctggcacgtagttagccggagcttccttcttcggtaccgtcactttcttcgtcccgaatgacagaggtttacaacccgaaggccgtcttccctcacgcggcgtcgctgcatcaggctttcgcccattgtgcaatatccccca

SEQ ID NO: 98 - DNA, 429 bp, Turicibacter sp.; identical to SEQ ID NO: 83

SEQ ID NO: 99 - DNA, 404 bp, Faecalibacterium prausnitzii; identical to SEQ ID NO: 21

SEQ ID NO: 100 - DNA, 428 bp, Allobaculum sp.
cctatttgctccccacgctttcgtgcctcagcgtcagtaacaggccaggcggccgccttcgccactggtgttcttccatatatctacgcattttaccgctacacatggagttccaccgccctctcctgtcctccagcctgccagtttccaaagcctgtaccggttgagccggtacctttcacttcagacttaacaggccgcctacgcaccctttacgcccaatcattccggataacgctcgccacctacgtattaccgcggctgctggcacgtagttagccgtgacttcctcgaaaggtaatatcacttcaccagcatttcctctggtgttcctttttccctctcaacagaactttacgatccgaagaccttcctcgttcacgcggcattgctcgttcaggcttgcgcccattgacgaaaattcccta

SEQ ID NO: 101 - DNA, 422 bp, Bifidobacterium sp.
cctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataaatccggataacgcttgcaccctacgtattaccgcggctgctggcacgtagttagccggtgcttattcgaacaatccactcaacacggccaaagaccgtgccttgcccttgaacaaaagcggtttacaacccgaaggcctccatcccgcacgcggcgtcgctgcatcaggcttgcgcccattgtgcaatattcccca

SEQ ID NO: 102 - DNA, 404 bp, Clostridium sp.
cctgtttgctccccacgctttcgagcctcagcgtcagttacagtccagagaatcgccttcgccactggtgttcttcctaatctctacgcatttcaccgctacactaggaattccattctcctctcctgcactctagacttccagtttgaaatgcagcacccaagttgagcccgggtatttcacatctcacttaaaagtccgcctacgctccctttacgcccagtaaatccggacaacgctcgccacctacgtattaccgcggctgctggcacgtagttagccgtggcttcctcctcaggtaccgtcattatcgtccctgaagacagagctttacaacccgaaggccgtcatcactcacgcggcgttgctgcatcagggtttcccccattgtgcaatattcccca

SEQ ID NO: 103 - DNA, 424 bp, Prevotella sp.
cctgtttgatacccgcactttcgagcctcagcgtcagttacactccagatacctgccttcgcgatcggagttcctcatgatatctgagcatttcaccgctacaccatgaattccagtatctctgcgtgtactcaagactcccagtatcaactgcagtccgacggttgagccgccgtatttcacaactgacttaagagtccgcctgcgctccctttaaacccaataaatccggataacgcctggaccttccgtattaccgcggctgctggcacggaattagccggtcctttttctgatggtacatacaaaacggtacacgtaccgcactttattcccaactaaaagcagtttacaacccagagggccgtcatcctgcacgctacttggctggttcagacttccgtccattgaccaatattcctca

SEQ ID NO: 104 - DNA, 404 bp, Ruminococcus sp.; identical to SEQ ID NO: 24

SEQ ID NO: 105 - DNA, 429 bp, Turicibacter sp.; identical to SEQ ID NO: 85

SEQ ID NO: 106 - DNA, 403 bp, Unknown
  Description of Unknown Clostridium hiranonis-like bacterium sequence
cctgtttgctccccacgctttcgtgcctcagcgtcagttacagtccagaaagccgccttcgctactggtgttcctcccaatatctacgcatttcaccgctacactgggaattccgctttcctctcctgcactcaagtcagacagtatcaggagcttactacggttgagccgtagcctttaactcctgacttgaaagaccgcctacgcaccctttacgcccagtaaatccggataacgctagccccctacgtattaccgcggctgctggcacgtagttagccggggcttcctcctcaagtaccgtcattatcttccttgaggacagagctttacgacccgaaggccttcatcgctcacgcggcattgctcgttcaggcttgcgcccattgacgaaaattcccta

SEQ ID NO: 107 - DNA, 404 bp, Blautia sp.
cctgtttgctccccacgctttcgagcctcaacgtcagttaccgtccagtaagccgccttcgccactggtgttcctcctaatatctacgcatttcaccgctacactaggaattccgcttacctctccggtactcaagatcaacagtttccaatgcagtccgggggttgagcccccgcctttcacatcagacttgctgctccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 108 - DNA, 404 bp, Collinsella sp.
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtaccgtcttgactcgtccctgctgaaagcggtttacgacccgaaggccttcgtcccgcacgcggcgtcgctgcgtcagggttgcccccattgcgcaagattcccca

SEQ ID NO: 109 - DNA, 407 bp, Fusobacterium sp.
cctgtttgctccccatgctttcgcgcttcagcgtcagtatctgtccagtgagctgacttctctatcggcattcctacaaatatctacgaatttcacctctacacttgtagttccgcccacctctccagtactctagtctggcagtttccaacgcaatacggagttgagccccgcatttccacatcagacttaccagaccgcctagacgcgctttacgcccaataaatccggataacgcttgcgacatacgtattaccgcggctgctggcacgtatttagccgtcgcttcttctgttggtaccgtcactttcttcttcccaactgaaagcactttacattccgaaaaacttcatcgtgcacacagaattgctggatcagacttttggtccattgtccaatattcccca

SEQ ID NO: 110 - DNA, 429 bp, Allobaculum sp.
cctatttgctccccacgctttcgtgcctcagcgtcagcaacaggccaggcggccgccttcgccactggtgttcttccatatatctacgcattttaccgctacacatggagttccaccgccctctcctgctctccagtcctccagtttccaaagccatgcatgagttgagctcatgcgtttcactccagacttgcaggaccgcctgcgcaccctttacgcccaatcattccggataacgctcgccacctacgtattaccgcggctgctggcacgtagttagccgtgactttctggcggggcaccatcagtcagcacccatttcctgatgctgccttttttcccccgcaacagagctttacgacccgaaggccttcttcactcacgcggcatcgctcgttcaggcttgcgcccattgacgaaaattcccta

SEQ ID NO: 111 - DNA, 407 bp, Fusobacterium sp.
cctgtttgctccccatgctttcgcgcttcagcgtcagtatctgtccagtgagctgacttctctatcggcattcctacaaatatctacgaatttcacctctacacttgtagttccgcccacctctccagtactctagtctggcagtttccaacgcaatacggagttgagccccgcatttccacatcagacttaccagaccgcctagacgcgctttacgcccaataaatccggataacgcttgcgacatacgtattaccgcggctgctggcacgtatttagccgtcgcttcttctatcggtaccatcactttctttttcccgattgaaagcactttacaatcctaagaccgtcatcgtgcacacagaattgctggatcagacttttggtccattgtccaatattcccca

SEQ ID NO: 112 - DNA, 403 bp, Adlercreutzia equolifaciens
cctgttcgctcccccagctttcgcgcctcagcgtcagtggcggcccagcaggctgccttcgccatcggtgttcttcccgatatctgcgcattccaccgctacaccgggaattccgcctgcctctaccgcactcgagccgcccagtccggaacccggcccgaggttgagccccgggattagaggttccgcttaggcggccgcctacgcgcgctttacgcccaatgaatccggataacgctcgccccctacgtattaccgcggctgctggcacgtagttagccggagcttcttctgcaggtaccgtctatgtcttccctgctgaaagcggtttacaacccgaaggccttcgtcccgcacgcggcgttgctgcgtcagggtttcccccattgcgcaaaattcccca

SEQ ID NO: 113 - DNA, 404 bp, Unknown; identical to SEQ ID NO: 48
  Description of Unknown Lachnospiraceae family bacterium sequence

SEQ ID NO: 114 - DNA, 429 bp, Unknown
  Description of Unknown Clostridiales order bacterium sequence
cctgtttgctccccacgctttcgcgcctcagcgtcagttgtcgtctagaaagtcgccttcgccaccggtgttcttcctaatctctacgcatttcaccgctacactaggaattccactttcccctccgacactccagccctgcagtttccatcccctcatggggttaagccccacgcttttaagatggacttgcacggccgcctgcgcgcgctttacgcccaataattccggacaacgcttgccacctacgtattaccgcggctgctggcacgtagttagccgtggcttgcttttccggtaccgtcaacatcaatcaatgttctcaatcaatgccttcgtcccggatcacagaactttacaatccgaagaccttcatcgttcacgcggcgttgctccgtcagactttcgtccattgcggaagattcccca

SEQ ID NO: 115 - DNA, 424 bp, Unknown
  Description of Unknown Bacteroidales order bacterium sequence
cctgtttgatacccgcactttcgagcctcagcgtcagttgcaccccggatacctgccttcgcgatcggagttcttcatgatatctgagcatttcaccgctacaccatgaattccagcatccctgtgtgcactcaagactcccagtatcaactgcagtccgacggttgagccgccgtatttcacaactgacttaagagtccgcctgcgctccctttaaacccaataaatccggataacgcctggaccttccgtattaccgcggctgctggcacggaattagccggtcctttttctgatggtacatacaaaacagctcacgagctgcactttattcccaactaaaagcagtttacaacccggagggccgtcatcctgcacgctacttggctggttcagacttgcgtccattgaccaatattcctca

SEQ ID NO: 116 - DNA, 404 bp, Blautia producta; identical to SEQ ID NO: 25

SEQ ID NO: 117 - DNA, 404 bp, Blautia obeum
cctgtttgctccccacgctttcgagcctcaacgtcagttaccgtccagtaagccgccttcgccactggtgttcctcctaatatctacgcatttcaccgctacactaggaattccgcttacccctccggtactcaagactgacagtttccaatgcagtccaggggttgagcccctgcctttcacatcagacttgccattccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 118 - DNA, 405 bp, Unknown
  Description of Unknown Ruminococcaceae family bacterium sequence
cctgtttgctacccacgctttcgagcctcagcgtcagttacagcccagtaggccgccttcgccactggtgttcctcctaatatctacgcatttcaccgctacactaggaattccgcctacctctactgcactcaagagtggcagttttgaacgcgactatcagttgagccgatagtttagacattcaacttgcctccccgcctacgctccctttacacccagtaattccggacaacgcttgccacctacgtattaccgcggctgctggcacgtagttagccgtggcttcctcctcgggtaccgtcatttattcgtccccgaagacagaggtttacaacccgaaggccgtcttccctcacgcggcgtcgctgcatcagggtttcccccattgtgcaatatccccca

SEQ ID NO: 119 - DNA, 403 bp, Clostridium hiranonis; identical to SEQ ID NO: 37

SEQ ID NO: 120 - DNA, 429 bp, Megasphaera sp.
cccgtttgctcccctggctttcgcgcctcagcgtcagttgtcgtccagaaagccgctttcgccactggtgttcctcctaatatctacgcatttcaccgctacactaggaattccgctttcctctccgatactctagcatcgcagtttcggtcccctcacggggttaagccccgcacttttaagaccgacttacgacgccgcctgcgcgccctttacgcccaataattccggacaacgcttgccacctacgtattaccgcggctgctggcacgtagttagccgtggctttctcttacggtaccgtcactcgtaacgggtattgaccgctacgccattcgtcccgtataacagaactttacaacccgaaggccgtcatcgttcacgcggcgttgctccgtcagactttcgtccattgcggaagattcccca

SEQ ID NO: 121 - DNA, 429 bp, Lactobacillus ruminis
cctgtttgctacccacgctttcgaacctcagcgtcagttacagaccagagagccgctttcgccactggtgttcttccatatatctacgcatttcaccgctacacatggagttccactctcctcttctgcactcaagtcttccagtttccaatgcactacttcggttaagccgaaggctttcacatcagacttaaaagaccgcctgcgttccctttacgcccaataaatccggacaacgcttgccacctacgtattaccgcggctgctggcacgtagttagccgtggctttctggtcagataccgtcaatacgtgaacagttactctcacgcacgttcttctctgacaacagaattttacgacccgaaggccttcttcattcacgcggcgttgctccatcagactttcgtccattgtggaagattcccta

SEQ ID NO: 122 - DNA, 424 bp, Bacteroides sp.
cctgtttgatacccgcactttcgagcatcagcgtcagttacggtccagtaagctgccttcgcaatcggagttcttcgtgatatctaagcatttcaccgctacaccacgaattccgcctacctataccgcactcaagaaatccagtatcaactgcaattttacggttgagccgcaaactttcacaactgacttaaacttccgcctacgctccctttaaacccaataaatccggataacgctcggatcctccgtattaccgcggctgctggcacggagttagccgatccttattcatacggtacatacaaaaaagcacacgtgcttcactttattcccgtataaaagaagtttacaacccatagggcagtcatccttcacgctacttggctggttcagactctcgtccattgaccaatattcctca

SEQ ID NO: 123 - DNA, 405 bp, Unknown
  Description of Unknown Eggerthellaceae family bacterium seqeunce
cctgttcgctcccccggctttcgcgcctcagcgtcagtgtcggcccagcaggctgccttcgccatcggtgttcctcccgatatctgcgcattccaccgctacaccgggaattccacctgcccctaccgcactcgagccgcccagttcggaacccggccgcggggttgagccccgggattagaggttccgcttaggcggccgcctacgcgcgctttacgcccaatgaatccggataacgctcgccccctacgtattaccgcggctgctggcacgtagttagccggggcttcttctgcaggtaccgtctcggttcttccctgctgaaagcggtttacaacccgaaggccttcgtcccgcacgcggcgttgctgcgtcagggttgcccccattgcgcaaaattcccca

SEQ ID NO: 124 - DNA, 428 bp, Unknown
  Description of Unknown Erysipelotrichaceae family bacterium seqeuence
cctatttgctccccacgctttcgggactgagcgtcagttatgcgccagatcgtcgccttcgccactggtgttcctccatatatctacgcatttcaccgctacacatggaattccacgatcctctcacacactctagctctacggtttccatggcttaccgaagttaagcttcgatctttcaccacagacccttagtgccgcctgctccctctttacgcccaataattccggataacgctcgccacctacgtattaccgcggctgctggcacgtagttagccgtggctttcttatagagtaccgtcacttggatatcattccctatatccaccgttcttcctctatgacagaagtttacataacgaattacttcttccttcacgcggcgttgctcggtcagggtttcccccattgccgaaaattcccta

SEQ ID NO: 125 - DNA, 404 bp, Ruminococcus gnavus; identical to SEQ ID NO: 50

SEQ ID NO: 126 - DNA, 429 bp, Eubacterium biforme; identical to SEQ ID NO: 41

SEQ ID NO: 127 - DNA, 428 bp, Allobaculum sp.
cctatttgctccccacgctttcgtgcctgagcgtcagtaacagtccaggcggccgccttcgccactggtgttcttccatatatctacgcattttaccgctacacatggagttccaccgccctctcctgtcctcgagcgtgacagtttccaaagcctgtacaggttgagcccgtacctttcacttcagacttgccacgccgcctgcgcaccctttacgcccaatcattccggataacgcttgccacctacgtattaccgcggctgctggcacgtagttagccgtgactttctggcaggttactatcaaaagaaaagcatttcctcttcccttcttttctgacctgcaacagagctttacgatccgaagaccttcctcactcacgcggcattgctcgttcaggcttgcgcccattgacgaaaattcccta

SEQ ID NO: 128 - DNA, 404 bp, Dorea sp.
cctgtttgctccccacgctttcgagcctcaacgtcagtcatcgtccagcaagccgccttcgccactggtgttcctcctaatatctacgcatttcaccgctacactaggaattccgcttgcctctccgacactctagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 129 - DNA, 429 bp, Lactobacillus reuteri; identical to SEQ ID NO: 15

SEQ ID NO: 130 - DNA, 407 bp, Fusobacterium mortiferum; identical to SEQ ID NO: 79

SEQ ID NO: 131 - DNA, 429 bp, Megamonas sp.
cccgttcgctaccctagctttcgagcctcagcgtcagttacagtccagaaagccgccttcgccactggtgttcttcccaatatctacgcatttcaccgctacactgggaattccgctttcctctcctgcactcaagaaaatcagttcggaccccctcacgaggttgagccccgcacttttaagatccgcttaatttcccgcctgcgctccctttacgcccaataattccggacaacgcttgccacctacgtattaccgcggctgctggcacgtagttagccgtggcttcctcgttaagtaccgtcaaatacttactgtattataataagcatccttcgcccttaacaacagaactttacgatccgaagaccttcttcgttcacgcggcgttgctccgtcaggctttcgcccattgcggaagattcccca

SEQ ID NO: 132 - DNA, 429 bp, Phascolarctobacterium sp.
cccgttcgctaccctggctttcgcatctcagcgtcagacacagtccagaaaggcgccttcgccactggtgttcctcccaatatctacgcatttcaccgctacactgggaattcccctttcctctcctgcactcaagtcttccagtatccagagccatacggggttgagccccgcattttcactccagacttaaaaaaccgcctacatgctctttacgcccaataattccggacaacgctcgccacctacgtattaccgcggctgctggcacgtagttagccgtggcttcctcgtctactaccgtcattacacgtcattgttcacaacatgcacattcgtcatagacaacagagctttacgggacgaatcccttcatcactcacgcggcattgctccgtcaggctttcgcccattgcggaagattcccca

SEQ ID NO: 133 - DNA, 404 bp, Blautia obeum
cctgtttgctccccacgctttcgagcctcaacgtcagttaccgtccagtaagccgccttcgccactggtgttcctcctaatatctacgcatttcaccgctacactaggaattccgcttacccctccggcactcaagtatgacagtttccaatgcagtccacaggttgagcccatgcctttcacatcagacttgccacaccgtctacgctccctttacacccagtaaatccggataacgcttgccccctacgtattaccgcggctgctggcacgtagttagccggggcttcttagtcaggtaccgtcactatcttccctgctgatagaagtttacataccgagatacttcttccttcacgcggcgtcgctgcatcagggtttcccccattgtgcaatattcccca

SEQ ID NO: 134 - DNA, 424 bp, Prevotella sp.
cctgttcgatacccgcactttcgagcttcagcgtcagttgcgctccagtgagctgccttcgcaatcggagttcttcgtgatatctaagcatttcaccgctacaccacgaattccgcccactttgtgcgtactcaaggaaaccagttcgcgctgcagtgcagacgttgagcgtctacatttcacaacacgcttaatctccggcctacgctccctttaaacccaataaatccggataacgcccggaccttccgtattaccgcggctgctggcacggaattagccggtccttattcataaggtacatgcaaaaagagtcacgactcccactttattcccttataaaagcagtttacaacccatagggccgtcatcctgcacgctacttggctggttcagactctcgtccattgaccaatattcctca

SEQ ID NO: 135 - DNA, 403 bp, Unknown; identical to SEQ ID NO: 55
  Description of Unknown Romboutsia sp./Clostridium sp. K39/Clostridium dakarense/ Peptostreptococcaceae TM5 bacterium sequence

SEQ ID NO: 136 - DNA, 428 bp, Allobaculum sp.
cctatttgctccccacgctttcgtgcctgagcgtcagcaacagtccaggcggccgccttcgccactggtgttcttccatatatctacgcattttaccgctacacatggagttccaccgccctctcctgtcctcgagcgcgccagtttccaaagcctgtacaggttgagcctgtacctttcacttcagacttgacgcgccgcctgcgcaccctttacgcccaatcattccggataacgcttgccacctacgtattaccgcggctgctggcacgtagttagccgtgactttctggcggggtaccatcaagaagaaatcatttcctcttccttccctttttcccccgcaacagagctttacgatccgaagaccttcctcactcacgcggcattgctcgttcaggcttgcgcccattgacgaaaattcccta

SEQ ID NO: 137 - DNA, 403 bp, Terrisporobacter sp.
cctgtttgctccccacgctttcgtgcctcagtgtcagttacagtccagaaagccgccttcgctactggtgttcctcctaatatctacgcatttcaccgctacactaggaattccactttcctctcctgcactcaagtttcccagtttcaagagcttactacggttaagccgtagcctttcactcctgacttaagaaaccacctacgcaccctttacgcccagtaaatccggataacgctagccccctacgtattaccgcggctgctggcacgtagttagccggggcttcctcctcaagtaccgtcattatcttccttgaggacagagttttacgacccgaaggccttcatcactcacgcggcgttgctgcatcaggctttcgcccattgtgcaatattcccca

SEQ ID NO: 138 - DNA, 422 bp, Bifidobacterium sp.
cctgttcgctccccacgctttcgctcctcagcgtcagtgacggcccagagacctgccttcgccattggtgttcttcccgatatctacacattccaccgttacaccgggaattccagtctcccctaccgcactccagcccgcccgtacccggcgcagatccaccgttaggcgatggactttcacaccggacgcgacgaaccgcctacgagccctttacgcccaataaatccggataacgctcgcaccctacgtattaccgcggctgctggcacgtagttagccggtgcttattcgaacaatccactcaacacggcccgaagccgtgccttgcccttgaacaaaagcggtttacaacccgaaggcctccatcccgcacgcggcgtcgctgcatcaggcttgcgcccattgtgcaatattcccca

SEQ ID NO: 139 - DNA, 404 bp, Dorea sp.
cctgtttgctccccacgctttcgagcctcaacgtcagttaccgtccagtaagccgccttcgccactggtgttcctcctaatatctacgcatttcaccgctacactaggaattccgcttacctctccggcactctagaagcacagtttccaatgcagtcccgtggttgagcctcgggttttcacatcagacttgcacttccgtctacgctccctttacacccagtaaatccggataacgcttgccccctacgtattaccgcggctgctggcacgtagttagccggggcttcttactcaggtaccgtcattttcttccctgctgatagaagtttacataccgaaatacttcatccttcacgcggcgtcgctgcatcagggtttcccccattgtgcaatattcccca

SEQ ID NO: 140 - DNA, 429 bp, Dubosiella newyorkensis
cctatttgctccccacgctttcgtgcctcagcgtcagatgcaggccagacggccgccttcgccaccggtgttcttccatatatctacgcattttaccgctacacatggagttccaccgtcctctcctgctctccagccggacagtttccgcagcccctgaaggttgagcctccagtttttactgcggacttgcccggccgcctgcgcaccctttacgcccaatcattccggataacgctcgccacctacgtattaccgcggctgctggcacgtagttagccgtgacttcctcgccgggtaccatcactcagaaagctttccactctttctgccttttgtccccggcaacagagctttacgatccgaagaccttcctcgctcacgcggcattgctcgttcagggttccccccattgacgaaaattcccta

SEQ ID NO: 141 - DNA, 429 bp, Lactobacillus sp.
cctgttcgctacccatgctttcgagcctcagcgtcagttgcagaccagagagccgccttcgccactggtgttcttccatatatctacgcattccaccgctacacatggagttccactctcctcttctgcactcaagaaaaacagtttccgatgcagttcctcggttaagccgagggctttcacatcagacttattcttccgcctgcgctcgctttacgcccaataaatccggacaacgcttgccacctacgtattaccgcggctgctggcacgtagttagccgtgactttctggttgattaccgtcaaataaaggccagttactacctctatccttcttcaccaacaacagagctttacgatccgaaaaccttcttcactcacgcggcgttgctccatcagactttcgtccattgtggaagattcccta

SEQ ID NO: 142 - DNA, 429 bp, Megamonas sp.; identical to SEQ ID NO: 3

SEQ ID NO: 143 - DNA, 404 bp, Collinsella stercoris; identical to SEQ ID NO: 73

SEQ ID NO: 144 - DNA, 429 bp, Lactobacillus sp.
cctgttcgctacccatgctttcgagcctcagcgtcagttgcagaccagagagccgccttcgccactggtgttcttccatatatctacgcattccaccgctacacatggagttccactctcctcttctgcactcaagttcaacagtttctgatgcaattctccggttgagccgaaggctttcacatcagacttattgaaccgcctgcactcgctttacgcccaataaatccggacaacgcttgccacctacgtattaccgcggctgctggcacgtagttagccgtgactttctaagtaattaccgtcaaataaaggccagttactacctctatctttcttcactaccaacagagctttacgagccgaaacccttcttcactcacgcggcgttgctccatcagactttcgtccattgtggaagattcccta

SEQ ID NO: 145 - DNA, 429 bp, Unknown
  Description of Unknown Escherichia coli/Shigella flexneri bacterium sequence
cctgtttgctccccacgctttcgcacctgagcgtcagtcttcgtccagggggccgccttcgccaccggtattcctccagatctctacgcatttcaccgctacacctggaattctacccccctctacgagactcaagcttgccagtatcagatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgggtaacgtcaatgagcaaaggtattaactttactcccttcctccccgctgaaagtactttacaacccgaaggccttcttcatacacgcggcatggctgcatcaggcttgcgcccattgtgcaatattcccca

SEQ ID NO: 146 - DNA, 428 bp, Allobaculum sp.
cctatttgctccccacgctttcgtgcctgagcgtcagttacaggccaggcggccgccttcgccactggtgttcttccacatctctacgcattttaccgctacacgtggagttccaccgccctctcctgtcctcgagcataccagtttccaaagcctgtacaggttgagcccgtacctttcacttcagacttgatatgccgcctgcgcaccctttacgcccaatcattccggataacgctcgccacctacgtattaccgcggctgctggcacgtagttagccgtgactttctggcgaggtaccatcaaaagagaatcattccctcttctcttcctttttccctcgcaacagagctttacgatccgaagaccttcttcacccacgcggcattgctcgttcaggcttgcgcccattgacgaaaattcccta

SEQ ID NO: 147 - DNA, 404 bp, Blautia producta; identical to SEQ ID NO: 34

SEQ ID NO: 148 - DNA, 429 bp, Lactobacillus sp.; identical to SEQ ID NO: 9

SEQ ID NO: 149 - DNA, 404 bp, Dorea sp.; identical to SEQ ID NO: 11

SEQ ID NO: 150 - DNA, 1491 bp, Prevotella sp.
gagtttgatcctggctcaggatgaacgctagctacaggcttaacacatgcaagtcgaggggcatcatgcaggttgcttgcgatctgtgatggcgaccggcgcacgggtgagtaacgcgtatccaacctaccttcggcaggggcataacccggtgaaagccggcctaattccccatggtccccgttgatgtcatctgattcggggtaaaggtgttttttccggccgttgatggggatgcgtccgattagttagttggcggggtaaaggcccaccaagacagtgatcggtaggggttctgagaggaagatcccccacattgggactgagacacggcccaaactcctacgggaggcagcagtgaggaatattggtcaatgggcgtaagcctgaaccagccaagtagcgtggaggacgaccgccctatgggttgtaaactccttttatgcgggaataaatttcgggacgcgttcccgttttgcatgtaccgcatgaataaggaccggctaattccgtgccagcagccgcggtaatacggaaggttccggtgttatccggatttattgggtttaaagggagcgcggactgcttgtcaagcgtgcagtgaaacgccgcggctcaaccgcggtcctgctgcgcgaactggcttgcttgagtgggctgtaggtacgcggaattcgtggtgtagcggtgaaatgcttagatatcacgaggaactccgattgcgaaggcagcgtaccatatcccgactgacgtttatgctcgaaggtgcgggtatcaaacaggattagataccctggtagtccgcactgtaaacgatggatgctcgctgtcggcgacatattgccggtggcccagcgaaagcgttaagcatcccacctggggagtacgccggcaacggtgaaactcaaaggaattgacgggggcccgcacaagcggaggaacatgtggtttaattcgatgatacgcgaggaaccttacccgggcttgaattgcaggagaacgatacagagatgttgaggcctttcggggctcctgtgaaggtgctgcatggttgtcgtcagctcgtgccgtgaggtgtcggcttaagtgccataacgagcgcaacccctttgcgtagttgccatcgggtgatgccgggcactcttcgcatactgccaccgcaaggtgtgaggaaggtggggatgacgtcaaatcagcacggcccttacgtccggggctacacacgtgttacaatggtgggtacagagtgttgttcgtgcgcaagcacgttccaatcacaaaatccctcctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctgaagtccgtgaccgcgagggtcggcctagggcgaaaccggtgattggggctaagtcgtaacaaggt

SEQ ID NO: 151 - DNA, 1520 bp, Prevotella sp.
gagtttgatcctggctcaggatgaacgctagctacaggcttaacacatgcaagtcgaggggcagcatgaagtcagcttgctgactttgatggcgaccggcgcacgggtgcgtaacgcgtatcaaacctgccgcatactcggggatagccttgcgaaagtaagattaatacccgatgttattatgccctcgcatgagggtataatcaaagattttatcggtatgcgatggtgatgcgtctgattaggtagtaggcggggtaacggcccacctagccaacgatcagtaggggttctgagaggaaggtcccccacactggaactgagacacggtccagactcctacgggaggcagcagtgaggaatattggtcaatggacggaagtctgaaccagccaagtagcgtgcaggatgacggccctccgggttgtaaactgcttttagttgggaataacggcggggacgcgtccccgaaaggaatgtaccatcagaaaaaggaccggctaattccgtgccagcagccgcggtaatacggaaggtccaggcgttatccggatttattgggtttaaagggagcgtaggcgggctattaagtcagcggttaaagcgtgtggctcaaccatacattgccgttgaaactggtggtcttgagtgcacacagggatgctggaactcgtggtgtagcggtgaaatgcttagatatcacgatgaactccgatcgcgaaggcaggtgtccggggtgctactgacgctgaggctcgaaagtgtgggtatcaaacaggattagataccctggtagtccacacagtaaacgatgtatactcgtagtttgcgatagattgtaagctaccaagcgaaagcattaagtataccacctggggagtacgccggcaacggtgaaactcaaaggaattgacgggggcccgcacaagcggaggaacatgtggtttaattcgatgatacgcgaggaaccttacccgggcttgaactagacaggacttaccaagagattggtatttcttcggacctgtttagaggtgctgcatggttgtcgtcagctcgtgccgtgaggtgtcggcttaagtgccataacgagcgcaacccttctcctcggttgccatcaggtgatgctgggcactccgtggacactgccatcgtaagatgtgaggaaggtggggatgacgtcaaatcagcacggcccttacgtccggggctacacacgtgttacaatggggggtacagagggtcgctacctggtgacaggatgctaatctcgaaaacctctctcagttcggattggagtctgcaacccgactccatgaagctggattcgctagtaatcgcgcatcagccatggcgcggtgaatacgttcccgggccttgtacacaccgcccgtcaagccatgaaagccgggggtgcctgaaagccgtgaccgcaagggtcggcctagggtaaaactggtgattggggctaagtcgtaacaaggtagccgtaccggaaggtgcggctggatcacctcctt

SEQ ID NO: 152 - DNA, 1522 bp, Blautia sp.
gagtttgatcctggctcaggatgaacgctggcggcgtgcttaacacatgcaagtcgaacgaagcacttgaatggaattcttcggaaggaagcccaagtgactgagtggcggacgggtgagtaacgcgtgggtaacctgcctcatacagggggataacagttagaaatgactgctaataccgcataagcacacgtgatcgcatgatcgagtgtgaaaaactccggtggtatgagatggacccgcgtctgattagctagttggtggggtaatggcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgaaggatgaagtatttcggtatgtaaacttctatcagcagggaagaaaatgacggtacctgactaagaagccccggctaactacgtgccagcagccgcggtaatacgtagggggcaagcgttatccggatttactgggtgtaaagggagcgtagacggcagtgcaagtctgaagtgaaagcccggggctcaaccccgggactgctttggaaactgtgcagctagagtgtcggagaggcaagcggaattcctagtgtagcggtgaaatgcgtagatattaggaggaacaccagtggcgaaggcggcttgctggacgatgactgacgttgaggctcgaaagcgtggggagcaaacaggattagataccctggtagtccacgccgtaaacgatgactactaggtgtcggggagcaaagctcttcggtgccgcagccaacgcaataagtagtccacctggggagtacgttcgcaagaatgaaactcaaaggaattgacggggacccgcacaagcggtggagcatgtggtttaattcgaagcaacgcgaagaaccttacctgctcttgacatccctctgaccgctctttaatcggagctttccttcgggacagaggagacaggtggtgcatggttgtcgtcagctcgtgtcgtgagatgttgggttaagtcccgcaacgagcgcaacccctatcttcagtagccagcggtaaggccgggcactctggagagactgccagggataacctggaggaaggtggggatgacgtcaaatcatcatgccccttatgagcagggctacacacgtgctacaatggcgtaaacaaagggaagcagagtcgtgaggccgagcaaatcccaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgcaaggagggagctgccgaaggtgggaccgataactggggtgaagtcgtaacaaggtagccgtatcggaaggtgcggctggatcacctcctt

SEQ ID NO: 153 - DNA, 1490 bp, Blautia sp.
gagtttgatcctggctcaggatgaacgctggcggcgtgcttaacacatgcaagtcgaacgaagcactggaaacggaattcttcggaaggaagtatttagtgactgagtggcggacgggtgagtaacgcgtgggtaacctgcctcatacagggggataacagttagaaatagctgctaataccgcataagaccacagagtcgcatgactcagtgggaaaaactccggtggtatgagatggacccgcgtctgattagctagttggtaaggtaacggcttaccaaggcgacgatcagtagccgacctgagagggtgaccggccacattgggactgagacacggcccaaactcctacgggaggcagcagtggggaatattgcacaatgggggaaaccctgatgcagcgacgccgcgtgagtgatgaagtatttcggtatgtaaagctctatcagcagggaagaaaatgacggtacctgactaagaagccccggctaactacgtgccagcagccgcggtaatacgtagggggcaagcgttatccggatttactgggtgtaaagggagtgtagacggtgatgtaagtctgatgtgaaaatttggggctcaaccccaaaactgcattggaaactatgtcactagagtgtcggagaggtaagtggaattcctagtgtagcggtgaaatgcgtagatattaggaggaacaccagtggcgaaggcggcttactggacgatgactgacgttgaggctcgaaagcgtggggagcaaacaggattagataccctggtagtccacgccgtaaacgatgaatactaggtgtcgggtggcaaagccattcggtgccgtcgcaaacgcaataagtattccacctggggagtacgttcgcaagaatgaaactcaaaggaattgacggggacccgcacaagcggtggagcatgtggtttaattcgaagcaacgcgaagaaccttacctggtcttgacatccccttgacagagtatgtaatgtacttttccttcgggacaagggagacaggtggtgcatggttgtcgtcagctcgtgtcgtgagatgttgggttaagtcccgcaacgagcgcaacccctatctttagtagccagcatatgaggtgggcactctagagagactgccagggataacctggaggaaggtggggatgacgtcaaatcatcatgccccttatgatcagggctacacacgtgctacaatggcgtaaacaaagggaagcgaccctgtgaaggcaagcaaatctcaaaaataacgtctcagttcggattgtagtctgcaactcgactacatgaagctggaatcgctagtaatcgcgaatcagaatgtcgcggtgaatacgttcccgggtcttgtacacaccgcccgtcacaccatgggagtcagtaacgcccgaagtcagtgacccaaccgaaaggagggagctgccgaaggtggaaccgataactggggtgaagtcgtaacaaggta

SEQ ID NO: 154 - DNA, 1479 bp, Allobaculum stercoricanis
gatgaacgctggcggcatgcctaatacatgcaagtcgaacgagctacttcggtagctagtggcgaacgggtgagtaacacgtagataacctgcccatacccgggggatacgctttggaaacgaagtctaaaaccccataggaagatttaaggcatcttaaatttttgaaataagctttggcttaggggatggatggatctgcggtgcattagctagttggtgaggtaacagctcaccaaggcgatgatgcatagccggcctgagagggcgatcggccacactgggactgagacacggcccagactcctacgggaggcagcagtagggaattttcgtcaatgggcgcaagcctgaacgagcaatgccgcgtgggtgaagaaggtcttcggatcgtaaagctctgttgcgagggaaaaaggaagagaagagggaatgattctcttttgatggtacctcgccagaaagtcacggctaactacgtgccagcagccgcggtaatacgtaggtggcgagcgttatccggaatgattgggcgtaaagggtgcgcaggcggcatatcaagtctgaagtgaaaggtacgggctcaacctgtacaggctttggaaactggtatgctcgaggacaggagagggcggtggaactccacgtgtagcggtaaaatgcgtagagatgtggaagaacaccagtggcgaaggcggccgcctggcctgtaactgacgctcaggcacgaaagcgtggggagcaaataggattagataccctagtagtccacgccctaaacgatgaggagcaggtgtcgggggtagtacctcggtgccgaagctaacgcaatgactcctccgcctggggagtatgcacgcaagtgtgaaactcaaaggaattgacgggggcccgcacaagcggtggagtatgtggtttaattcgaagcaacgcgaagaaccttaccaggccttgacatcccgagcaaagacatagagatatgttagaggttatctcggtgacaggtggtgcatggttgtcgtcagctcgtgtcgtgagatgttcagttaagtctggcaacgagcgcaaccctcgtgatgtgttactaccattcagttgaggactcacatcagactgccggtgacaaaccggaggaaggcggggatgacgtcaaatcatcatgccccttatggcctgggctacacacgtactacaatggcatctacagacggaagcgaacctgtgaaggcaagccaatccgagaaaagatgtcccagttcggattgaagtctgcaactcgacttcatgaagttggaatcgctagtaatcgcggatcagcatgccgcggtgaatacgttcccgggccttgtacacaccgcccgtcaaaccatggaagccggtaacgcccgaagccgatggcataacccgtaagggagtgagtcgtcgaaggcgggaccgatgactggggttaagtcgtaacaaggtatccctacgggaacg

SEQ ID NO: 155 - DNA, 1494 bp, Clostridium hiranonis
gagtttgatcctggctcaggatgaacgctggcggcgtgcctaacacatgcaagtcgagcgattctcttcggagaagagcggcggacgggtgagtaacgcgtgggtaacctgccctgtacacacggataacataccgaaaggtatgctaatacgggataatatataagagtcgcatgacttttatatcaaagatttttcggtacaggatggacccgcgtctgattagcttgttggcggggtaacggcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgagcgatgaaggccttcgggtcgtaaagctctgtcctcaaggaagataatgacggtacttgaggaggaagccccggctaactacgtgccagcagccgcggtaatacgtagggggctagcgttatccggatttactgggcgtaaagggtgcgtaggcggtctttcaagtcaggagttaaaggctacggctcaaccgtagtaagctcctgatactgtctgacttgagtgcaggagaggaaagcggaattcccagtgtagcggtgaaatgcgtagatattgggaggaacaccagtagcgaaggcggctttctggactgtaactgacgctgaggcacgaaagcgtggggagcaaacaggattagataccctggtagtccacgctgtaaacgatgagtactaggtgtcggaggttacccccttcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaggcttgacatccttctgaccgaggactaatctcctctttccctccggggacagaagtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcctagggctacacacgtgctacaatgggtggtacagagggcagccaaaccgtgaggtggagcaaatcccttaaagccattctcagttcggattgtaggctgaaactcgcctacatgaagctggagttactagtaatcgcagatcagaatgctgcggtgaatgcgttcccgggtcttgtacacaccgcccgtcacaccatgggagttggagacacccgaagccgactatctaaccttttgggagaagtcgtcgaaggtggaatcaataactggggtgaagtcgtaacaaggtagccgtatcggaaggtgcggctggatcacctcctt

SEQ ID NO: 156 - DNA, 1536 bp, Peptostreptococcus sp.
gagtttgatcctggctcaggatgaacgctggcggcgtgcctaacacatgcaagtcgagcgcggttgtgcttagtattgagtgttttattgatataaaacattgaattctatgcacaactgagcggcggacgggtgagtaacgcgtgggtaacctgccctatacacatggataacatactgaaaagtttactaatacatgataaaatagtttttcggcatcgaagaattatcaaagtgtttgcggtataggatggacccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgaacgatgaaggtcttcggatcgtaaagttctgttgcaggggaagataatgacggtaccctgtgaggaagccccggctaactacgtgccagcagccgcggtaatacgtagggggctagcgttatccggatttactgggcgtaaagggtgcgtaggtggtctttcaagtcggtggttaaaggctacggctcaaccgtagtaagcctccgaaacggttagacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgccgtaaacgatgagtactaggtgtcgggggttacccccctcggtgccgcagctaacgcattaagtactccgcctggggagtacgcacgcaagtgtgaaactcaaaggaattgacggggacccgcacaggtagcggagcatgtggtttaattcgaagcaacgcgaagaaccttacctaagcttgacatccctcggaccggtgtttaatcacacctttccttcgggactgaggagacaggtggtgcatggttgtcgtcagctcgtgtcgtgagatgttgggttaagtcccgcaacgagcgcaacccttgtctttagttgccatcattaagttgggcactctagagagactgccagggacaacctggaggaaggtggggatgacgtcaaatcatcatgccccttatgcttagggctacacacgtgctacaatgggtggtacagagggttgccaaaccgtgaggtggagccaatcccttaaagccactctcagttcggattgtaggctgaaactcgcctacatgaagctggagttactagtaatcgcagatcagaatgctgcggtgaatgcgttcccgggtcttgtacacaccgcccgtcacaccatgggagtcggaagcacccgaagccgattatctaaccgcaaggaggagatcgtcgaaggtggcgtcgataactggggtgaagtcgtaacaaggtagccgtatcggaaggtgcggctggatcacctcctt

SEQ ID NO: 157 - DNA, 1509 bp, Peptostreptococcus sp.
gagtttgatcctggctcaggatgaacgctggcggcgtgcctaacacatgcaagtcgagcgcgactgatttgatgcttgcatcgatgaaagttgagcggcggacgggtgagtaacgcgtgggcaacctgccctgtacacatggataacatactgaaaagtttactaatacatgataatatagtttttcggcatcgaagaattatcaaagtgttagcggtacaggatgggcccgcgtctgattagctagttggtgagataactgcccaccaaggcgacgatcagtagccgacctgagagggtgatcggccacattggaactgagacacggtccaaactcctacgggaggcagcagtggggaatattgcacaatgggcgcaagcctgatgcagcaacgccgcgtgaacgatgaaggtcttcggatcgtaaagttctgttgcaggggaagacaatgacggtaccctgtgaggaagccccggctaactacgtgccagcagccgcggtaatacgtagggggctagcgttatccggatttactgggcgtaaagggtgcgtaggtggtccttcaagtcggtggttaaaggctacggctcaaccgtagtaagcctccgaaactgttggacttgagtgcaggagaggaaagtggaattcccagtgtagcggtgaaatgcgtagatattgggaggaacaccagtagcgaaggcggctttctggactgcaactgacactgaggcacgaaagcgtgggtagcaaacaggattagataccctggtagtccacgctgtaaacgatgagtactaggtgtcgggggttacccccctcggtgccgcagctaacgcattaagtactccgcctggggagtacgcacgcaagtgtgaaactcaaaggaattgacggggacccgcacaagtagcggagcatgtggtttaattcgaagcaacgcgaagaaccttacctaagcttgacatccctcggaccggtgtttaatcacacctttccttcgggactgaggtgacaggtggtgcatggttgtcgtcagctcgtgtcgtgagatgttgggttaagtcccgcaacgagcgcaacccttgtctttagttgccatcattaagttgggcactctagagagactgccagggataacctggaggaaggtggggatgacgtcaaatcatcatgccccttatgcttagggctacacacgtgctacaatgggtggtacagagggttgccaaaccgtgaggtggagctaatcccttaaagccattctcagttcggattgtaggctgaaactcgcctacatgaagctggagttactagtaatcgcagatcggaatgctgcggtgaatgcgttcccgggtcttgtacacaccgcccgtcacaccatgggagtcggaaacacccgaagccgattatctaaccgcaaggaggaagtcgtcgaaggtggcgtcgataactggggtgaagtcgtaacaaggtagccgtatcggaaggtgcggctggatcacctcctt

SEQ ID NO: 158 - DNA, 1510 bp, Peptostreptococcaceae bacterium
gagtttgatcctggctcaggaggaacgctggcggcgtgcctaacacatgcaagtcgagcgagaaataaagaaacggagaattcgttcaaagattctttatggaaagcggcggacgggtgagtaacgcgtaggcaacctgcctcatacaaagggatagcctcgggaaaccgggattaaaaccttataaaaccgaaggagcacatgcttcatcggtcaaagatttatcggtatgagatgggcctgcgtctgattagctggttggtgaggtaacggctcaccaaggcgacgatcagtagccgacctgagagggtaaacggccacattggaactgagacccggtccaaactcctacgggaggcagcagtggggaatattgcacaatgggcgaaagcctgatgcagcaacgccgcgtgagtgatgaaggcccttgggtcgtaaaactctgttgagagggaagaaaaaaatgacggtacctcttgaggaagccccggctaactacgtgccagcagccgcggtaatacgtagggggcgagcgttatccggaattattgggcgtaaagagtgcgtaggtggttatctaagcgtggggtgaaaggcagtggcttaaccattgtaagccttgcgaactggatagcttgagtgcaggaggggaaagtggaattcctagtgtagcggtgaaatgcgtagatattaggaggaacaccggtggcgaaggcggctttctggactgtaactgacactgaggcacgaaagcgtgggtagcaaacaggattagataccctggtagtccacgccgtaaacgatgagcactaggtgttggggggagaactctcagtgccgcagtcaacgcaataagtgctccgcctggggagtacgcacgcaagtgtaaaactcaaaggaattgacggggacccgcacaagcagcggagcatgtggtttaattcgaagcaacgcgaagaaccttaccgggacttgacatccgcctgacgtctccttaaccggagatttcttcggacaggcaagacaggtggtgcatggttgtcgtcagctcgtgtcgtgagatgttgggttaagtcccgcaacgagcgcaacccttgtcaatagttgccagcagtaagatgggcactctattgagactgccgtggataacacggaggaaggtggggatgacgtcaaatcatcatgccccttatgttccgggctacacacgtgctacaatggccggtacaacgagaagcaagaccgcaaggtggagcaaatcttaaaaagccggtcccagttcggattgtaggctgcaactcgcctacatgaagatggagttgctagtaatcgcagatcagaatgctgcggtgaatgcgttcccgggtcttgtacacaccgcccgtcacaccatggaagttgggggtgcccgaagtcggttagaaaataggctgccgaaggcaaaaccaatgactggggtgaagtcgtaacaaggtagccgtatcggaaggtgcggctggatcacctcctt

SEQ ID NO: 159 - DNA, 1510 bp, Peptostreptococcaceae bacterium
gagtttgatcctggctcaggatgaacgctggcggcgtgcctaacacatgcaagtcgagcgaaaaatccataatcgaatcttcggacaagagagtggatggaaagcggcggacgggtgagtaacgcgtaggtaacctgccctgtacagagggatagccaccggaaacggtgattaatacctcataacaccgaaagttcacatggacagtcggtcaaagatttatcggtacaggatggacctgcgtctgattagttagttggtgaggtaacggctcaccaaggcgacgatcagtagccgacctgagagggtgatcggccacattggaactgagacacggtccaaactcctacgggaggcagcagtggggaatattgcacaatgggggaaaccctgatgcagcaacgccgcgtgaatgaagaaggcctttgggttgtaaaattctgttctgagggaagaagaaagtgacggtacctcaggagaaagccccggctaactacgtgccagcagccgcggtaatacgtagggggcaagcgttgtccggaatcattgggcgtaaagagtacgtaggcggtttggcaagcgtaaggtttaaggcaacagctcaactgttgttcgccttgtgaactgtcaaacttgagtgcgggagaggaaagcggaattcctggtgtagcggtgaaatgcgtagatatcaggaggaataccggtggcgaaggcggctttctggaccgtaactgacgctgaggtacgaaagcgtggggagcaaacaggattagataccctggtagtccacgccgtaaacgatgagcactaggtgtcggggctttagagcttcggtgccgcagttaacgcaataagtgctccgcctggggagtacgcacgcaagtgtgaaactcaaaggaattgacggggacccgcacaagcagcggagcatgtggtttaattcgaagcaacgcgaagaaccttaccagggcttgacatccttctgacgtatccttaatcggatatttctacggacagaagagacaggtggtgcatggttgtcgtcagctcgtgtcgtgagatgttgggttaagtcccgcaacgagcgcaacccttgtcattagttactaacgatataagtcgaggactttaatgagactgccggggagaactcggaggaaggtggggatgacgtcaaatcatcatgccccttatgttctgggctacacacgtgctacaatggtcggtacaaagagaagcgagactgtgaagtggagcaaaactcaaaagccgatcccagttcggactgtaggctgaaacccgcctacacgaagtcggagttgctagtaatcgtggatcagaatgccacggtgaatgcgttcccgggtcttgtacacaccgcccgtcacaccatggaagttgggggcgcccgaagtcggtcgaaaaatagactgcctaaggtgaaaccaatgactggggtgaagtcgtaacaaggtagccgtatcggaaggtgcggctggatcacctcctt

SEQ ID NO: 160 - DNA, 1493 bp, Prevotella copri
agagtttgatcctggctcaggatgaacgctagctacaggcttaacacatgcaagtcgaggggaaacgacatcgaaagcttgcttttgatgggcgtcgaccggcgcacgggtgagtaacgcgtatccaacctgcccaccacttggggataaccttgcgaaagtaagactaatacccaatgatatctctagaagacatctgaaagagattaaagatttatcggtgatggatggggatgcgtctgattagcttgttggcggggtaacggcccaccaaggcgacgatcagtaggggttctgagaggaaggtcccccacattggaactgagacacggtccaaactcctacgggaggcagcagtgaggaatattggtcaatgggcgagagcctgaaccagccaagtagcgtgcaggatgacggccctatgggttgtaaactgcttttataagggaataaagtgagcctcgtgaggctttttgcatgtaccttatgaataaggaccggctaattccgtgccagcagccgcggtaatacggaaggtccgggcgttatccggatttattgggtttaaagggagcgtaggccggagattaagcgtgttgtgaaatgtagacgctcaacgtctgcactgcagcgcgaactggtttccttgagtacgcacaaagtgggcggaattcgtggtgtagcggtgaaatgcttagatatcacgaagaactccgattgcgaaggcagctcactggagcgcaactgacgctgaagctcgaaagtgcgggtatcgaacaggattagataccctggtagtccgcacggtaaacgatggatgcccgctgttggtctgaacaggtcagcggccaagcgaaagcattaagcatcccacctggggagtacgccggcaacggtgaaactcaaaggaattgacgggggcccgcacaagcggaggaacatgtggtttaattcgatgatacgcgaggaaccttacccgggcttgaattgcagaggaaggatttggagacaatgacgcccttcggggcctctgtgaaggtgctgcatggttgtcgtcagctcgtgccgtgaggtgtcggcttaagtgccataacgagcgcaacccctctccttagttgccatcaggttaagctgggcactctggggacactgccaccgtaaggtgtgaggaaggtggggatgacgtcaaatcagcacggcccttacgtccggggctacacacgtgttacaatggcaggtacagagagacggtystatgyaaatasgatcaaatccttaaagcctgtctcagttcggactggggtctgcaacccgaccccacgaagctggattcgctagtaatcgcgcatcagccatggcgcggtgaatacgttcccgggccttgtacacaccgcccgtcaagccatgaaagccgggggcgcctaaagtccgtgaccgtaaggagcggcctagggcgaaactggtaattggggctaagtcgtaacaaggtaacc

SEQ ID NO: 161 - DNA, 1475 bp, Mogibacterium sp.
gagtttgatcctggctcaggatgaacgctggcggcgtgcctaatacatgcaagtcgagcgagatgttagtgcatgaaccttcgggggattatactaacggacagcggcggacgggtgagtaacgcgtaggcaacctgcccctgacagagggatagccattggaaacgatgattaaaacctcatgacaccgtagaagcacatgcttcatcggtcaaagatttatcggtcggggatgggcctgcgtctgattaactagttggtgaggtaacggctcaccaaggtgacgatcagtagccgacctgagagggtgatcggccacattggaactgagacacggtccaaacttctacggaaggcagcagtagggaatcttgcacaatgggcgaaagcctgatgcagcaacgccgcgtgaaggatgaaggccttcgggttgtaaacttctgttctaagggaagaaagaaatgacggtaccttaggagcaagccccggctaactacgtgccagcagccgcggtaatacgtagggggcaagcgttatccggaattattgggcgtaaagagtgcgtaggtggttacctaagcgcaaggtttaatttagaggctcaacctctacttgccttgcgaactgggctacttgagtgcaggaggggaaagcggaattcctagtgtagcggtgaaatgcgtagatattaggaggaacaccagcggcgaaggcggctttctggactgtaactgacactgaggcacgaaagcgtgggtagcaaacaggattagataccctggtagtccacgccgtaaacgatgagcactaggtgttgggtccgttaggactcagtgccgcagttaacgcaataagtgctccgcctggggagtacgctcgcaagagtaaaactcaaaggaattgacggggacccgcacaagcagcggagcatgtggtttaattcgaagcaacgcgaagaaccttaccagggcttgacatcctgctgacaggactttaacaggttccttcttcggacagcagagacaggtggtgcatggttgtcgtcagctcgtgtcgtgagatgttgggttaagtcccgcaacgagcgcaacccttgtcgctagttactaacattcagttgaggactctagcgagactgccgaggtcaactcggaggaaggtggggatgacgtcaaatcatcatgccccttatgttctgggctacacacgtgctacaatggtcggtacaatgagaggcaatactgcgaagtggagcgaatcaccaaaaccgatcccagttcggattgtaggctgcaactcgcctacatgaagttggagttgctagtaatcgcagatcagaatgctgcggtgaatgcgttcccgggtcttgtacacaccgcccgtcacaccatggaagttgggggtgcccaaagtcggttaattaatctatcgcctaaggcaaaaccaatgactggggtgaagtcgtaacaaggtag

SEQ ID NO: 162 - DNA, 1508 bp, Mogibacterium timidum
gagtttgatcctggctcaggatgaacgctggcggcgtgcctaatacatgcaagtcgagcgagaagcttggaaatgacgcttcggttgattttccaagcggacagcggcggacgggtgagtaacgcgtaggcaacctgcccctgacagagggatagccattggaaacgatgattaaaacctcatgacaccgtagtagcacatgctacatcggtcaaagatttatcggtcagggatgggcctgcgtctgattaactggttggtgaggtaacggctcaccaaggtgacgatcagtagccgacctgagagggtgatcggccacattggaactgagacacggtccaaacttctacggaaggcagcagtagggaatcttgcacaatgggcgaaagcctgatgcagcaacgccgcgtgaaggatgaaggccttcgggttgtaaacttctgttctaagggaagaaagaaatgacggtaccttaggagcaagccccggctaactacgtgccagcagccgcggtaatacgtagggggcaagcgttatccggaattattgggcgtaaagagtgcgtaggtggttacctaagcgcaaggtttaaattagaggctcaacctctacatgccttgcgaactgggctacttgagtgcaggaggggaaagcggaattcctagtgtagcggtgaaatgcgtagatattaggaggaacaccggcggcgaaggcggctttctggactgtaactgacactgaggcacgaaagcgtgggtagcaaacaggattagataccctggtagtccacgccgtaaacgatgagcactaggtgttgggtccgttaggactcagtgccgcagttaacgcaataagtgctccgcctggggagtacgctcgcaagagtaaaactcaaaggaattgacggggacccgcacaagcagcggagcatgtggtttaattcgaagcaacgcgaagaaccttaccagggcttgacatcctgctgacagaaccttaatcggctttttcttcggacagcagagacaggtggtgcatggttgtcgtcagctcgtgtcgtgagatgttgggttaagtcccgcaacgagcgcaacccttgtcgctagttactaacattcagttgaggactctagcgagactgccgaggtcaactcggaggaaggtggggatgacgtcagatcatcatgccccttatgttctgggctacacacgtgctacaatggtcggtacaatgagatgcaatactgcgaagtggagcgaaacaccaaaaccgatcccagttcggattgtaggctgcaactcgcctacatgaagtcggagttgctagtaatcgcagatcagaatgctgcggtgaatgcgttcccgggtcttgtacacaccgcccgtcacaccatggaagttgggggtgcccaaagtcggttaattaatctatcgcctaaggcaaaaccaatgactggggtgaagtcgtaacaaggtagccgttcgagaacgagcggctggatcacctcctt

SEQ ID NO: 163 - DNA, 1476 bp, Fusobacterium canifelinum
gagtttgatcctggctcaggatgaacgctgacagaatgcttaacacatgcaagtctacttgaatttgggttttttaacttcgatttgggtggcggacgggtgagtaacgcgtaaagaacttgcctcacagctagggacaacatttggaaacgaatgctaatacctgatattatgattttagggcatcctagaattatgaaagctatatgcgctgtgagagagctttgcgtcccattagctagttggagaggtaacggctcaccaaggcgatgatgggtagccggcctgagagggtgatcggccacaaggggactgagacacggcccttactcctacgggaggcagcagtggggaatattggacaatggaccaagagtctgatccagcaattccgtgtgcacgatgaagtttttcggaatgtaaagtgctttcagttgggaagaaaaaaatgacggtaccaacagaagaagtgacggctaaatacgtgccagcagccgcggtaatacgtatgtcacaagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctgaagcgcgaaagcgtgggtagcaaacaggattagataccctggtagtccacgccgtaaacgatgattactaggtgttgggggtcgaacctcagcgcccaagcaaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcttaggaatgagacagagatgtttcagtgtcccttcggggaaacctaaagacaggtggtgcatggctgtcgtcagctcgtgtcgtgagatgttgggttaagtcccgcaacgagcgcaacccctttcgtatgttaccatcattaagttggggactcatgcgatactgcctacgatgagtaggaggaaggtggggatgacgtcaagtcatcatgccccttatacgctgggctacacacgtgctacaatgggtagtacagagagtcgcaaagccgtgaggtagagctaatctcagaaaactattcttagttcggattgtactctgcaactcgagtacatgaagttggaatcgctagtaatcgcgaatcagcaatgtcgcggtgaatacgttctcgggtcttgtacacaccgcccgtcacaccacgagagttggttgcacctgaagtagcaggcctaaccgcaaggagggatgctccgagggtgtgattagcgattggggtgaagtcgtaacaaggt

SEQ ID NO: 164 - DNA, 1464 bp, Fusobacterium necrophorum
gagtttgatcctggctcaggatgaacgctgacagaatgcttaacacatgcaagtcgactcgagtcttcggacttgggtggcgcacgggtgagtaacgcgtaaagaacttgcctcttagaccgggacaacatctggaaacggatgctaataccggatattatggttttttcgcatggaggaatcatgaaagctagatgcgctaagagagagctttgcgtcccattagctagttggtgaggtaacggcccaccaaggcaatgatgggtagccggcctgagagggtgaacggccacaaggggactgagacacggcccttactcctacgggaggcagcagtggggaatattggacaatggaccacaagtctgatccagcaattctgtgtgcacgatgacgtttttcggaatgtaaagtgctttcagtcgggaagaagtcagtgacggtaccgacagaagaagcgacggctaaatacgtgccagcagccgcggtaatacgtatgtcgcaagcgttatccggatttattgggcgtaaagcgcgtctaggcggcaaggaaagtctgatgtgaaaatgcggagctcaactccgtatggcgttggaaactgccttactagagtactggagaggtaggcggaactacaagtgtagaggtgaaattcttagatatttgtaggaatgccgatggggaagccagcctactggacagatactgacgctaaagcgcgaaagcgtgggtagcaaacaggattagataccctggtagtccacgctgtaaacgatgattactaggtgttgggggtcaaacctcagcgcccaagctaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcctacgaacggagcagagatgcgccggtgccctttcgggggaacgtagtgacaggtggtgcatggctgtcgtcagctcgtgtcgtgagatgttgggttaagtcccgcaacgagcgcaacccctatcgtatgttaccatccttcagttggggactcatgcggtactgcctgcgacgagcaggaggaaggtggggatgacgtcaagtcatcatgccccttatacgctgggctacacacgtgctacaatgggtagtacagagagcagcaaacccgcgagggggagcaaatctcagaaaactattcttagttcggattgtactctgcgactcgagtacatgaagttggaatcgctagtaatcgcaaatcagcaatgttgcggtgaatacgttctcgggtcttgtacacaccgcccgtcacaccacgagagttggttgcacctgaagtagcaggcctaaccttagggaaggatgctccgagggtgtggttagcgattggggtgaagtcgtaacaaggt

SEQ ID NO: 165 - DNA, 1475 bp, Fusobacterium nucleatum
gagtttgatcctggctcaggatgaacgctgacagaatgcttaacacatgcaagtcaacttgaatttgggtttttaacttagatttgggtggcggacgggtgagtaacgcgtaaagaacttgcctcacagctagggacaacatttagaaatgaatgctaatacctgatattatgattttaaggcatcttagaattatgaaagctataagcactgtgagagagctttgcgtcccattagctagttggagaggtaacagctcaccaaggcgatgatgggtagccggcctgagagggtgaacggccacaaggggactgagacacggcccttactcctacgggaggcagcagtggggaatattggacaatggaccgagagtctgatccagcaattctgtgtgcacgatgaagtttttcggaatgtaaagtgctttcagttgggaagaaataaatgacggtaccaacagaagaagtgacggctaaatacgtgccagcagccgcggtaatacgtatgtcacgagcgttatccggatttattgggcgtaaagcgcgtctaggtggttatgtaagtctgatgtgaaaatgcagggctcaactctgtattgcgttggaaactgtgtaactagagtactggagaggtaagcggaactacaagtgtagaggtgaaattcgtagatatttgtaggaatgccgatggggaagccagcttactggacagatactgacgctaaagcgcgaaagcgtgggtagcaaacaggattagataccctggtagtccacgctgtaaacgatgattactaggtgttgggggtcgaacctcagcgcccaagcaaacgcgataagtaatccgcctggggagtacgtacgcaagtatgaaactcaaaggaattgacggggacccgcacaagcggtggagcatgtggtttaattcgacgcaacgcgaggaaccttaccagcgtttgacatcttaggaatgagatagagatatttcagtgtcccttcggggaaacctaaagacaggtggtgcatggctgtcgtcagctcgtgtcgtgagatgttgggttaagtcccgcaacgagcgcaacccctttcgtatgttaccatcattaagttggggactcatgcgatactgcctacgatgagtaggaggaaggtggggatgacgtcaagtcatcatgccccttatacgctgggctacacacgtgctacaatgggtagaacagagagttgcaaagccgtgaggtgaagctaatctcagaaaactattcttagttcggattgtactctgcaactcgagtacatgaagttggaatcgctagtaatcgcgaatcagcaatgtcgcggtgaatacgttctcgggtcttgtacacaccgcccgtcacaccacgagagttggttgcacctgaagtagcaggcctaaccgtaaggagggatgctccgagggtgtgattagcgattggggtgaagtcgtaacaaggt

SEQ ID NO: 166 - DNA, 1533 bp, Unknown
  Description of Unknown Escherichia/Shigella COT277 bacterium sequence
gagtttgatcatggctcagattgaacgctggcggcaggcctaacacatgcaagtcgaacggtaacaggaagaagcttgcttctttgctgacgagtggcggacgggtgagtaatgtctgggaaactgcctgatggagggggataactactggaaacggtagctaataccgcataacgtcgcaagaccaaagagggggaccttcgggcctcttgccatcggatgtgcccagatgggattagctagtaggtggggtaacggctcacctaggcgacgatccctagctggtctgagaggatgaccagccacactggaactgagacacggtccagactcctacgggaggcagcagtggggaatattgcacaatgggcgcaagccagatgcagccatgccgcgtgtatgaagaaggccttcgggttgtaaagtactttcagcggggaggaagggagtaaagttaatacctttgctcattgacgttacccgcagaagaagcaccggctaactccgtgccagcagccgcggtaatacggagggtgcaagcgttaatcggaattactgggcgtaaagcgcacgcaggcggtttgttaagtcagatgtgaaatccccgggctcaacctgggaactgcatctgatactggcaagcttgagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggtcttgacatccacagaacttcccagagatggattggtgccttcgggaactgtgagacaggtgctgcatggctgtcgtcagctcgtgttgtgaaatgttgggttaagtcccgcaacgagcgcaacccttatcctttgttgccagcggtccggccgggaactcaaaggagactgccagtgataaactggaggaaggtggggatgacgtcaagtcatcatggcccttacgaccagggctacacacgtgctacaatggcgcatacaaagagaagcgacctcgcgagagcaagcggacctcataaagtgcgtcgtagtccggattggagtctgcaactcgactccatgaagtcggaatcgctagtaatcgtggatcagaatgccacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctt